(19) United States
(12) Patent Application Publication
(10) Pub. No.: US 2025/0359474 A1
(43) Pub. Date: Nov. 20, 2025

(54) ORGANIC COMPOUND, ORGANIC ELECTROLUMINESCENT DEVICE, AND ELECTRONIC APPARATUS

(71) Applicant: SHAANXI LIGHTE OPTOELECTRONICS MATERIAL CO., LTD., Xi'an, Shaanxi (CN)

(72) Inventors: Min YANG, Xi'an, Shaanxi (CN); Yun LIU, Xi'an, Shaanxi (CN); Youngkook KIM, Xi'an, Shaanxi (CN)

(21) Appl. No.: 18/871,362

(22) PCT Filed: Sep. 22, 2023
(86) PCT No.: PCT/CN2023/120869

§ 371 Date: Dec. 3, 2024

(30) Foreign Application Priority Data
Feb. 1, 2023 (CN) 202310071960.6
Apr. 3, 2023 (CN) 202310350635.3

(51) Int. Cl. H10K 85/40; C07B 59/00; C07F 7/08; C09K 11/02; H10K 50/11; H10K 85/60

(52) U.S. Cl. CPC H10K 85/40; C07B 59/004; C07F 7/0816; C09K 11/02; H10K 85/615; H10K 85/654; H10K 85/6572; H10K 85/6574; H10K 85/6576; C07B 2200/05; H10K 50/11

(57) ABSTRACT

An organic compound, an organic electroluminescent device comprising same, and an electronic apparatus. The compound uses silafluorenyl and carbazolyl substituted triazine as a core structure, and when the compound is used as a host material of an organic light-emitting layer, carrier balance in the organic light-emitting layer can be improved, the carrier recombination area can be widened, the generation and utilization efficiency of excitons can be improved, the luminous efficiency of a device can be improved, and the service life of the device can be prolonged.

### CROSS-REFERENCE TO RELATED APPLICATIONS

The present disclosure claims the priorities of Chinese patent application No. 202310071960.6 filed on Feb. 1, 2023, and Chinese patent application No. 202310350635.3 Apr. 3, 2023, which are incorporated herein by reference in their entirety as a part of this disclosure.

### FIELD OF THE INVENTION

The present disclosure relates to the technical field of organic electroluminescent materials, in particular to an organic compound, an organic electroluminescent device comprising the same, and an electronic apparatus.

### BACKGROUND OF THE INVENTION

With the development of electronic technology and the progress of material science, the application range of electronic components and devices used to realize electroluminescence or photoelectric conversion is increasingly extensive. An organic electroluminescent device (OLED) usually comprises a cathode and an anode disposed opposite to each other, and a functional layer disposed between the cathode and the anode. The functional layer is composed of multiple organic or inorganic film layers, and generally includes an organic light-emitting layer, a hole transport layer, an electron transport layer, etc. When a voltage is applied to the cathode and the anode, an electric field is generated between the two electrodes. Under the influence of the electric field, electrons on the cathode side move towards the electroluminescent layer, and holes on the anode side also move towards the luminescent layer. Electrons and holes combine in the electroluminescent layer to form excitons, which are in an excited state and release energy outward, thereby causing the electroluminescent layer to emit light externally.

In existing organic electroluminescent devices, the most important problems are service life and efficiency. With the large-area display trend, the driving voltage has been correspondingly increased, and enhancements in luminous efficiency and current efficiency are also requisite.

Therefore, there is a necessity for the continued development of novel materials to further improve the performance of organic electroluminescent devices.

### SUMMARY OF THE INVENTION

Against the above problem in the existing technology, the objective of the present disclosure is to provide an organic compound, as well as an organic electroluminescent device comprising the same and an electronic apparatus. The organic compound, when utilized in the organic electroluminescent device, can improve the performance of the device.

According to a first aspect of the present disclosure, there is provided an organic compound having a structure represented by Formula 1 as follows:

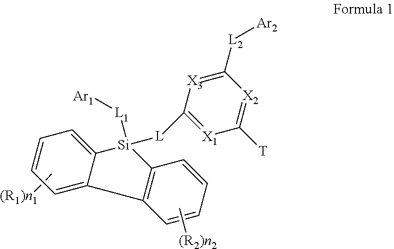

wherein X₁, X₂, and X₃ are each independently selected from N and C(H), and at least two of X₁ to X₃ are N;
Ar₁ and Ar₂ are the same or different, and are each independently selected from a substituted or unsubstituted aryl having 6 to 40 carbon atoms, and a substituted or unsubstituted heteroaryl having 3 to 40 carbon atoms;
L is selected from a single bond, a substituted or unsubstituted arylene having 6 to 30 carbon atoms, and a substituted or unsubstituted heteroarylene having 3 to 30 carbon atoms;
L₁ and L₂ are the same or different, and are each independently a single bond, a substituted or unsubstituted arylene having 6 to 30 carbon atoms, and a substituted or unsubstituted heteroarylene having 3 to 30 carbon atoms;
group T is a substituted or unsubstituted carbazolyl;
substituent(s) in L₁, L₂, Ar₁, and Ar₂ are the same or different, and are each independently selected from a deuterium, a cyano, a halogen group, an alkyl having 1 to 10 carbon atoms, a haloalkyl having 1 to 10 carbon atoms, a deuterated alkyl having 1 to 10 carbon atoms, a trialkylsilyl having 3 to 12 carbon atoms, a triphenylsilyl, an aryl having 6 to 20 carbon atoms, an alkylaryl having 6 to 20 carbon atoms, a deuterated aryl having 6 to 20 carbon atoms, a haloaryl having 6 to 20 carbon atoms, a heteroaryl having 3 to 20 carbon atoms, and a cycloalkyl having 3 to 10 carbon atoms; optionally, any two adjacent substituents form a substituted or unsubstituted ring A; the ring A is a saturated or unsaturated 5 to 13 membered ring;
substituent(s) in the group T, substituent(s) on the ring A, and each of R₁ and R₂ are the same or different, and are each independently selected from a deuterium, a cyano, a halogen group, an alkyl having 1 to 10 carbon atoms, a haloalkyl having 1 to 10 carbon atoms, a deuterated alkyl having 1 to 10 carbon atoms, a trialkylsilyl having 3 to 12 carbon atoms, a triphenylsilyl, an aryl having 6 to 20 carbon atoms, a deuterated aryl having 6 to 20 carbon atoms, a haloaryl having 6 to 20 carbon atoms, a heteroaryl having 3 to 20 carbon atoms, and a cycloalkyl having 3 to 10 carbon atoms; n₁ is selected from 0, 1, 2, 3, and 4;
n₂ is selected from 0, 1, 2, 3, and 4.

According to a second aspect of the present disclosure, there is provided an organic electroluminescent device, comprising an anode and a cathode disposed opposite to each other, and a functional layer disposed between the anode and the cathode; the functional layer comprises the above-described organic compound.

According to a third aspect of the present disclosure, there is provided an electronic apparatus, comprising the organic electroluminescent device described in the second aspect.

The compound provided in the present disclosure uses silafluorenyl and carbazolyl substituted triazine as a core structure, in which the silafluorenyl has a relatively stable spatial three-dimensionality, and the silicon-containing heterocycle and its two substituents at position 9 situate on three distinct planes, resulting in a significant degree of molecular distortion. This enhances the film-forming properties of the structure to a certain extent. The triazinyl moiety containing a carbazolyl cause the entire molecule to have a higher first excited triplet energy level, thereby exhibiting stronger luminescent properties, and significantly enhancing the material's thermal stability and electron affinity. The compound of the present disclosure, when utilized in the light-emitting layer of an organic electroluminescent device, can improve the carrier balance within the light-emitting layer, widening the carrier recombination area, improving the generation and utilization efficiency of excitons, improving the luminous efficiency of the devices, and prolonging the service life of the devices.

### BRIEF DESCRIPTION OF THE DRAWINGS

The drawings are used for a further understanding of the present disclosure and constitute a part of the specification and are used to explain the present disclosure together with the following detailed description, but do not constitute a limitation of the present disclosure.

FIG. 1 is a schematic structural diagram of an organic electroluminescent device according to one embodiment of the present disclosure.

FIG. 2 is a schematic structural diagram of an electronic apparatus according to one embodiment of the present disclosure.

### REFERENCE SIGNS

100: Anode 200: Cathode 300: Functional layer 310: Hole injection layer 321: Hole transport layer 322: Electron blocking layer 330: Organic light-emitting layer 340: Electron transport layer 350: Electron injection layer 400: Electronic apparatus

### DETAILED DESCRIPTION OF THE EMBODIMENTS

Exemplary embodiments will now be described more comprehensively with reference to the accompanying drawings. The exemplary embodiments, however, can be implemented in a variety of forms and should not be interpreted as being limited to the examples set forth herein.

On the contrary, these embodiments are provided to make the present disclosure more comprehensive and complete, and to convey the concepts of these exemplary embodiments fully to those skill in the art. Features, structures, or characteristics described herein can be combined in one or more embodiment(s) in any suitable manner. In the following description, many specific details are provided to give a full understanding of the examples of the present disclosure.

In a first aspect, the present disclosure provides an organic compound having a structure represented by Formula 1 as follows:

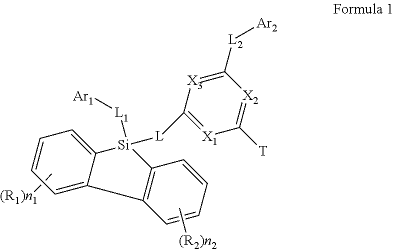

wherein X₁, X₂, and X₃ are each independently selected from N and C(H), and at least two of X₁ to X₃ are N;
Ar₁ and Ar₂ are the same or different, and are each independently selected from a substituted or unsubstituted aryl having 6 to 40 carbon atoms, and a substituted or unsubstituted heteroaryl having 3 to 40 carbon atoms;
L is selected from a single bond, a substituted or unsubstituted arylene having 6 to 30 carbon atoms, and a substituted or unsubstituted heteroarylene having 3 to 30 carbon atoms;
L₁ and L₂ are the same or different, and are each independently a single bond, a substituted or unsubstituted arylene having 6 to 30 carbon atoms, and a substituted or unsubstituted heteroarylene having 3 to 30 carbon atoms;
group T is a substituted or unsubstituted carbazolyl;
substituent(s) in L₁, L₂, Ar₁, and Ar₂ are the same or different, and are each independently selected from a deuterium, a cyano, a halogen group, an alkyl having 1 to 10 carbon atoms, a haloalkyl having 1 to 10 carbon atoms, a deuterated alkyl having 1 to 10 carbon atoms, a trialkylsilyl having 3 to 12 carbon atoms, a triphenylsilyl, an aryl having 6 to 20 carbon atoms, an alkylaryl having 6 to 20 carbon atoms, a deuterated aryl having 6 to 20 carbon atoms, a haloaryl having 6 to 20 carbon atoms, a heteroaryl having 3 to 20 carbon atoms, and a cycloalkyl having 3 to 10 carbon atoms; optionally, any two adjacent substituents form a substituted or unsubstituted ring A; the ring A is a saturated or unsaturated 5 to 13 membered ring;
substituent(s) in the group T, substituent(s) on the ring A, and each of R₁ and R₂ are the same or different, and are each independently selected from a deuterium, a cyano, a halogen group, an alkyl having 1 to 10 carbon atoms, a haloalkyl having 1 to 10 carbon atoms, a deuterated alkyl having 1 to 10 carbon atoms, a trialkylsilyl having 3 to 12 carbon atoms, a triphenylsilyl, an aryl having 6 to 20 carbon atoms, a deuterated aryl having 6 to 20 carbon atoms, a haloaryl having 6 to 20 carbon atoms, a heteroaryl having 3 to 20 carbon atoms, and a cycloalkyl having 3 to 10 carbon atoms;
n₁ is selected from 0, 1, 2, 3, and 4;
n₂ is selected from 0, 1, 2, 3, and 4.

In the present disclosure, the terms “optional” and “optionally” mean that the event or circumstance described later may or may not occur. For example, “optionally, any two adjacent substituents form a ring” means that these two substituents may or may not form a ring, including scenarios both where two adjacent substituents form a ring and where two adjacent substituents do not form a ring. For instance, “any two adjacent substituents form a substituted or unsubstituted ring A” means that any two adjacent substituents are interconnected to form a substituted or unsubstituted ring A, or any two adjacent substituents may exist independently of each other. “Any two adjacent” can include having two substituents on the same atom, and can also include having one substituent on each of adjacent atoms; among them, when there are two substituents on the same atom, the two substituents can form a saturated or unsaturated spiro-ring with the atom they are connected to together; when two adjacent atoms each have a substituent, these two substituents can be fused into a ring.

In the present disclosure, the descriptive expressions “each . . . independently” and e . . . independently“and”. . . each independently” can be interchanged and all these expressions should be interpreted in a broad sense. They can both refer to specific options expressed by the same symbol in separate groups are mutually non-influential, and to specific options expressed by the same symbols within the same group are mutually non-influential. For example,

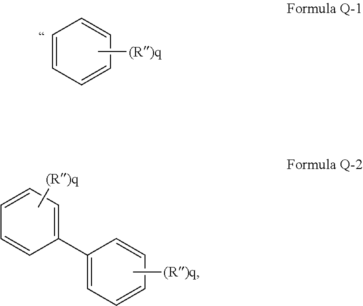

in which each q is independently 0, 1, 2, or 3, and each R″ is independently selected from a hydrogen, a deuterium, a fluorine, and a chlorine” means that Formula Q-1 represents that there are q substituents R″ on the benzene ring, and each R″ can be the same or different, with mutual non-influence between the options for each R”; Formula Q-2 represents that there are q substituents R″ on each benzene ring of biphenyl, and the number q of R″ substituents on the two benzene rings can be the same or different, with mutual non-influence between the options for each R”.

In the present disclosure, the term “substituted or unsubstituted” means that the functional group defined by the term may or may not have a substituent (hereinafter referred to as Rc for ease of description). For example, “a substituted or unsubstituted aryl” refers to an aryl having a substituent Rc or an unsubstituted aryl. Among them, the above substituent, i.e., Rc, may be, for example, a deuterium, a halogen group, a cyano, a heteroaryl, an aryl, an alkylaryl, a trialkylsilyl, an alkyl, a haloalkyl, a deuterated alkyl, a haloaryl, a cycloalkyl, etc. The number of substitutions may be one or more.

In the present disclosure, “more” refers to two or more, for example, 2, 3, 4, 5, or 6, etc.

In the structure of the compound of the present disclosure, a hydrogen atom includes various isotopic atoms of the hydrogen element, such as hydrogen (H), deuterium (D), or tritium (T).

In the present disclosure, the number of carbon atoms of a substituted or unsubstituted functional group refers to the total number of carbon atoms. For example, if L is a substituted arylene having 12 carbon atoms, the total number of carbon atoms in the arylene and its substituents is 12.

In the present disclosure, an aryl refers to an optional functional group or a substituent derived from an aromatic carbon ring. An aryl may be a monocyclic aryl (e.g., phenyl) or a polycyclic aryl. In other words, an aryl may be a monocyclic aryl, a fused-ring aryl, two or more monocyclic aryls linked by carbon-carbon single bond, a monocyclic aryl and a fused-ring aryl linked by carbon-carbon single bond, or two or more fused-ring aryls linked by carbon-carbon single bond. That is, unless otherwise specified, two or more aromatic groups linked by carbon-carbon single bond may also be regarded as an aryl in the present disclosure. Among them, a fused-ring aryl may include, for example, a bicyclic fused aryl (e.g., naphthyl), a tricyclic fused aryl (e.g., phenanthryl, fluorenyl, and anthryl), etc. Examples of an aryl include, but are not limited to, a phenyl, a naphthyl, a fluorenyl, a phenyl-naphthyl, a spirobifluorenyl, an anthryl, a phenanthryl, a biphenyl, a terphenyl, a triphenylene, a perylenyl, a benzo[9,10]phenanthryl, a pyrenyl, a benzofluoranthryl, a chrysenyl, a tetrahydronaphthyl, etc.

In the present disclosure, “an arylene” involved refers to a divalent or multivalent group formed by further removing one or more hydrogen atom(s) from an aryl.

In the present disclosure, a terphenyl includes

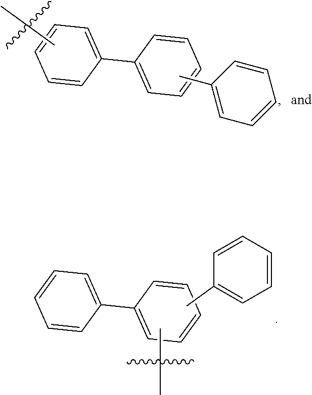

In the present disclosure, the number of carbon atoms in a substituted aryl refers to the total number of carbon atoms of an aryl and the substituents on the aryl. For example, a substituted aryl having 18 carbon atoms, refers to the total number of carbon atoms of the aryl and the substituents thereof is 18.

In the present disclosure, the number of carbon atoms of a substituted or unsubstituted aryl (arylene) may be 6, 8, 10, 12, 13, 14, 15, 16, 17, 18, 19, 20, 21, 22, 23, 24, 25, 26, 28, 30, 31, 33, 34, 35, 36, 38, or 40, etc. In some embodiments, a substituted or unsubstituted aryl is a substituted or unsubstituted aryl having 6 to 40 carbon atoms; in other embodiments, a substituted or unsubstituted aryl is a substituted or unsubstituted aryl having 6 to 30 carbon atoms; in other embodiments, a substituted or unsubstituted aryl is a substituted or unsubstituted aryl having 6 to 25 carbon atoms; and in other embodiments, a substituted or unsubstituted aryl is a substituted or unsubstituted aryl having 6 to 15 carbon atoms.

In the present disclosure, a fluorenyl may be substituted by one or more substituent(s). In the case that the above-mentioned fluorenyl is substituted, the substituted fluorenyl may be:

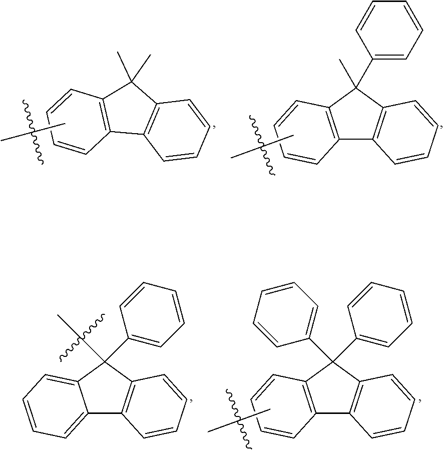

etc, but are not limited thereto.

In the present disclosure, an aryl as the substituent of L, L₁, L₂, Ar₁, and Ar₂ is for example, but is not limited to, a phenyl, a naphthyl, a phenanthryl, a biphenyl, a fluorenyl, a dimethylfluorenyl, etc.

In the present disclosure, “a heteroaryl” refers to a monovalent aromatic ring containing 1, 2, 3, 4, 5, or 6 heteroatoms or a derivative thereof. The heteroatoms may be one or more selected from B, O, N, P, Si, Se, and S. A heteroaryl may be a monocyclic heteroaryl or a polycyclic heteroaryl. In other words, a heteroaryl may be a single aromatic ring system, or multiple aromatic ring systems linked by carbon-carbon single bond, with any of the aromatic ring systems being an aromatic monocyclic ring or an aromatic fused ring. For example, aheteroaryl may include, thienyl, furyl, pyrrolyl, imidazolyl, thiazolyl, oxazolyl, oxadiazolyl, triazolyl, pyridyl, dipyridyl, pyrimidinyl, triazinyl, acridinyl, pyridazinyl, pyrazinyl, quinolyl, quinazolinyl, quinoxalinyl, phenoxazinyl, phthalazinyl, pyridopyrimidinyl, pyridopyrazinyl, pyrazinopyrazinyl, isoquinolyl, indolyl, carbazolyl, benzoxazolyl, benzimidazolyl, benzothiazolyl, benzocarbazolyl, benzothienyl, dibenzothienyl, thienothienyl, benzofuranyl, phenanthrolinyl, isoxazolyl, thiadiazolyl, phenothiazinyl, silafluorenyl, dibenzofuranyl, etc, but not limited thereto.

In the present disclosure, “a heteroarylene” involved refers to a divalent or multivalent group formed by further removing one or more hydrogen atom(s) from a heteroaryl.

In the present disclosure, the number of carbon atoms of a substituted or unsubstituted heteroaryl (heteroarylene) may be selected from 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, etc. In some embodiments, a substituted or unsubstituted heteroaryl is a substituted or unsubstituted heteroaryl having 3 to 40 carbon atoms in total; in other embodiments, a substituted or unsubstituted heteroaryl is a substituted or unsubstituted heteroaryl having 3 to 30 carbon atoms in total; and in other embodiments, a substituted or unsubstituted heteroaryl is a substituted or unsubstituted heteroaryl having 5 to 12 carbon atoms in total.

In the present disclosure, a heteroaryl as the substituent of L, L₁, L₂, Ar₁, and Ar₂ is for example, but is not limited to a pyridyl, a carbazolyl, a quinolyl, an isoquinolyl, a phenanthrolinyl, a benzoxazolyl, a benzothiazolyl, a benzimidazolyl, a dibenzothienyl, and a dibenzofuranyl.

In the present disclosure, a substituted heteroaryl may mean that one or more than two hydrogen atom(s) in the heteroaryl are replaced by a group such as a deuterium atom, a halogen group, a cyano, an aryl, a heteroaryl, a trialkylsilyl, an alkyl, a cycloalkyl, and a haloalkyl. It should be understood that the number of carbon atoms in the substituted heteroaryl refers to the total number of carbon atoms in the heteroaryl and the substituents thereon.

In the present disclosure, an alkyl having 1 to 10 carbon atoms may include a straight-chain alkyl having 1 to 10 carbon atoms, and a branched alkyl having 3 to 10 carbon atoms. The number of carbon atoms of an alkyl is for example 1, 2, 3, 4, 5, 6, 7, 8, 9, or 10, and the specific examples of the alkyl include but are not limited to, a methyl, an ethyl, a n-propyl, an isopropyl, a n-butyl, an isobutyl, a tert-butyl, a n-pentyl, an isopentyl, a neopentyl, a n-hexyl, etc.

In the present disclosure, a halogen group is for example, a fluorine, a chlorine, a bromine, or an iodine.

In the present disclosure, the specific examples of a trialkylsilyl include, but are not limited to, a trimethylsilyl, a triethylsilyl, etc.

In the present disclosure, the specific examples of a haloalkyl include, but are not limited to, a trifluoromethyl.

In the present disclosure, the specific examples of a deuterated alkyl include, but are not limited to, a trideuterated methyl.

In the present disclosure, the specific examples of a deuterated aryl include, but are not limited to a deuterated phenyl, a deuterated naphthyl, a deuterated biphenyl, etc.

In the present disclosure, a haloaryl refers to an aryl with a halogen substituent, which is for example, but is not limited to a fluorophenyl, a fluoronaphthyl, a fluorobiphenyl, etc.

In the present disclosure, a saturated or unsaturated 5 to 13 membered ring refers to a carbon ring or a hetero ring comprising 5 to 13 ring atoms; it is for example, but is not limited to a cyclopentane, a cyclohexane, a benzene ring, a fluorene ring, a pyran ring, tetrahydropyran ring, a piperidine ring, a tetrahydropiperidine ring, etc.

In the present disclosure, the number of carbon atoms of a cycloalkyl having 3 to 10 carbon atoms is for example 3, 4, 5, 6, 7, 8, or 10. The specific examples of a cycloalkyl include, but are not limited to, a cyclopentyl, a cyclohexyl, an adamantyl, etc.

In the present disclosure, a non-positioned bond involves a single bond

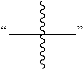

extending from the ring system, which represents that one end of the connection bond can connect to any position in the ring system through which the bond passes, and the other end connects to the rest of the compound molecule. For example, as shown in Formula (f) below, the naphthyl represented by Formula (f) is connected to other positions of the molecule through two non-positioned bonds passing through the two rings, which indicates any of possible connection forms shown in Formulae (f-1) to (f-10):

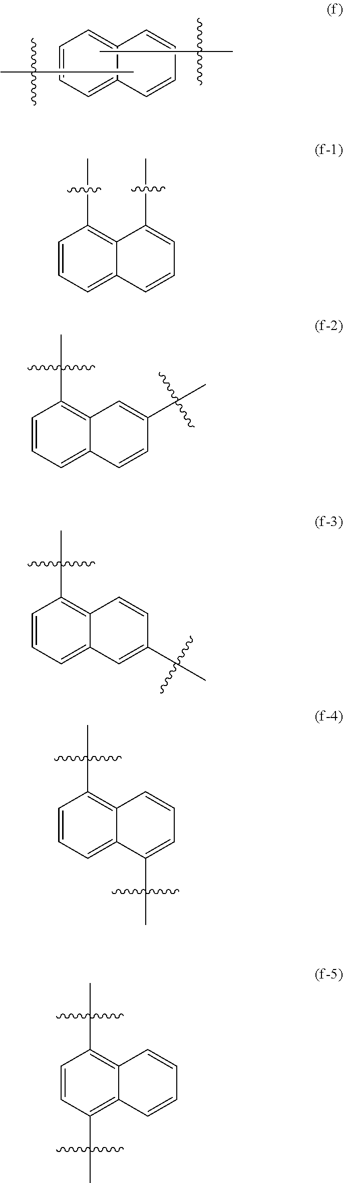

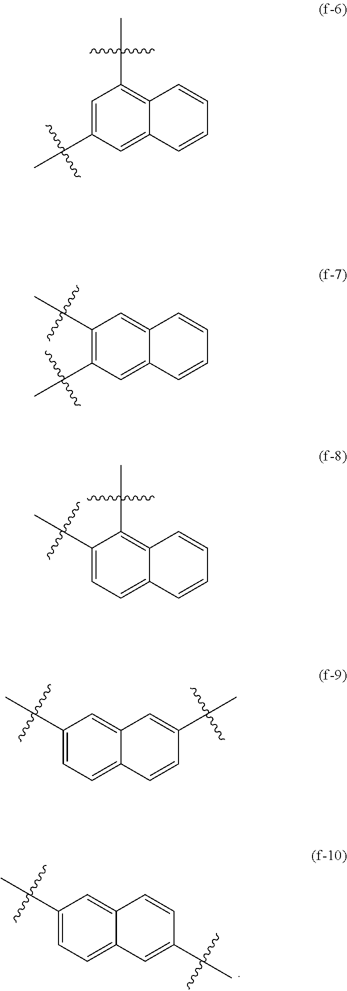 [SMILES: *c1cc(*)c2ccccc2c1 ; *c1cc2ccccc2cc1* ; *c1ccc2ccccc2c1* ; *c1ccc2ccc(*)cc2c1 ; *c1ccc2cc(*)ccc2c1]

As another example, as shown in Formula (X′) below, the dibenzofuranyl represented by Formula (X′) is connected to other positions of the molecule via a non-positioned connection bond extending from the center of a side benzene ring, which indicates any of possible connection forms shown in Formulae (X′-1) to (X′-4):

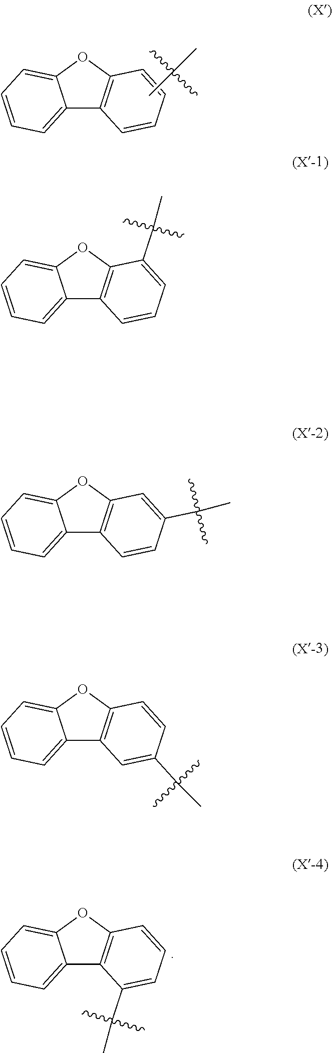

The non-positioned substituent in the present disclosure refers to a substituent connected by a single bond extending from the center of the ring system, indicating that the substituent can be connected to any possible position in the ring system. For example, as represented by Formula (Y) below, the substituent R′ represented by Formula (Y) is linked to a quinoline ring via a non-positioned connection bond, which indicates any of possible connecting mode shown in Formulae (Y-1) to (Y-7):

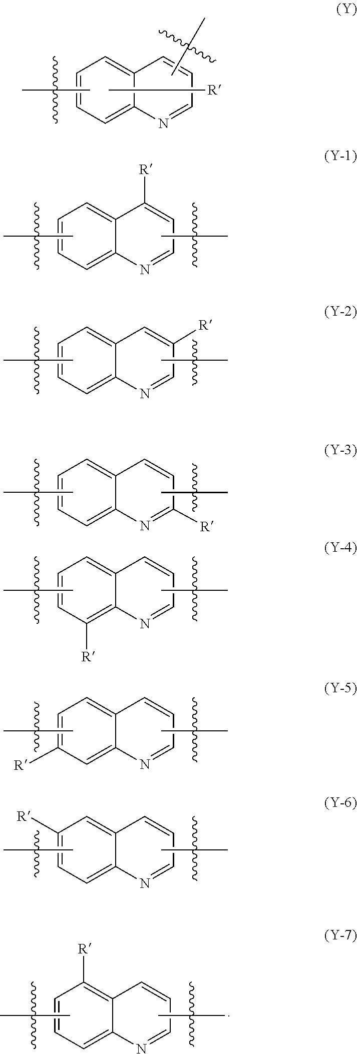

In some embodiments, the organic compound is selected from the structure represented by the following Formula (1-1):

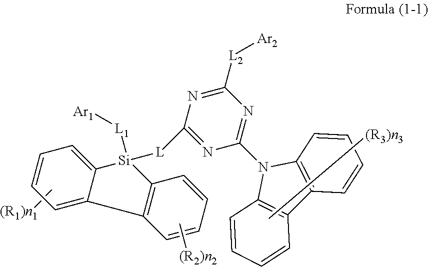

wherein, each R₃ is the same or different, and is independently selected from a deuterium, a cyano, a halogen group, an alkyl having 1 to 5 carbon atoms, a haloalkyl having 1 to 5 carbon atoms, a deuterated alkyl having 1 to 5 carbon atoms, a trialkylsilyl having 3 to 8 carbon atoms, an aryl having 6 to 15 carbon atoms, a deuterated aryl having 6 to 15, a haloaryl having 6 to 15 carbon atoms, a heteroaryl having 5 to 12 carbon atoms, and a cycloalkyl having 3 to 8 carbon atoms; n₃ represents the number of R₃, and n₃ is selected from 0, 1, 2, 3, 4, 5, 6, 7, or 8.

In some embodiments, the group T is selected from the group consisting of the following groups:

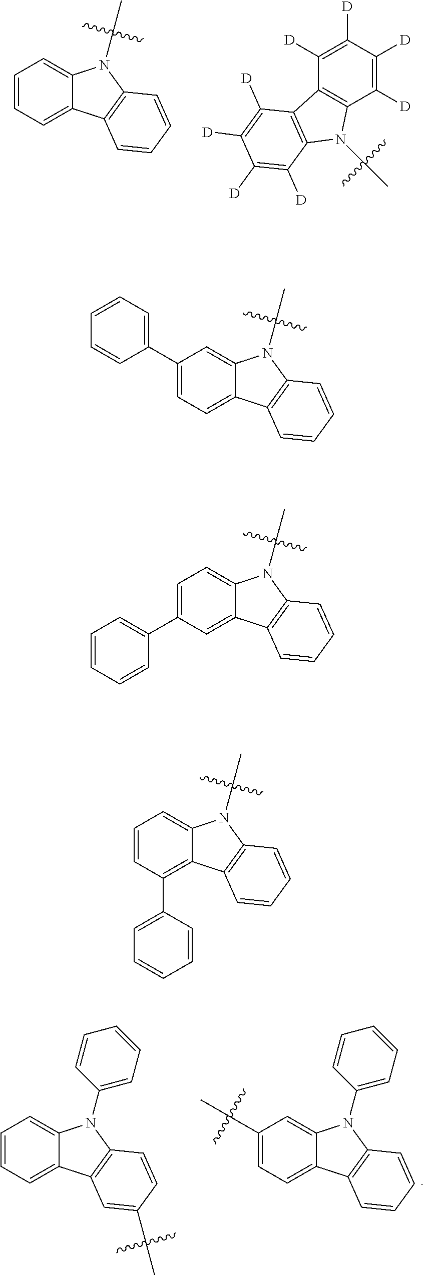

In some embodiments, Ar₁ and Ar₂ are the same or different, and are each independently selected from a substituted or unsubstituted aryl having 6 to 25 carbon atoms, and a substituted or unsubstituted heteroaryl having 5 to 18 carbon atoms.

In some embodiments, Ar₁ and Ar₂ are the same or different, and are each independently selected from a substituted or unsubstituted aryl having 6, 7, 8, 9, 10, 11, 12, 13, 14, 15, 16, 17, 18, 19, 20, 21, 22, 23, 24, or 25 carbon atoms, and a substituted or unsubstituted heteroaryl having 5, 6, 7, 8, 9, 10, 11, 12, 13, 14, 15, 16, 17, or 18 carbon atoms.

In some embodiments, substituent(s) in Ar₁ and Ar₂ are each independently selected from a deuterium, a halogen group, a cyano, a haloalkyl having 1 to 4 carbon atoms, a deuterated alkyl having 1 to 4 carbon atoms, an alkyl having 1 to 4 carbon atoms, a cycloalkyl having 5 to 10 carbon atoms, an aryl having 6 to 15 carbon atoms, a deuterated aryl having 6 to 12 carbon atoms, a heteroaryl having 5 to 12 carbon atoms, and a trialkylsilyl having 3 to 8 carbon atoms; optionally, any two adjacent substituents in Ar₁ and Ar₂ form a substituted or unsubstituted 5 to 10 membered ring, and the substituent(s) on the 5 to 10 membered ring are each independently selected from a deuterium and an alkyl having 1 to 5 carbon atoms.

In some embodiments, Ar₁ and Ar₂ are the same or different, and are each independently selected from a substituted or unsubstituted phenyl, a substituted or unsubstituted naphthyl, a substituted or unsubstituted tetrahydronaphthyl, a substituted or unsubstituted biphenyl, a substituted or unsubstituted terphenyl, a substituted or unsubstituted fluorenyl, a substituted or unsubstituted phenanthryl, a substituted or unsubstituted anthryl, a substituted or unsubstituted triphenylene, a substituted or unsubstituted dibenzofuranyl, a substituted or unsubstituted dibenzothienyl, and a substituted or unsubstituted carbazolyl.

Optionally, substituent(s) in Ar₁ and Ar₂ are each independently selected from a deuterium, a fluorine, a cyano, a trimethylsilyl, a trideuterated methyl, a trifluoromethyl, a cyclopentyl, a cyclohexyl, a methyl, an ethyl, an isopropyl, a tert-butyl, a phenyl, a pentadeuterated phenyl, a naphthyl, a biphenyl, a 9,9-dimethylfluorenyl, a phenanthryl, a dibenzofuranyl, a dibenzothienyl, and a carbazolyl; any two adjacent substituents in Ar₁ and Ar₂ form a substituted or unsubstituted cyclohexane, a substituted or unsubstituted cyclopentane, and a substituted or unsubstituted benzene ring, wherein the substituent(s) on the cyclopentane, cyclohexane, or benzene ring are each independently selected from a deuterium and a methyl.

In some embodiments, Ar₁ and Ar₂ are the same or different, and are each independently selected from a substituted or unsubstituted group V; wherein the unsubstituted group V is selected from the following groups:

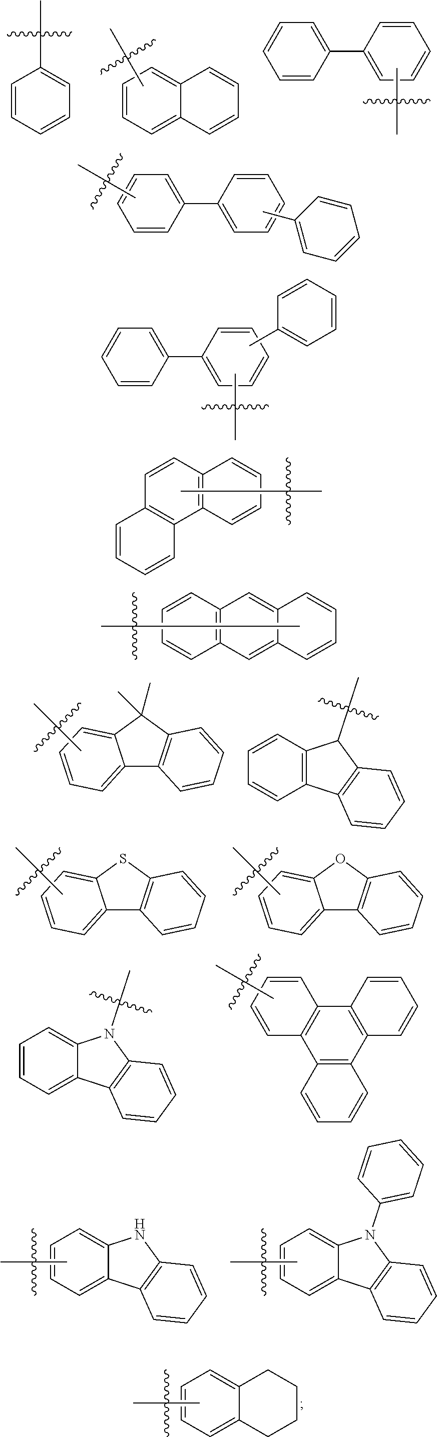

the substituted group V has one or more substituent(s), and the substituent(s) are each independently selected from a deuterium, a fluorine, a cyano, a trimethylsilyl, a trideuterated methyl, a trifluoromethyl, a cyclopentyl, a cyclohexyl, a methyl, an ethyl, an isopropyl, a tert-butyl, a phenyl, a naphthyl, a biphenyl, a fluorenyl, a phenanthryl, a dibenzofuranyl, a dibenzothienyl, and a carbazolyl, and when the number of substituents on the group V is greater than 1, the substituents are the same or different.

In some embodiments, Ar₁ and Ar₂ are the same or different, and are each independently selected from the group consisting of the following groups:

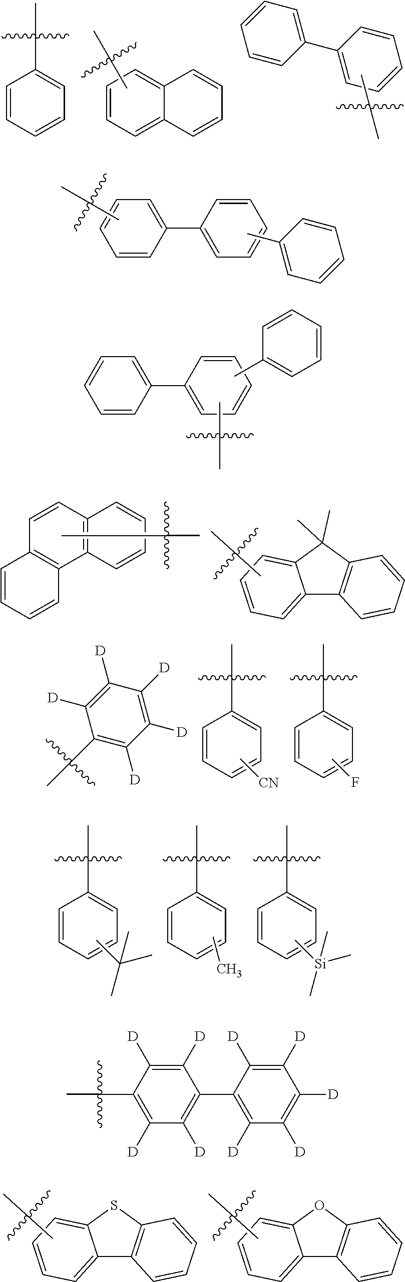

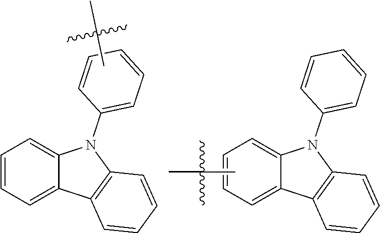

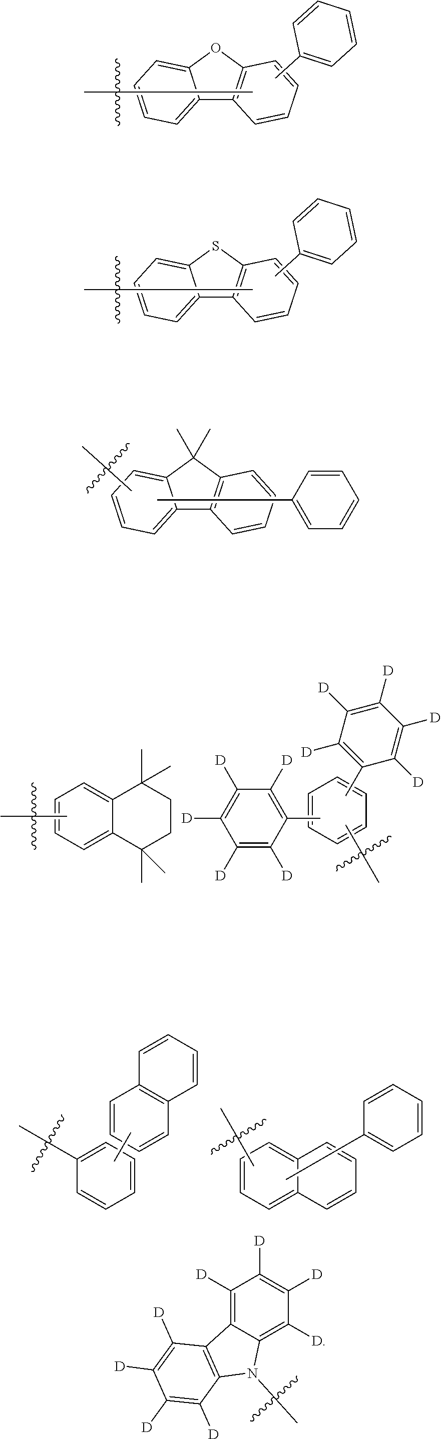

In some embodiments, Ar₁ and Ar₂ are the same or different, and are each independently selected from the group consisting of the following groups:

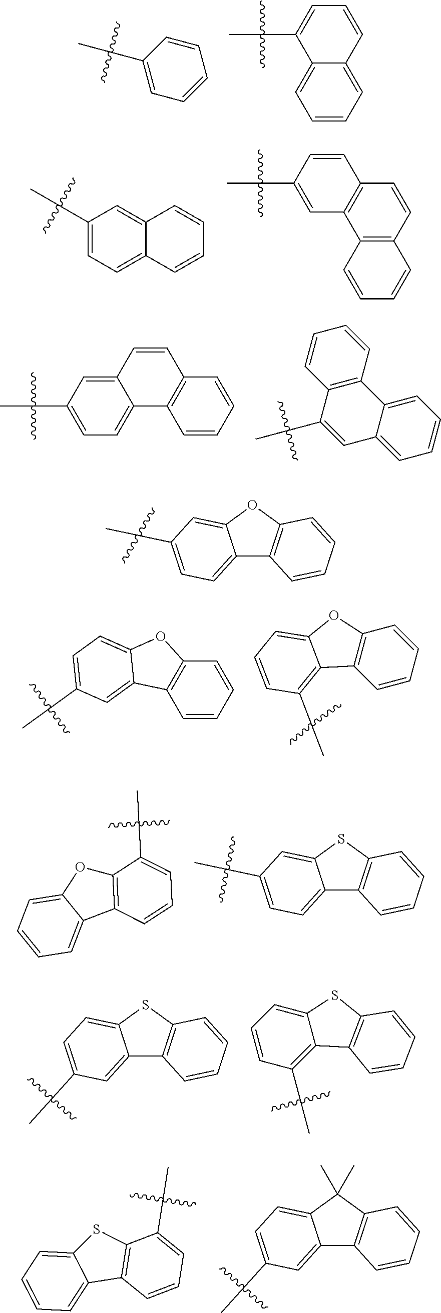

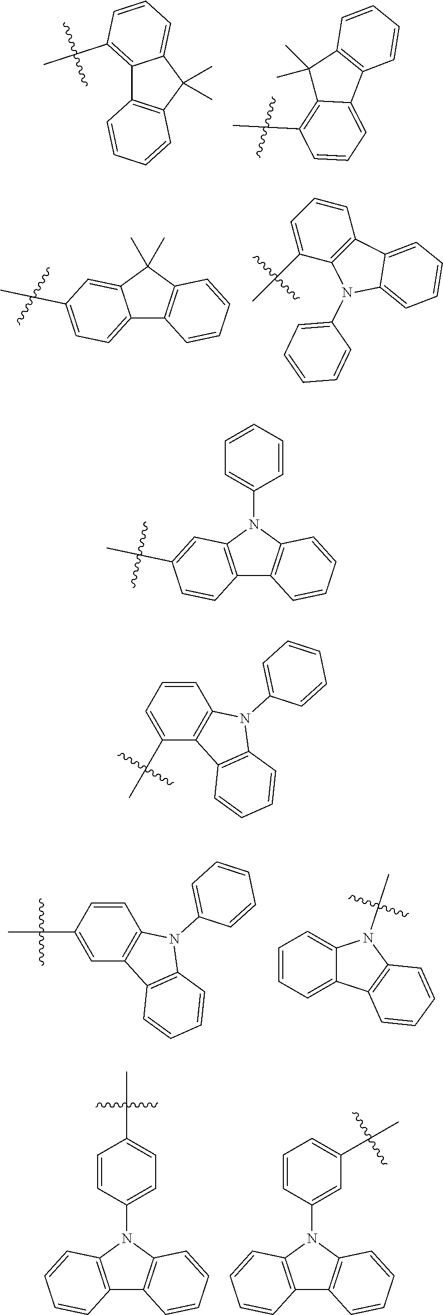

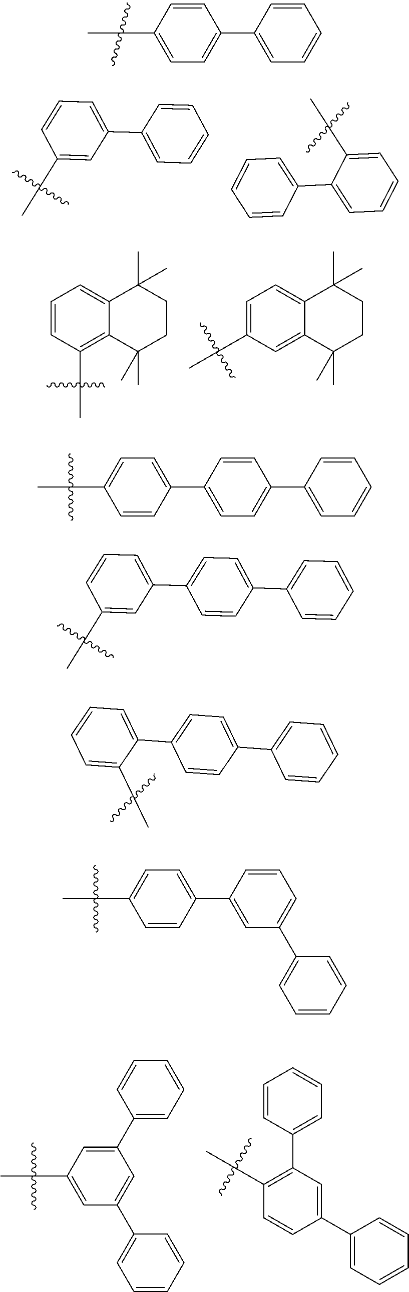

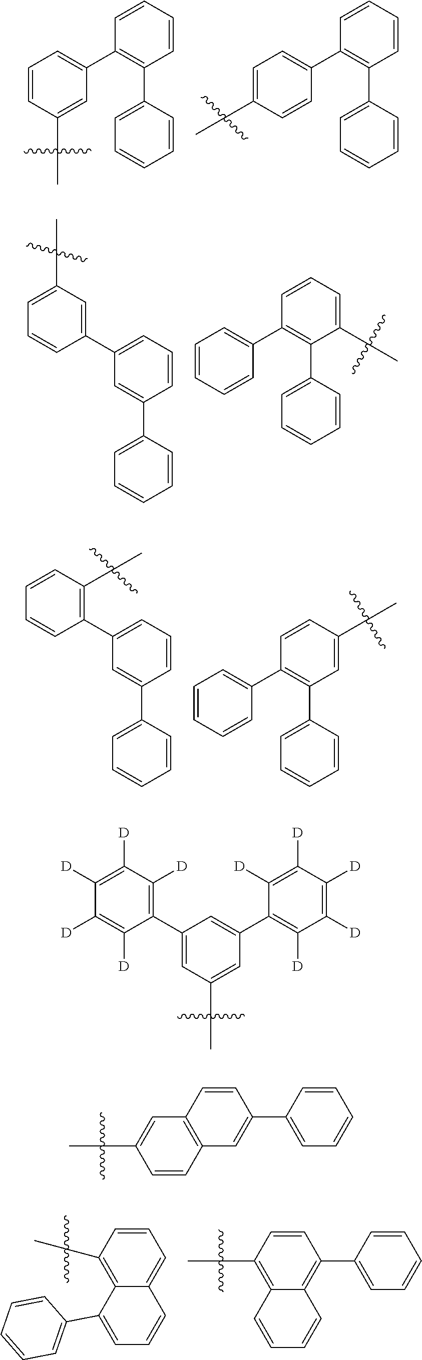

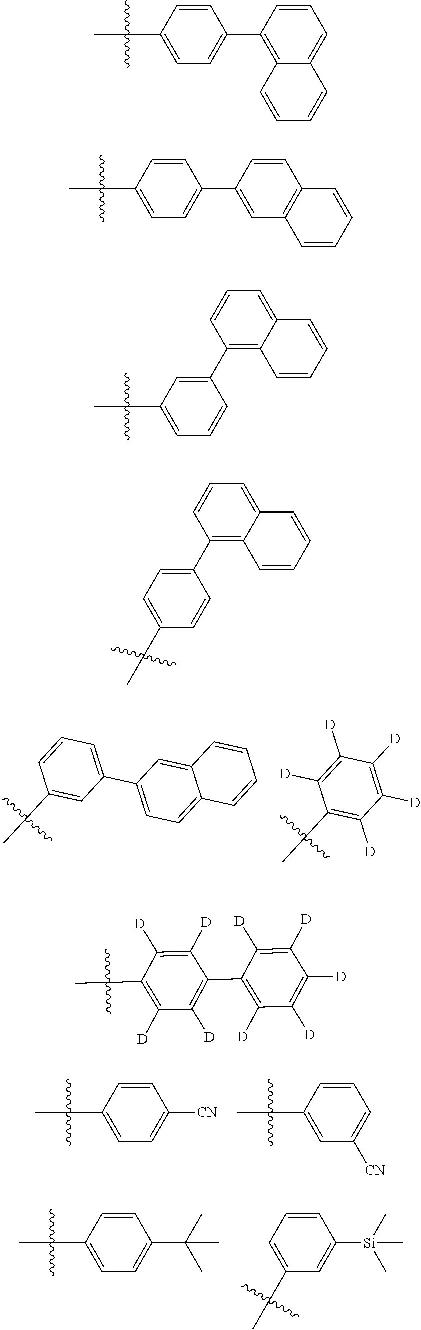

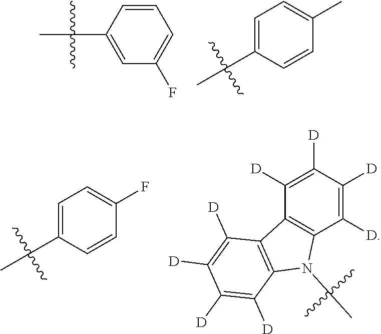

In some embodiments, L₁ and L₂ are the same or different, and are each independently selected from a single bond, a substituted or unsubstituted arylene having 6 to 15 carbon atoms, and a substituted or unsubstituted heteroarylene having 5 to 12 carbon atoms.

In some embodiments, L is selected from a substituted or unsubstituted arylene having 6 to 15 carbon atoms, and a substituted or unsubstituted heteroarylene having 5 to 18 carbon atoms.

In some embodiments, L₁ and L₂ are the same or different, and are each independently selected from a single bond, a substituted or unsubstituted arylene having 6, 7, 8, 9, 10, 11, 12, 13, 14, or 15, and a substituted or unsubstituted heteroarylene having 5, 6, 7, 8, 9, 10, 11, or 12.

Optionally, substituent(s) in L, L₁, and L₂ are each independently selected from a deuterium, a fluorine, a cyano, an alkyl having 1 to 5 carbon atoms, a trialkylsilyl having 3 to 8 carbon atoms, a fluoroalkyl having 1 to 4 carbon atoms, a deuterated alkyl having 1 to 4 carbon atoms, a phenyl, a tolyl, and a pentadeuterated phenyl.

In some embodiments, L₁ and L₂ are the same or different, and are each independently selected from a single bond, a substituted or unsubstituted phenylene, a substituted or unsubstituted naphthylene, a substituted or unsubstituted biphenylene, a substituted or unsubstituted anthrylene, a substituted or unsubstituted phenanthrylene, a substituted or unsubstituted fluorenylene, a substituted or unsubstituted pyridylene, a substituted or unsubstituted dibenzothienylene, a substituted or unsubstituted dibenzofuranylene, and a substituted or unsubstituted carbazolylene.

Optionally, substituent(s) in L₁ and L₂ are the same or different, and are each independently selected from a deuterium, a fluorine, a cyano, a methyl, an ethyl, an isopropyl, a tert-butyl, a trifluoromethyl, a trideuterated methyl, a trimethylsilyl, a phenyl, a methylphenyl, and a pentadeuterated phenyl.

In some embodiments, L is selected from a substituted or unsubstituted phenylene, a substituted or unsubstituted naphthylene, a substituted or unsubstituted biphenylene, a substituted or unsubstituted anthrylene, a substituted or unsubstituted phenanthrylene, a substituted or unsubstituted fluorenylene, a substituted or unsubstituted pyridylene, a substituted or unsubstituted dibenzothienylene, a substituted or unsubstituted dibenzofuranylene, and a substituted or unsubstituted carbazolylene.

Optionally, substituent(s) in L are the same or different, and are each independently selected from a deuterium, a fluorine, a cyano, a methyl, an ethyl, an isopropyl, a tert-butyl, a trifluoromethyl, a trideuterated methyl, a trimethylsilyl, a phenyl, a methylphenyl, and a pentadeuterated phenyl.

In some embodiments, L is selected from the group consisting of the following groups:

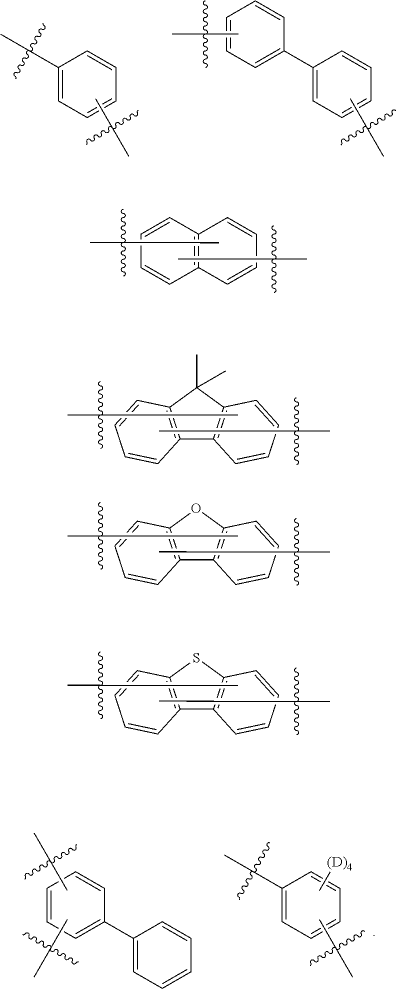

In some embodiments of the present disclosure, L is selected from the group consisting of the following groups:

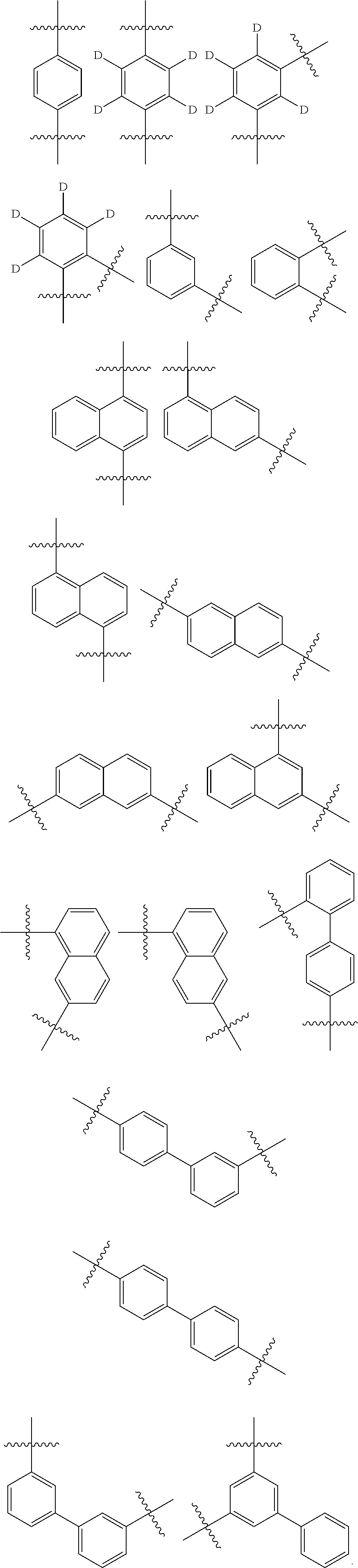

In some embodiments, L₁ and L₂ are each independently selected from a single bond and the group consisting of the following groups:

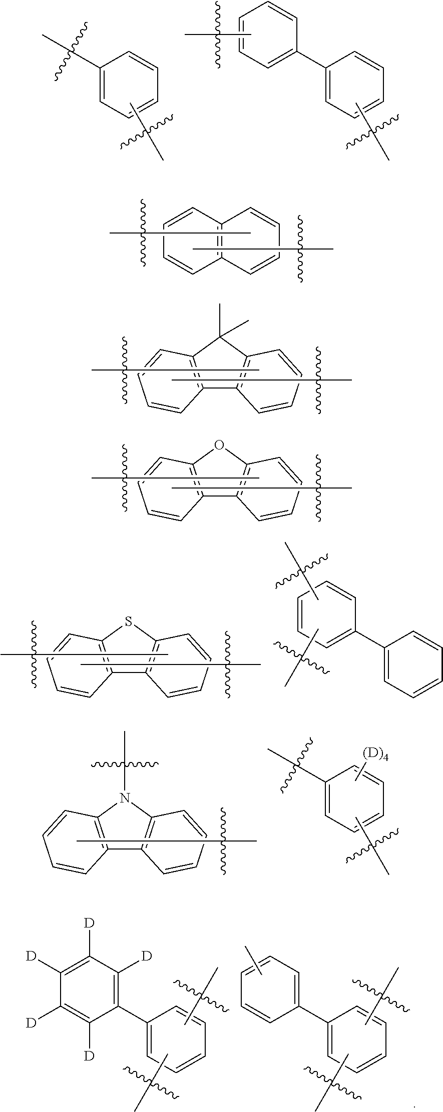

In some embodiments, L₁ and L₂ are each independently selected from a single bond and the following groups:

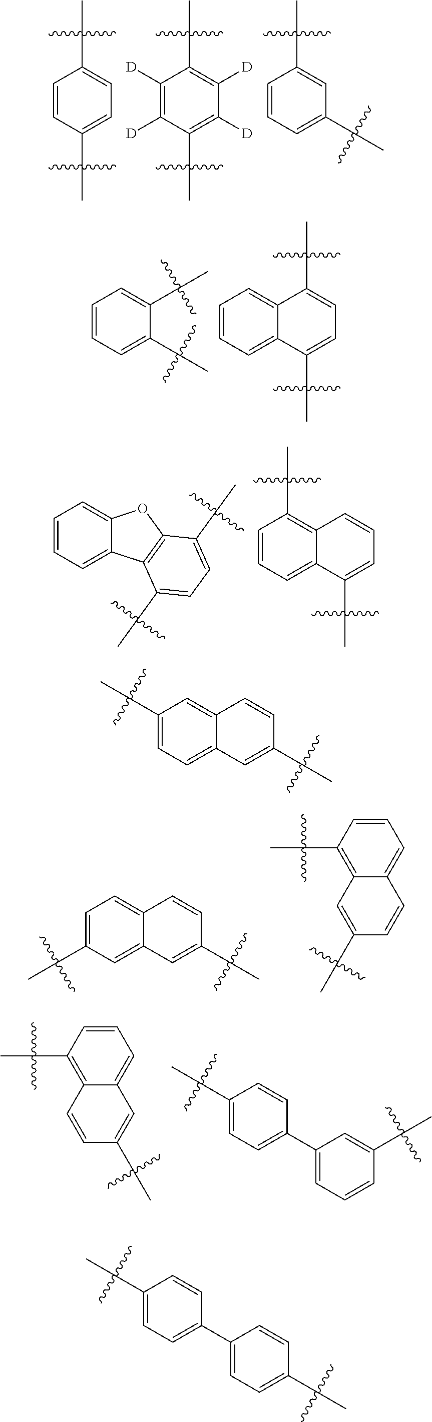

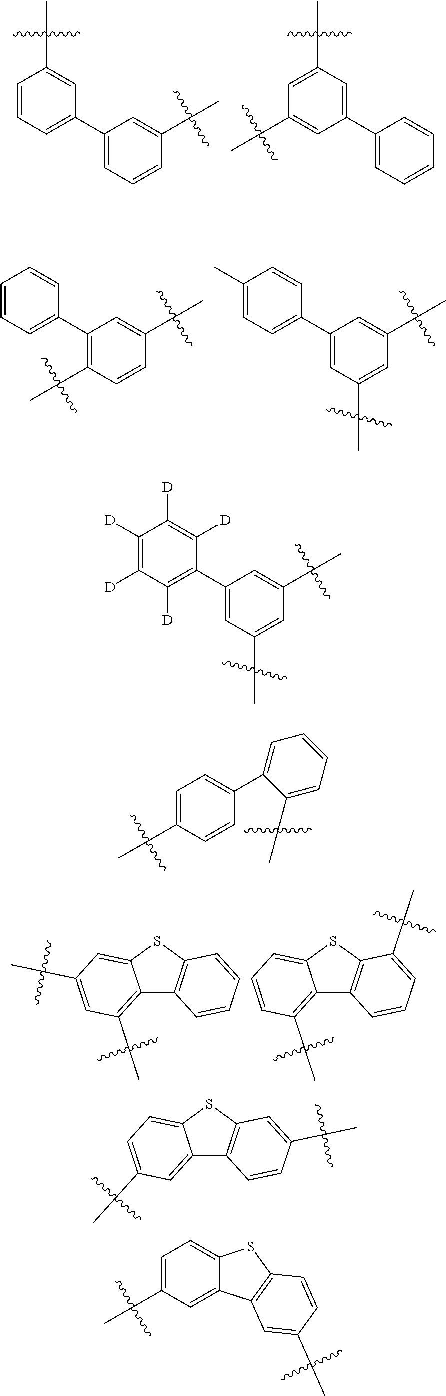

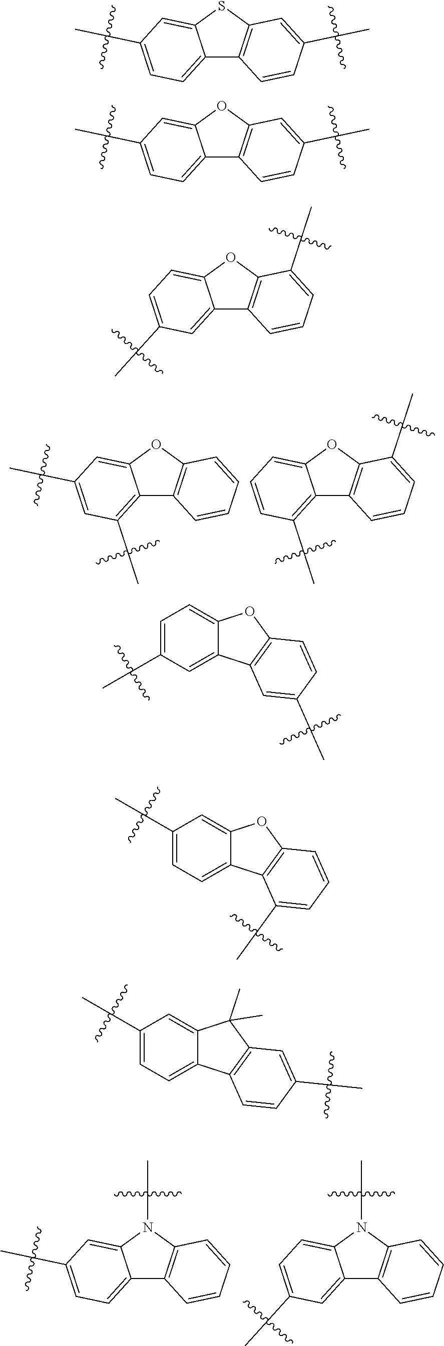

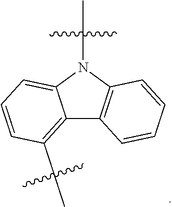 [SMILES: *c1cccc2c1c1ccccc1n2*]

In some embodiments,

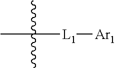

in Formula 1 is selected from the group consisting of the following groups:

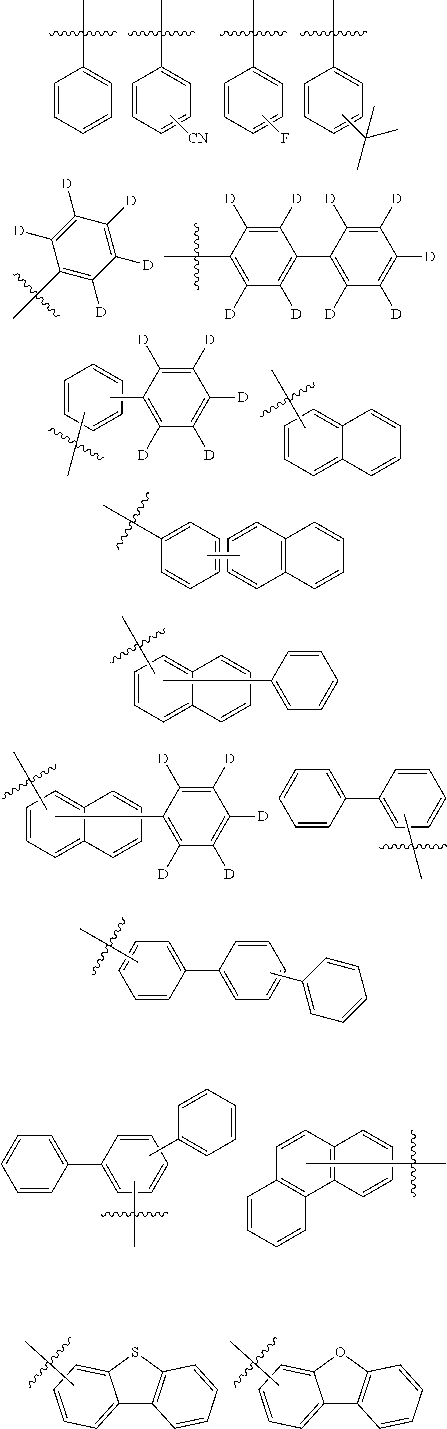

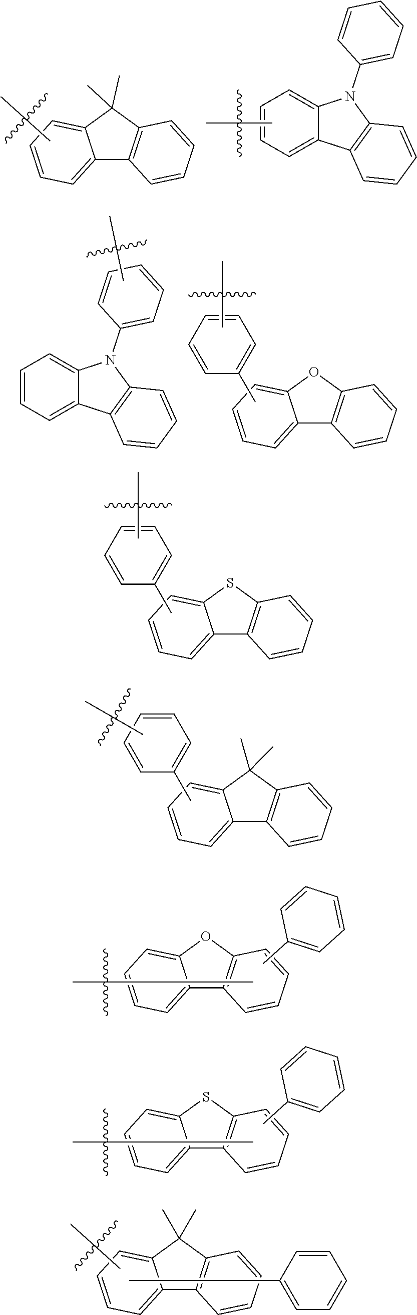

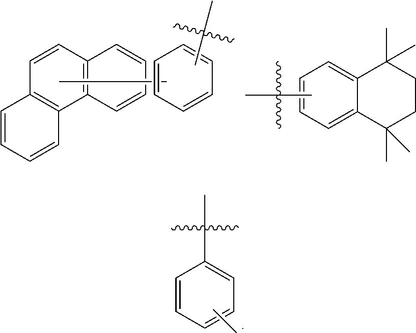

In some embodiments,

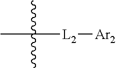

is selected from the following groups:

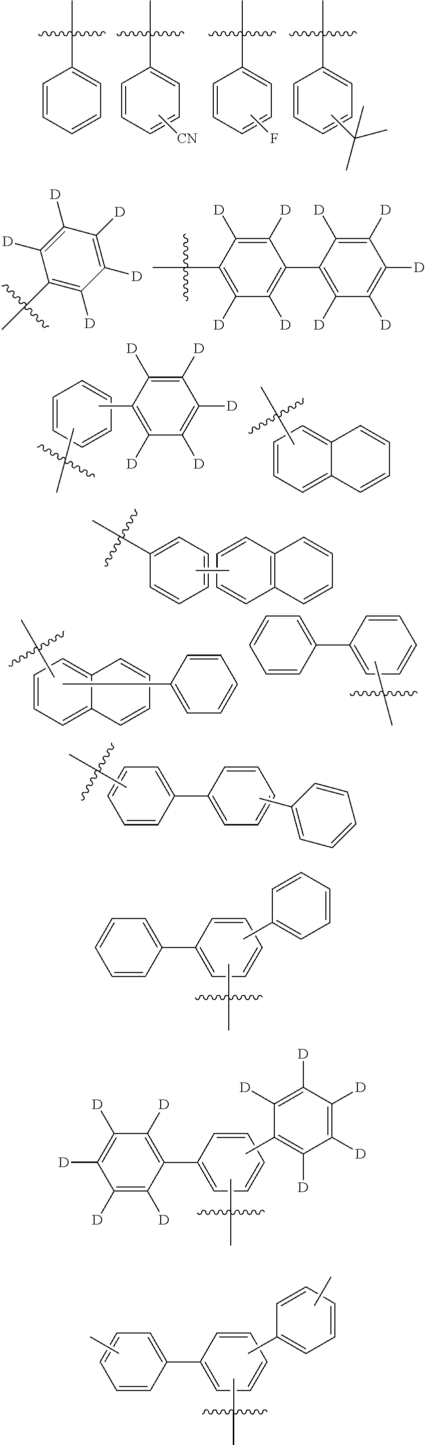

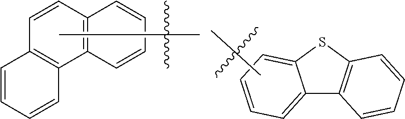

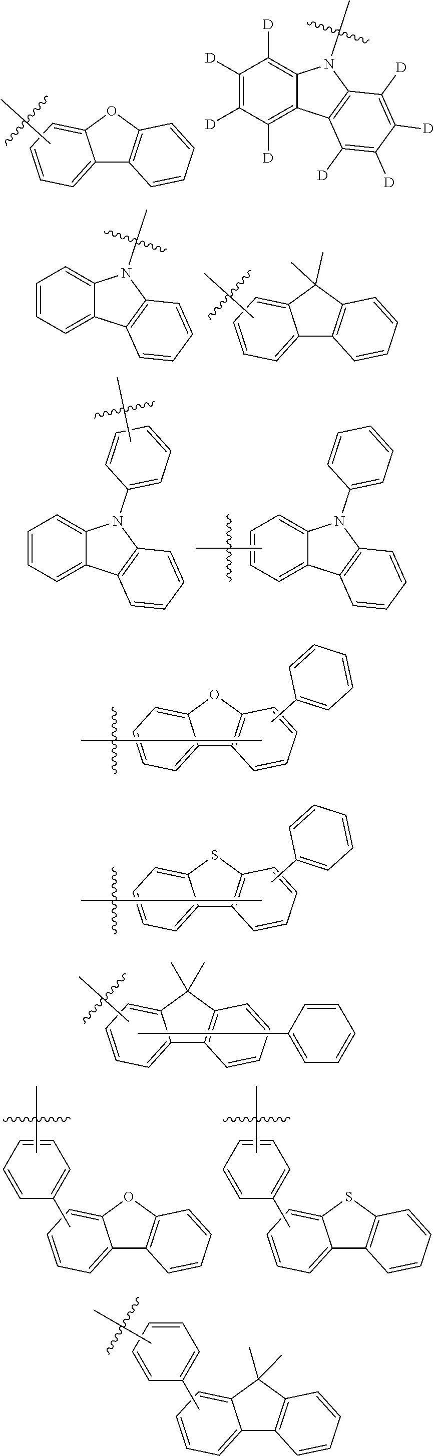

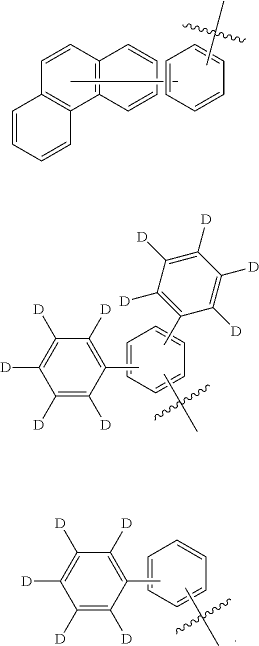

In some embodiments, each R₃ is the same or different, and is independently selected from a deuterium, a cyano, a fluorine, a methyl, an ethyl, an isopropyl, a tert-butyl, a trifluoromethyl, a trideuterated methyl, a trimethylsilyl, a phenyl, a pentadeuterated phenyl, a biphenyl, and a naphthyl.

In some embodiments, each R₁ and R₂ are the same or different, and are each independently selected from a deuterium, a cyano, a fluorine, a methyl, an ethyl, an isopropyl, a tert-butyl, a trifluoromethyl, a trideuterated methyl, a trimethylsilyl, a phenyl, a pentadeuterated phenyl, a biphenyl, and a naphthyl.

In some embodiments, the group Ar₂ is selected from the group consisting of the following groups:

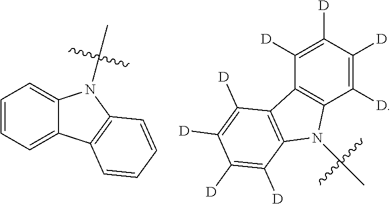

In some embodiments, the organic compound of the present disclosure is selected from the group consisting of the following compounds:

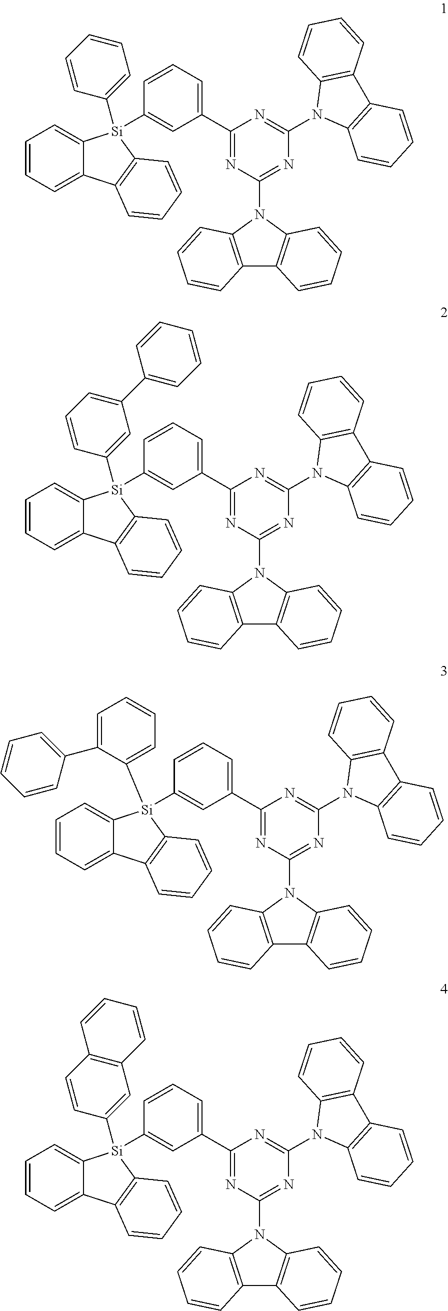 [SMILES: c1ccc([Si]2(c3cccc(-c4nc(-n5c6ccccc6c6ccccc65)nc(-n5c6ccccc6c6ccccc65)n4)c3)c3ccccc3-c3ccccc32)cc1 ; c1ccc(-c2cccc([Si]3(c4cccc(-c5nc(-n6c7ccccc7c7ccccc76)nc(-n6c7ccccc7c7ccccc76)n5)c4)c4ccccc4-c4ccccc43)c2)cc1 ; c1ccc(-c2ccccc2[Si]2(c3cccc(-c4nc(-n5c6ccccc6c6ccccc65)nc(-n5c6ccccc6c6ccccc65)n4)c3)c3ccccc3-c3ccccc32)cc1 ; c1cc(-c2nc(-n3c4ccccc4c4ccccc43)nc(-n3c4ccccc4c4ccccc43)n2)cc([Si]2(c3ccc4ccccc4c3)c3ccccc3-c3ccccc32)c1]

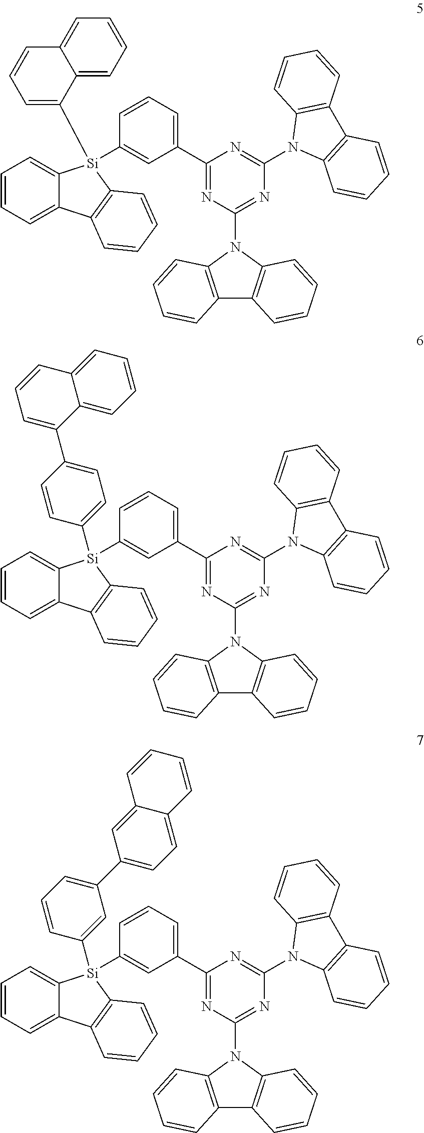

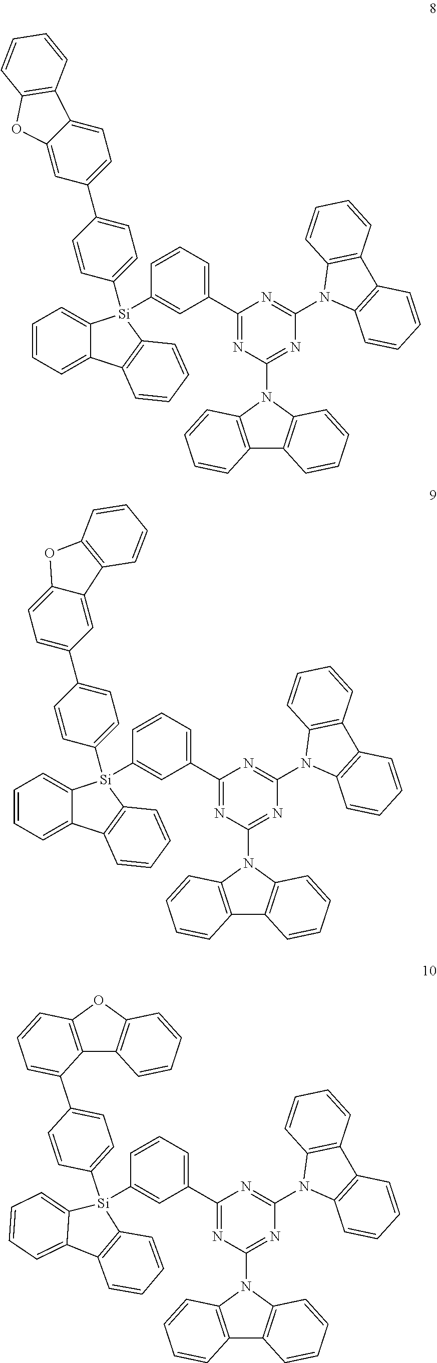 [SMILES: c1cc(-c2nc(-n3c4ccccc4c4ccccc43)nc(-n3c4ccccc4c4ccccc43)n2)cc([Si]2(c3ccc(-c4ccc5c(c4)oc4ccccc45)cc3)c3ccccc3-c3ccccc32)c1 ; c1cc(-c2nc(-n3c4ccccc4c4ccccc43)nc(-n3c4ccccc4c4ccccc43)n2)cc([Si]2(c3ccc(-c4ccc5oc6ccccc6c5c4)cc3)c3ccccc3-c3ccccc32)c1 ; c1cc(-c2nc(-n3c4ccccc4c4ccccc43)nc(-n3c4ccccc4c4ccccc43)n2)cc([Si]2(c3ccc(-c4cccc5oc6ccccc6c45)cc3)c3ccccc3-c3ccccc32)c1]

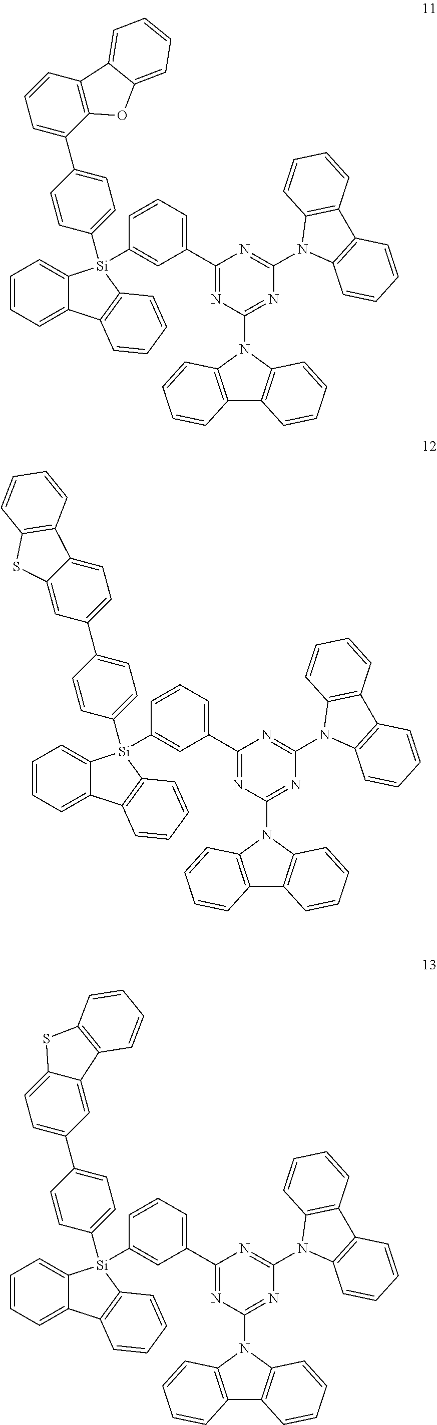 [SMILES: c1cc(-c2nc(-n3c4ccccc4c4ccccc43)nc(-n3c4ccccc4c4ccccc43)n2)cc([Si]2(c3ccc(-c4cccc5c4oc4ccccc45)cc3)c3ccccc3-c3ccccc32)c1 ; c1cc(-c2nc(-n3c4ccccc4c4ccccc43)nc(-n3c4ccccc4c4ccccc43)n2)cc([Si]2(c3ccc(-c4ccc5c(c4)sc4ccccc45)cc3)c3ccccc3-c3ccccc32)c1 ; c1cc(-c2nc(-n3c4ccccc4c4ccccc43)nc(-n3c4ccccc4c4ccccc43)n2)cc([Si]2(c3ccc(-c4ccc5sc6ccccc6c5c4)cc3)c3ccccc3-c3ccccc32)c1]

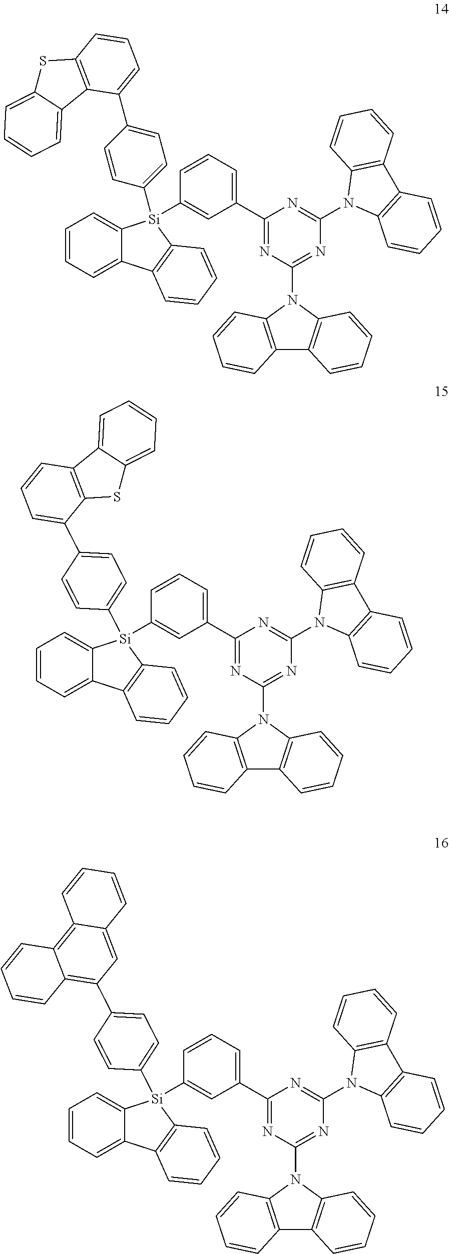 [SMILES: c1cc(-c2nc(-n3c4ccccc4c4ccccc43)nc(-n3c4ccccc4c4ccccc43)n2)cc([Si]2(c3ccc(-c4cccc5sc6ccccc6c45)cc3)c3ccccc3-c3ccccc32)c1 ; c1cc(-c2nc(-n3c4ccccc4c4ccccc43)nc(-n3c4ccccc4c4ccccc43)n2)cc([Si]2(c3ccc(-c4cccc5c4sc4ccccc45)cc3)c3ccccc3-c3ccccc32)c1 ; c1cc(-c2nc(-n3c4ccccc4c4ccccc43)nc(-n3c4ccccc4c4ccccc43)n2)cc([Si]2(c3ccc(-c4cc5ccccc5c5ccccc45)cc3)c3ccccc3-c3ccccc32)c1]

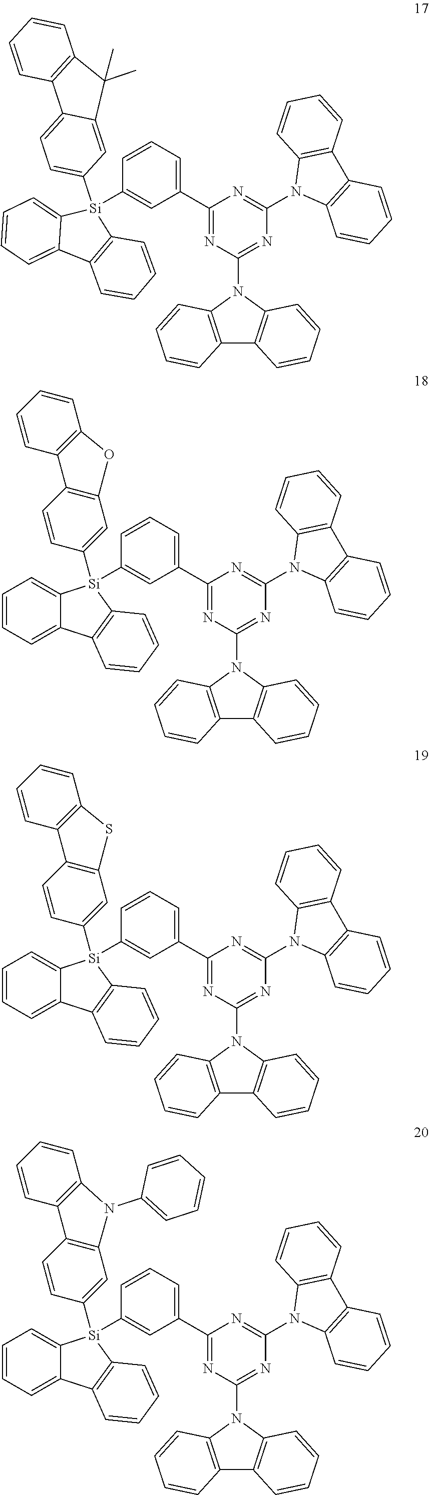

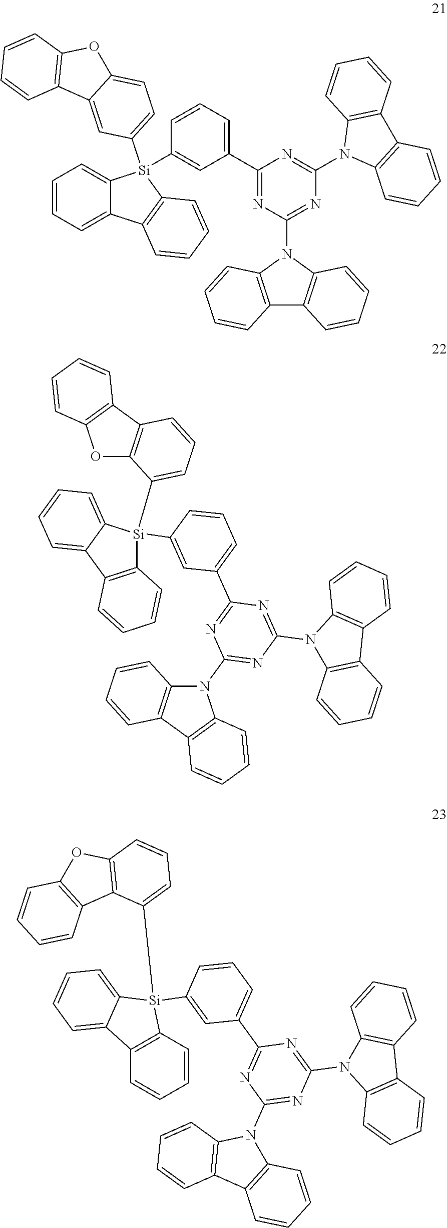 [SMILES: c1cc(-c2nc(-n3c4ccccc4c4ccccc43)nc(-n3c4ccccc4c4ccccc43)n2)cc([Si]2(c3ccc4oc5ccccc5c4c3)c3ccccc3-c3ccccc32)c1 ; c1cc(-c2nc(-n3c4ccccc4c4ccccc43)nc(-n3c4ccccc4c4ccccc43)n2)cc([Si]2(c3cccc4c3oc3ccccc34)c3ccccc3-c3ccccc32)c1 ; c1cc(-c2nc(-n3c4ccccc4c4ccccc43)nc(-n3c4ccccc4c4ccccc43)n2)cc([Si]2(c3cccc4oc5ccccc5c34)c3ccccc3-c3ccccc32)c1]

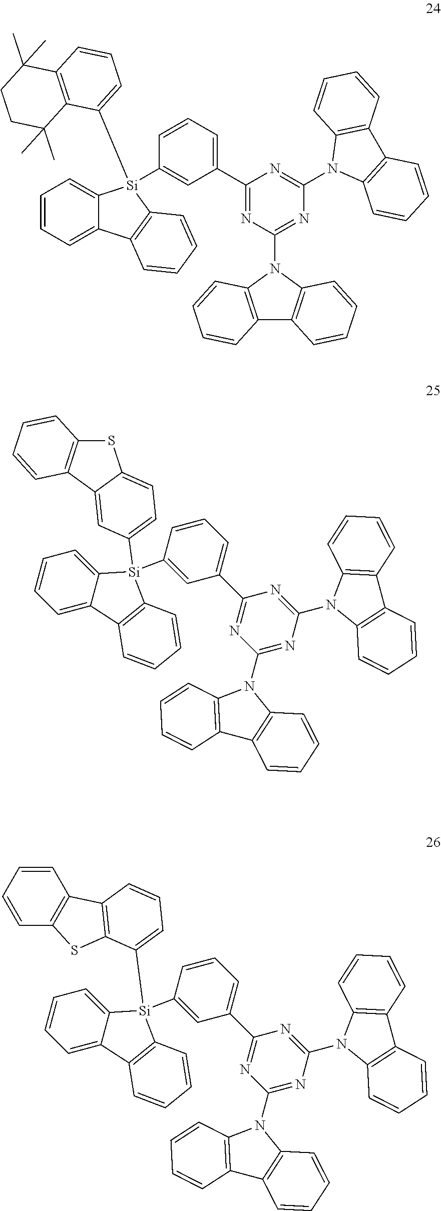 [SMILES: CC1(C)CCC(C)(C)c2c1cccc2[Si]1(c2cccc(-c3nc(-n4c5ccccc5c5ccccc54)nc(-n4c5ccccc5c5ccccc54)n3)c2)c2ccccc2-c2ccccc21 ; c1cc(-c2nc(-n3c4ccccc4c4ccccc43)nc(-n3c4ccccc4c4ccccc43)n2)cc([Si]2(c3ccc4sc5ccccc5c4c3)c3ccccc3-c3ccccc32)c1 ; c1cc(-c2nc(-n3c4ccccc4c4ccccc43)nc(-n3c4ccccc4c4ccccc43)n2)cc([Si]2(c3cccc4c3sc3ccccc34)c3ccccc3-c3ccccc32)c1]

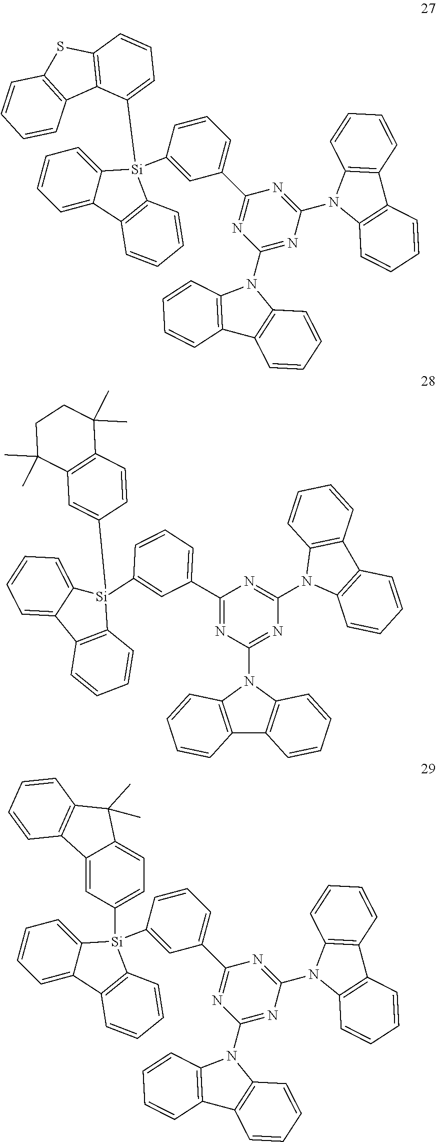 [SMILES: c1cc(-c2nc(-n3c4ccccc4c4ccccc43)nc(-n3c4ccccc4c4ccccc43)n2)cc([Si]2(c3cccc4sc5ccccc5c34)c3ccccc3-c3ccccc32)c1 ; CC1(C)CCC(C)(C)c2cc([Si]3(c4cccc(-c5nc(-n6c7ccccc7c7ccccc76)nc(-n6c7ccccc7c7ccccc76)n5)c4)c4ccccc4-c4ccccc43)ccc21 ; CC1(C)c2ccccc2-c2cc([Si]3(c4cccc(-c5nc(-n6c7ccccc7c7ccccc76)nc(-n6c7ccccc7c7ccccc76)n5)c4)c4ccccc4-c4ccccc43)ccc21]

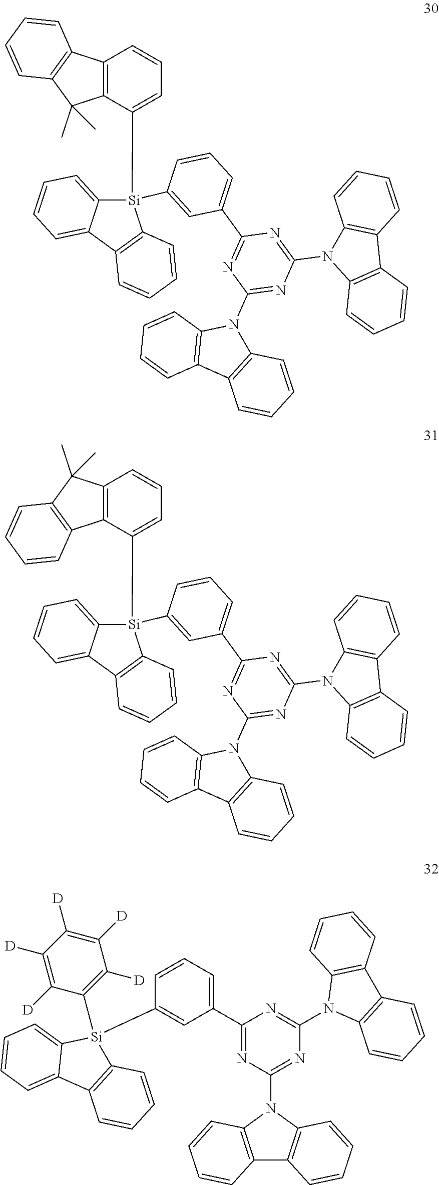 [SMILES: CC1(C)c2ccccc2-c2cccc([Si]3(c4cccc(-c5nc(-n6c7ccccc7c7ccccc76)nc(-n6c7ccccc7c7ccccc76)n5)c4)c4ccccc4-c4ccccc43)c21 ; CC1(C)c2ccccc2-c2c1cccc2[Si]1(c2cccc(-c3nc(-n4c5ccccc5c5ccccc54)nc(-n4c5ccccc5c5ccccc54)n3)c2)c2ccccc2-c2ccccc21 ; [2H]c1c([2H])c([2H])c([Si]2(c3cccc(-c4nc(-n5c6ccccc6c6ccccc65)nc(-n5c6ccccc6c6ccccc65)n4)c3)c3ccccc3-c3ccccc32)c([2H])c1[2H]]

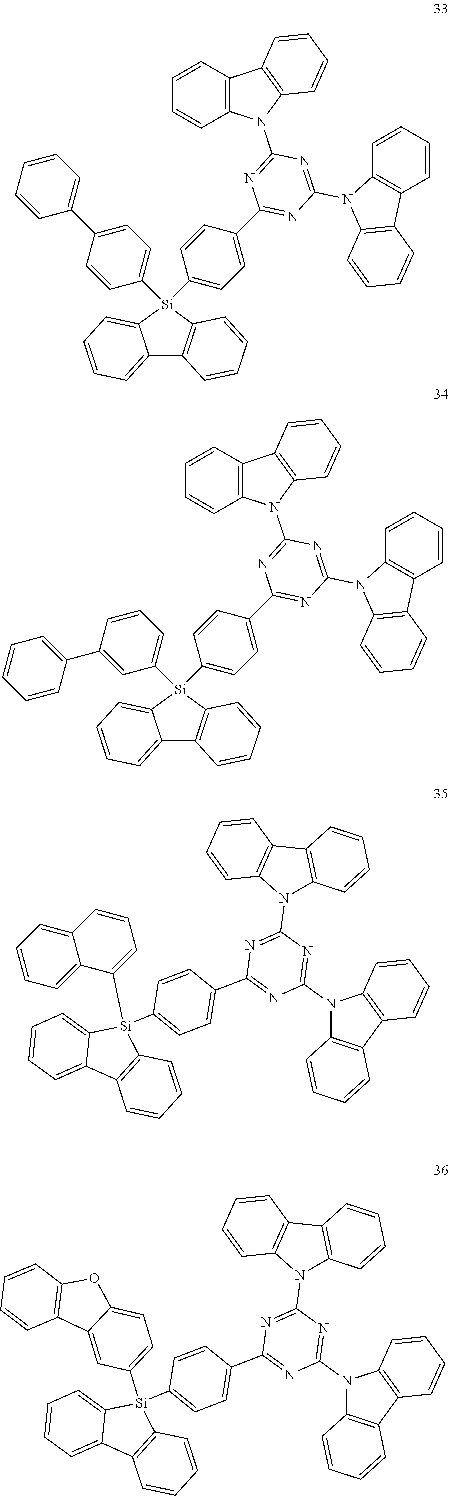 [SMILES: c1ccc(-c2ccc([Si]3(c4ccc(-c5nc(-n6c7ccccc7c7ccccc76)nc(-n6c7ccccc7c7ccccc76)n5)cc4)c4ccccc4-c4ccccc43)cc2)cc1 ; c1ccc(-c2cccc([Si]3(c4ccc(-c5nc(-n6c7ccccc7c7ccccc76)nc(-n6c7ccccc7c7ccccc76)n5)cc4)c4ccccc4-c4ccccc43)c2)cc1 ; c1ccc2c(c1)-c1ccccc1[Si]2(c1ccc(-c2nc(-n3c4ccccc4c4ccccc43)nc(-n3c4ccccc4c4ccccc43)n2)cc1)c1cccc2ccccc12 ; c1ccc2c(c1)-c1ccccc1[Si]2(c1ccc(-c2nc(-n3c4ccccc4c4ccccc43)nc(-n3c4ccccc4c4ccccc43)n2)cc1)c1ccc2oc3ccccc3c2c1]

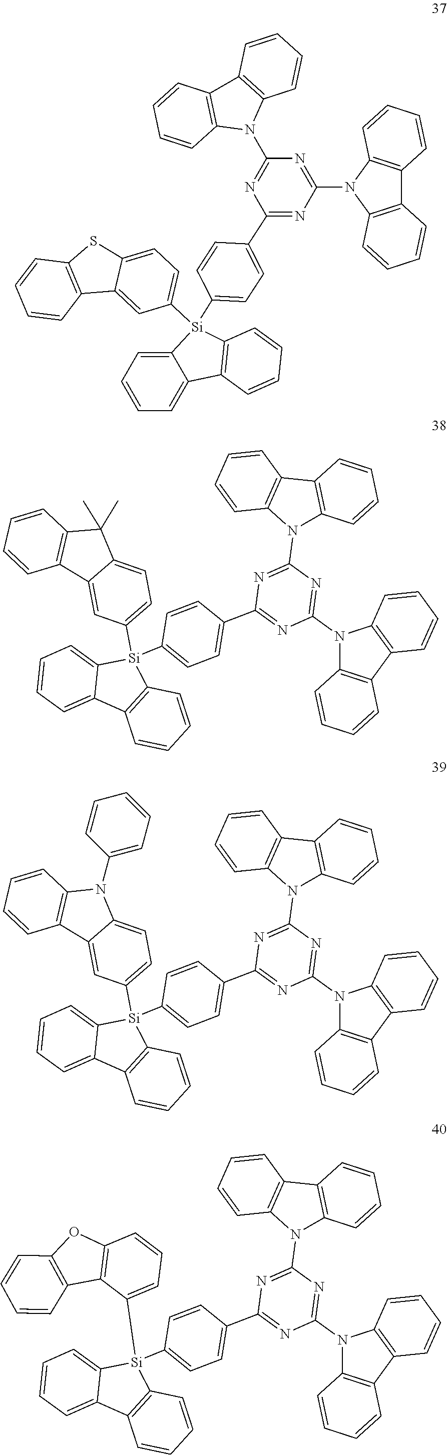 [SMILES: c1ccc2c(c1)-c1ccccc1[Si]2(c1ccc(-c2nc(-n3c4ccccc4c4ccccc43)nc(-n3c4ccccc4c4ccccc43)n2)cc1)c1ccc2sc3ccccc3c2c1 ; CC1(C)c2ccccc2-c2cc([Si]3(c4ccc(-c5nc(-n6c7ccccc7c7ccccc76)nc(-n6c7ccccc7c7ccccc76)n5)cc4)c4ccccc4-c4ccccc43)ccc21 ; c1ccc(-n2c3ccccc3c3cc([Si]4(c5ccc(-c6nc(-n7c8ccccc8c8ccccc87)nc(-n7c8ccccc8c8ccccc87)n6)cc5)c5ccccc5-c5ccccc54)ccc32)cc1 ; c1ccc2c(c1)-c1ccccc1[Si]2(c1ccc(-c2nc(-n3c4ccccc4c4ccccc43)nc(-n3c4ccccc4c4ccccc43)n2)cc1)c1cccc2oc3ccccc3c12]

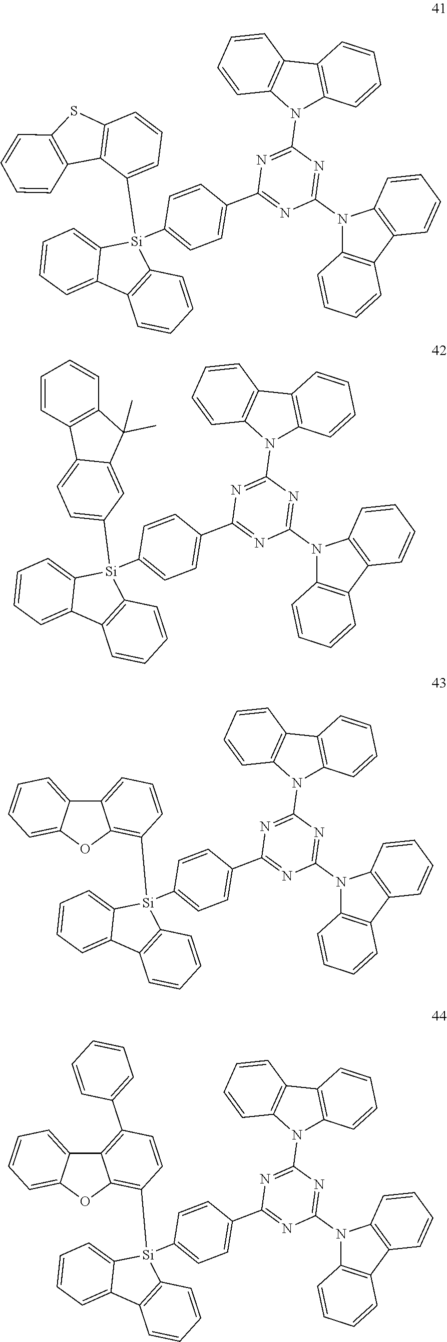 [SMILES: c1ccc2c(c1)-c1ccccc1[Si]2(c1ccc(-c2nc(-n3c4ccccc4c4ccccc43)nc(-n3c4ccccc4c4ccccc43)n2)cc1)c1cccc2sc3ccccc3c12 ; CC1(C)c2ccccc2-c2ccc([Si]3(c4ccc(-c5nc(-n6c7ccccc7c7ccccc76)nc(-n6c7ccccc7c7ccccc76)n5)cc4)c4ccccc4-c4ccccc43)cc21 ; c1ccc2c(c1)-c1ccccc1[Si]2(c1ccc(-c2nc(-n3c4ccccc4c4ccccc43)nc(-n3c4ccccc4c4ccccc43)n2)cc1)c1cccc2c1oc1ccccc12 ; c1ccc(-c2ccc([Si]3(c4ccc(-c5nc(-n6c7ccccc7c7ccccc76)nc(-n6c7ccccc7c7ccccc76)n5)cc4)c4ccccc4-c4ccccc43)c3oc4ccccc4c23)cc1]

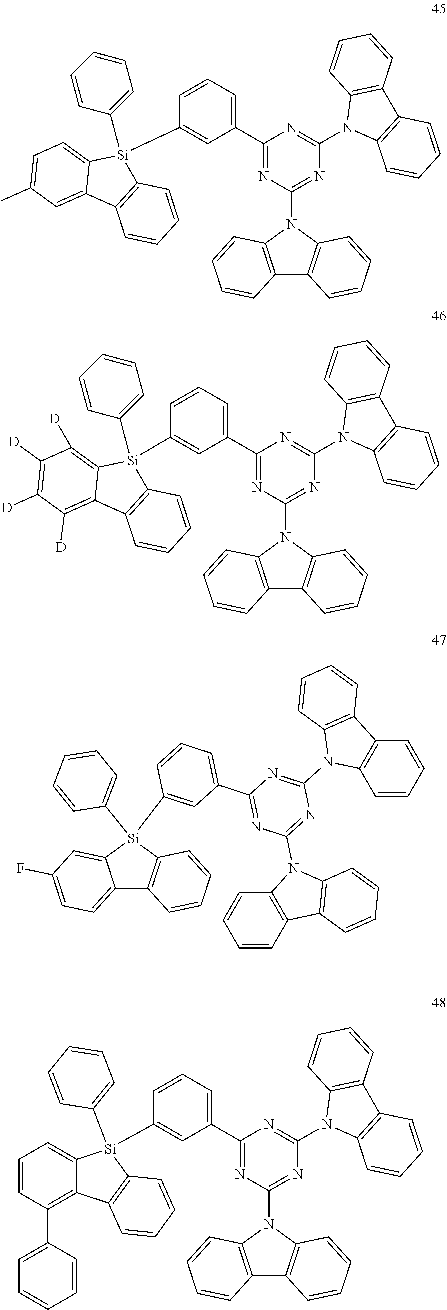 [SMILES: Cc1ccc2c(c1)-c1ccccc1[Si]2(c1ccccc1)c1cccc(-c2nc(-n3c4ccccc4c4ccccc43)nc(-n3c4ccccc4c4ccccc43)n2)c1 ; [2H]c1c([2H])c([2H])c2c(c1[2H])-c1ccccc1[Si]2(c1ccccc1)c1cccc(-c2nc(-n3c4ccccc4c4ccccc43)nc(-n3c4ccccc4c4ccccc43)n2)c1 ; Fc1ccc2c(c1)[Si](c1ccccc1)(c1cccc(-c3nc(-n4c5ccccc5c5ccccc54)nc(-n4c5ccccc5c5ccccc54)n3)c1)c1ccccc1-2 ; c1ccc(-c2cccc3c2-c2ccccc2[Si]3(c2ccccc2)c2cccc(-c3nc(-n4c5ccccc5c5ccccc54)nc(-n4c5ccccc5c5ccccc54)n3)c2)cc1]

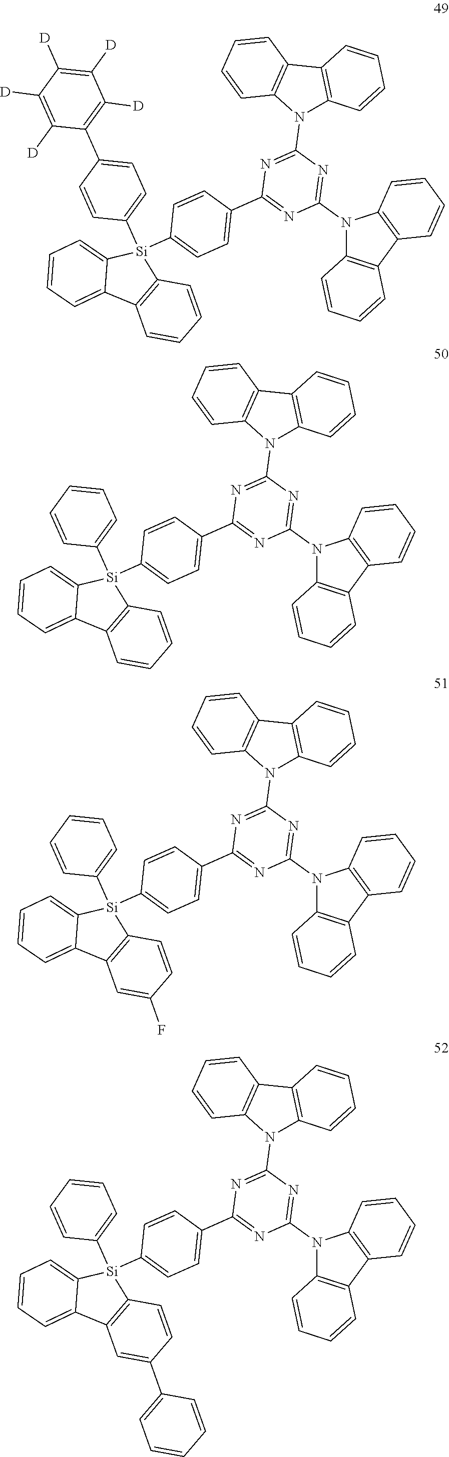

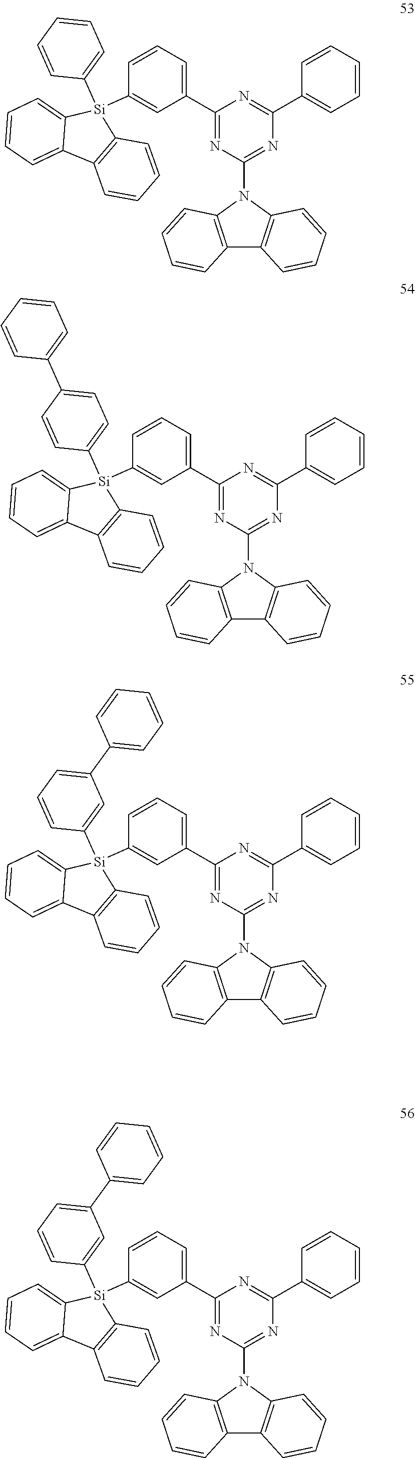 [SMILES: c1ccc(-c2nc(-c3cccc([Si]4(c5ccccc5)c5ccccc5-c5ccccc54)c3)nc(-n3c4ccccc4c4ccccc43)n2)cc1 ; c1ccc(-c2ccc([Si]3(c4cccc(-c5nc(-c6ccccc6)nc(-n6c7ccccc7c7ccccc76)n5)c4)c4ccccc4-c4ccccc43)cc2)cc1 ; c1ccc(-c2cccc([Si]3(c4cccc(-c5nc(-c6ccccc6)nc(-n6c7ccccc7c7ccccc76)n5)c4)c4ccccc4-c4ccccc43)c2)cc1 ; c1ccc(-c2cccc([Si]3(c4cccc(-c5nc(-c6ccccc6)nc(-n6c7ccccc7c7ccccc76)n5)c4)c4ccccc4-c4ccccc43)c2)cc1]

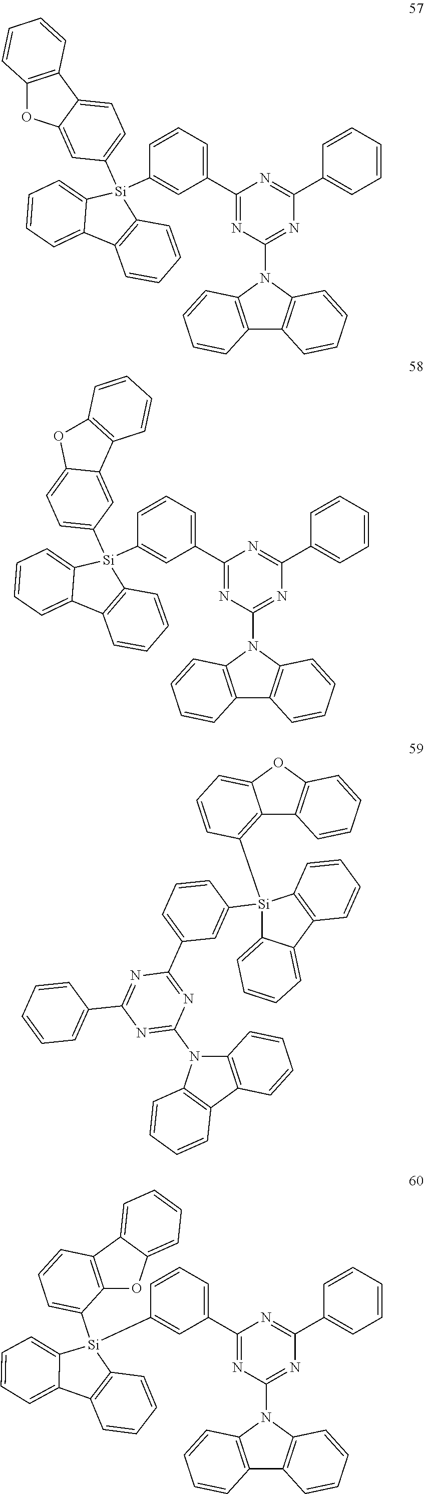

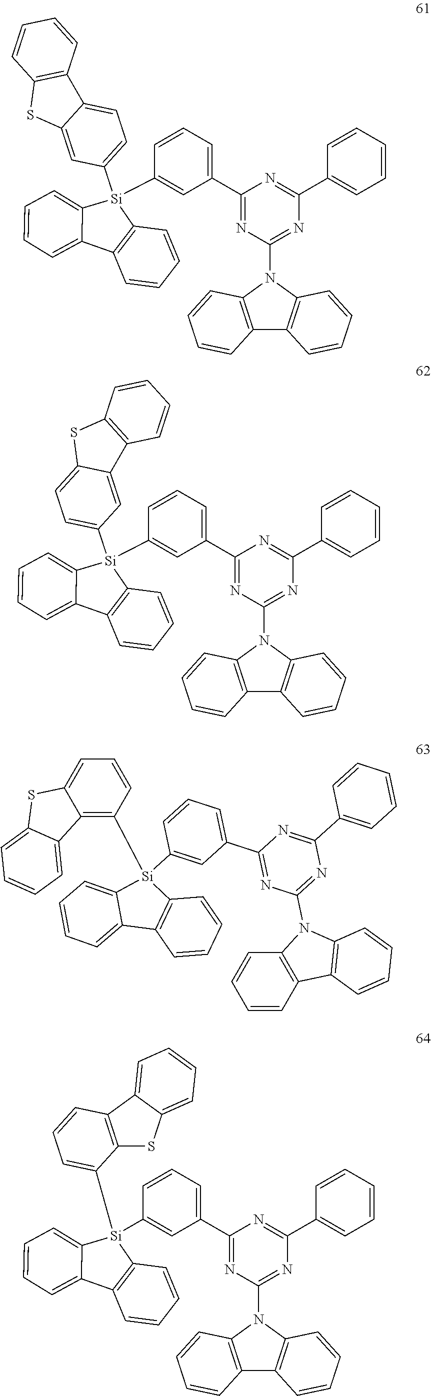 [SMILES: c1ccc(-c2nc(-c3cccc([Si]4(c5ccc6c(c5)sc5ccccc56)c5ccccc5-c5ccccc54)c3)nc(-n3c4ccccc4c4ccccc43)n2)cc1 ; c1ccc(-c2nc(-c3cccc([Si]4(c5ccc6sc7ccccc7c6c5)c5ccccc5-c5ccccc54)c3)nc(-n3c4ccccc4c4ccccc43)n2)cc1 ; c1ccc(-c2nc(-c3cccc([Si]4(c5cccc6sc7ccccc7c56)c5ccccc5-c5ccccc54)c3)nc(-n3c4ccccc4c4ccccc43)n2)cc1 ; c1ccc(-c2nc(-c3cccc([Si]4(c5cccc6c5sc5ccccc56)c5ccccc5-c5ccccc54)c3)nc(-n3c4ccccc4c4ccccc43)n2)cc1]

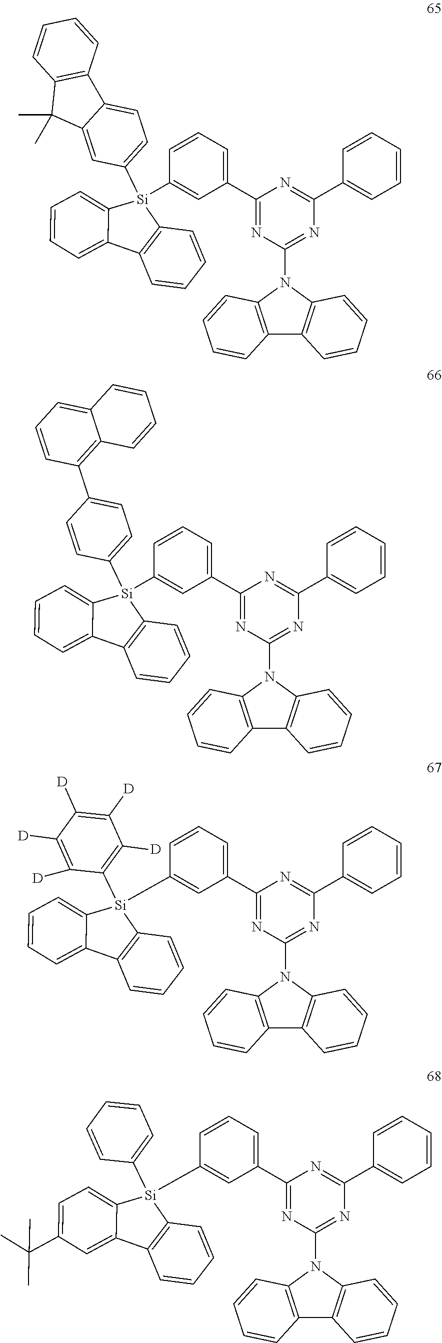 [SMILES: CC1(C)c2ccccc2-c2ccc([Si]3(c4cccc(-c5nc(-c6ccccc6)nc(-n6c7ccccc7c7ccccc76)n5)c4)c4ccccc4-c4ccccc43)cc21 ; c1ccc(-c2nc(-c3cccc([Si]4(c5ccc(-c6cccc7ccccc67)cc5)c5ccccc5-c5ccccc54)c3)nc(-n3c4ccccc4c4ccccc43)n2)cc1 ; [2H]c1c([2H])c([2H])c([Si]2(c3cccc(-c4nc(-c5ccccc5)nc(-n5c6ccccc6c6ccccc65)n4)c3)c3ccccc3-c3ccccc32)c([2H])c1[2H] ; CC(C)(C)c1ccc2c(c1)-c1ccccc1[Si]2(c1ccccc1)c1cccc(-c2nc(-c3ccccc3)nc(-n3c4ccccc4c4ccccc43)n2)c1]

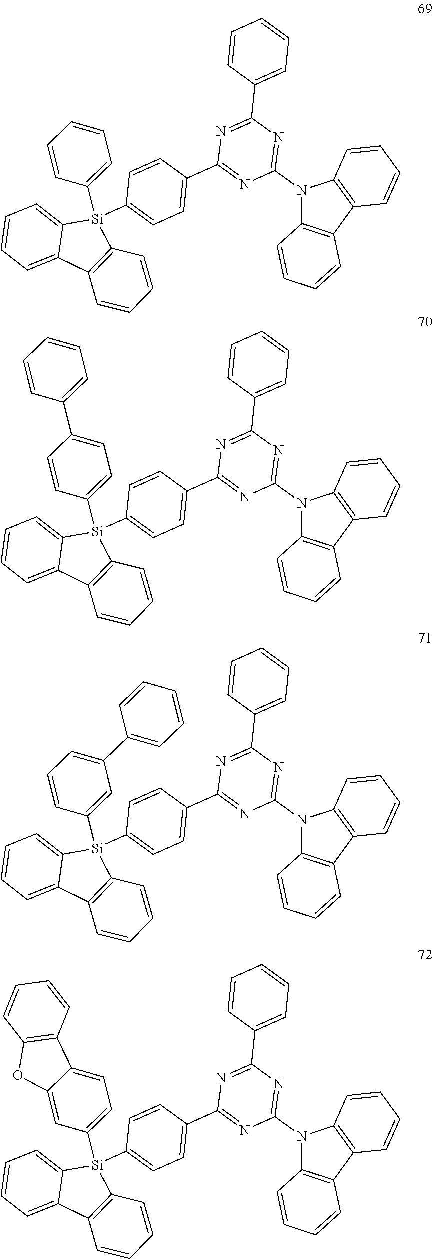

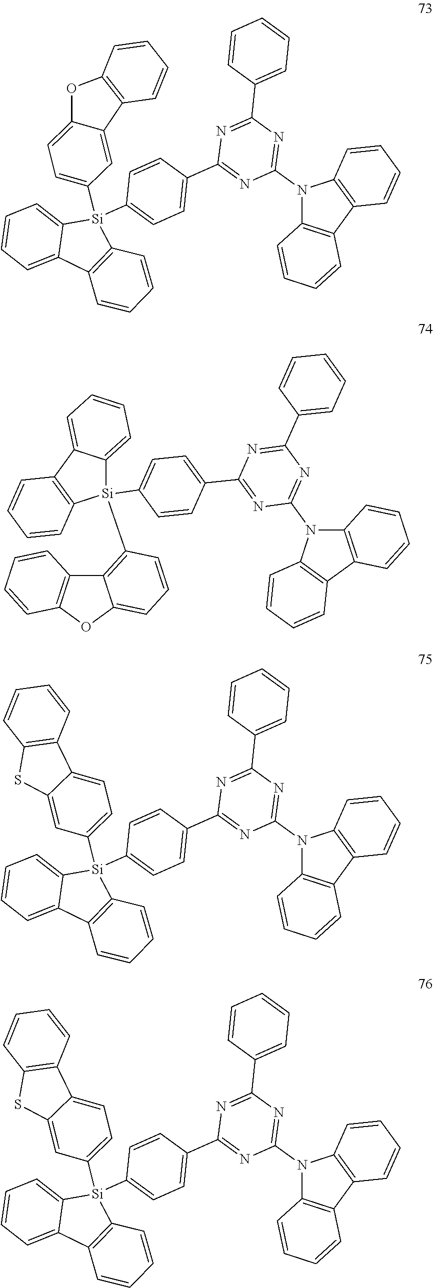 [SMILES: c1ccc(-c2nc(-c3ccc([Si]4(c5ccc6oc7ccccc7c6c5)c5ccccc5-c5ccccc54)cc3)nc(-n3c4ccccc4c4ccccc43)n2)cc1 ; c1ccc(-c2nc(-c3ccc([Si]4(c5cccc6oc7ccccc7c56)c5ccccc5-c5ccccc54)cc3)nc(-n3c4ccccc4c4ccccc43)n2)cc1 ; c1ccc(-c2nc(-c3ccc([Si]4(c5ccc6c(c5)sc5ccccc56)c5ccccc5-c5ccccc54)cc3)nc(-n3c4ccccc4c4ccccc43)n2)cc1 ; c1ccc(-c2nc(-c3ccc([Si]4(c5ccc6c(c5)sc5ccccc56)c5ccccc5-c5ccccc54)cc3)nc(-n3c4ccccc4c4ccccc43)n2)cc1]

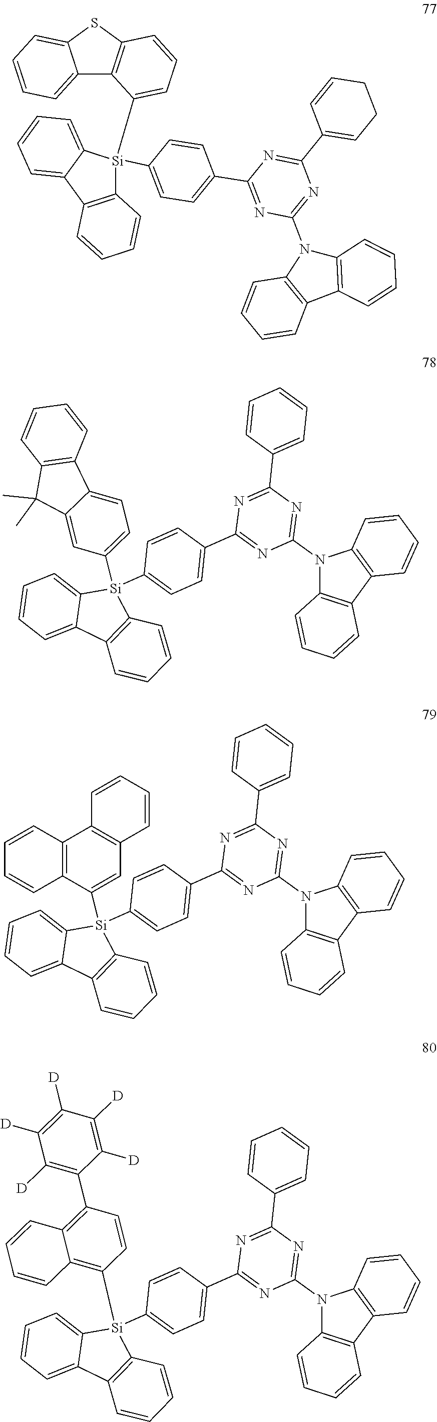 [SMILES: C1=CC(c2nc(-c3ccc([Si]4(c5cccc6sc7ccccc7c56)c5ccccc5-c5ccccc54)cc3)nc(-n3c4ccccc4c4ccccc43)n2)=CCC1 ; CC1(C)c2ccccc2-c2ccc([Si]3(c4ccc(-c5nc(-c6ccccc6)nc(-n6c7ccccc7c7ccccc76)n5)cc4)c4ccccc4-c4ccccc43)cc21 ; c1ccc(-c2nc(-c3ccc([Si]4(c5cc6ccccc6c6ccccc56)c5ccccc5-c5ccccc54)cc3)nc(-n3c4ccccc4c4ccccc43)n2)cc1 ; [2H]c1c([2H])c([2H])c(-c2ccc([Si]3(c4ccc(-c5nc(-c6ccccc6)nc(-n6c7ccccc7c7ccccc76)n5)cc4)c4ccccc4-c4ccccc43)c3ccccc23)c([2H])c1[2H]]

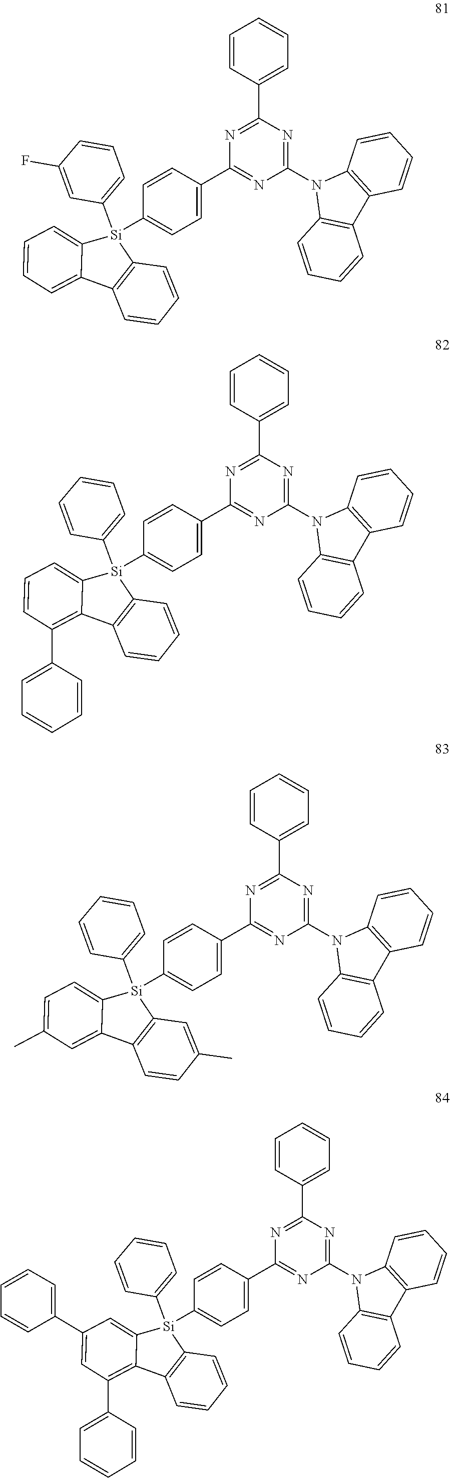 [SMILES: Fc1cccc([Si]2(c3ccc(-c4nc(-c5ccccc5)nc(-n5c6ccccc6c6ccccc65)n4)cc3)c3ccccc3-c3ccccc32)c1 ; c1ccc(-c2nc(-c3ccc([Si]4(c5ccccc5)c5ccccc5-c5c(-c6ccccc6)cccc54)cc3)nc(-n3c4ccccc4c4ccccc43)n2)cc1 ; Cc1ccc2c(c1)-c1ccc(C)cc1[Si]2(c1ccccc1)c1ccc(-c2nc(-c3ccccc3)nc(-n3c4ccccc4c4ccccc43)n2)cc1 ; c1ccc(-c2cc(-c3ccccc3)c3c(c2)[Si](c2ccccc2)(c2ccc(-c4nc(-c5ccccc5)nc(-n5c6ccccc6c6ccccc65)n4)cc2)c2ccccc2-3)cc1]

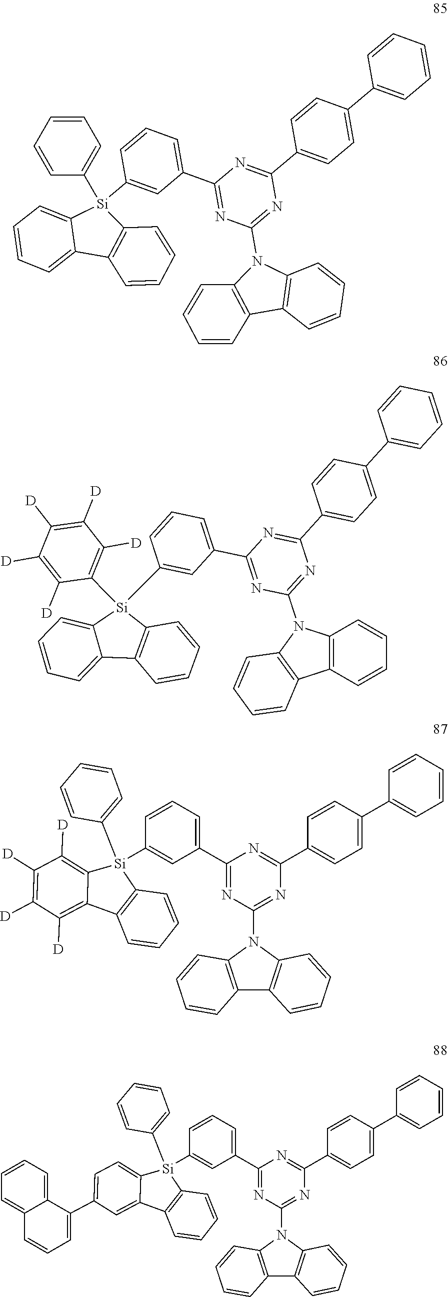 [SMILES: c1ccc(-c2ccc(-c3nc(-c4cccc([Si]5(c6ccccc6)c6ccccc6-c6ccccc65)c4)nc(-n4c5ccccc5c5ccccc54)n3)cc2)cc1 ; [2H]c1c([2H])c([2H])c([Si]2(c3cccc(-c4nc(-c5ccc(-c6ccccc6)cc5)nc(-n5c6ccccc6c6ccccc65)n4)c3)c3ccccc3-c3ccccc32)c([2H])c1[2H] ; [2H]c1c([2H])c([2H])c2c(c1[2H])-c1ccccc1[Si]2(c1ccccc1)c1cccc(-c2nc(-c3ccc(-c4ccccc4)cc3)nc(-n3c4ccccc4c4ccccc43)n2)c1 ; c1ccc(-c2ccc(-c3nc(-c4cccc([Si]5(c6ccccc6)c6ccccc6-c6cc(-c7cccc8ccccc78)ccc65)c4)nc(-n4c5ccccc5c5ccccc54)n3)cc2)cc1]

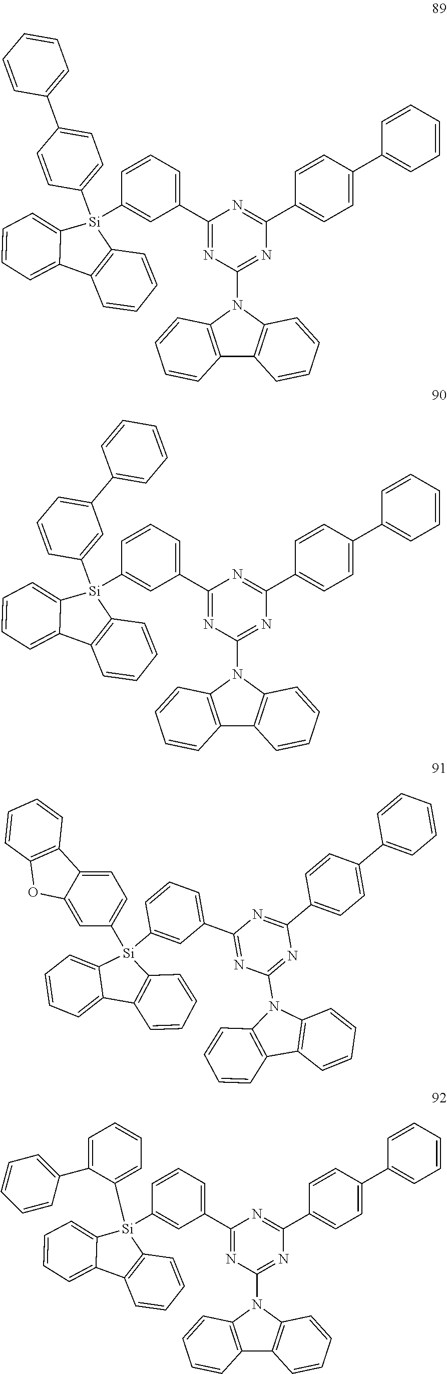 [SMILES: c1ccc(-c2ccc(-c3nc(-c4cccc([Si]5(c6ccc(-c7ccccc7)cc6)c6ccccc6-c6ccccc65)c4)nc(-n4c5ccccc5c5ccccc54)n3)cc2)cc1 ; c1ccc(-c2ccc(-c3nc(-c4cccc([Si]5(c6cccc(-c7ccccc7)c6)c6ccccc6-c6ccccc65)c4)nc(-n4c5ccccc5c5ccccc54)n3)cc2)cc1 ; c1ccc(-c2ccc(-c3nc(-c4cccc([Si]5(c6ccc7c(c6)oc6ccccc67)c6ccccc6-c6ccccc65)c4)nc(-n4c5ccccc5c5ccccc54)n3)cc2)cc1 ; c1ccc(-c2ccc(-c3nc(-c4cccc([Si]5(c6ccccc6-c6ccccc6)c6ccccc6-c6ccccc65)c4)nc(-n4c5ccccc5c5ccccc54)n3)cc2)cc1]

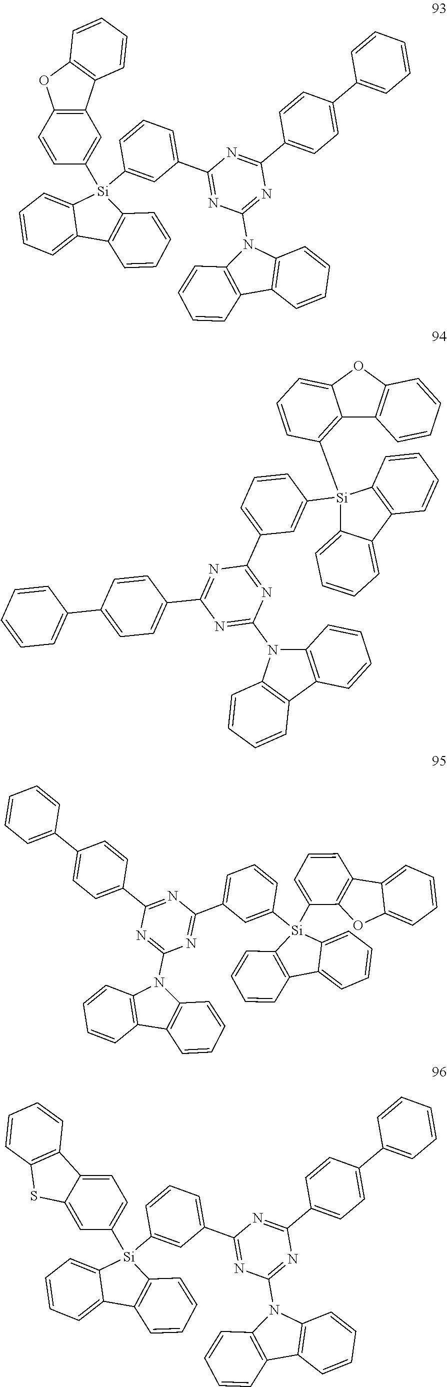 [SMILES: c1ccc(-c2ccc(-c3nc(-c4cccc([Si]5(c6ccc7oc8ccccc8c7c6)c6ccccc6-c6ccccc65)c4)nc(-n4c5ccccc5c5ccccc54)n3)cc2)cc1 ; c1ccc(-c2ccc(-c3nc(-c4cccc([Si]5(c6cccc7oc8ccccc8c67)c6ccccc6-c6ccccc65)c4)nc(-n4c5ccccc5c5ccccc54)n3)cc2)cc1 ; c1ccc(-c2ccc(-c3nc(-c4cccc([Si]5(c6cccc7c6oc6ccccc67)c6ccccc6-c6ccccc65)c4)nc(-n4c5ccccc5c5ccccc54)n3)cc2)cc1 ; c1ccc(-c2ccc(-c3nc(-c4cccc([Si]5(c6ccc7c(c6)sc6ccccc67)c6ccccc6-c6ccccc65)c4)nc(-n4c5ccccc5c5ccccc54)n3)cc2)cc1]

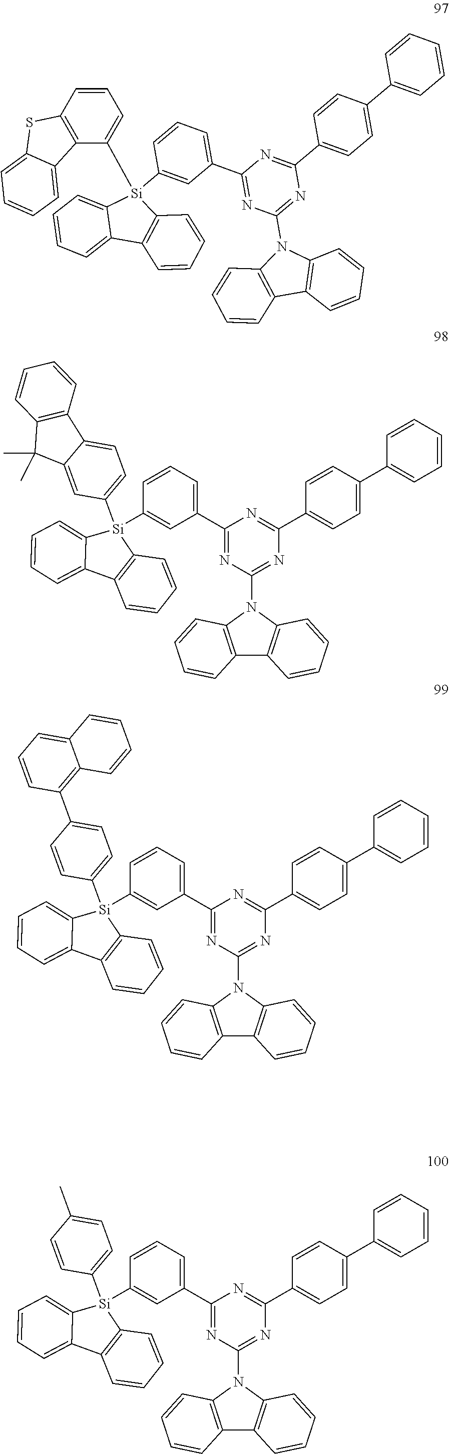 [SMILES: c1ccc(-c2ccc(-c3nc(-c4cccc([Si]5(c6cccc7sc8ccccc8c67)c6ccccc6-c6ccccc65)c4)nc(-n4c5ccccc5c5ccccc54)n3)cc2)cc1 ; CC1(C)c2ccccc2-c2ccc([Si]3(c4cccc(-c5nc(-c6ccc(-c7ccccc7)cc6)nc(-n6c7ccccc7c7ccccc76)n5)c4)c4ccccc4-c4ccccc43)cc21 ; c1ccc(-c2ccc(-c3nc(-c4cccc([Si]5(c6ccc(-c7cccc8ccccc78)cc6)c6ccccc6-c6ccccc65)c4)nc(-n4c5ccccc5c5ccccc54)n3)cc2)cc1 ; Cc1ccc([Si]2(c3cccc(-c4nc(-c5ccc(-c6ccccc6)cc5)nc(-n5c6ccccc6c6ccccc65)n4)c3)c3ccccc3-c3ccccc32)cc1]

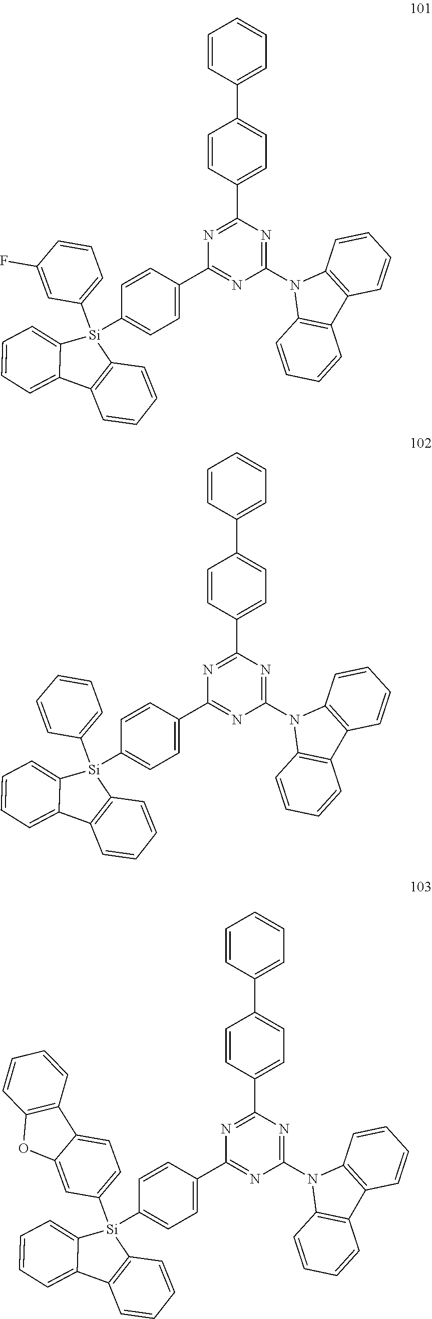 [SMILES: Fc1cccc([Si]2(c3ccc(-c4nc(-c5ccc(-c6ccccc6)cc5)nc(-n5c6ccccc6c6ccccc65)n4)cc3)c3ccccc3-c3ccccc32)c1 ; c1ccc(-c2ccc(-c3nc(-c4ccc([Si]5(c6ccccc6)c6ccccc6-c6ccccc65)cc4)nc(-n4c5ccccc5c5ccccc54)n3)cc2)cc1 ; c1ccc(-c2ccc(-c3nc(-c4ccc([Si]5(c6ccc7c(c6)oc6ccccc67)c6ccccc6-c6ccccc65)cc4)nc(-n4c5ccccc5c5ccccc54)n3)cc2)cc1]

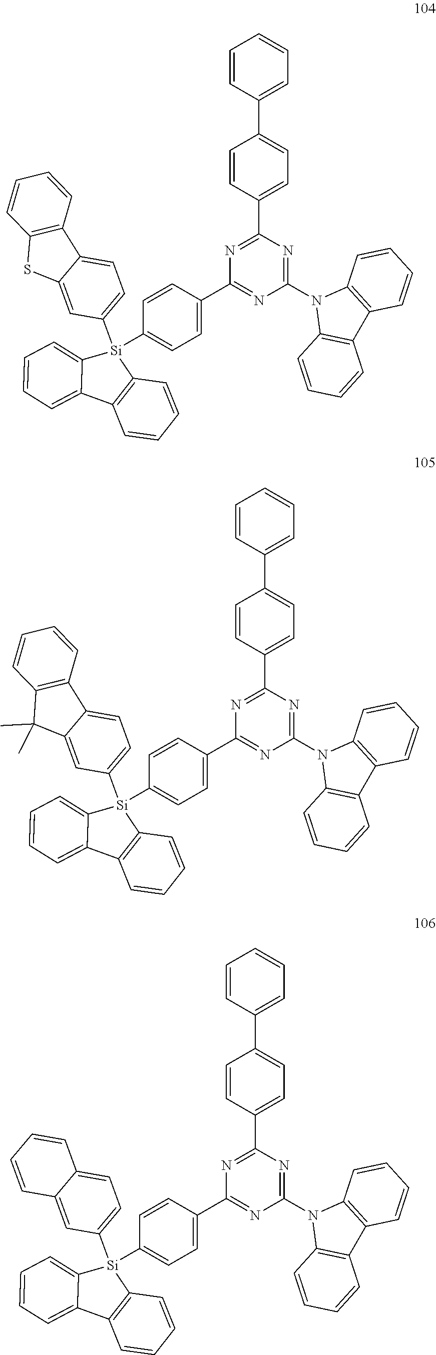 [SMILES: c1ccc(-c2ccc(-c3nc(-c4ccc([Si]5(c6ccc7c(c6)sc6ccccc67)c6ccccc6-c6ccccc65)cc4)nc(-n4c5ccccc5c5ccccc54)n3)cc2)cc1 ; CC1(C)c2ccccc2-c2ccc([Si]3(c4ccc(-c5nc(-c6ccc(-c7ccccc7)cc6)nc(-n6c7ccccc7c7ccccc76)n5)cc4)c4ccccc4-c4ccccc43)cc21 ; c1ccc(-c2ccc(-c3nc(-c4ccc([Si]5(c6ccc7ccccc7c6)c6ccccc6-c6ccccc65)cc4)nc(-n4c5ccccc5c5ccccc54)n3)cc2)cc1]

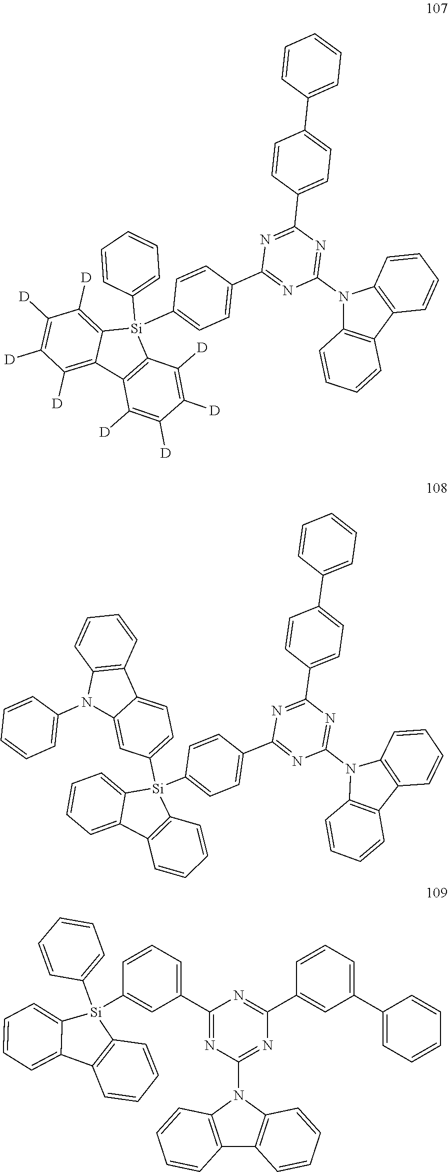 [SMILES: [2H]c1c([2H])c([2H])c2c(c1[2H])-c1c([2H])c([2H])c([2H])c([2H])c1[Si]2(c1ccccc1)c1ccc(-c2nc(-c3ccc(-c4ccccc4)cc3)nc(-n3c4ccccc4c4ccccc43)n2)cc1 ; c1ccc(-c2ccc(-c3nc(-c4ccc([Si]5(c6ccc7c8ccccc8n(-c8ccccc8)c7c6)c6ccccc6-c6ccccc65)cc4)nc(-n4c5ccccc5c5ccccc54)n3)cc2)cc1 ; c1ccc(-c2cccc(-c3nc(-c4cccc([Si]5(c6ccccc6)c6ccccc6-c6ccccc65)c4)nc(-n4c5ccccc5c5ccccc54)n3)c2)cc1]

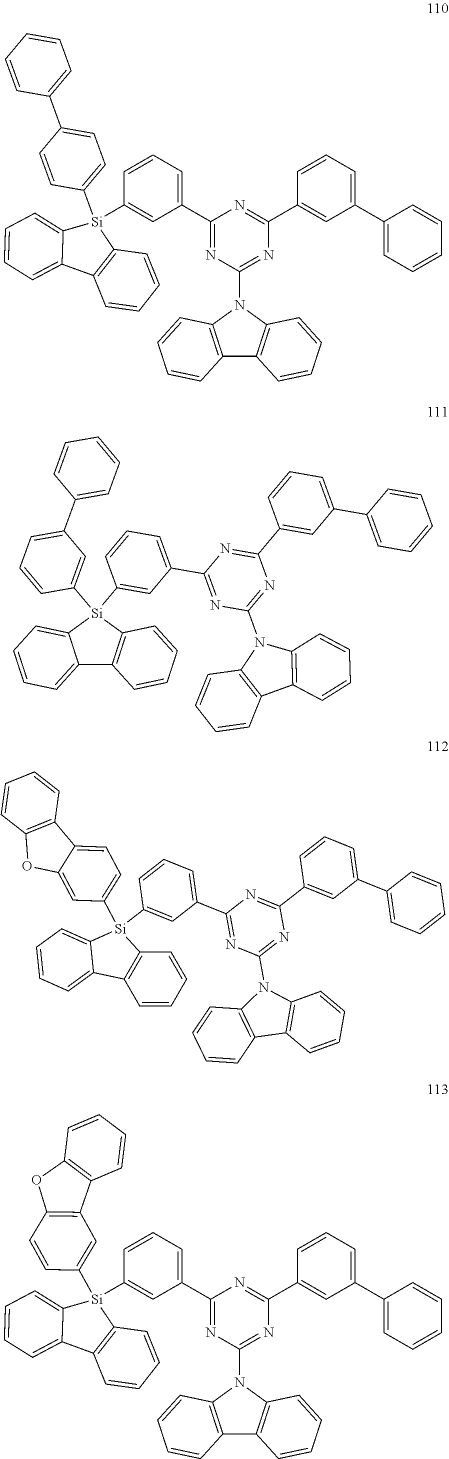 [SMILES: c1ccc(-c2ccc([Si]3(c4cccc(-c5nc(-c6cccc(-c7ccccc7)c6)nc(-n6c7ccccc7c7ccccc76)n5)c4)c4ccccc4-c4ccccc43)cc2)cc1 ; c1ccc(-c2cccc(-c3nc(-c4cccc([Si]5(c6cccc(-c7ccccc7)c6)c6ccccc6-c6ccccc65)c4)nc(-n4c5ccccc5c5ccccc54)n3)c2)cc1 ; c1ccc(-c2cccc(-c3nc(-c4cccc([Si]5(c6ccc7c(c6)oc6ccccc67)c6ccccc6-c6ccccc65)c4)nc(-n4c5ccccc5c5ccccc54)n3)c2)cc1 ; c1ccc(-c2cccc(-c3nc(-c4cccc([Si]5(c6ccc7oc8ccccc8c7c6)c6ccccc6-c6ccccc65)c4)nc(-n4c5ccccc5c5ccccc54)n3)c2)cc1]

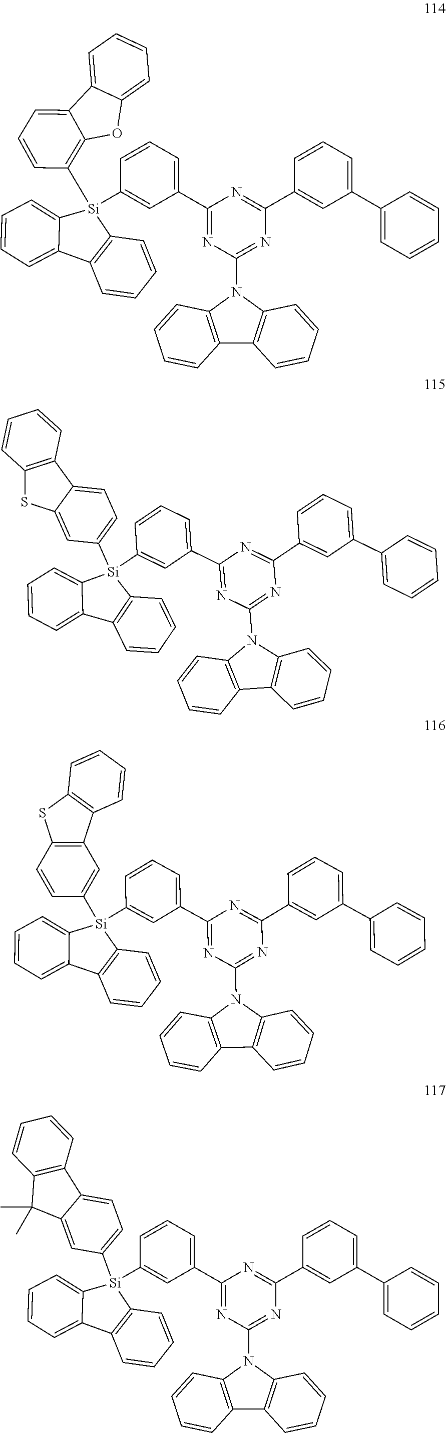 [SMILES: c1ccc(-c2cccc(-c3nc(-c4cccc([Si]5(c6cccc7c6oc6ccccc67)c6ccccc6-c6ccccc65)c4)nc(-n4c5ccccc5c5ccccc54)n3)c2)cc1 ; c1ccc(-c2cccc(-c3nc(-c4cccc([Si]5(c6ccc7c(c6)sc6ccccc67)c6ccccc6-c6ccccc65)c4)nc(-n4c5ccccc5c5ccccc54)n3)c2)cc1 ; c1ccc(-c2cccc(-c3nc(-c4cccc([Si]5(c6ccc7sc8ccccc8c7c6)c6ccccc6-c6ccccc65)c4)nc(-n4c5ccccc5c5ccccc54)n3)c2)cc1 ; CC1(C)c2ccccc2-c2ccc([Si]3(c4cccc(-c5nc(-c6cccc(-c7ccccc7)c6)nc(-n6c7ccccc7c7ccccc76)n5)c4)c4ccccc4-c4ccccc43)cc21]

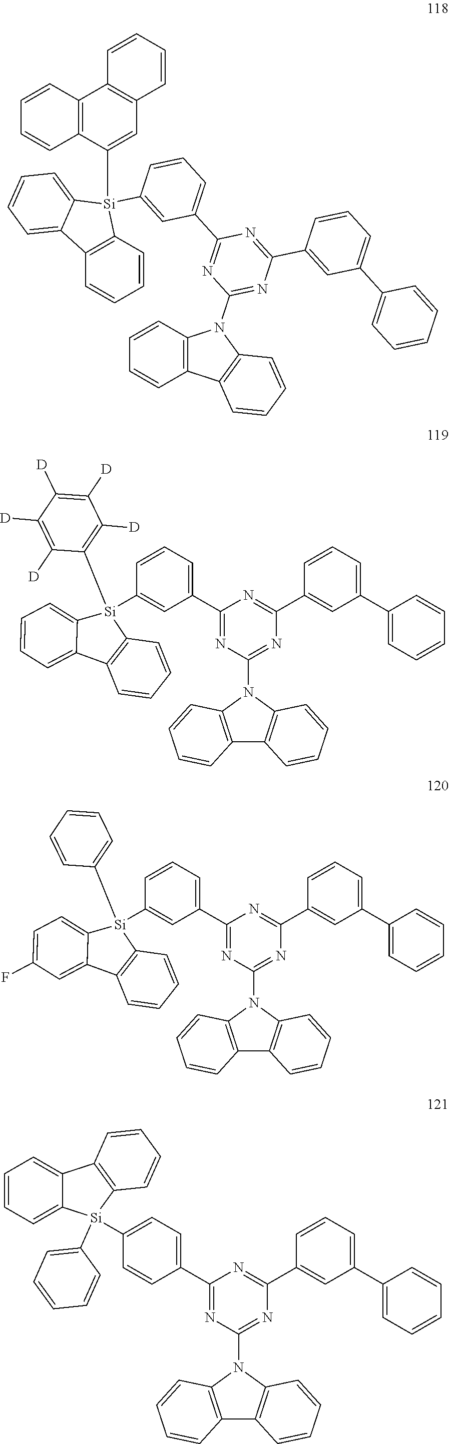 [SMILES: c1ccc(-c2cccc(-c3nc(-c4cccc([Si]5(c6cc7ccccc7c7ccccc67)c6ccccc6-c6ccccc65)c4)nc(-n4c5ccccc5c5ccccc54)n3)c2)cc1 ; [2H]c1c([2H])c([2H])c([Si]2(c3cccc(-c4nc(-c5cccc(-c6ccccc6)c5)nc(-n5c6ccccc6c6ccccc65)n4)c3)c3ccccc3-c3ccccc32)c([2H])c1[2H] ; Fc1ccc2c(c1)-c1ccccc1[Si]2(c1ccccc1)c1cccc(-c2nc(-c3cccc(-c4ccccc4)c3)nc(-n3c4ccccc4c4ccccc43)n2)c1 ; c1ccc(-c2cccc(-c3nc(-c4ccc([Si]5(c6ccccc6)c6ccccc6-c6ccccc65)cc4)nc(-n4c5ccccc5c5ccccc54)n3)c2)cc1]

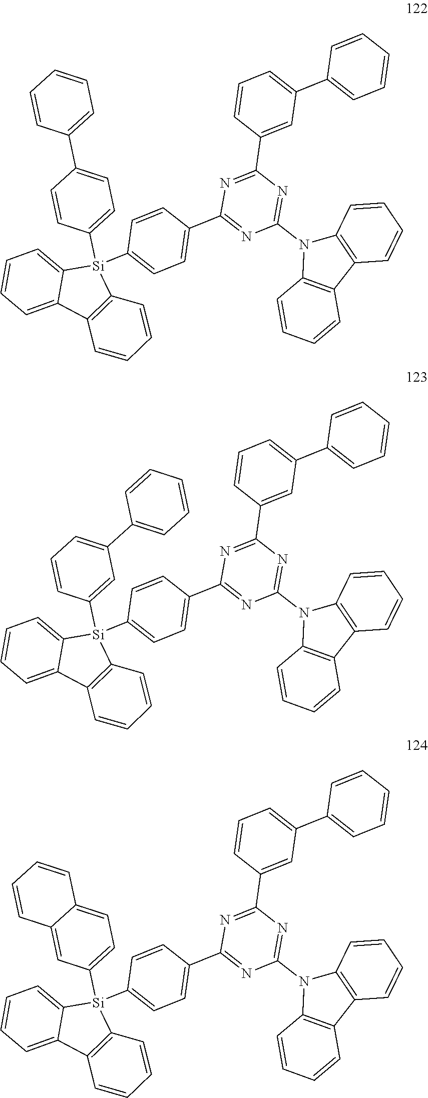 [SMILES: c1ccc(-c2ccc([Si]3(c4ccc(-c5nc(-c6cccc(-c7ccccc7)c6)nc(-n6c7ccccc7c7ccccc76)n5)cc4)c4ccccc4-c4ccccc43)cc2)cc1 ; c1ccc(-c2cccc(-c3nc(-c4ccc([Si]5(c6cccc(-c7ccccc7)c6)c6ccccc6-c6ccccc65)cc4)nc(-n4c5ccccc5c5ccccc54)n3)c2)cc1 ; c1ccc(-c2cccc(-c3nc(-c4ccc([Si]5(c6ccc7ccccc7c6)c6ccccc6-c6ccccc65)cc4)nc(-n4c5ccccc5c5ccccc54)n3)c2)cc1]

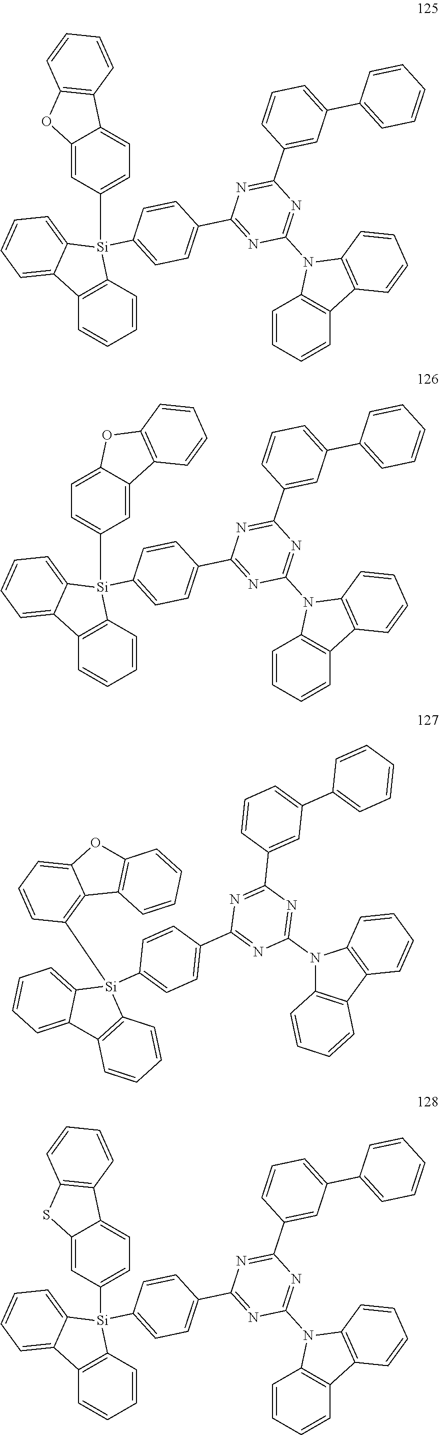 [SMILES: c1ccc(-c2cccc(-c3nc(-c4ccc([Si]5(c6ccc7c(c6)oc6ccccc67)c6ccccc6-c6ccccc65)cc4)nc(-n4c5ccccc5c5ccccc54)n3)c2)cc1 ; c1ccc(-c2cccc(-c3nc(-c4ccc([Si]5(c6ccc7oc8ccccc8c7c6)c6ccccc6-c6ccccc65)cc4)nc(-n4c5ccccc5c5ccccc54)n3)c2)cc1 ; c1ccc(-c2cccc(-c3nc(-c4ccc([Si]5(c6cccc7oc8ccccc8c67)c6ccccc6-c6ccccc65)cc4)nc(-n4c5ccccc5c5ccccc54)n3)c2)cc1 ; c1ccc(-c2cccc(-c3nc(-c4ccc([Si]5(c6ccc7c(c6)sc6ccccc67)c6ccccc6-c6ccccc65)cc4)nc(-n4c5ccccc5c5ccccc54)n3)c2)cc1]

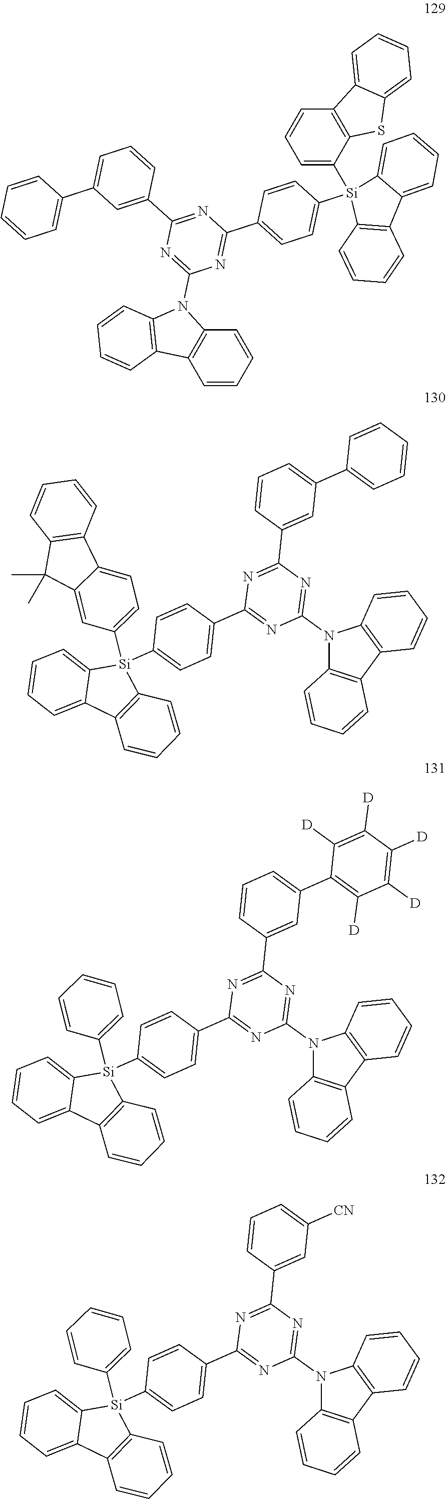 [SMILES: c1ccc(-c2cccc(-c3nc(-c4ccc([Si]5(c6cccc7c6sc6ccccc67)c6ccccc6-c6ccccc65)cc4)nc(-n4c5ccccc5c5ccccc54)n3)c2)cc1 ; CC1(C)c2ccccc2-c2ccc([Si]3(c4ccc(-c5nc(-c6cccc(-c7ccccc7)c6)nc(-n6c7ccccc7c7ccccc76)n5)cc4)c4ccccc4-c4ccccc43)cc21 ; [2H]c1c([2H])c([2H])c(-c2cccc(-c3nc(-c4ccc([Si]5(c6ccccc6)c6ccccc6-c6ccccc65)cc4)nc(-n4c5ccccc5c5ccccc54)n3)c2)c([2H])c1[2H] ; N#Cc1cccc(-c2nc(-c3ccc([Si]4(c5ccccc5)c5ccccc5-c5ccccc54)cc3)nc(-n3c4ccccc4c4ccccc43)n2)c1]

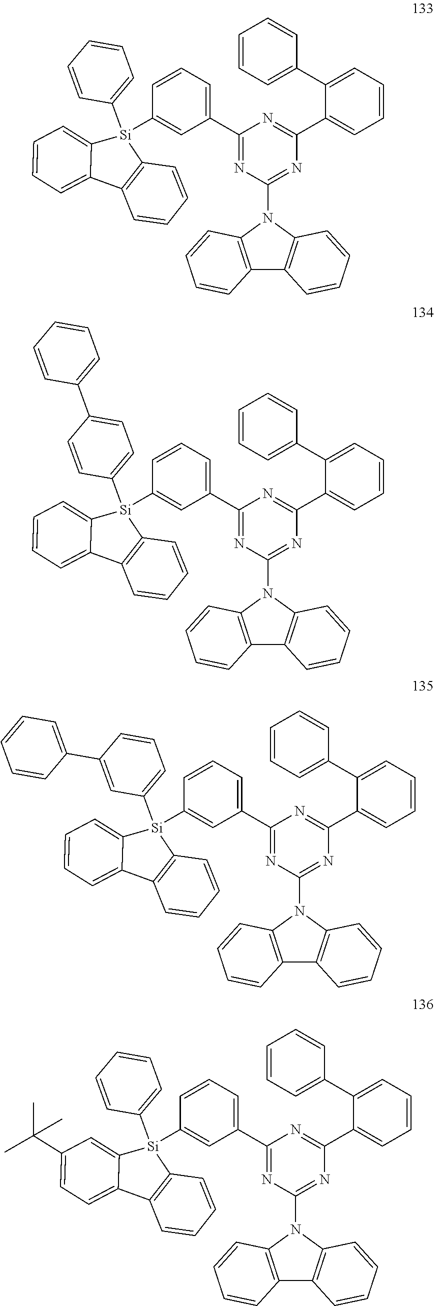

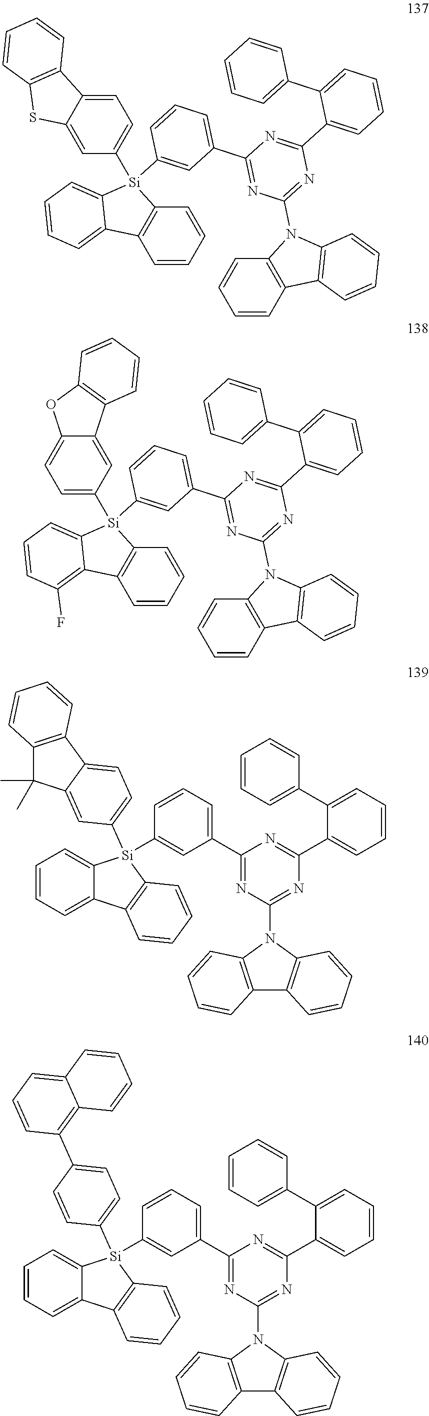 [SMILES: c1ccc(-c2ccccc2-c2nc(-c3cccc([Si]4(c5ccc6c(c5)sc5ccccc56)c5ccccc5-c5ccccc54)c3)nc(-n3c4ccccc4c4ccccc43)n2)cc1 ; Fc1cccc2c1-c1ccccc1[Si]2(c1cccc(-c2nc(-c3ccccc3-c3ccccc3)nc(-n3c4ccccc4c4ccccc43)n2)c1)c1ccc2oc3ccccc3c2c1 ; CC1(C)c2ccccc2-c2ccc([Si]3(c4cccc(-c5nc(-c6ccccc6-c6ccccc6)nc(-n6c7ccccc7c7ccccc76)n5)c4)c4ccccc4-c4ccccc43)cc21 ; c1ccc(-c2ccccc2-c2nc(-c3cccc([Si]4(c5ccc(-c6cccc7ccccc67)cc5)c5ccccc5-c5ccccc54)c3)nc(-n3c4ccccc4c4ccccc43)n2)cc1]

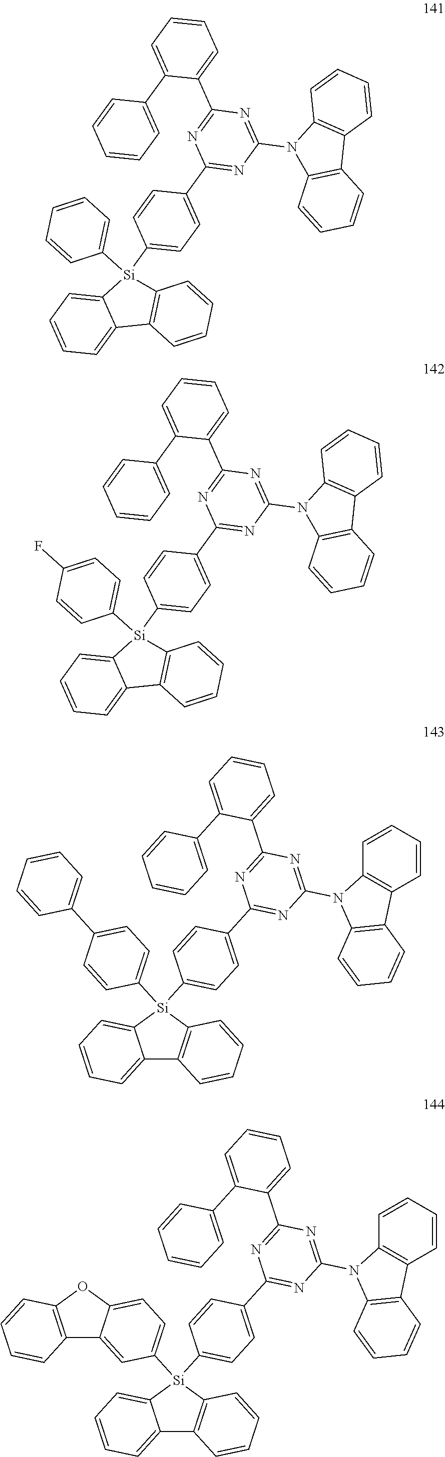

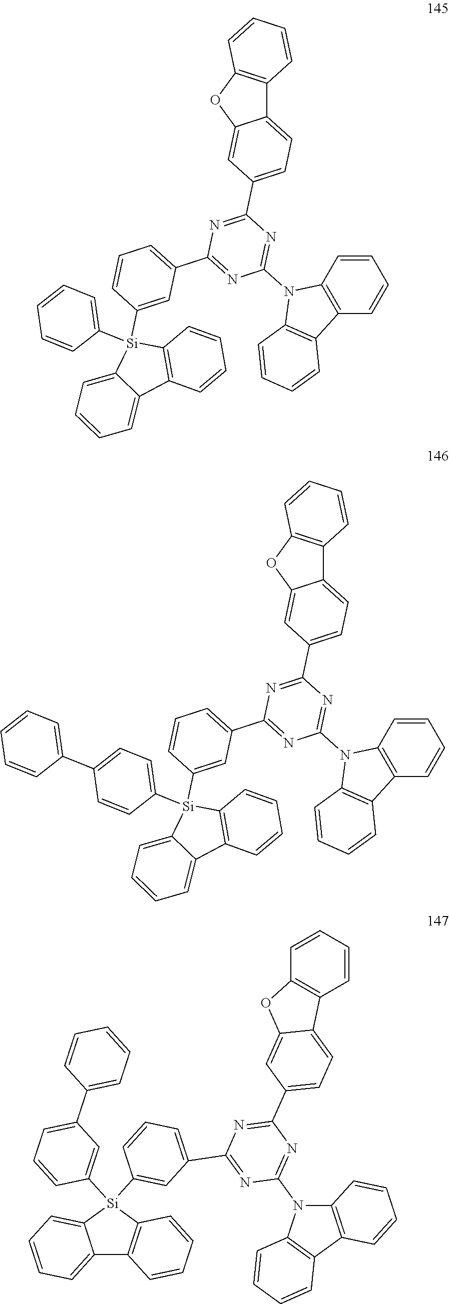 [SMILES: c1ccc([Si]2(c3cccc(-c4nc(-c5ccc6c(c5)oc5ccccc56)nc(-n5c6ccccc6c6ccccc65)n4)c3)c3ccccc3-c3ccccc32)cc1 ; c1ccc(-c2ccc([Si]3(c4cccc(-c5nc(-c6ccc7c(c6)oc6ccccc67)nc(-n6c7ccccc7c7ccccc76)n5)c4)c4ccccc4-c4ccccc43)cc2)cc1 ; c1ccc(-c2cccc([Si]3(c4cccc(-c5nc(-c6ccc7c(c6)oc6ccccc67)nc(-n6c7ccccc7c7ccccc76)n5)c4)c4ccccc4-c4ccccc43)c2)cc1]

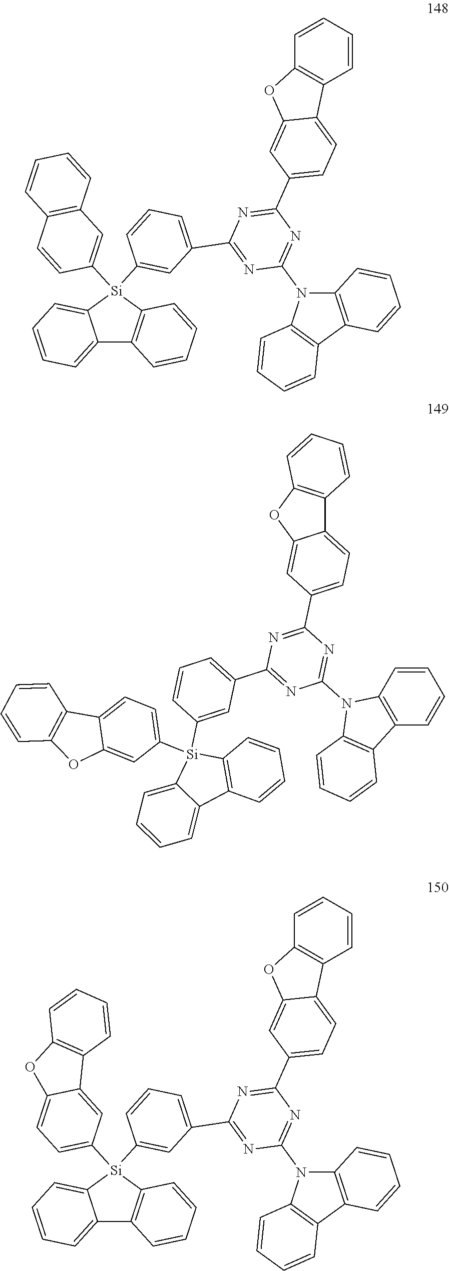 [SMILES: c1cc(-c2nc(-c3ccc4c(c3)oc3ccccc34)nc(-n3c4ccccc4c4ccccc43)n2)cc([Si]2(c3ccc4ccccc4c3)c3ccccc3-c3ccccc32)c1 ; c1cc(-c2nc(-c3ccc4c(c3)oc3ccccc34)nc(-n3c4ccccc4c4ccccc43)n2)cc([Si]2(c3ccc4c(c3)oc3ccccc34)c3ccccc3-c3ccccc32)c1 ; c1cc(-c2nc(-c3ccc4c(c3)oc3ccccc34)nc(-n3c4ccccc4c4ccccc43)n2)cc([Si]2(c3ccc4oc5ccccc5c4c3)c3ccccc3-c3ccccc32)c1]

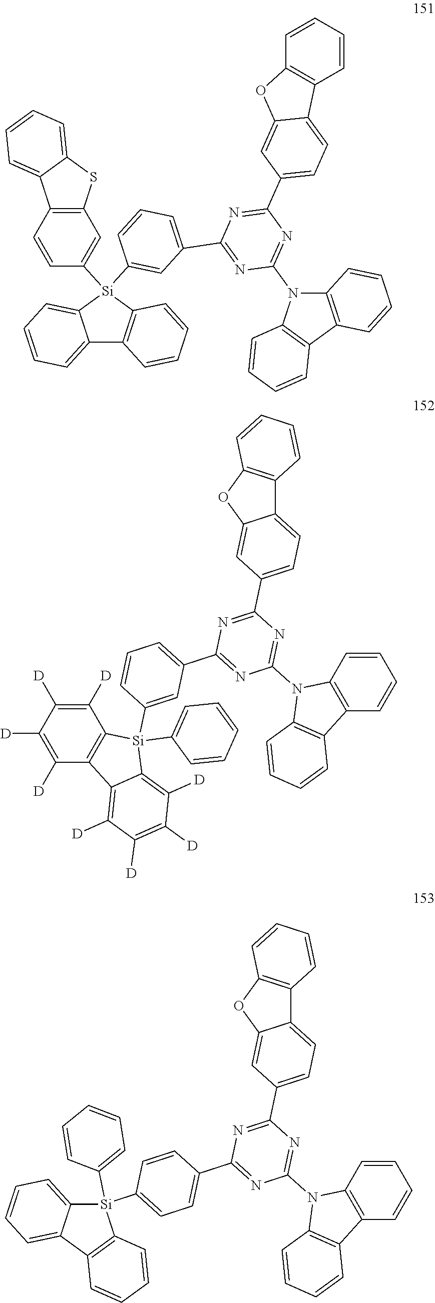

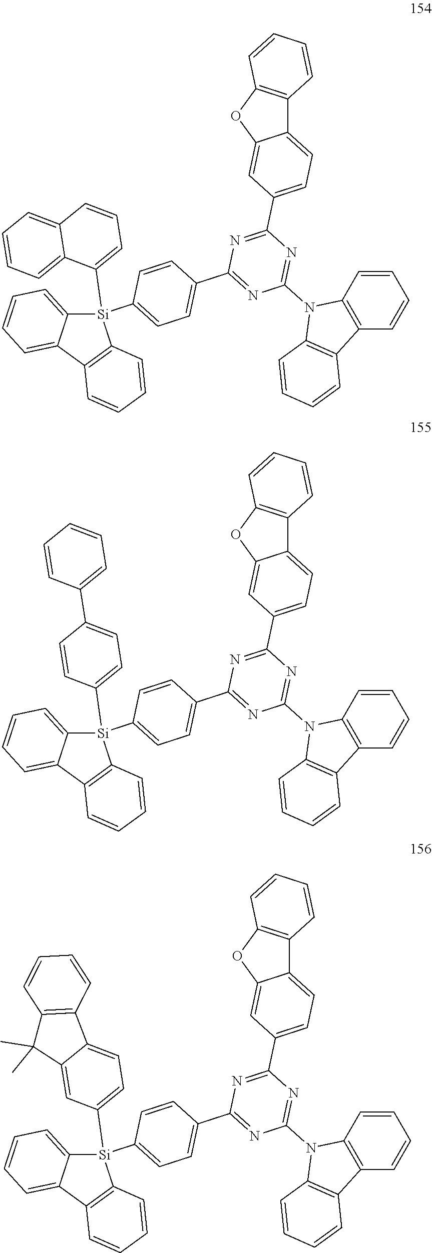 [SMILES: c1ccc2c(c1)-c1ccccc1[Si]2(c1ccc(-c2nc(-c3ccc4c(c3)oc3ccccc34)nc(-n3c4ccccc4c4ccccc43)n2)cc1)c1cccc2ccccc12 ; c1ccc(-c2ccc([Si]3(c4ccc(-c5nc(-c6ccc7c(c6)oc6ccccc67)nc(-n6c7ccccc7c7ccccc76)n5)cc4)c4ccccc4-c4ccccc43)cc2)cc1 ; CC1(C)c2ccccc2-c2ccc([Si]3(c4ccc(-c5nc(-c6ccc7c(c6)oc6ccccc67)nc(-n6c7ccccc7c7ccccc76)n5)cc4)c4ccccc4-c4ccccc43)cc21]

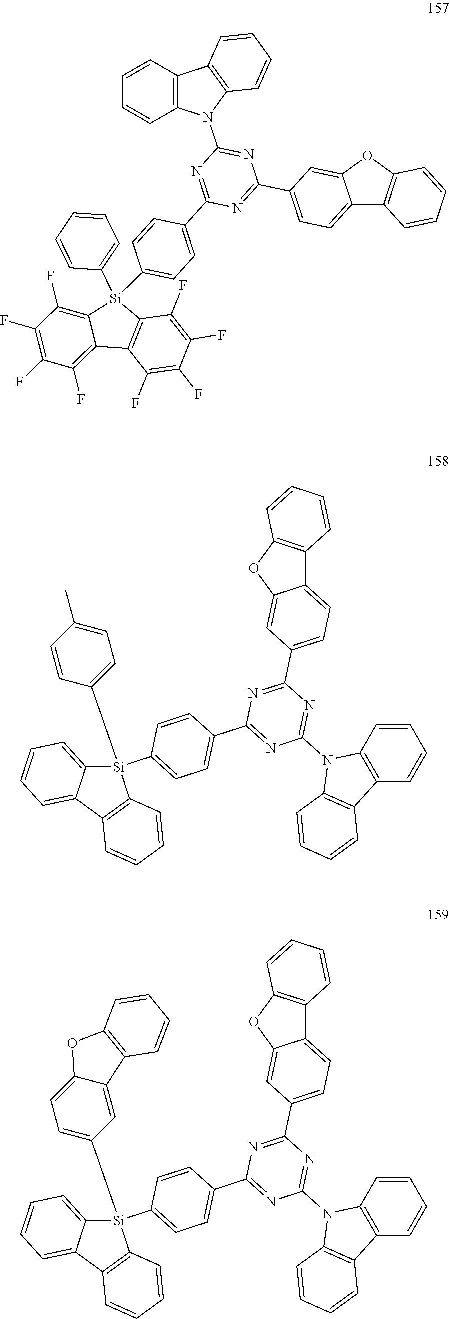 [SMILES: Fc1c(F)c(F)c2c(c1F)-c1c(F)c(F)c(F)c(F)c1[Si]2(c1ccccc1)c1ccc(-c2nc(-c3ccc4c(c3)oc3ccccc34)nc(-n3c4ccccc4c4ccccc43)n2)cc1 ; Cc1ccc([Si]2(c3ccc(-c4nc(-c5ccc6c(c5)oc5ccccc56)nc(-n5c6ccccc6c6ccccc65)n4)cc3)c3ccccc3-c3ccccc32)cc1 ; c1ccc2c(c1)-c1ccccc1[Si]2(c1ccc(-c2nc(-c3ccc4c(c3)oc3ccccc34)nc(-n3c4ccccc4c4ccccc43)n2)cc1)c1ccc2oc3ccccc3c2c1]

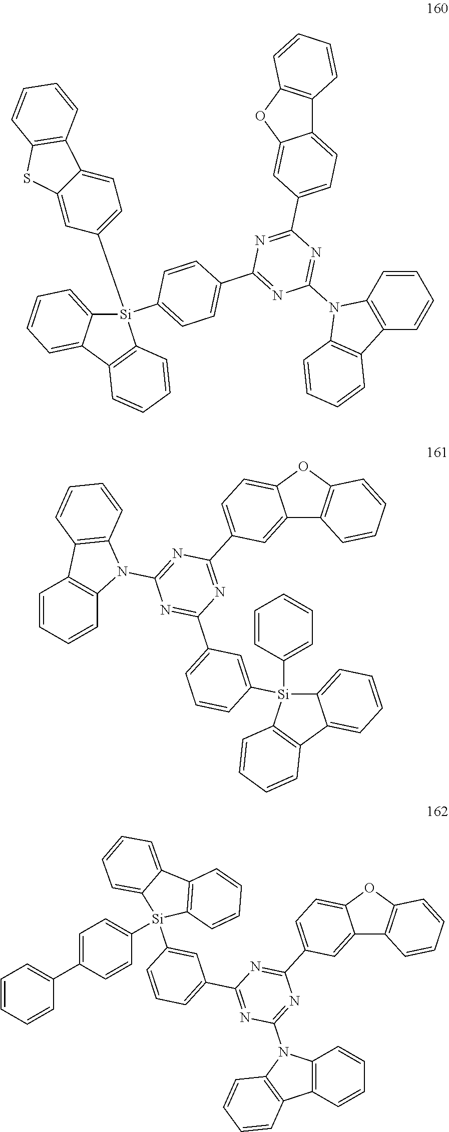 [SMILES: c1ccc2c(c1)-c1ccccc1[Si]2(c1ccc(-c2nc(-c3ccc4c(c3)oc3ccccc34)nc(-n3c4ccccc4c4ccccc43)n2)cc1)c1ccc2c(c1)sc1ccccc12 ; c1ccc([Si]2(c3cccc(-c4nc(-c5ccc6oc7ccccc7c6c5)nc(-n5c6ccccc6c6ccccc65)n4)c3)c3ccccc3-c3ccccc32)cc1 ; c1ccc(-c2ccc([Si]3(c4cccc(-c5nc(-c6ccc7oc8ccccc8c7c6)nc(-n6c7ccccc7c7ccccc76)n5)c4)c4ccccc4-c4ccccc43)cc2)cc1]

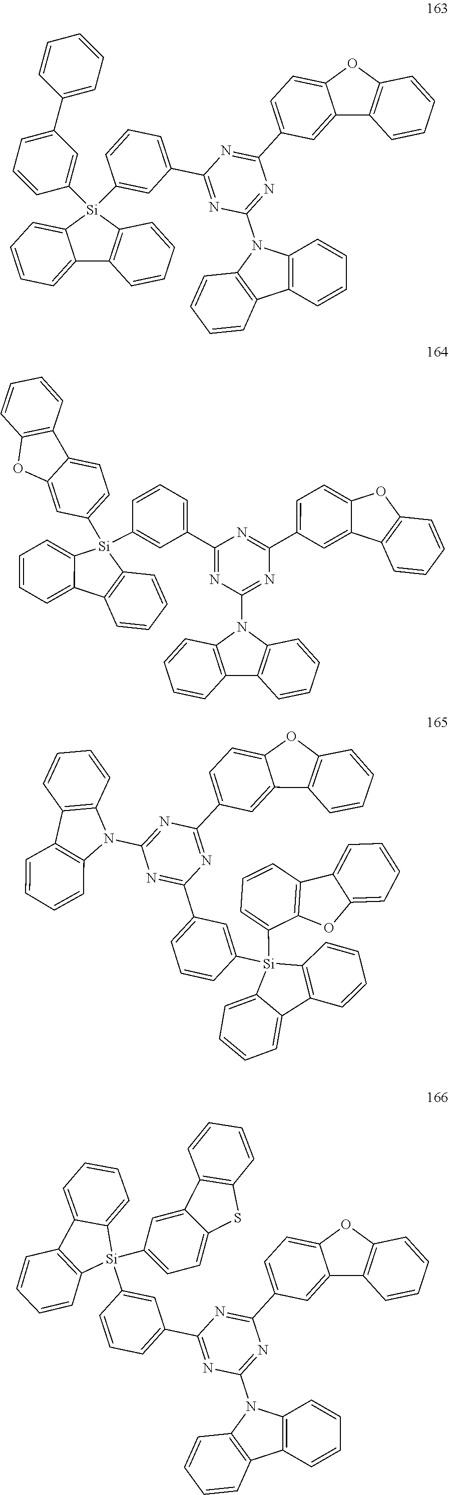

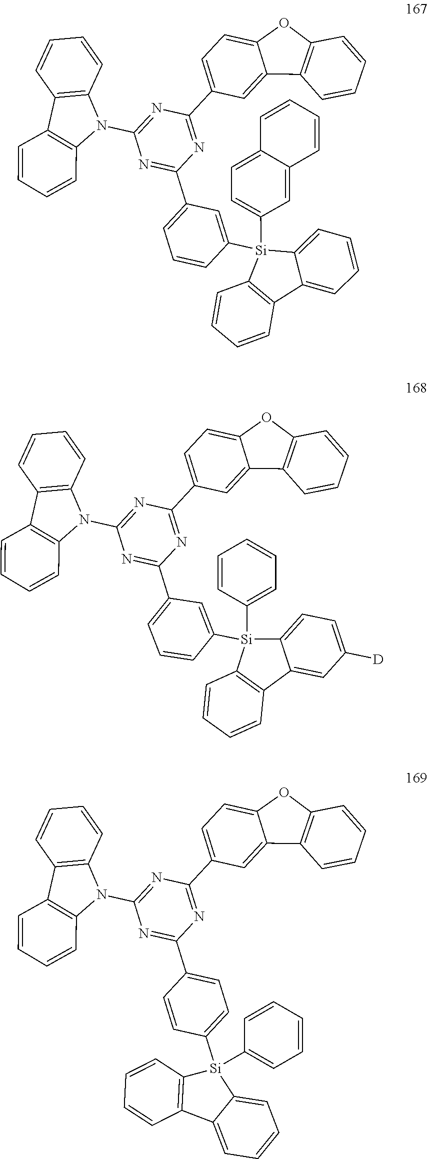 [SMILES: c1cc(-c2nc(-c3ccc4oc5ccccc5c4c3)nc(-n3c4ccccc4c4ccccc43)n2)cc([Si]2(c3ccc4ccccc4c3)c3ccccc3-c3ccccc32)c1 ; [2H]c1ccc2c(c1)-c1ccccc1[Si]2(c1ccccc1)c1cccc(-c2nc(-c3ccc4oc5ccccc5c4c3)nc(-n3c4ccccc4c4ccccc43)n2)c1 ; c1ccc([Si]2(c3ccc(-c4nc(-c5ccc6oc7ccccc7c6c5)nc(-n5c6ccccc6c6ccccc65)n4)cc3)c3ccccc3-c3ccccc32)cc1]

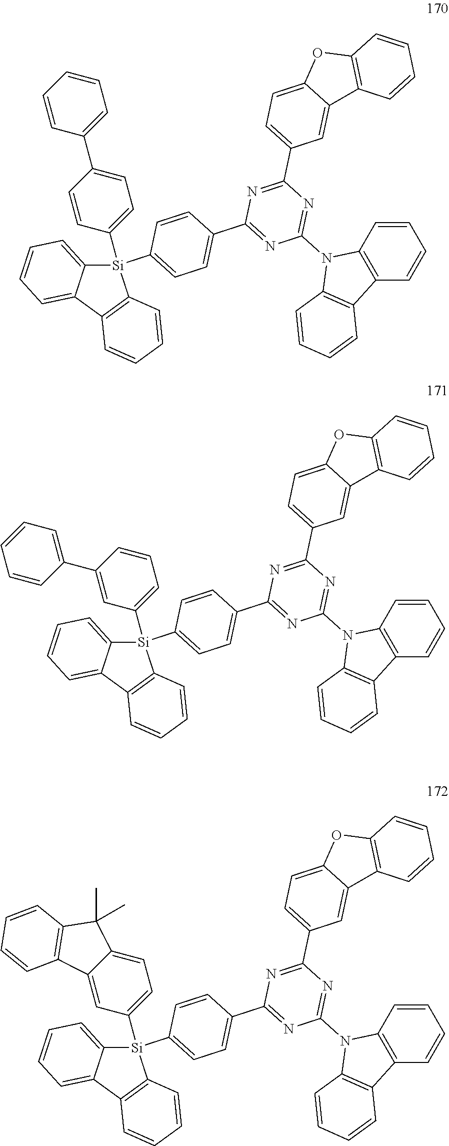 [SMILES: c1ccc(-c2ccc([Si]3(c4ccc(-c5nc(-c6ccc7oc8ccccc8c7c6)nc(-n6c7ccccc7c7ccccc76)n5)cc4)c4ccccc4-c4ccccc43)cc2)cc1 ; c1ccc(-c2cccc([Si]3(c4ccc(-c5nc(-c6ccc7oc8ccccc8c7c6)nc(-n6c7ccccc7c7ccccc76)n5)cc4)c4ccccc4-c4ccccc43)c2)cc1 ; CC1(C)c2ccccc2-c2cc([Si]3(c4ccc(-c5nc(-c6ccc7oc8ccccc8c7c6)nc(-n6c7ccccc7c7ccccc76)n5)cc4)c4ccccc4-c4ccccc43)ccc21]

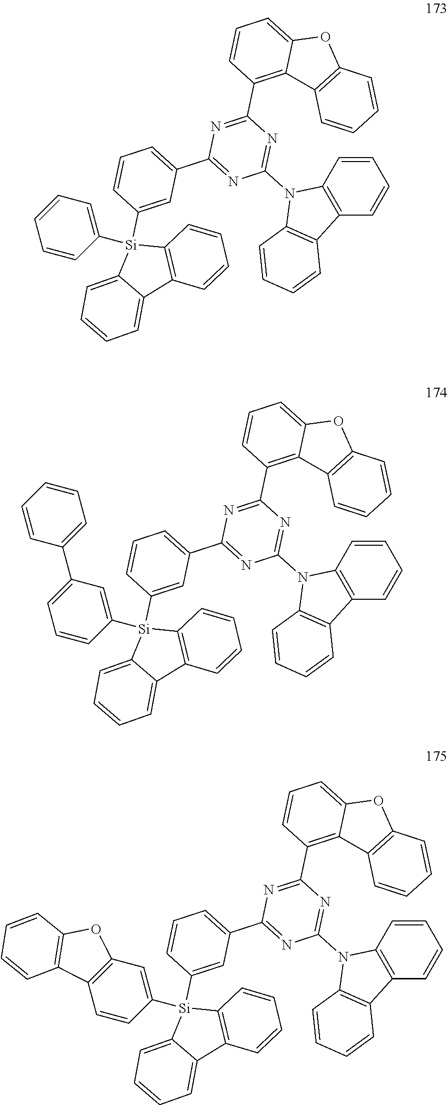 [SMILES: c1ccc([Si]2(c3cccc(-c4nc(-c5cccc6oc7ccccc7c56)nc(-n5c6ccccc6c6ccccc65)n4)c3)c3ccccc3-c3ccccc32)cc1 ; c1ccc(-c2cccc([Si]3(c4cccc(-c5nc(-c6cccc7oc8ccccc8c67)nc(-n6c7ccccc7c7ccccc76)n5)c4)c4ccccc4-c4ccccc43)c2)cc1 ; c1cc(-c2nc(-c3cccc4oc5ccccc5c34)nc(-n3c4ccccc4c4ccccc43)n2)cc([Si]2(c3ccc4c(c3)oc3ccccc34)c3ccccc3-c3ccccc32)c1]

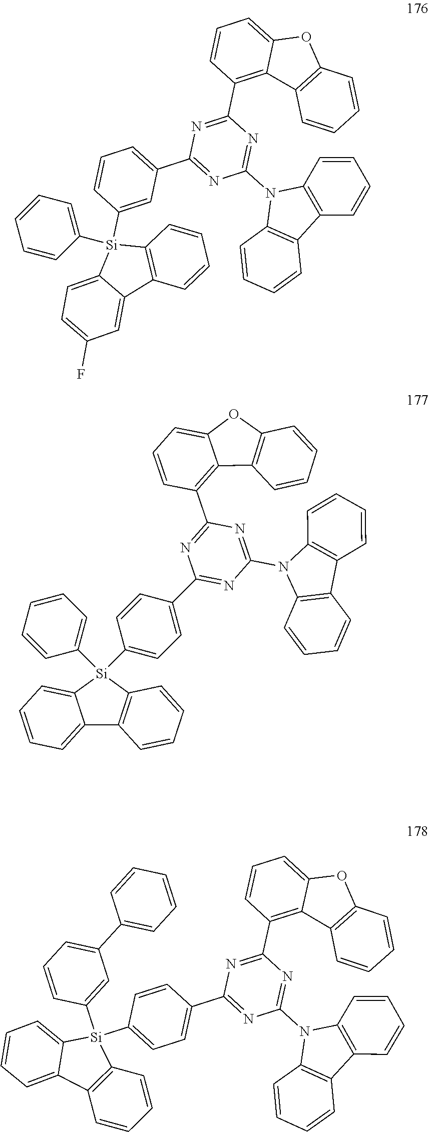 [SMILES: Fc1ccc2c(c1)-c1ccccc1[Si]2(c1ccccc1)c1cccc(-c2nc(-c3cccc4oc5ccccc5c34)nc(-n3c4ccccc4c4ccccc43)n2)c1 ; c1ccc([Si]2(c3ccc(-c4nc(-c5cccc6oc7ccccc7c56)nc(-n5c6ccccc6c6ccccc65)n4)cc3)c3ccccc3-c3ccccc32)cc1 ; c1ccc(-c2cccc([Si]3(c4ccc(-c5nc(-c6cccc7oc8ccccc8c67)nc(-n6c7ccccc7c7ccccc76)n5)cc4)c4ccccc4-c4ccccc43)c2)cc1]

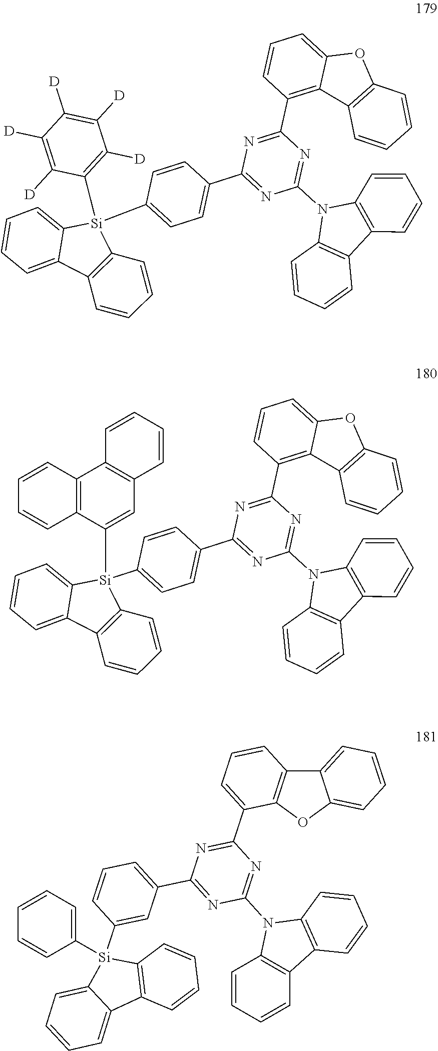 [SMILES: [2H]c1c([2H])c([2H])c([Si]2(c3ccc(-c4nc(-c5cccc6oc7ccccc7c56)nc(-n5c6ccccc6c6ccccc65)n4)cc3)c3ccccc3-c3ccccc32)c([2H])c1[2H] ; c1ccc2c(c1)-c1ccccc1[Si]2(c1ccc(-c2nc(-c3cccc4oc5ccccc5c34)nc(-n3c4ccccc4c4ccccc43)n2)cc1)c1cc2ccccc2c2ccccc12 ; c1ccc([Si]2(c3cccc(-c4nc(-c5cccc6c5oc5ccccc56)nc(-n5c6ccccc6c6ccccc65)n4)c3)c3ccccc3-c3ccccc32)cc1]

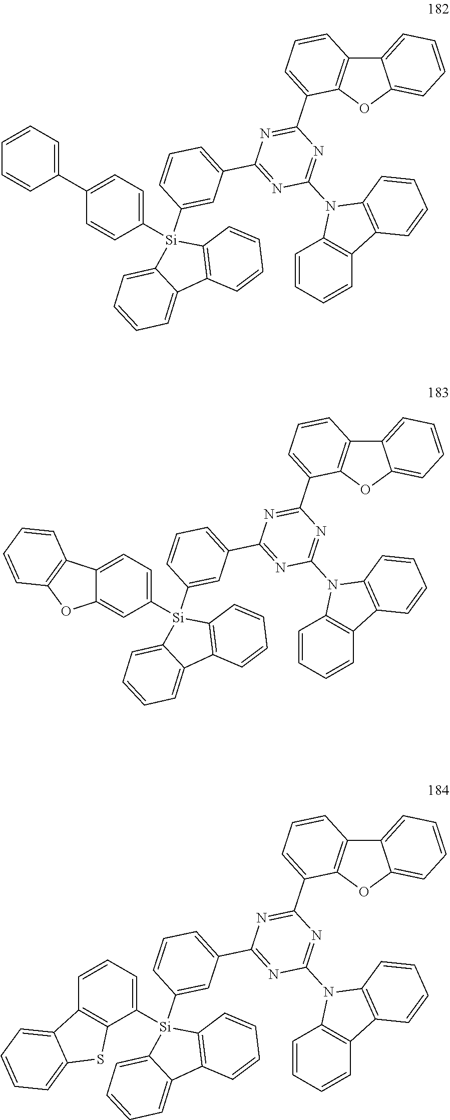 [SMILES: c1ccc(-c2ccc([Si]3(c4cccc(-c5nc(-c6cccc7c6oc6ccccc67)nc(-n6c7ccccc7c7ccccc76)n5)c4)c4ccccc4-c4ccccc43)cc2)cc1 ; c1cc(-c2nc(-c3cccc4c3oc3ccccc34)nc(-n3c4ccccc4c4ccccc43)n2)cc([Si]2(c3ccc4c(c3)oc3ccccc34)c3ccccc3-c3ccccc32)c1 ; c1cc(-c2nc(-c3cccc4c3oc3ccccc34)nc(-n3c4ccccc4c4ccccc43)n2)cc([Si]2(c3cccc4c3sc3ccccc34)c3ccccc3-c3ccccc32)c1]

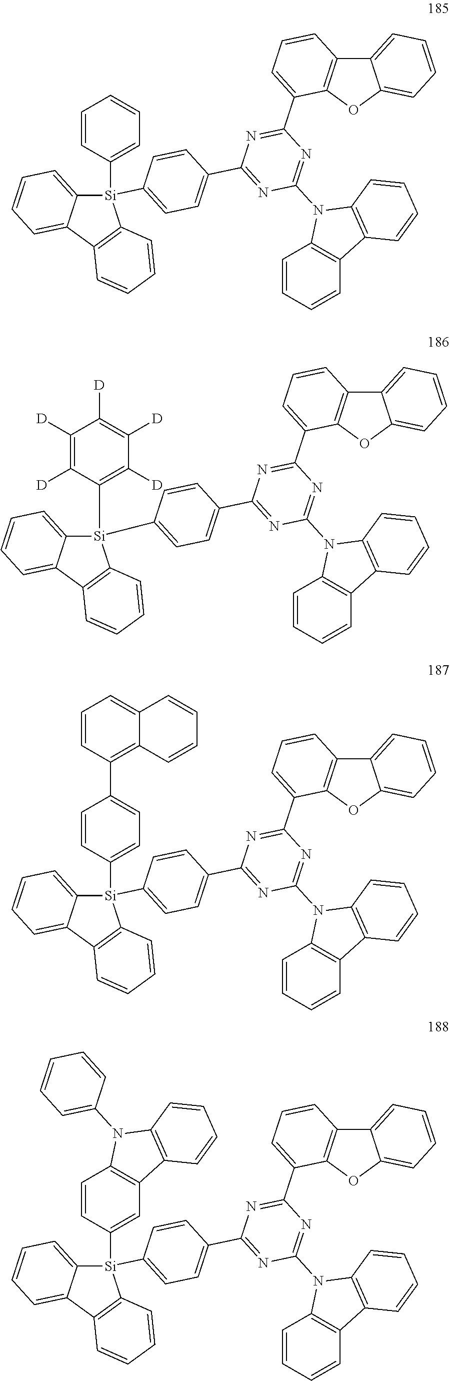 [SMILES: c1ccc([Si]2(c3ccc(-c4nc(-c5cccc6c5oc5ccccc56)nc(-n5c6ccccc6c6ccccc65)n4)cc3)c3ccccc3-c3ccccc32)cc1 ; [2H]c1c([2H])c([2H])c([Si]2(c3ccc(-c4nc(-c5cccc6c5oc5ccccc56)nc(-n5c6ccccc6c6ccccc65)n4)cc3)c3ccccc3-c3ccccc32)c([2H])c1[2H] ; c1ccc2c(c1)-c1ccccc1[Si]2(c1ccc(-c2nc(-c3cccc4c3oc3ccccc34)nc(-n3c4ccccc4c4ccccc43)n2)cc1)c1ccc(-c2cccc3ccccc23)cc1 ; c1ccc(-n2c3ccccc3c3cc([Si]4(c5ccc(-c6nc(-c7cccc8c7oc7ccccc78)nc(-n7c8ccccc8c8ccccc87)n6)cc5)c5ccccc5-c5ccccc54)ccc32)cc1]

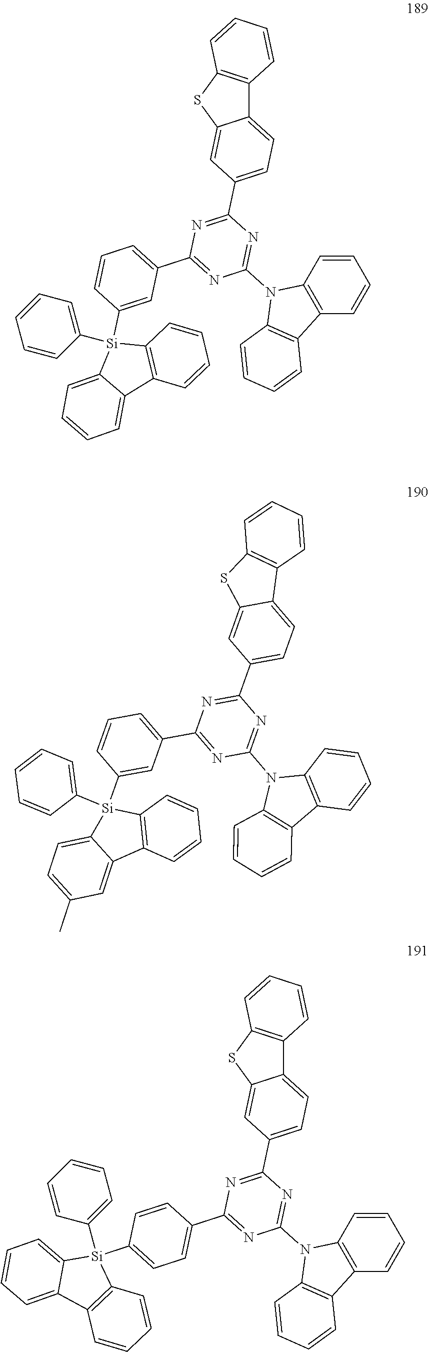 [SMILES: c1ccc([Si]2(c3cccc(-c4nc(-c5ccc6c(c5)sc5ccccc56)nc(-n5c6ccccc6c6ccccc65)n4)c3)c3ccccc3-c3ccccc32)cc1 ; Cc1ccc2c(c1)-c1ccccc1[Si]2(c1ccccc1)c1cccc(-c2nc(-c3ccc4c(c3)sc3ccccc34)nc(-n3c4ccccc4c4ccccc43)n2)c1 ; c1ccc([Si]2(c3ccc(-c4nc(-c5ccc6c(c5)sc5ccccc56)nc(-n5c6ccccc6c6ccccc65)n4)cc3)c3ccccc3-c3ccccc32)cc1]

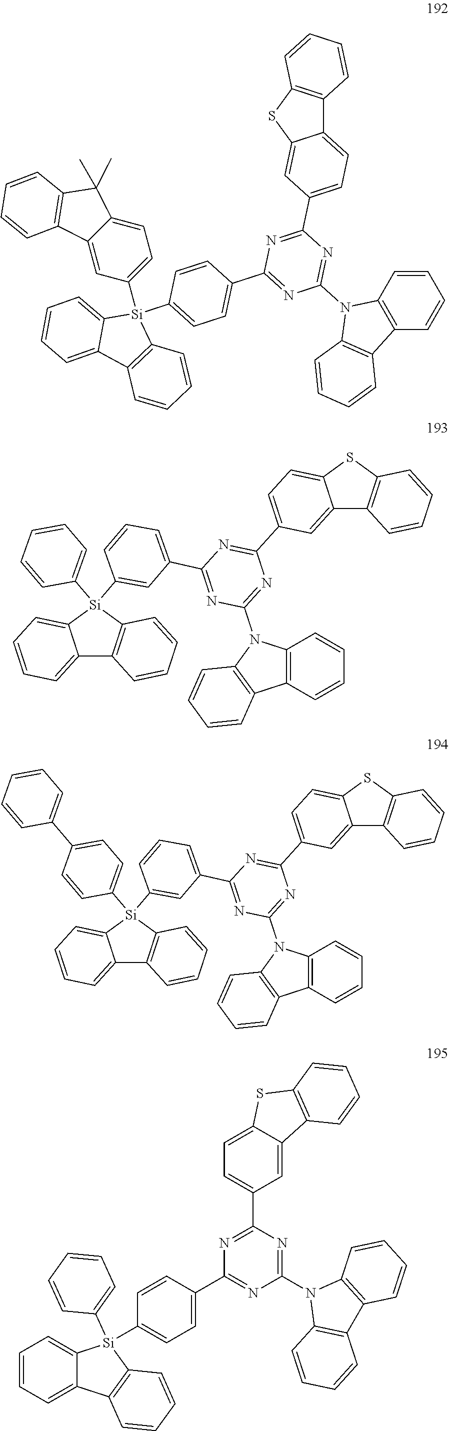 [SMILES: CC1(C)c2ccccc2-c2cc([Si]3(c4ccc(-c5nc(-c6ccc7c(c6)sc6ccccc67)nc(-n6c7ccccc7c7ccccc76)n5)cc4)c4ccccc4-c4ccccc43)ccc21 ; c1ccc([Si]2(c3cccc(-c4nc(-c5ccc6sc7ccccc7c6c5)nc(-n5c6ccccc6c6ccccc65)n4)c3)c3ccccc3-c3ccccc32)cc1 ; c1ccc(-c2ccc([Si]3(c4cccc(-c5nc(-c6ccc7sc8ccccc8c7c6)nc(-n6c7ccccc7c7ccccc76)n5)c4)c4ccccc4-c4ccccc43)cc2)cc1 ; c1ccc([Si]2(c3ccc(-c4nc(-c5ccc6sc7ccccc7c6c5)nc(-n5c6ccccc6c6ccccc65)n4)cc3)c3ccccc3-c3ccccc32)cc1]

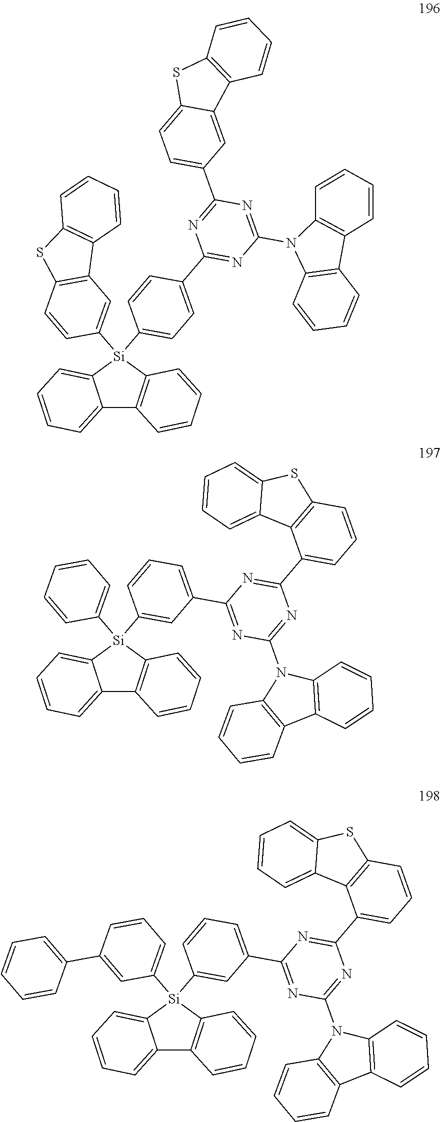 [SMILES: c1ccc2c(c1)-c1ccccc1[Si]2(c1ccc(-c2nc(-c3ccc4sc5ccccc5c4c3)nc(-n3c4ccccc4c4ccccc43)n2)cc1)c1ccc2sc3ccccc3c2c1 ; c1ccc([Si]2(c3cccc(-c4nc(-c5cccc6sc7ccccc7c56)nc(-n5c6ccccc6c6ccccc65)n4)c3)c3ccccc3-c3ccccc32)cc1 ; c1ccc(-c2cccc([Si]3(c4cccc(-c5nc(-c6cccc7sc8ccccc8c67)nc(-n6c7ccccc7c7ccccc76)n5)c4)c4ccccc4-c4ccccc43)c2)cc1]

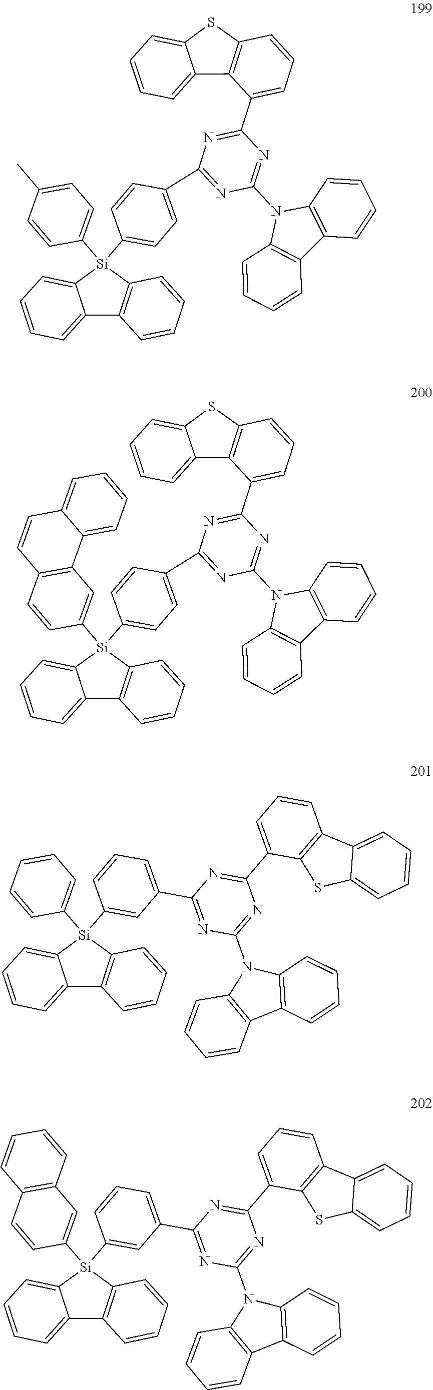 [SMILES: Cc1ccc([Si]2(c3ccc(-c4nc(-c5cccc6sc7ccccc7c56)nc(-n5c6ccccc6c6ccccc65)n4)cc3)c3ccccc3-c3ccccc32)cc1 ; c1ccc2c(c1)-c1ccccc1[Si]2(c1ccc(-c2nc(-c3cccc4sc5ccccc5c34)nc(-n3c4ccccc4c4ccccc43)n2)cc1)c1ccc2ccc3ccccc3c2c1 ; c1ccc([Si]2(c3cccc(-c4nc(-c5cccc6c5sc5ccccc56)nc(-n5c6ccccc6c6ccccc65)n4)c3)c3ccccc3-c3ccccc32)cc1 ; c1cc(-c2nc(-c3cccc4c3sc3ccccc34)nc(-n3c4ccccc4c4ccccc43)n2)cc([Si]2(c3ccc4ccccc4c3)c3ccccc3-c3ccccc32)c1]

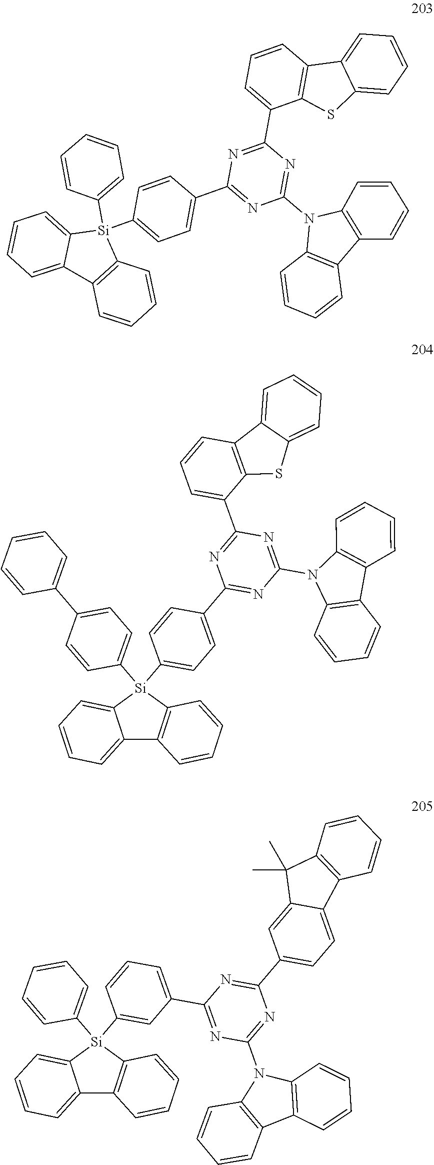 [SMILES: c1ccc([Si]2(c3ccc(-c4nc(-c5cccc6c5sc5ccccc56)nc(-n5c6ccccc6c6ccccc65)n4)cc3)c3ccccc3-c3ccccc32)cc1 ; c1ccc(-c2ccc([Si]3(c4ccc(-c5nc(-c6cccc7c6sc6ccccc67)nc(-n6c7ccccc7c7ccccc76)n5)cc4)c4ccccc4-c4ccccc43)cc2)cc1 ; CC1(C)c2ccccc2-c2ccc(-c3nc(-c4cccc([Si]5(c6ccccc6)c6ccccc6-c6ccccc65)c4)nc(-n4c5ccccc5c5ccccc54)n3)cc21]

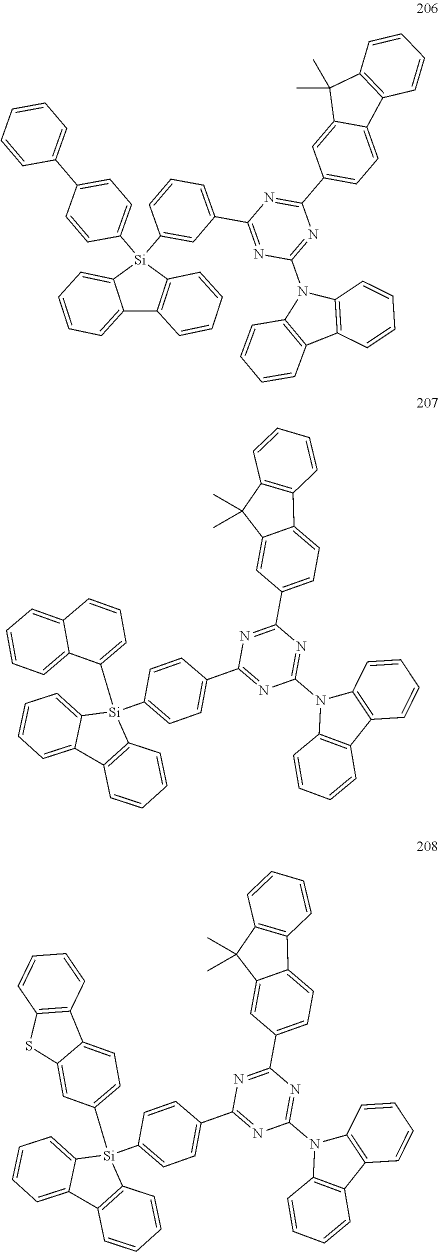 [SMILES: CC1(C)c2ccccc2-c2ccc(-c3nc(-c4cccc([Si]5(c6ccc(-c7ccccc7)cc6)c6ccccc6-c6ccccc65)c4)nc(-n4c5ccccc5c5ccccc54)n3)cc21 ; CC1(C)c2ccccc2-c2ccc(-c3nc(-c4ccc([Si]5(c6cccc7ccccc67)c6ccccc6-c6ccccc65)cc4)nc(-n4c5ccccc5c5ccccc54)n3)cc21 ; CC1(C)c2ccccc2-c2ccc(-c3nc(-c4ccc([Si]5(c6ccc7c(c6)sc6ccccc67)c6ccccc6-c6ccccc65)cc4)nc(-n4c5ccccc5c5ccccc54)n3)cc21]

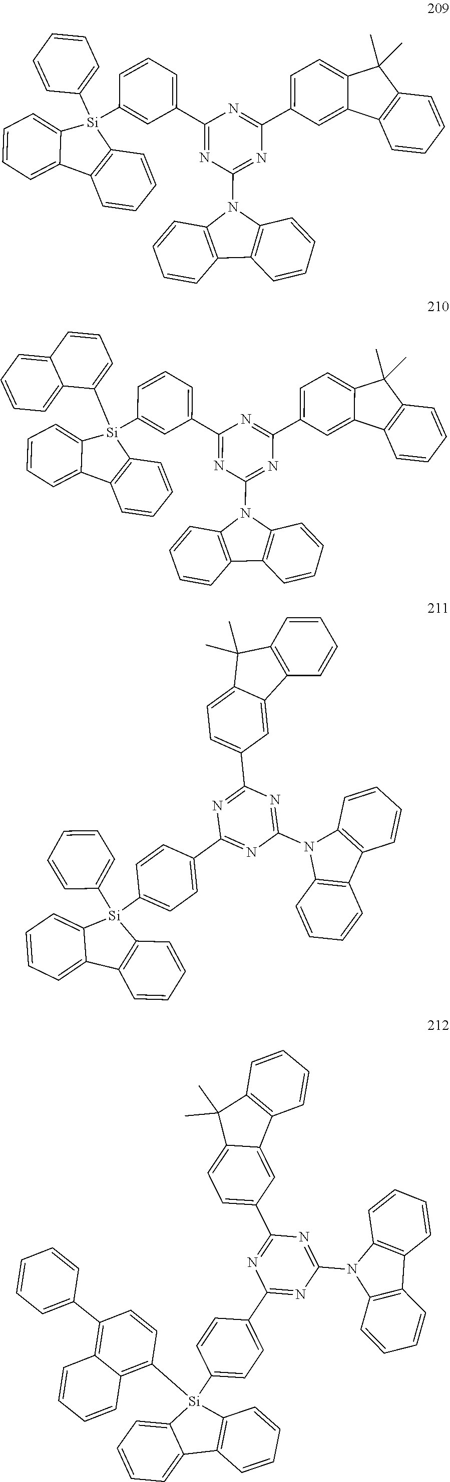

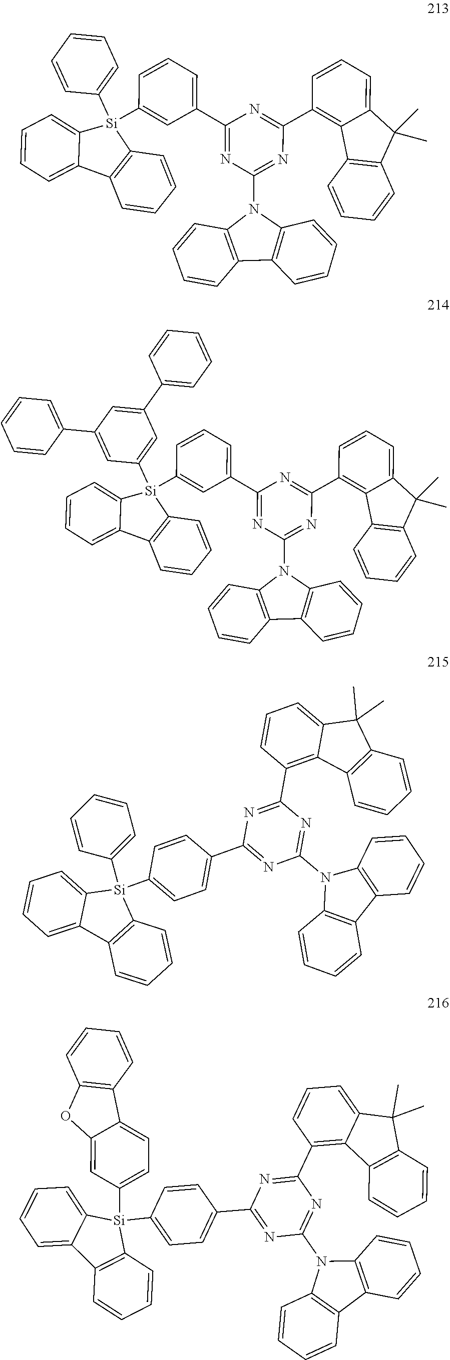 [SMILES: CC1(C)c2ccccc2-c2c(-c3nc(-c4cccc([Si]5(c6ccccc6)c6ccccc6-c6ccccc65)c4)nc(-n4c5ccccc5c5ccccc54)n3)cccc21 ; CC1(C)c2ccccc2-c2c(-c3nc(-c4cccc([Si]5(c6cc(-c7ccccc7)cc(-c7ccccc7)c6)c6ccccc6-c6ccccc65)c4)nc(-n4c5ccccc5c5ccccc54)n3)cccc21 ; CC1(C)c2ccccc2-c2c(-c3nc(-c4ccc([Si]5(c6ccccc6)c6ccccc6-c6ccccc65)cc4)nc(-n4c5ccccc5c5ccccc54)n3)cccc21 ; CC1(C)c2ccccc2-c2c(-c3nc(-c4ccc([Si]5(c6ccc7c(c6)oc6ccccc67)c6ccccc6-c6ccccc65)cc4)nc(-n4c5ccccc5c5ccccc54)n3)cccc21]

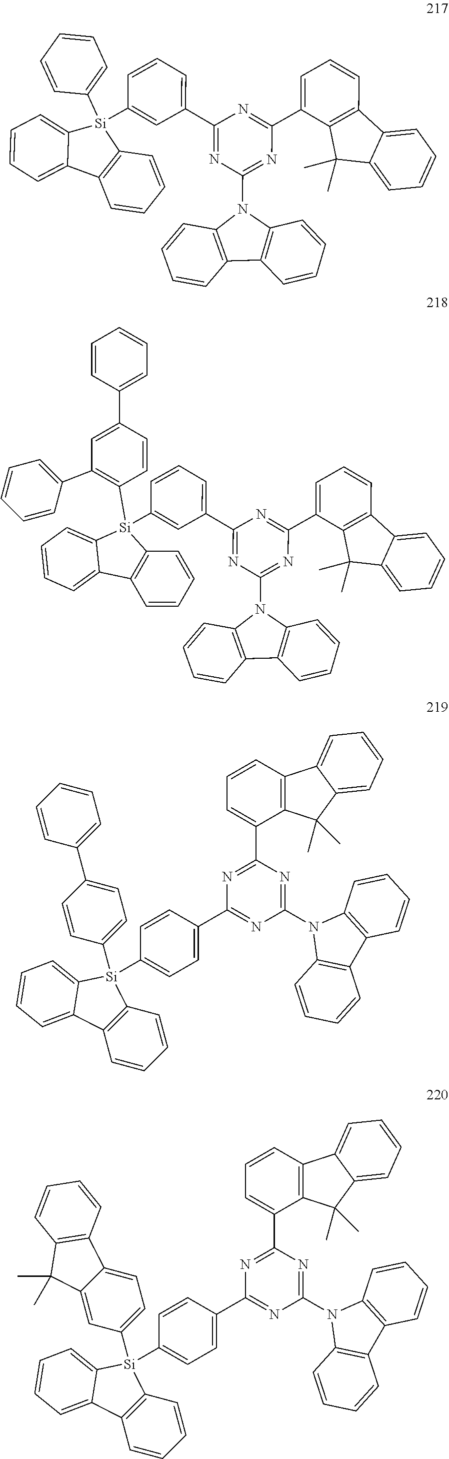 [SMILES: CC1(C)c2ccccc2-c2cccc(-c3nc(-c4cccc([Si]5(c6ccccc6)c6ccccc6-c6ccccc65)c4)nc(-n4c5ccccc5c5ccccc54)n3)c21 ; CC1(C)c2ccccc2-c2cccc(-c3nc(-c4cccc([Si]5(c6ccc(-c7ccccc7)cc6-c6ccccc6)c6ccccc6-c6ccccc65)c4)nc(-n4c5ccccc5c5ccccc54)n3)c21 ; CC1(C)c2ccccc2-c2cccc(-c3nc(-c4ccc([Si]5(c6ccc(-c7ccccc7)cc6)c6ccccc6-c6ccccc65)cc4)nc(-n4c5ccccc5c5ccccc54)n3)c21 ; CC1(C)c2ccccc2-c2ccc([Si]3(c4ccc(-c5nc(-c6cccc7c6C(C)(C)c6ccccc6-7)nc(-n6c7ccccc7c7ccccc76)n5)cc4)c4ccccc4-c4ccccc43)cc21]

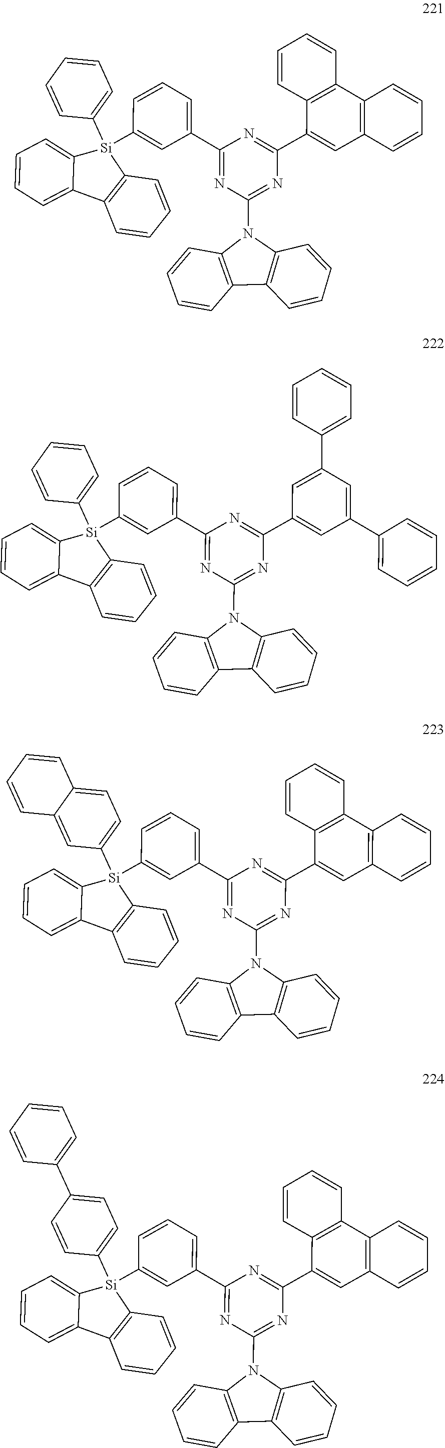 [SMILES: c1ccc([Si]2(c3cccc(-c4nc(-c5cc6ccccc6c6ccccc56)nc(-n5c6ccccc6c6ccccc65)n4)c3)c3ccccc3-c3ccccc32)cc1 ; c1ccc(-c2cc(-c3ccccc3)cc(-c3nc(-c4cccc([Si]5(c6ccccc6)c6ccccc6-c6ccccc65)c4)nc(-n4c5ccccc5c5ccccc54)n3)c2)cc1 ; c1cc(-c2nc(-c3cc4ccccc4c4ccccc34)nc(-n3c4ccccc4c4ccccc43)n2)cc([Si]2(c3ccc4ccccc4c3)c3ccccc3-c3ccccc32)c1 ; c1ccc(-c2ccc([Si]3(c4cccc(-c5nc(-c6cc7ccccc7c7ccccc67)nc(-n6c7ccccc7c7ccccc76)n5)c4)c4ccccc4-c4ccccc43)cc2)cc1]

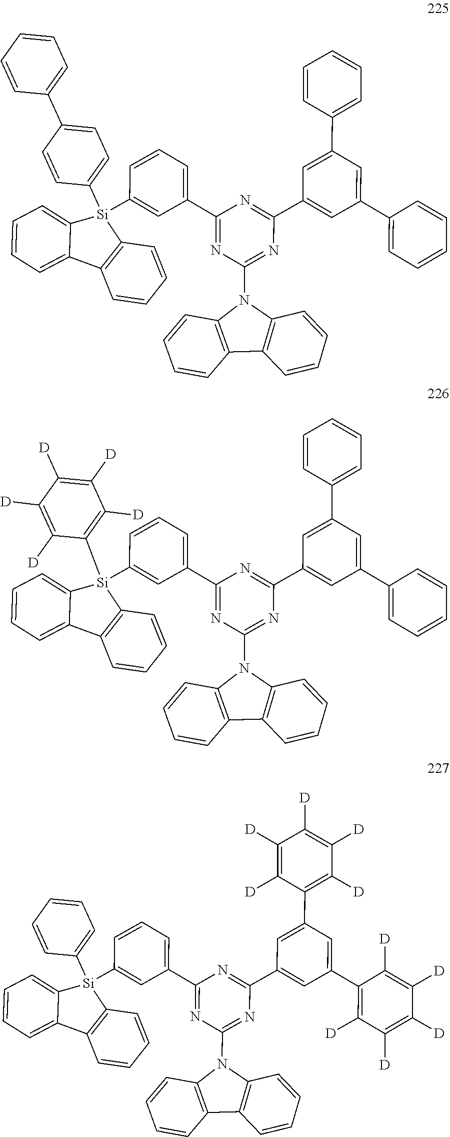 [SMILES: c1ccc(-c2ccc([Si]3(c4cccc(-c5nc(-c6cc(-c7ccccc7)cc(-c7ccccc7)c6)nc(-n6c7ccccc7c7ccccc76)n5)c4)c4ccccc4-c4ccccc43)cc2)cc1 ; [2H]c1c([2H])c([2H])c([Si]2(c3cccc(-c4nc(-c5cc(-c6ccccc6)cc(-c6ccccc6)c5)nc(-n5c6ccccc6c6ccccc65)n4)c3)c3ccccc3-c3ccccc32)c([2H])c1[2H] ; [2H]c1c([2H])c([2H])c(-c2cc(-c3nc(-c4cccc([Si]5(c6ccccc6)c6ccccc6-c6ccccc65)c4)nc(-n4c5ccccc5c5ccccc54)n3)cc(-c3c([2H])c([2H])c([2H])c([2H])c3[2H])c2)c([2H])c1[2H]]

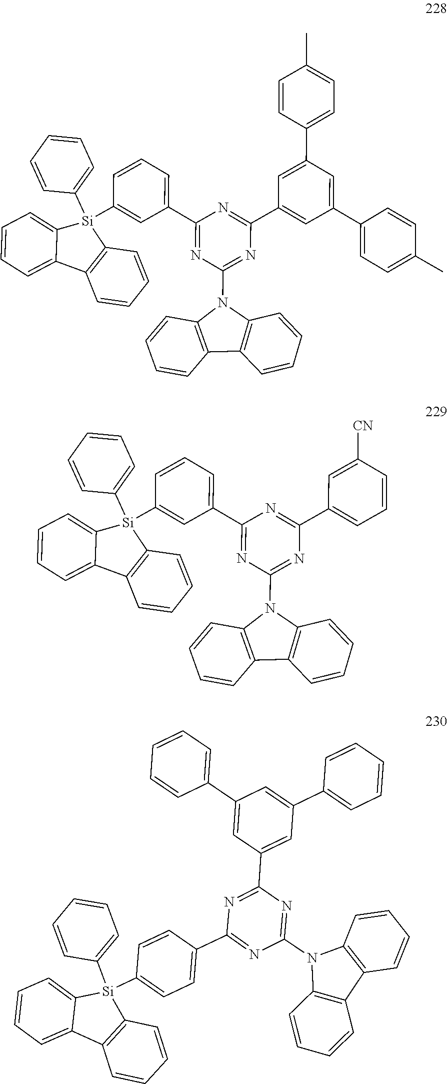 [SMILES: Cc1ccc(-c2cc(-c3ccc(C)cc3)cc(-c3nc(-c4cccc([Si]5(c6ccccc6)c6ccccc6-c6ccccc65)c4)nc(-n4c5ccccc5c5ccccc54)n3)c2)cc1 ; N#Cc1cccc(-c2nc(-c3cccc([Si]4(c5ccccc5)c5ccccc5-c5ccccc54)c3)nc(-n3c4ccccc4c4ccccc43)n2)c1 ; c1ccc(-c2cc(-c3ccccc3)cc(-c3nc(-c4ccc([Si]5(c6ccccc6)c6ccccc6-c6ccccc65)cc4)nc(-n4c5ccccc5c5ccccc54)n3)c2)cc1]

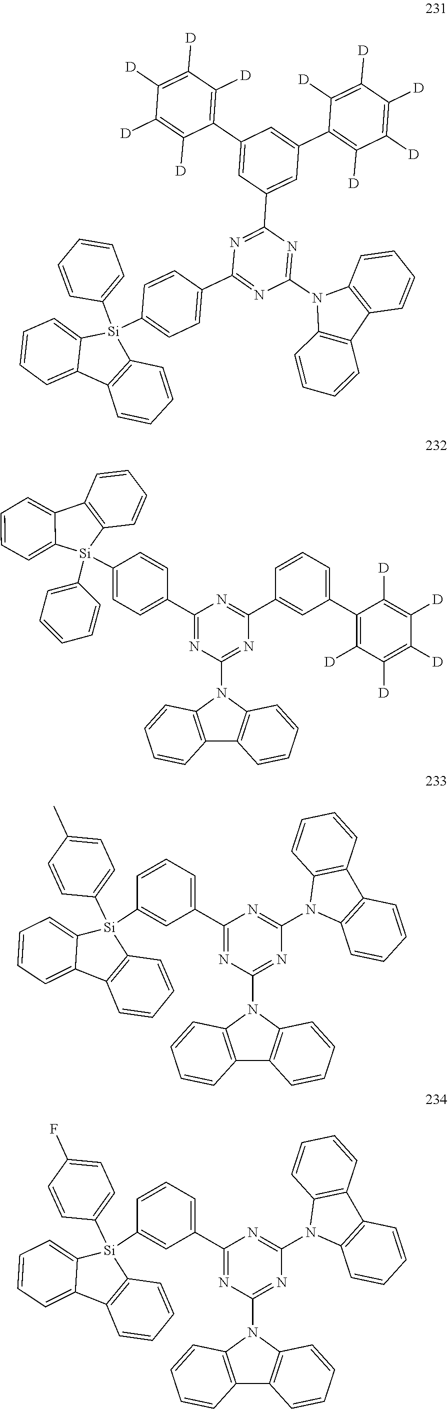 [SMILES: [2H]c1c([2H])c([2H])c(-c2cc(-c3nc(-c4ccc([Si]5(c6ccccc6)c6ccccc6-c6ccccc65)cc4)nc(-n4c5ccccc5c5ccccc54)n3)cc(-c3c([2H])c([2H])c([2H])c([2H])c3[2H])c2)c([2H])c1[2H] ; [2H]c1c([2H])c([2H])c(-c2cccc(-c3nc(-c4ccc([Si]5(c6ccccc6)c6ccccc6-c6ccccc65)cc4)nc(-n4c5ccccc5c5ccccc54)n3)c2)c([2H])c1[2H] ; Cc1ccc([Si]2(c3cccc(-c4nc(-n5c6ccccc6c6ccccc65)nc(-n5c6ccccc6c6ccccc65)n4)c3)c3ccccc3-c3ccccc32)cc1 ; Fc1ccc([Si]2(c3cccc(-c4nc(-n5c6ccccc6c6ccccc65)nc(-n5c6ccccc6c6ccccc65)n4)c3)c3ccccc3-c3ccccc32)cc1]

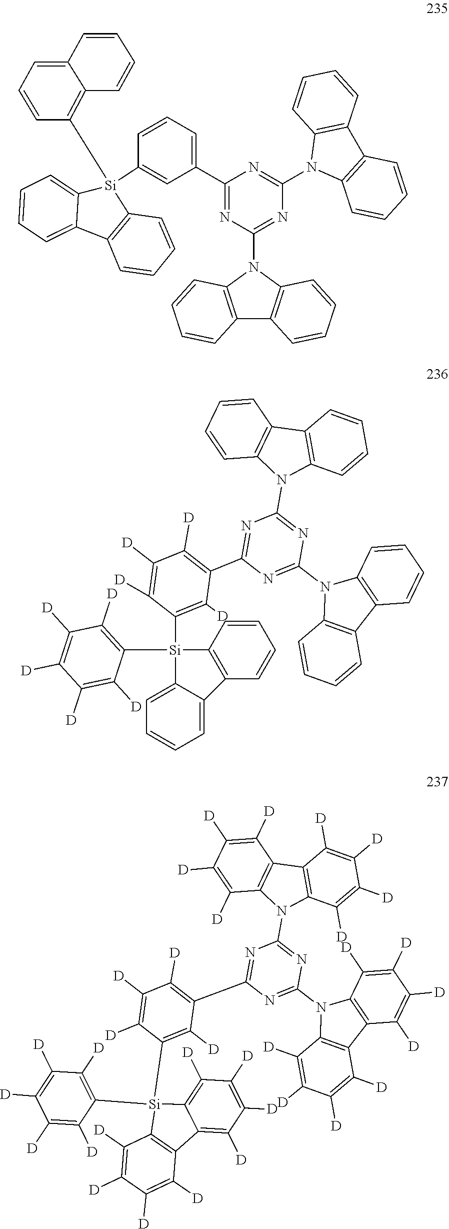

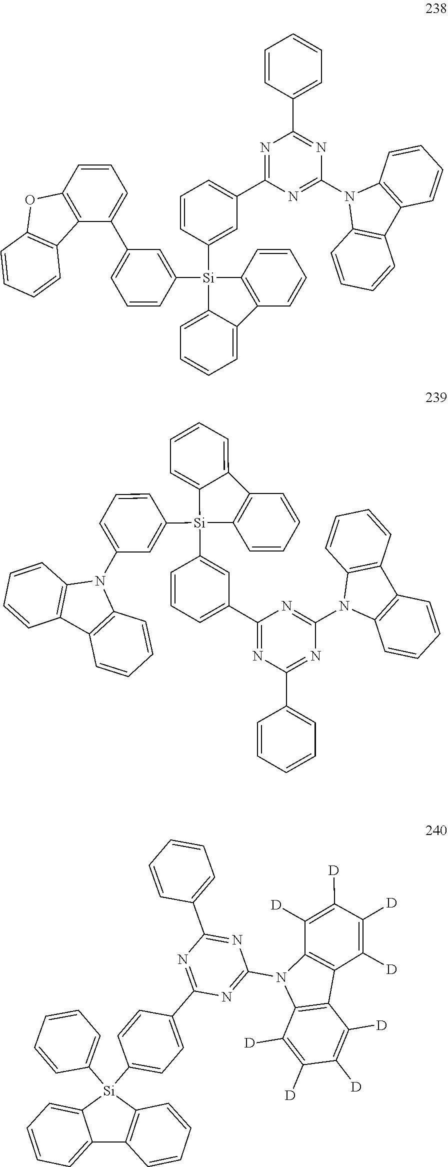 [SMILES: c1ccc(-c2nc(-c3cccc([Si]4(c5cccc(-c6cccc7oc8ccccc8c67)c5)c5ccccc5-c5ccccc54)c3)nc(-n3c4ccccc4c4ccccc43)n2)cc1 ; c1ccc(-c2nc(-c3cccc([Si]4(c5cccc(-n6c7ccccc7c7ccccc76)c5)c5ccccc5-c5ccccc54)c3)nc(-n3c4ccccc4c4ccccc43)n2)cc1 ; [2H]c1c([2H])c([2H])c2c(c1[2H])c1c([2H])c([2H])c([2H])c([2H])c1n2-c1nc(-c2ccccc2)nc(-c2ccc([Si]3(c4ccccc4)c4ccccc4-c4ccccc43)cc2)n1]

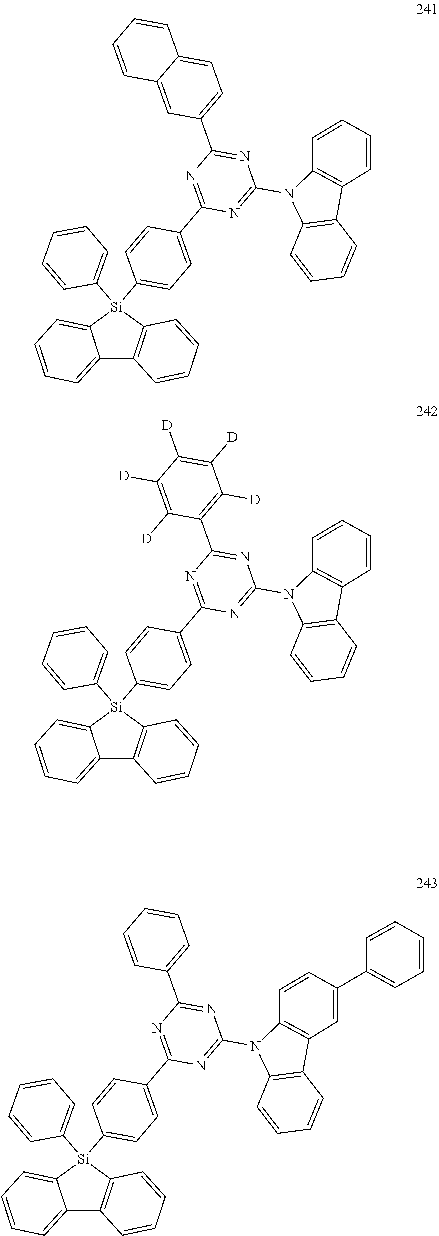

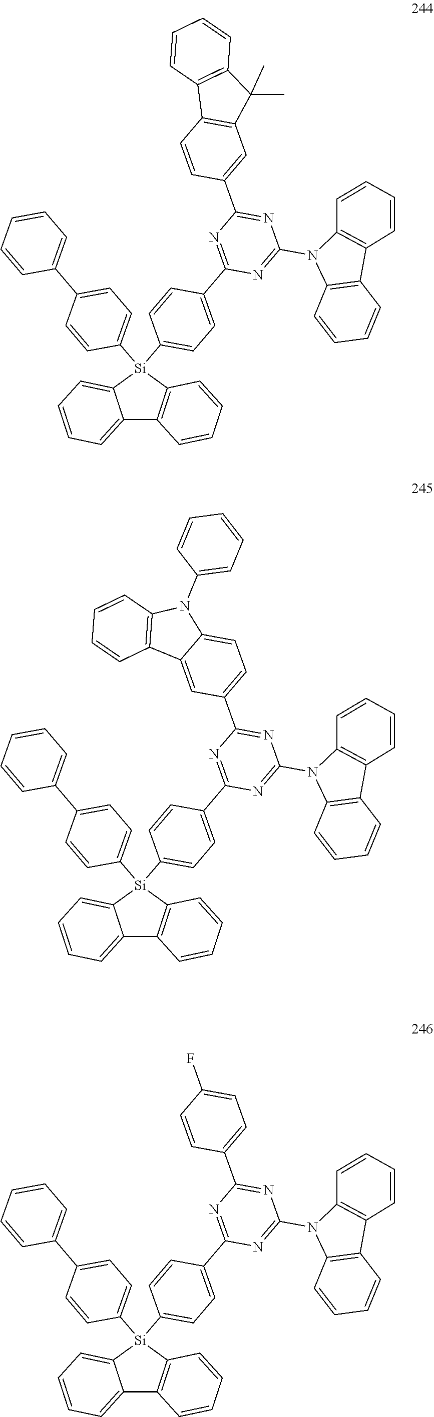 [SMILES: CC1(C)c2ccccc2-c2ccc(-c3nc(-c4ccc([Si]5(c6ccc(-c7ccccc7)cc6)c6ccccc6-c6ccccc65)cc4)nc(-n4c5ccccc5c5ccccc54)n3)cc21 ; c1ccc(-c2ccc([Si]3(c4ccc(-c5nc(-c6ccc7c(c6)c6ccccc6n7-c6ccccc6)nc(-n6c7ccccc7c7ccccc76)n5)cc4)c4ccccc4-c4ccccc43)cc2)cc1 ; Fc1ccc(-c2nc(-c3ccc([Si]4(c5ccc(-c6ccccc6)cc5)c5ccccc5-c5ccccc54)cc3)nc(-n3c4ccccc4c4ccccc43)n2)cc1]

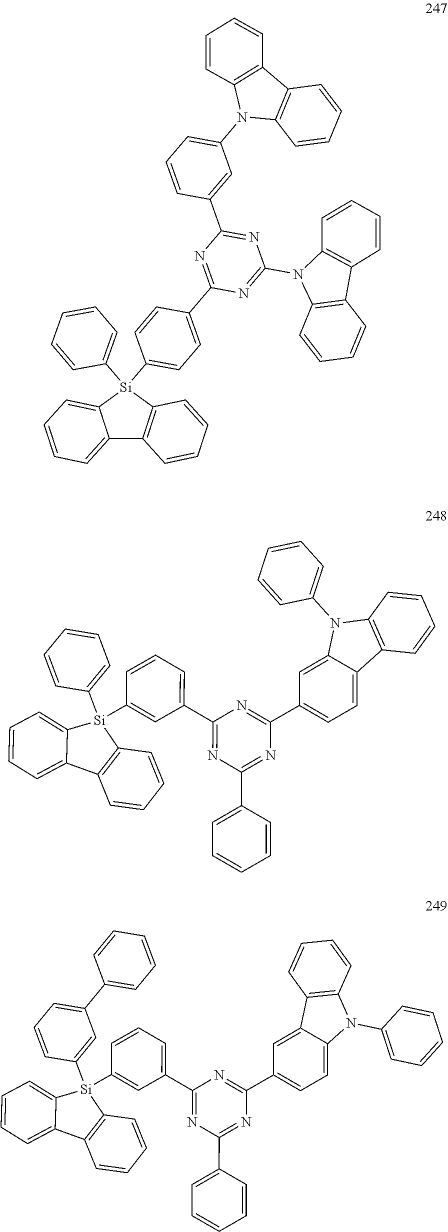 [SMILES: c1ccc([Si]2(c3ccc(-c4nc(-c5cccc(-n6c7ccccc7c7ccccc76)c5)nc(-n5c6ccccc6c6ccccc65)n4)cc3)c3ccccc3-c3ccccc32)cc1 ; c1ccc(-c2nc(-c3cccc([Si]4(c5ccccc5)c5ccccc5-c5ccccc54)c3)nc(-c3ccc4c5ccccc5n(-c5ccccc5)c4c3)n2)cc1 ; c1ccc(-c2cccc([Si]3(c4cccc(-c5nc(-c6ccccc6)nc(-c6ccc7c(c6)c6ccccc6n7-c6ccccc6)n5)c4)c4ccccc4-c4ccccc43)c2)cc1]

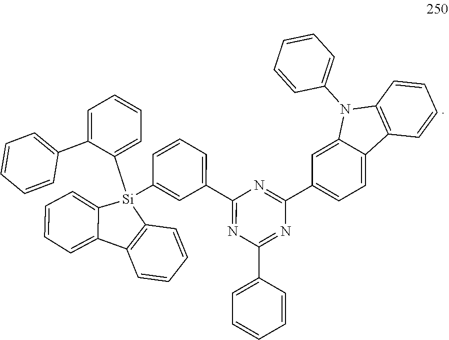 [SMILES: c1ccc(-c2nc(-c3cccc([Si]4(c5ccccc5-c5ccccc5)c5ccccc5-c5ccccc54)c3)nc(-c3ccc4c5ccccc5n(-c5ccccc5)c4c3)n2)cc1]

In a second aspect, the present disclosure provides an organic electroluminescent device, comprising an anode, a cathode, and a functional layer disposed between the anode and the cathode; wherein the functional layer comprises the organic compound of the first aspect of the present disclosure.

Optionally, the organic electroluminescent device is a blue phosphorescence organic electroluminescent device.

The organic compounds provided in the present disclosure may be utilized for the formation of at least one organic film layer within the functional layer, in order to enhance the luminous efficiency and the service life, among other characteristics, of organic electroluminescent devices.

Optionally, the functional layer comprises an organic light-emitting layer, which comprises the organic compound. Among them, the organic light-emitting layer can be composed either of the organic compounds provided by the present disclosure or composed of the organic compounds provided by the present disclosure and other materials collectively.

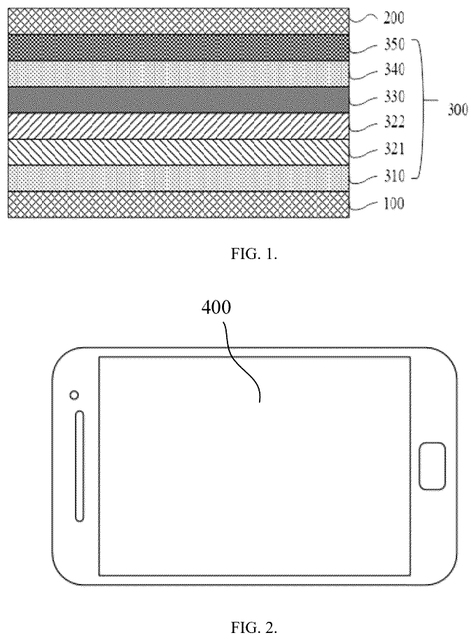

According to one specific embodiment, the organic electroluminescent device is as shown in FIG. 1, and the organic electroluminescent device may comprise an anode 100, a hole injection layer 310, a hole transport layer 321, an electron blocking layer (also referred to as hole auxiliary layer) 322, an organic light-emitting layer 330, an electron transport layer 340, an electron injection layer 350, and a cathode 200 that are stacked sequentially.

In the present disclosure, the anode 100 comprises anode materials, which are preferably a high work function material contributing to injection of holes into the functional layer. The specific examples of the anode material include: metals such as nickel, platinum, vanadium, chromium, copper, zinc, and gold, or alloys thereof; metal oxides such as zinc oxide, indium oxide, indium tin oxide (ITO), and indium zinc oxide (IZO); combinations of metals and oxides, such as ZnO:Al or SnO₂:Sb; or conductive polymers such as poly(3-methylthiophene), poly [3,4-(ethylene-1,2-dioxy) thiophene](PEDT), polypyrrole, and polyaniline, but are not limited thereto. Preferably, a transparent electrode comprising indium tin oxide (ITO) as the anode is included.

In the present disclosure, the hole transport layer may include one or more hole transport material(s). The hole transport materials may be selected from carbazole multimers, carbazole-connected triarylamine based compounds, and other types of compounds. Specifically, the hole transport materials may be selected from the following compounds or any combination thereof:

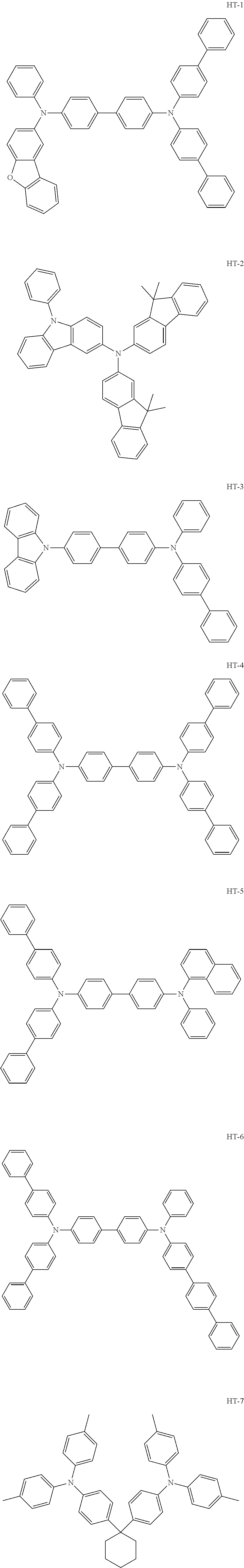

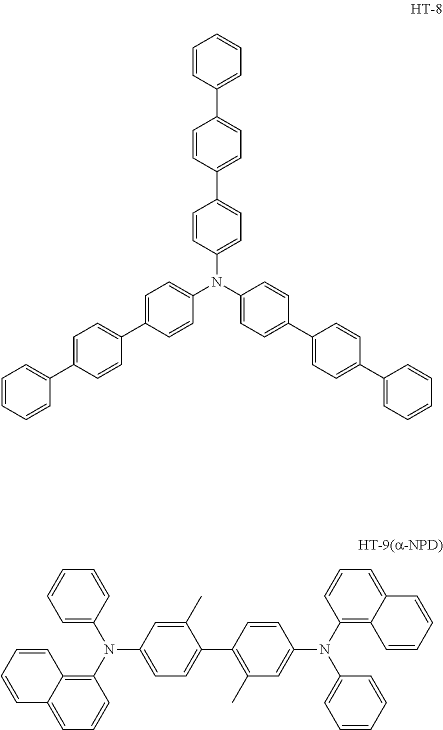 [SMILES: c1ccc(-c2ccc(-c3ccc(N(c4ccc(-c5ccc(-c6ccccc6)cc5)cc4)c4ccc(-c5ccc(-c6ccccc6)cc5)cc4)cc3)cc2)cc1 ; Cc1cc(N(c2ccccc2)c2cccc3ccccc23)ccc1-c1ccc(N(c2ccccc2)c2cccc3ccccc23)cc1C]

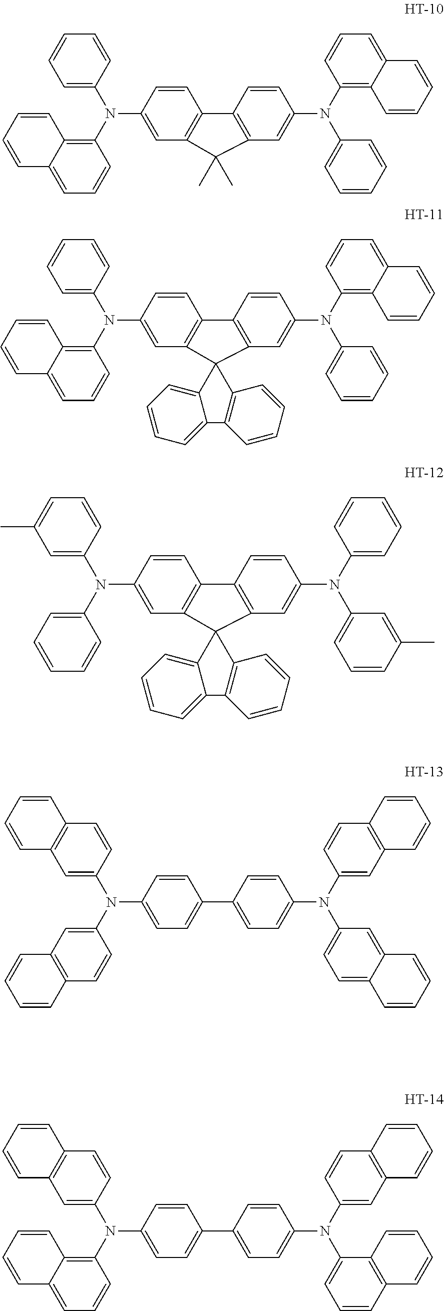

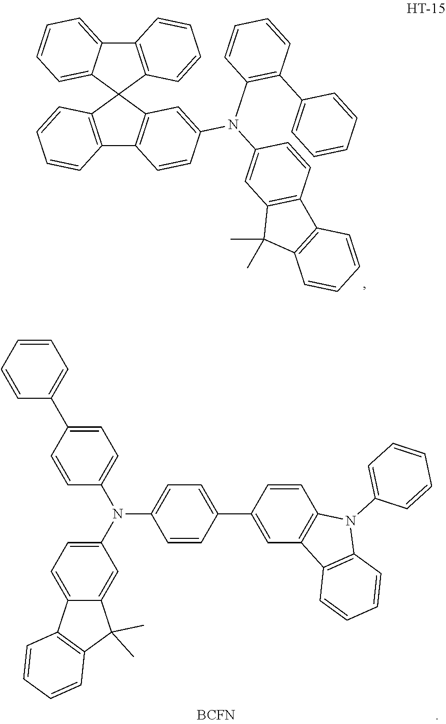 [SMILES: CC1(C)c2ccccc2-c2ccc(N(c3ccc4c(c3)C3(c5ccccc5-c5ccccc53)c3ccccc3-4)c3ccccc3-c3ccccc3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; "BCFN"]

In one embodiment, the hole transport layer 321 may be composed of HT-2.

In one embodiment, the electron blocking layer 322 is composed of HT-3.

Optionally, a hole injection layer 310 may be further provided between the anode 100 and the hole transport layer 321 to enhance the ability to inject holes into the hole transport layer 321.

The hole injection layer 310 may choose to use a benzidine derivative, a starburst arylamine-based compound, a phthalocyanine derivative or other materials. It is not particularly limited in the present disclosure. The material of the hole injection layer 310 is selected, for example, from the following compounds or any combination thereof:

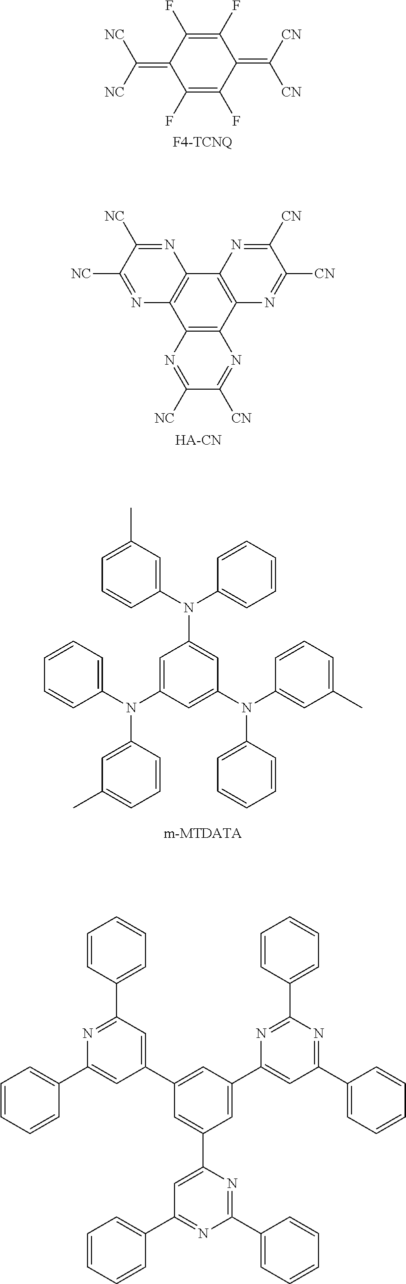 [SMILES: N#CC(C#N)=c1c(F)c(F)c(=C(C#N)C#N)c(F)c1F ; N#Cc1nc2c3nc(C#N)c(C#N)nc3c3nc(C#N)c(C#N)nc3c2nc1C#N ; Cc1cccc(N(c2ccccc2)c2cc(N(c3ccccc3)c3cccc(C)c3)cc(N(c3ccccc3)c3cccc(C)c3)c2)c1 ; c1ccc(-c2cc(-c3cc(-c4cc(-c5ccccc5)nc(-c5ccccc5)n4)cc(-c4cc(-c5ccccc5)nc(-c5ccccc5)n4)c3)cc(-c3ccccc3)n2)cc1]

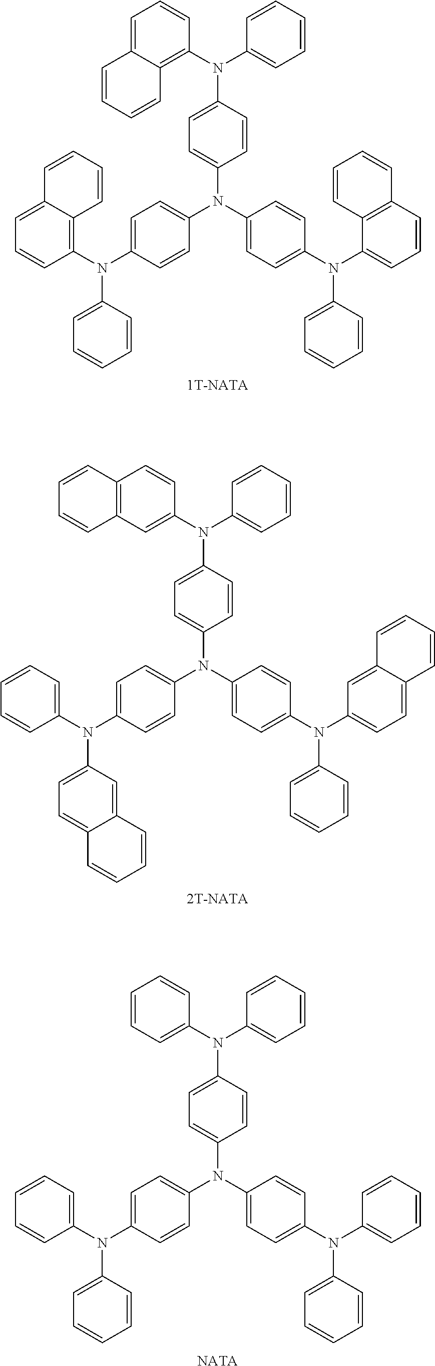

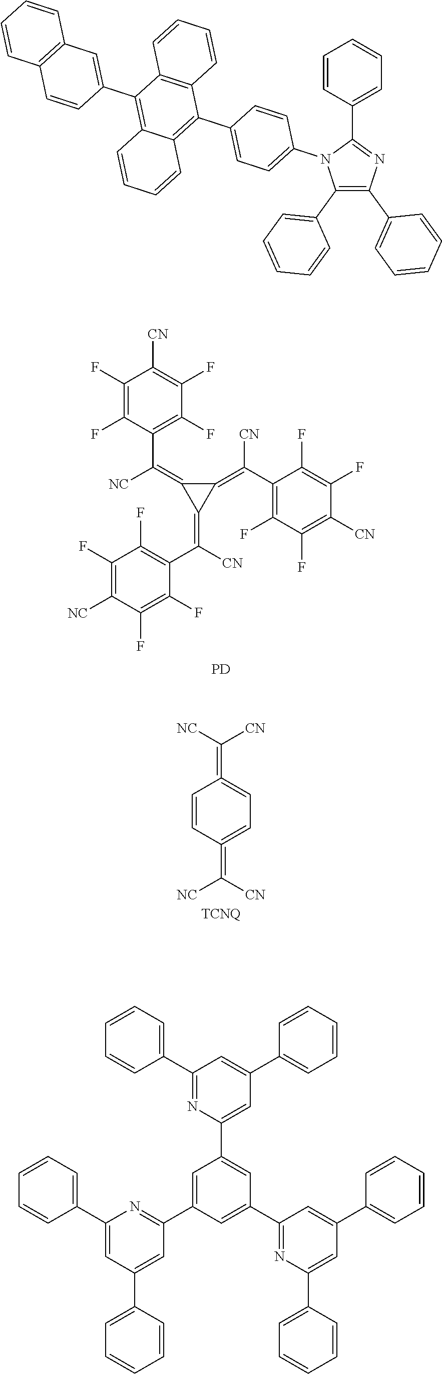

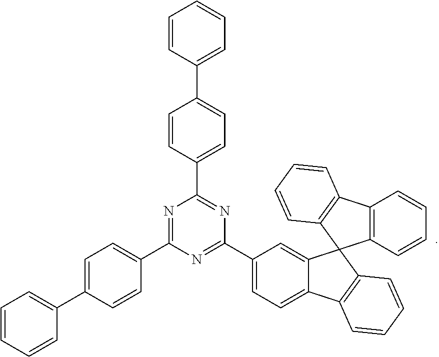 [SMILES: c1ccc(-c2ccc(-c3nc(-c4ccc(-c5ccccc5)cc4)nc(-c4ccc5c(c4)C4(c6ccccc6-c6ccccc64)c4ccccc4-5)n3)cc2)cc1]

In one embodiment, the hole injection layer 310 is composed of PD and HT-2.

In the present disclosure, the organic light-emitting layer 330 may be composed of a single luminescent material or may comprise a host material and a dopant material. Optionally, the organic light-emitting layer 330 is composed of a host material and a dopant material. The holes injected into the organic light-emitting layer 330 and the electrons injected into the organic light-emitting layer 330 can recombine in the organic light-emitting layer 330 to form excitons. The excitons transmit energy to the host material, and the host material transmits the energy to the dopant material, thereby enabling the dopant material to emit light.

The host material of the organic light-emitting layer 330 may include a metal chelating compound, a stilbene-based derivative, an aromatic amine derivative, a dibenzofuran derivative, or other types of materials. Optionally, the host material includes the organic compounds of the present disclosure. In some examples, the body of the light-emitting layer comprises the organic compound of the present disclosure and BH-HT.

The dopant material of the organic light-emitting layer 330 may be a compound having a condensed aryl ring or its derivative, a compound having a heteroaryl ring or its derivative, an aromatic amine derivative, or other materials. It is not particularly limited in the present disclosure.

The dopant material is also known as a doping material or a dopant. The dopant can be categorized into fluorescent and phosphorescent dopants on their luminescence mechanisms. The specific examples of the phosphorescent dopant include but are not limited to,

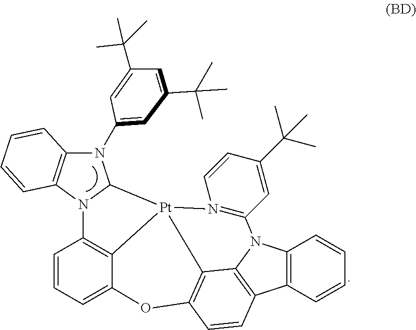

In one embodiment of the present disclosure, the organic electroluminescent device is a blue organic electroluminescent device. In one embodiment, the host material of the organic light-emitting layer 330 comprises the organic compound of the present disclosure. The dopant material is, for example, BD. In another embodiment, the host material of the organic light-emitting layer 330 comprises the organic compound of the present disclosure and BH-HT

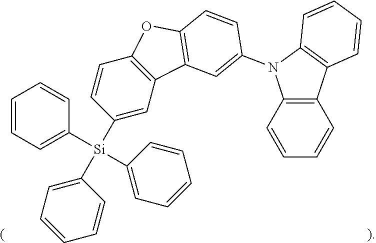 [SMILES: c1ccc([Si](c2ccccc2)(c2ccccc2)c2ccc3oc4ccc(-n5c6ccccc6c6ccccc65)cc4c3c2)cc1]

The dopant material is, for example, BD.

In one embodiment of the present disclosure, the organic electroluminescent device is a blue organic electroluminescent device. In one more specific embodiment, the host material of the organic light-emitting layer 330 comprises the organic compound of the present disclosure.

The electron transport layer 340 may be a single-layer structure or a multi-layer structure and may comprise one or more electron transport material(s). The electron transport materials may be selected from, but not limited to, ET-1, ET-2, ET-6, BTB, LiQ, mSiTrz, benzimidazole derivatives, oxadiazole derivatives, quinoxaline derivatives, and other electron transport materials, and it is not particularly limited in the present disclosure. The material of the electron transport layer 340 includes but is not limited to the following compounds:

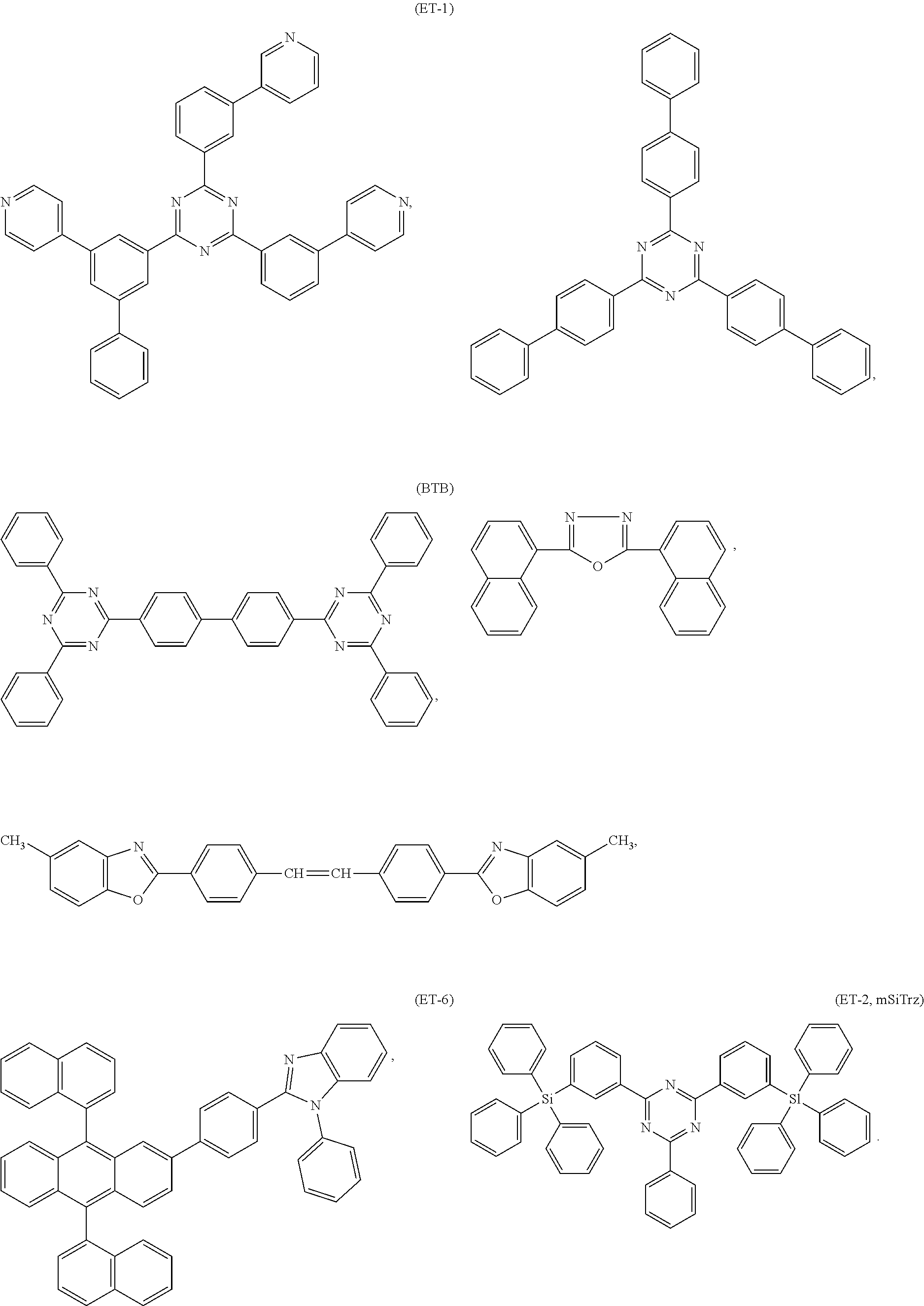

In one embodiment of the present disclosure, the electron transport layer 340 may be composed of ET-2 and LiQ.

In the present disclosure, the cathode 200 may comprise a cathode material, which is a low work function material contributing to injection of electrons into the functional layer. Specific examples of the cathode material include, but are not limited to, metals such as magnesium, calcium, sodium, potassium, titanium, indium, yttrium, lithium, gadolinium, aluminum, silver, tin, and lead, or alloys thereof; or multilayer materials such as LiF/Al, Liq/Al, LiO₂/Al, LiF/Ca, LiF/Al, and BaF₂/Ca. Optionally, a metal electrode comprising magnesium and silver as the cathode is included.

Optionally, an electron injection layer 350 may be further provided between the cathode 200 and the electron transport layer 340 to enhance the ability to inject electrons into the electron transport layer 340. The electron injection layer 350 may comprise an inorganic material such as an alkali metal sulfide and an alkali metal halide or may comprise a complex of an alkali metal and an organic compound. In an embodiment of the present disclosure, the electron injection layer 350 may comprise ytterbium (Yb).

In a third aspect, the present disclosure provides an electronic apparatus, comprising the organic electroluminescent device described in the second aspect of the present disclosure.

According to an embodiment, as shown in FIG. 2, the electronic apparatus is an electronic apparatus 400 comprising the above-described organic electroluminescent device. The electronic apparatus 400 may be a display device, a lighting device, an optical communication device, or other type of electronic apparatus, examples of which may include, for example but be not limited to, computer screens, mobile phone screens, televisions, electronic paper, emergency lamps, optical modules, etc.

The synthesis method of the organic compound in the present disclosure will be demonstrated in detail with the following synthesis examples, but the present disclosure is not limited in any way by this.

### Synthetic Example

Professionals in their field should realize that the chemical reactions described in this present disclosure can be used to properly prepare many of the organic compounds in this present disclosure, and other methods used to prepare the compounds in this present disclosure are considered to be within the scope of this present disclosure. For example, according to the present disclosure, the synthesis of those non-exemplified compounds can be successfully completed by the technicians in the field through modification methods, such as appropriate protection of interfering groups, by using other known reagents in addition to the ones described in the present disclosure, or by making some conventional modifications to the reaction conditions. Compounds for which synthesis methods are not mentioned in the present disclosure may be obtained through commercial sources.

### Synthesis of Intermediate B-1

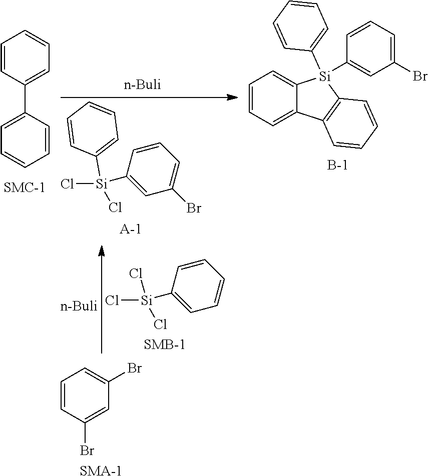

Biphenyl (20 g, 129.7 mmol) was dissolved in 200 mL of THF, and n-butyllithium (51.9 mL, a 2.5 mol/L THF solution, 129.7 mmol) was added dropwise at −78° C., followed by stirring for 1 hour to yield Solution A. Simultaneously, in another reaction vessel, SMA-1 (20 g, 84.8 mmol) was dissolved in 200 mL of THF, and n-butyllithium (33.9 mL, a 2.5 mol/L THF solution, 84.8 mmol) was added dropwise at −78° C., followed by the addition of SMB-1 (17.9 g, 84.8 mmol) and reaction at −78° C. for 1 hour to yield Solution B. Subsequently, Solution B was added to Solution A and stirred for an additional hour at −78° C., after which the system was allowed to warm naturally to room temperature; the reaction mixture was extracted with dichloromethane (200 mL×3 times), the organic phases were combined and dried over anhydrous magnesium sulfate, followed by filtering and removing the solvent under reduced pressure to obtain a crude product. The crude product was purified via silica gel column chromatography using dichloromethane/n-heptane as the mobile phase, to obtain the Intermediate B-1 (18.7g, a total yield of 53.5% over two steps).

A-X and B-X as shown in Table 1 was synthesized in accordance with the synthesis of B-1 except that SMA-X was used instead of SMA-1, SMB-X was used instead of SMB-1, and SMC-X was used instead of SMC-1.

TABLE 1
Synthesis of Intermediates B-2 to B-11

| | | | | | Yield |
|---|---|---|---|---|---|
| | | | | | over |
| | | | | | two |
| | | SMC- | | | steps |
| SMA-X | SMB-X | X | A-X | B-X | (%) |
| 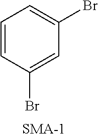 | 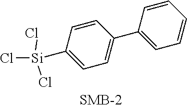 [SMILES: Cl[Si](Cl)(Cl)c1ccc(-c2ccccc2)cc1] | 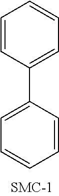 [SMILES: c1ccc(-c2ccccc2)cc1] | 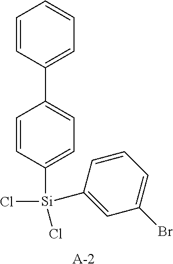 | 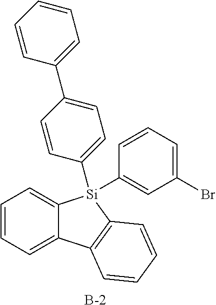 | 30 |
| 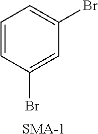 | 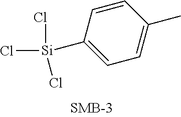 | 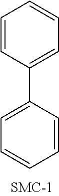 [SMILES: c1ccc(-c2ccccc2)cc1] | 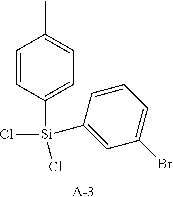 | 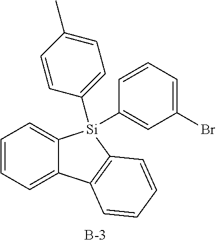 | 32 |
| 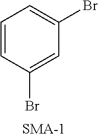 | 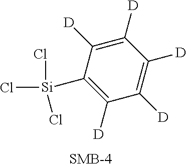 | 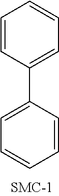 [SMILES: c1ccc(-c2ccccc2)cc1] | 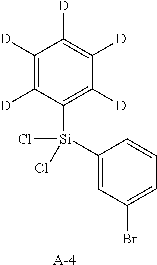 [SMILES: [2H]c1c([2H])c([2H])c([Si](Cl)(Cl)c2cccc(Br)c2)c([2H])c1[2H] ; "A-4"] | 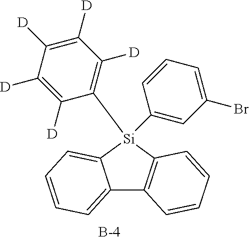 | 35 |
| 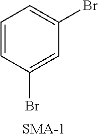 | 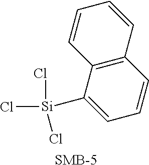 | 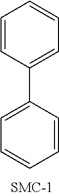 [SMILES: c1ccc(-c2ccccc2)cc1] | 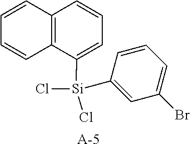 | 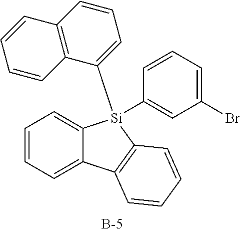 | 28 |
| 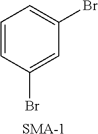 | 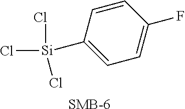 | 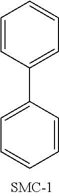 [SMILES: c1ccc(-c2ccccc2)cc1] | 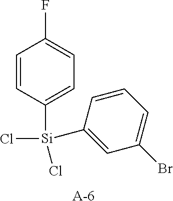 | 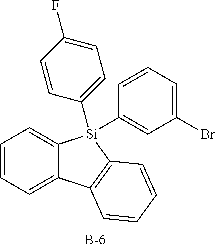 | 31 |
| 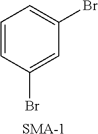 | 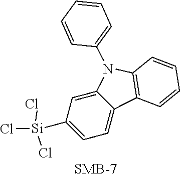 | 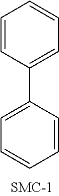 [SMILES: c1ccc(-c2ccccc2)cc1] | 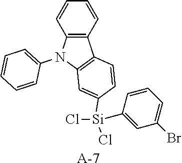 | 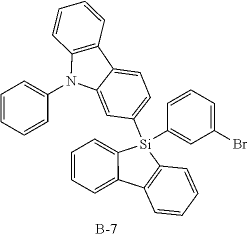 | 24 |
| 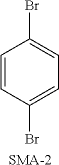 | 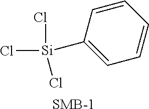 | 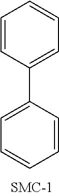 [SMILES: c1ccc(-c2ccccc2)cc1] | 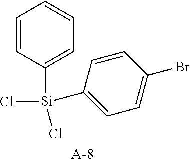 | 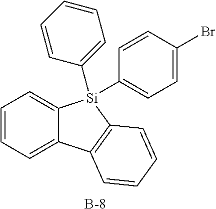 | 34 |
| 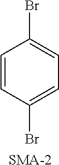 | 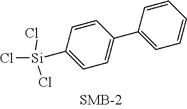 [SMILES: Cl[Si](Cl)(Cl)c1ccc(-c2ccccc2)cc1] | 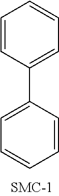 [SMILES: c1ccc(-c2ccccc2)cc1] | 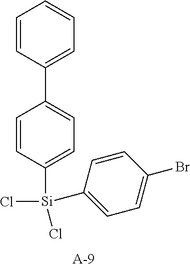 | 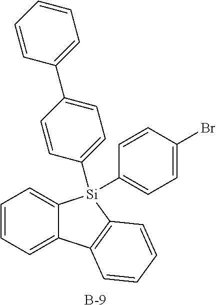 | 33 |
| 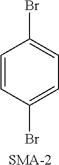 | 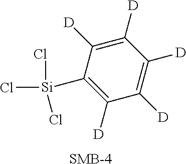 | 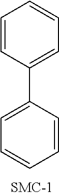 [SMILES: c1ccc(-c2ccccc2)cc1] | 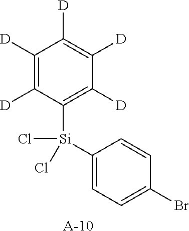 | 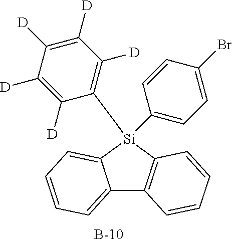 | 30 |
| 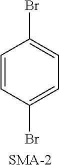 | 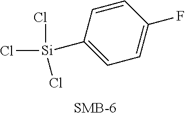 | 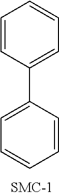 [SMILES: c1ccc(-c2ccccc2)cc1] | 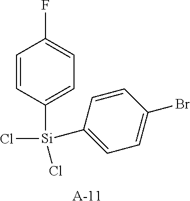 | 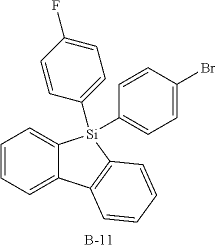 | 34 |

### Synthesis of Intermediate C-1

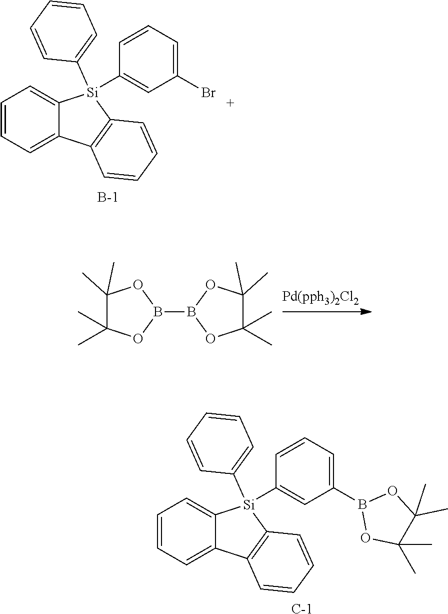

B-1 (20 g, 48.4 mmol) was dissolved in toluene, followed by the addition of Pd(pph)₃Cl₂ (1.8 g, 2.4 mmol), potassium acetate (16.6 g, 169.3 mmol), and boric acid pinacol ester (14.7 g, 58.1 mmol). The reaction mixture was stirred and heated to reflux for 16 hours of reaction. After the system was cooled to room temperature, the reaction mixture was extracted with dichloromethane (100 mL×3 times). The organic phases were combined and dried over anhydrous sodium sulfate, followed by filtering, and removing the solvent under reduced pressure to obtain a crude product.

The crude product was purified via silica gel column chromatography using n-heptane/dichloromethane as the mobile phase, to obtain C-1 (15.6 g, yield:70%).

C-X as shown in Table 2 was synthesized in accordance with the synthesis of C-1 except that B-X as shown in Table 2 was used instead of B1.

TABLE 2
Synthesis of Intermediates C-2 to C-11

| B-X | C-X | Yield (%) |
|---|---|---|
| 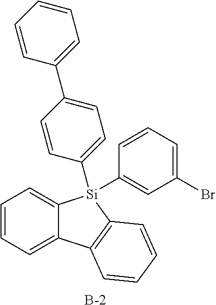 | 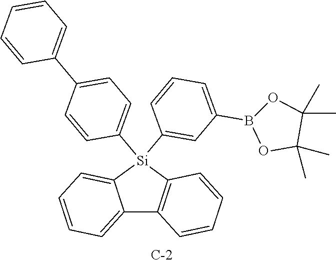 | 68 |
| 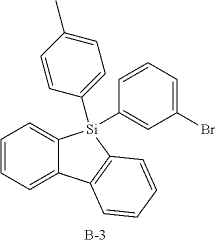 | 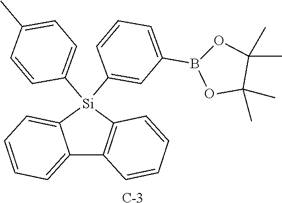 | 65 |
| 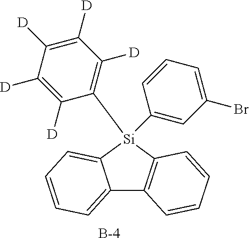 | 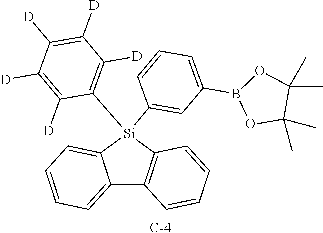 | 70 |
| 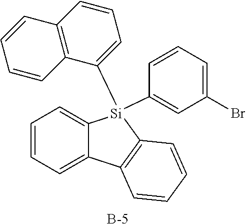 | 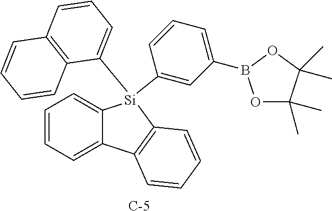 | 67 |
| 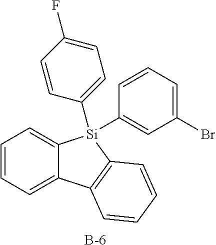 | 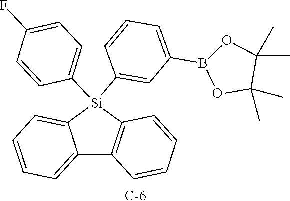 | 65 |
| 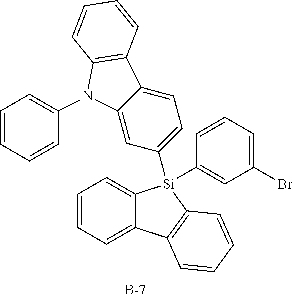 | 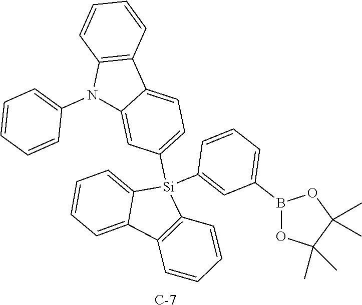 | 61 |
| 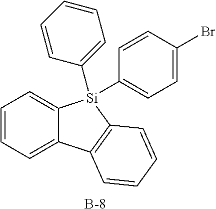 | 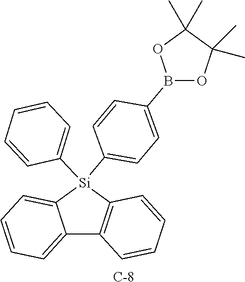 | 68 |
| 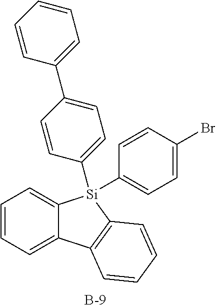 | 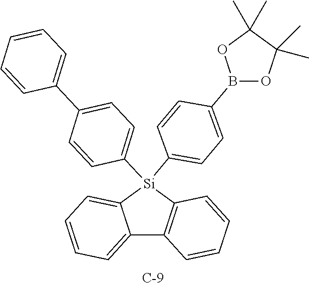 | 65 |
| 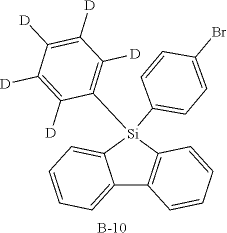 | 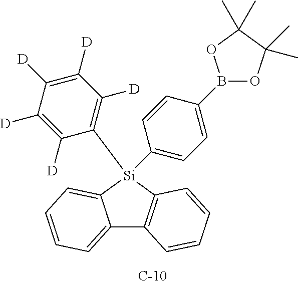 | 64 |
| 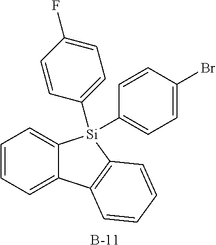 | 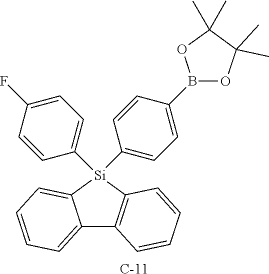 | 67 |

### Synthesis of Compound 1

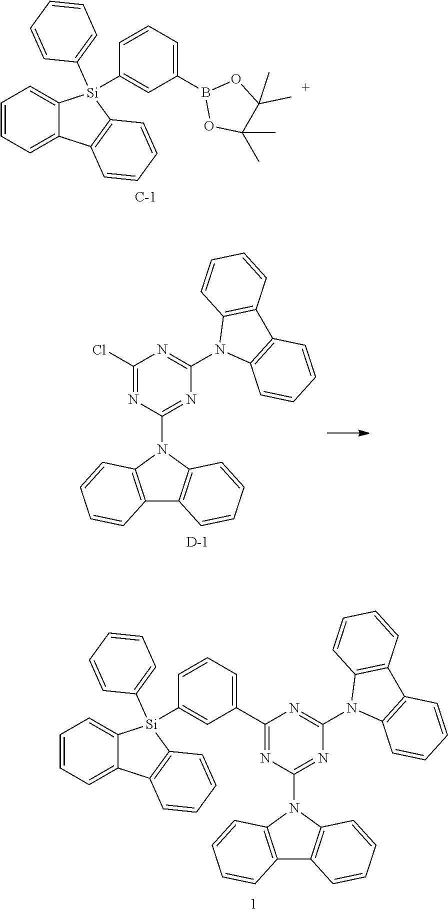

Under a nitrogen atmosphere, C-1 (20 g, 43.4 mmol), D-1 (23.2 g, 52.1 mmol), tetrakis(triphenylphosphine)palladium (2.5 g, 2.2 mmol), anhydrous potassium carbonate (21 g, 152 mmol), tetrabutylammonium bromide (9.8 g, 30.4 mmol), toluene (160 mL), anhydrous ethanol (80 mL), and deionized water (40 mL) were added sequentially to a three-necked flask. The reaction mixture was stirred and heated to reflux for 16 hours of reaction. After the system was cooled to room temperature, the reaction mixture was extracted with dichloromethane (100 mL×3 times). The organic phases were combined and dried over anhydrous sodium sulfate, followed by filtering, and removing the solvent under reduced pressure to obtain a crude product. The crude product was purified by silica gel column chromatography using n-heptane/dichloromethane as the mobile phase, to obtain Compound 1 (21 g, yield 65%).

Compound Y as shown in Table 3 was synthesized in accordance with the synthesis of Compound 1 except that C-X and D-X as shown in Table 3 were respectively used instead of C-1 and D-1.

TABLE 3
Synthesis of Compounds

| | | | Yield |
|---|---|---|---|
| C-X | D-X | compoundY | (%) |
| 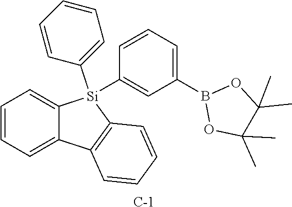 | 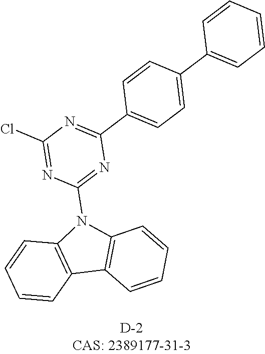 | 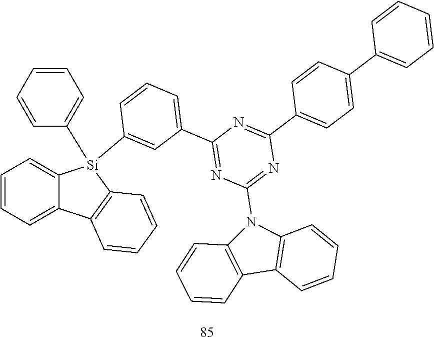 | 67 |
| 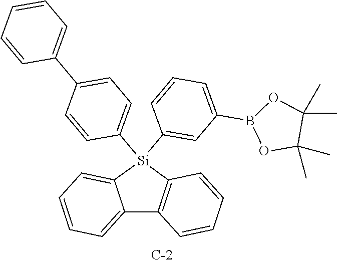 | 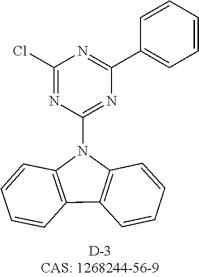 | 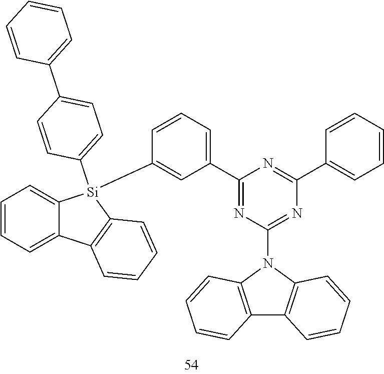 [SMILES: c1ccc(-c2ccc([Si]3(c4cccc(-c5nc(-c6ccccc6)nc(-n6c7ccccc7c7ccccc76)n5)c4)c4ccccc4-c4ccccc43)cc2)cc1] | 65 |
| 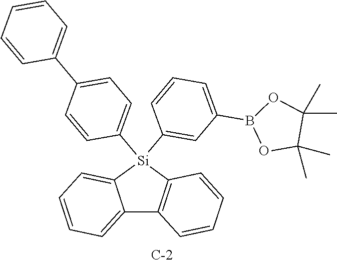 | 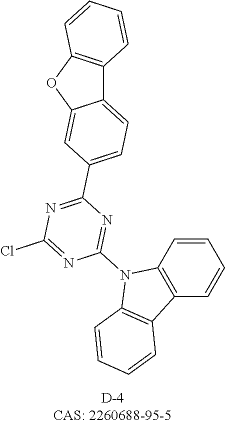 | 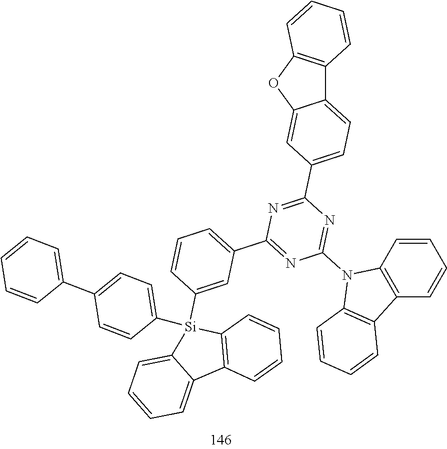 [SMILES: c1ccc(-c2ccc([Si]3(c4cccc(-c5nc(-c6ccc7c(c6)oc6ccccc67)nc(-n6c7ccccc7c7ccccc76)n5)c4)c4ccccc4-c4ccccc43)cc2)cc1] | 62 |
| 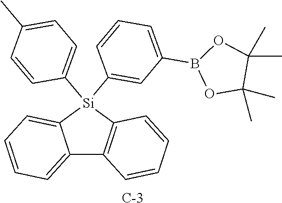 | 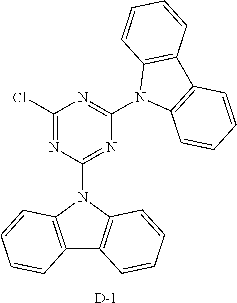 | 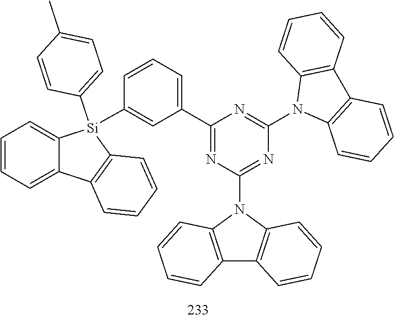 [SMILES: Cc1ccc([Si]2(c3cccc(-c4nc(-n5c6ccccc6c6ccccc65)nc(-n5c6ccccc6c6ccccc65)n4)c3)c3ccccc3-c3ccccc32)cc1] | 64 |
| 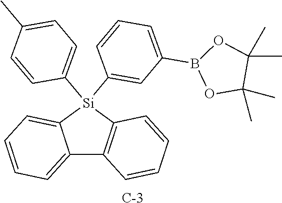 | 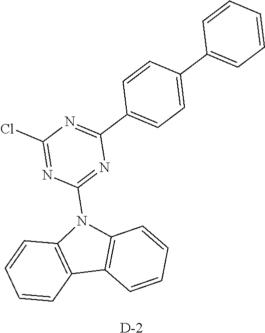 | 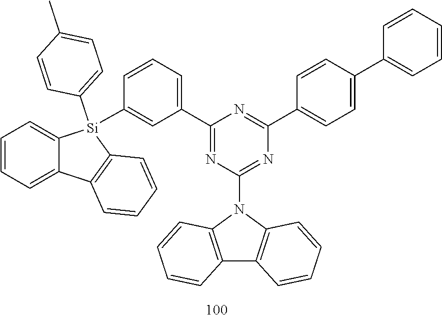 [SMILES: Cc1ccc([Si]2(c3cccc(-c4nc(-c5ccc(-c6ccccc6)cc5)nc(-n5c6ccccc6c6ccccc65)n4)c3)c3ccccc3-c3ccccc32)cc1] | 63 |
| 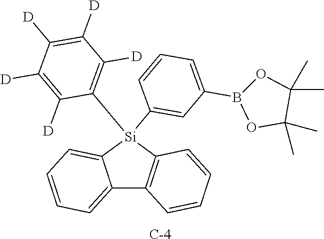 | 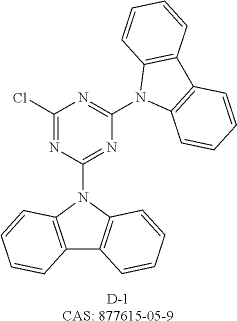 | 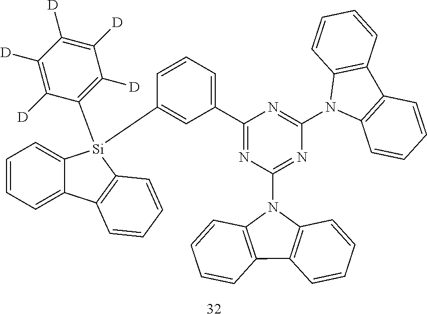 | 67 |
| 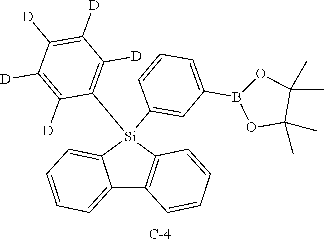 | 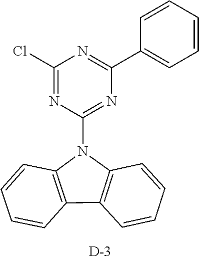 | 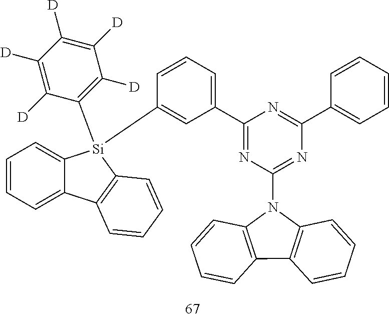 | 66 |
| 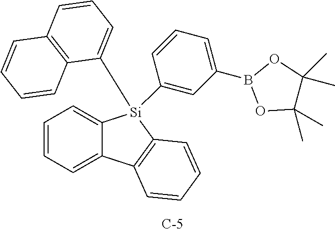 | 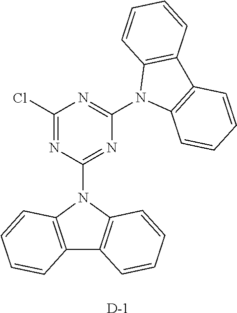 | 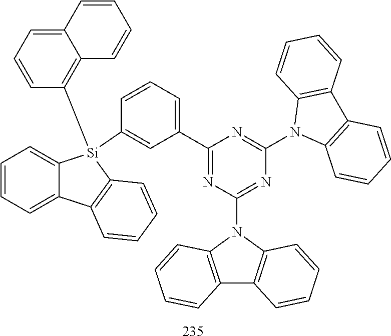 | 61 |
| 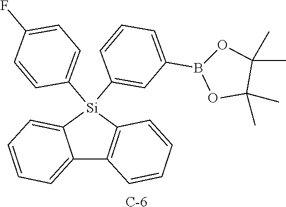 | 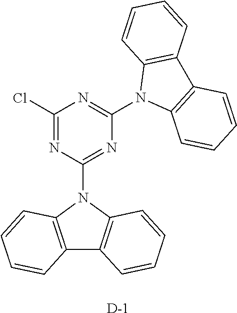 | 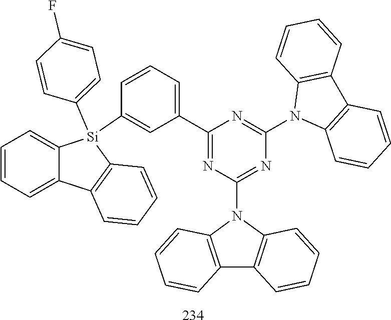 | 64 |
| 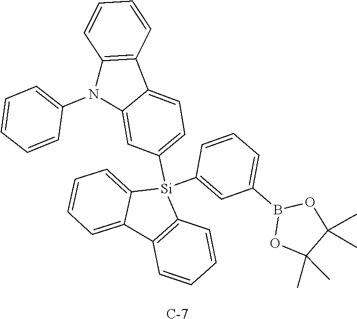 | 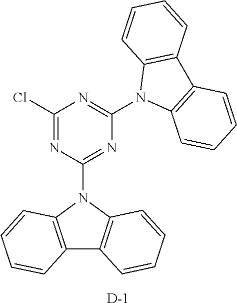 | 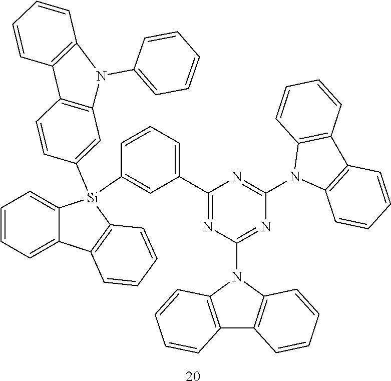 | 60 |
| 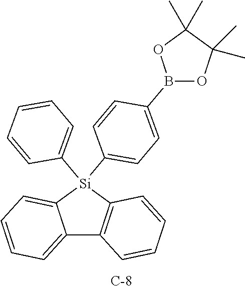 | 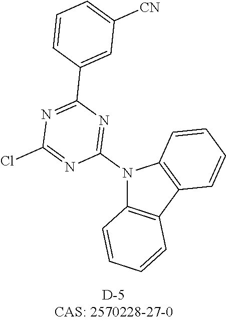 | 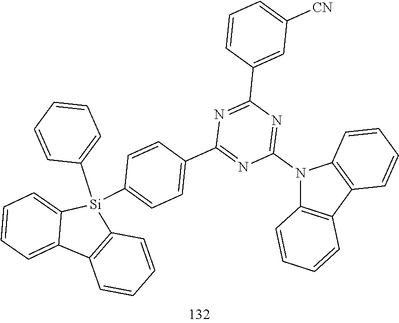 [SMILES: N#Cc1cccc(-c2nc(-c3ccc([Si]4(c5ccccc5)c5ccccc5-c5ccccc54)cc3)nc(-n3c4ccccc4c4ccccc43)n2)c1] | 64 |
| 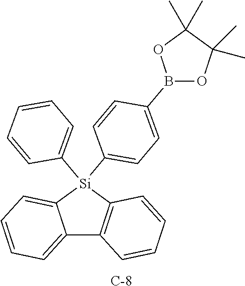 | 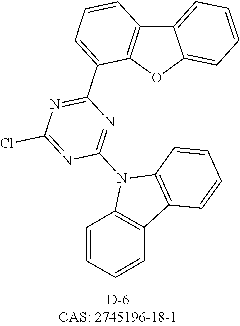 | 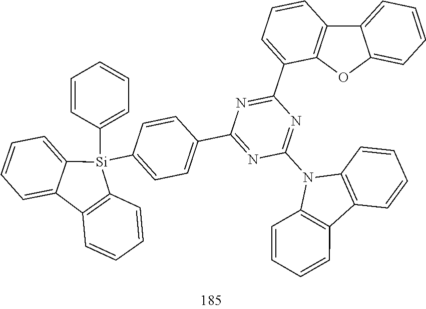 [SMILES: c1ccc([Si]2(c3ccc(-c4nc(-c5cccc6c5oc5ccccc56)nc(-n5c6ccccc6c6ccccc65)n4)cc3)c3ccccc3-c3ccccc32)cc1] | 61 |
| 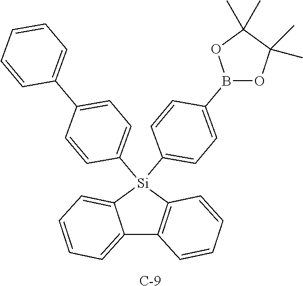 | 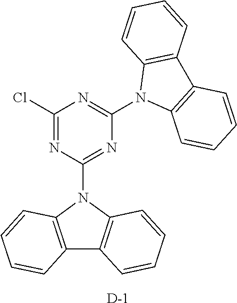 | 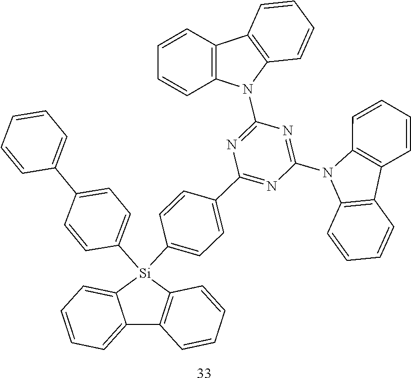 | 64 |
| 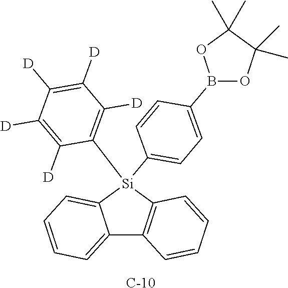 | 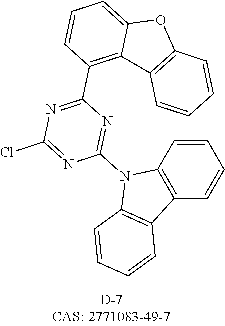 | 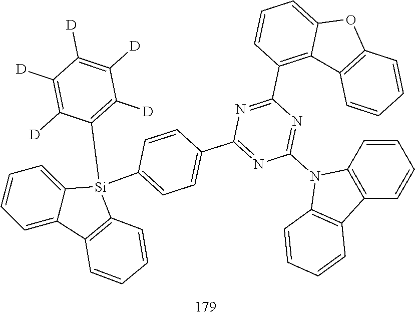 [SMILES: [2H]c1c([2H])c([2H])c([Si]2(c3ccc(-c4nc(-c5cccc6oc7ccccc7c56)nc(-n5c6ccccc6c6ccccc65)n4)cc3)c3ccccc3-c3ccccc32)c([2H])c1[2H]] | 65 |
| 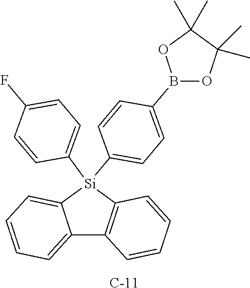 | 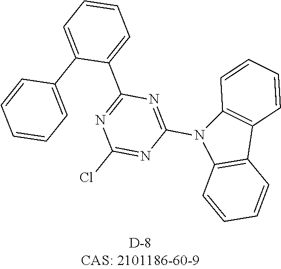 | 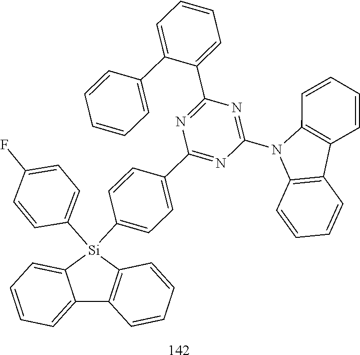 | 63 |
| 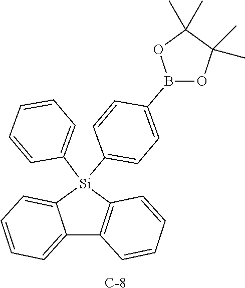 | 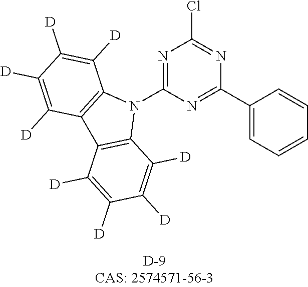 | 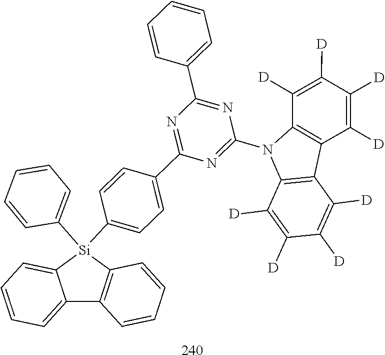 [SMILES: [2H]c1c([2H])c([2H])c2c(c1[2H])c1c([2H])c([2H])c([2H])c([2H])c1n2-c1nc(-c2ccccc2)nc(-c2ccc([Si]3(c4ccccc4)c4ccccc4-c4ccccc43)cc2)n1 ; "240"] | 65 |
| 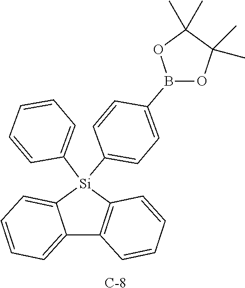 | 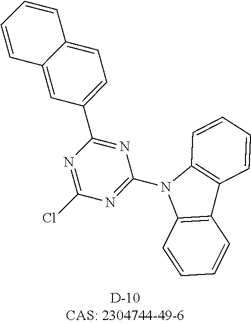 | 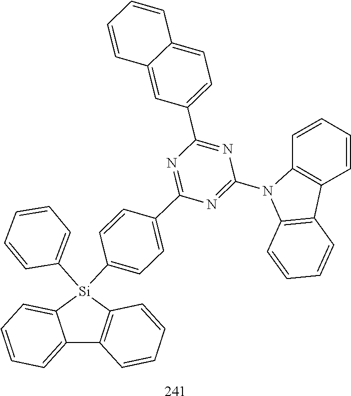 | 62 |
| 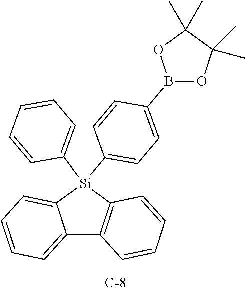 | 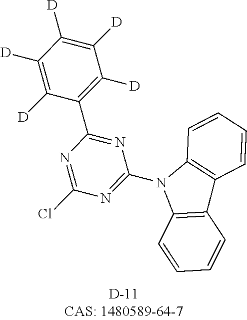 | 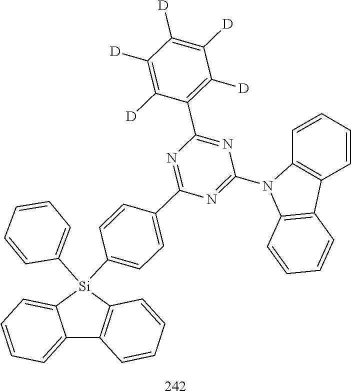 | 53 |
| 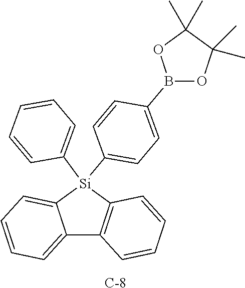 | 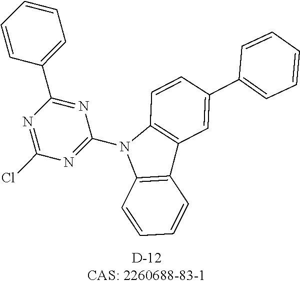 | 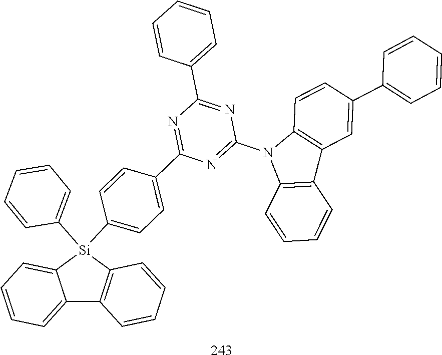 [SMILES: c1ccc(-c2ccc3c(c2)c2ccccc2n3-c2nc(-c3ccccc3)nc(-c3ccc([Si]4(c5ccccc5)c5ccccc5-c5ccccc54)cc3)n2)cc1 ; "243"] | 64 |
| 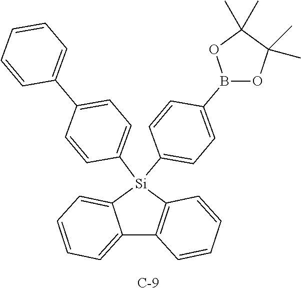 | 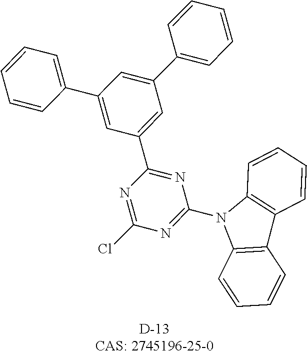 | 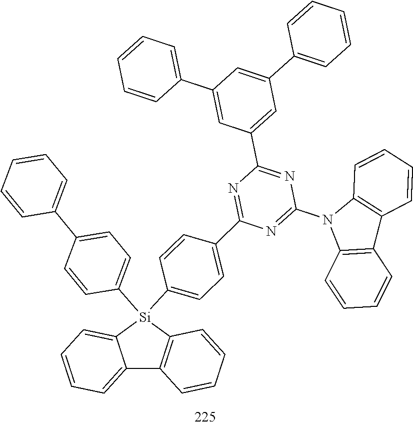 | 59 |
| 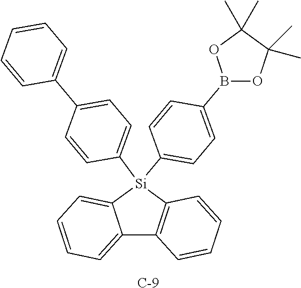 | 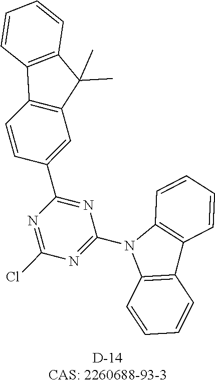 | 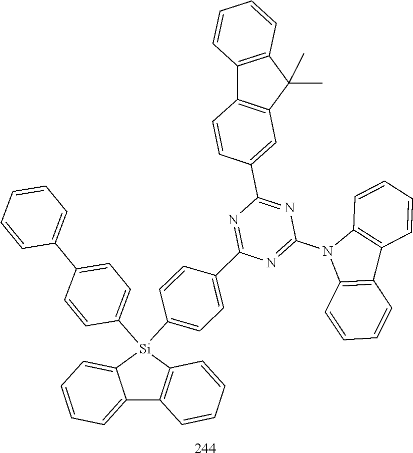 | 58 |
| 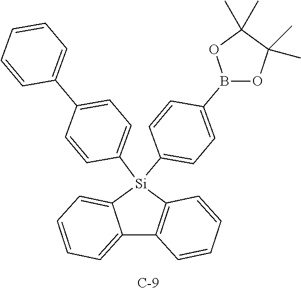 | 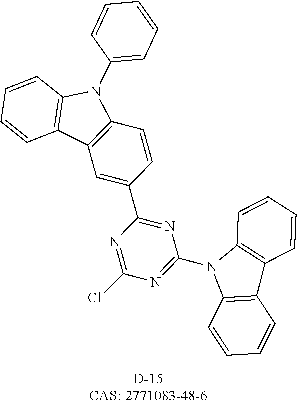 | 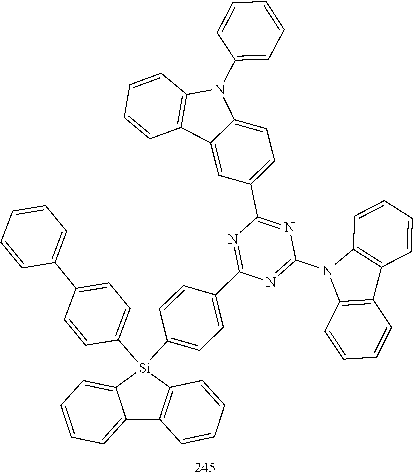 | 66 |
| 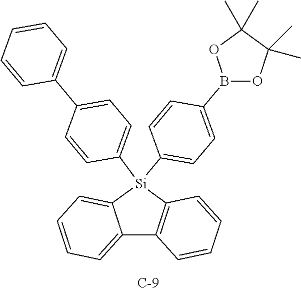 | 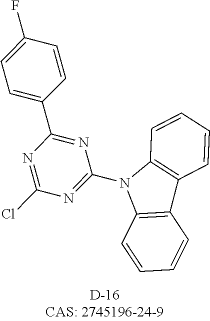 | 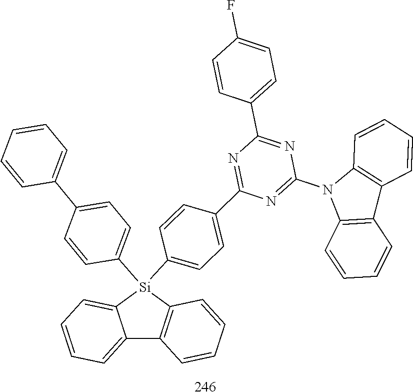 | 62 |
| 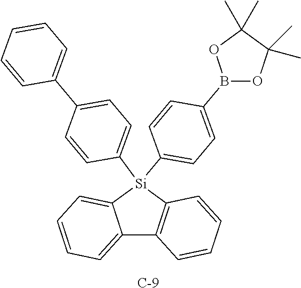 | 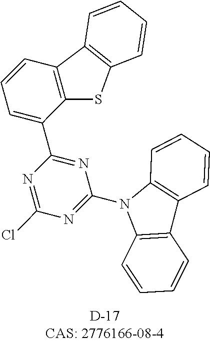 | 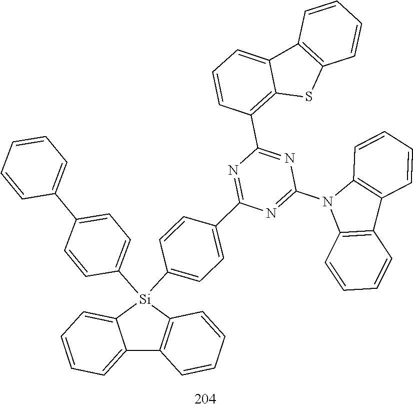 | 58 |
| 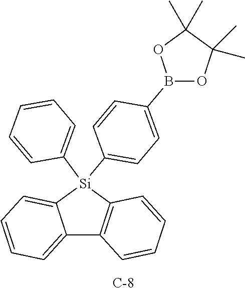 | 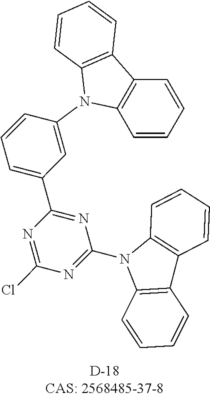 | 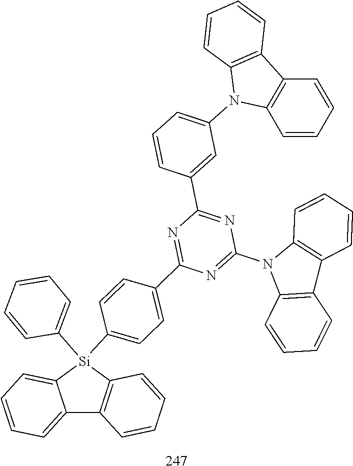 | 54 |

The mass spectral data of the aforementioned-synthesized compounds are presented in Table 4.

TABLE 4
Mass spectral data of the compounds
synthesized in the present disclosure

| Compound No. | m/z([M + H]⁺) | Compound No. | m/z([M + H]⁺) |
|---|---|---|---|
| Compound 1 | 744.3 | Compound 85 | 731.3 |
| Compound 54 | 731.3 | Compound 146 | 821.3 |
| Compound 233 | 758.3 | Compound 100 | 745.3 |
| Compound 32 | 749.3 | Compound 67 | 660.3 |
| Compound 235 | 794.3 | Compound 234 | 762.3 |
| Compound 20 | 909.3 | Compound 132 | 680.3 |
| Compound 185 | 745.3 | Compound 33 | 820.3 |
| Compound 179 | 750.3 | Compound 142 | 749.3 |
| Compound 240 | 663.3 | Compound 241 | 705.2 |
| Compound 242 | 660.3 | Compound 243 | 731.3 |
| Compound 225 | 883.3 | Compound 244 | 847.3 |
| Compound 245 | 896.3 | Compound 246 | 749.2 |
| Compound 204 | 837.2 | Compound 247 | 820.3 |

NMR of Compound 1: ¹H-NMR (400 MHz, Methylene-Chloride-D₂) δ ppm 8.85 (d, 1H), 8.80 (s, 1H), 8.67 (d, 4H), 8.50 (d, 4H), 8.32 (d, 2H), 7.88 (d, 2H), 7.79-7.63 (m, 2H), 7.59-7.53 (m, 7H), 7.47 (d, 1H), 7.43-7.31 (m, 5H), 7.38-7.34 (m, 4H).

### Fabrication and Evaluation of Organic Electroluminescent Device:

### Example 1: Blue Organic Electroluminescent Devices

Substrate treatment: ITO/Ag/ITO substrates with thicknesses of 100Å/1000Å/100Å are subjected to a pretreatment process, wherein the substrate surfaces are cleaned using organic solvents and deionized water to eliminate any floating debris. Surface treatment is then performed utilizing ultraviolet ozone and O₂:N₂ plasma to enhance the work function of the anode of the substrate.

On the test substrate anode, PD and HT-2 were vapor deposited in a ratio of 2%: 98% to form a hole injection layer with a thickness of 100 Å, and on the hole injection layer, HT-2 was vapor deposited to form a hole transport layer with a thickness of 950 Å.

On the hole transport layer, HT-3 with a thickness of 60 Å was vapor deposited as an electron blocking layer.

Subsequently, on the electron blocking layer, BH-HT: Compound 1: BD were vapor deposited in a vapor deposition rate ratio of 59%: 29%: 12% to form a blue organic light-emitting layer with a thickness of 220 Å.

On the organic light-emitting layer, ET-2 (mSiTrz) and LiQ were vapor deposited in a rate ratio of 1:1 to form an electron transport layer with a thickness of 300 Å. Subsequently, a 10 Å thick of Yb was vapor deposited on the electron transport layer as an electron injection layer. On the electron injection layer, magnesium and silver were vapor deposited in a ratio of 1:9 to form a cathode with a thickness of 130 Å.

Last, on the cathode, CP-2 with a thickness of 720 Å was vapor deposited to form an organic capping layer, thereby completing the fabrication of the blue organic light-emitting device.

### Examples 2 to 26

Blue organic electroluminescent devices were fabricated using the same method as in Example 1, except that the compound BH-ET as shown in Table 5 was used instead of Compound 1 in Example 1 as the host material for the electron-transporting light-emitting layer.

### Comparative Examples 1 to 4

Blue organic electroluminescent devices were fabricated using the same method as in Example 1, except that the Compounds A, B, C, and D were used instead of Compound 1 in Example 1 as the host material for the electron-transporting light-emitting layer.

In Examples 1 to 26 and Comparative Examples 1 to 4, the structural formulas of the respective materials utilized are as follows:

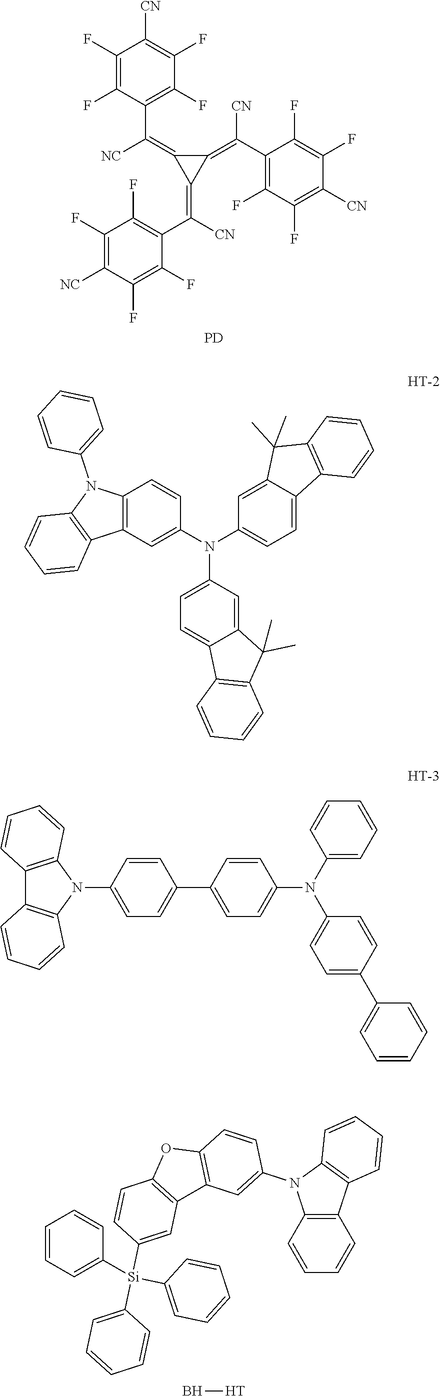

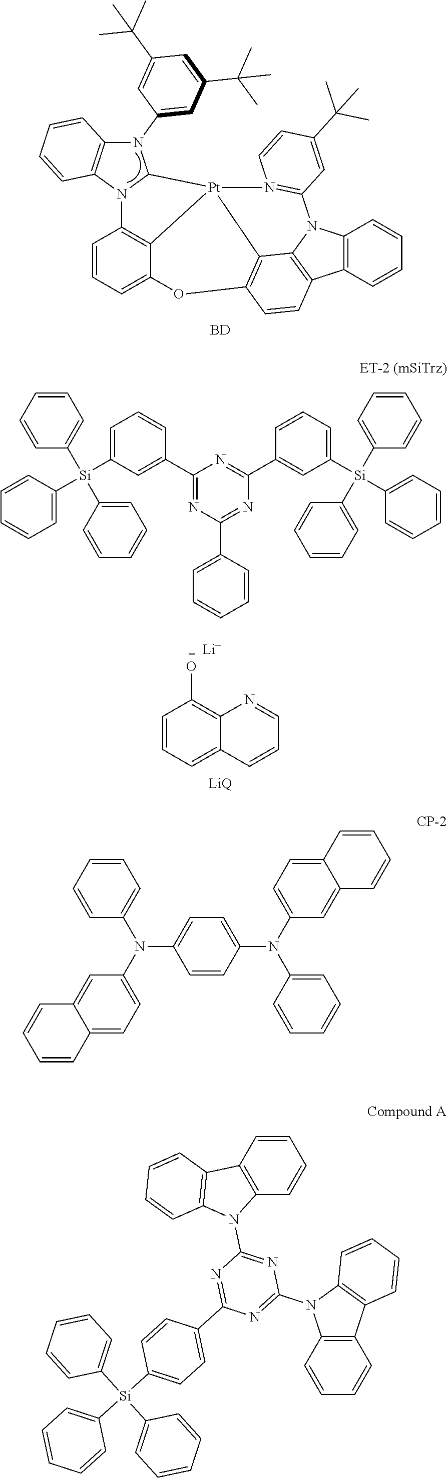

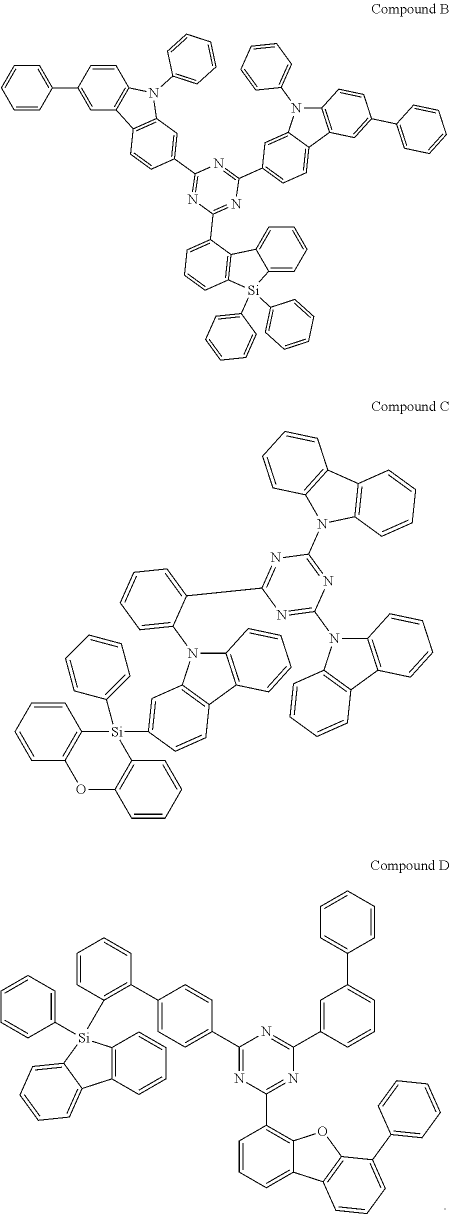 [SMILES: c1ccc(-c2ccc3c(c2)c2ccc(-c4nc(-c5ccc6c7cc(-c8ccccc8)ccc7n(-c7ccccc7)c6c5)nc(-c5cccc6c5-c5ccccc5[Si]6(c5ccccc5)c5ccccc5)n4)cc2n3-c2ccccc2)cc1 ; c1ccc([Si]2(c3ccc4c5ccccc5n(-c5ccccc5-c5nc(-n6c7ccccc7c7ccccc76)nc(-n6c7ccccc7c7ccccc76)n5)c4c3)c3ccccc3Oc3ccccc32)cc1 ; c1ccc(-c2cccc(-c3nc(-c4ccc(-c5ccccc5[Si]5(c6ccccc6)c6ccccc6-c6ccccc65)cc4)nc(-c4cccc5c4oc4c(-c6ccccc6)cccc45)n3)c2)cc1]

The performances of the blue organic electroluminescent devices fabricated in Examples 1 to 26 and Comparative Examples 1 to 4 were tested specifically at a luminance of 1000 nit, and the results are presented in Table 5 below:

TABLE 5
The performance test results of the devices

| | Light-emitting | Operating | | | | |
|---|---|---|---|---|---|---|
| | layer | voltage | | | | T₉₅ |
| No. | BH-ET | Volt (V) | Cd/A | CIEx | CIEy | (h) |
| Example 1 | Compound 1 | 3.84 | 26.75 | 0.140 | 0.140 | 176 |
| Example 2 | Compound 85 | 3.86 | 26.21 | 0.140 | 0.140 | 172 |
| Example 3 | Compound 54 | 3.88 | 25.11 | 0.140 | 0.140 | 158 |
| Example 4 | Compound 146 | 3.81 | 26.25 | 0.140 | 0.140 | 171 |
| Example 5 | Compound 233 | 3.86 | 25.68 | 0.140 | 0.140 | 159 |
| Example 6 | Compound 100 | 3.81 | 26.19 | 0.140 | 0.140 | 167 |
| Example 7 | Compound 32 | 3.85 | 26.86 | 0.140 | 0.140 | 188 |
| Example 8 | Compound 67 | 3.84 | 26.25 | 0.140 | 0.140 | 183 |
| Example 9 | Compound 235 | 3.83 | 26.64 | 0.140 | 0.140 | 174 |
| Example 10 | Compound 234 | 3.85 | 26.67 | 0.140 | 0.140 | 176 |
| Example 11 | Compound 20 | 3.95 | 26.49 | 0.140 | 0.140 | 157 |
| Example 12 | Compound 132 | 3.85 | 25.53 | 0.140 | 0.140 | 159 |
| Example 13 | Compound 185 | 3.87 | 25.62 | 0.140 | 0.140 | 166 |
| Example 14 | Compound 33 | 3.82 | 26.78 | 0.140 | 0.140 | 168 |
| Example 15 | Compound 179 | 3.86 | 26.24 | 0.140 | 0.140 | 186 |
| Example 16 | Compound 142 | 3.86 | 25.29 | 0.140 | 0.140 | 157 |
| Example 17 | Compound 240 | 3.83 | 26.23 | 0.140 | 0.140 | 187 |
| Example 18 | Compound 241 | 3.83 | 25.25 | 0.140 | 0.140 | 158 |
| Example 19 | Compound 242 | 3.85 | 26.26 | 0.140 | 0.140 | 163 |
| Example 20 | Compound 243 | 3.84 | 26.47 | 0.140 | 0.140 | 168 |
| Example 21 | Compound 225 | 3.84 | 26.26 | 0.140 | 0.140 | 171 |
| Example 22 | Compound 244 | 3.86 | 26.17 | 0.140 | 0.140 | 165 |
| Example 23 | Compound 245 | 3.88 | 25.26 | 0.140 | 0.140 | 155 |
| Example 24 | Compound 246 | 3.87 | 26.28 | 0.140 | 0.140 | 168 |
| Example 25 | Compound 204 | 3.86 | 25.22 | 0.140 | 0.140 | 153 |
| Example 26 | Compound 247 | 3.88 | 26.25 | 0.140 | 0.140 | 173 |
| Comparative | Compound A | 4.18 | 15.15 | 0.140 | 0.140 | 121 |
| Example 1 | | | | | | |
| Comparative | Compound B | 4.20 | 17.18 | 0.140 | 0.140 | 132 |
| Example 2 | | | | | | |
| Comparative | Compound C | 4.16 | 18.17 | 0.140 | 0.140 | 126 |
| Example 3 | | | | | | |
| Comparative | Compound D | 4.01 | 21.39 | 0.140 | 0.140 | 131 |
| Example 4 | | | | | | |

It can be seen from Table 5 above that, compared to the devices of Comparative examples 1 to 4, the devices in which the compounds of the present disclosure were used as the electron transporting host material in the light-emitting layer of Examples 1 to 26 exhibited an enhancement in the current efficiency (Cd/A) of the organic electroluminescent devices by at least 17.39%, and extension in the service life by at least 15.91%.

The preferred embodiments of the present invention are described in detail above in conjunction with the accompanying drawings. However, the present invention is not limited to the specific details of the above embodiments. Within the scope of the technical concept of the present invention, various simple modifications can be made to the technical solutions of the present invention, and these simple modifications all fall within the scope of protection of the present invention.

1. An organic compound, having the structure represented by the following Formula 1: 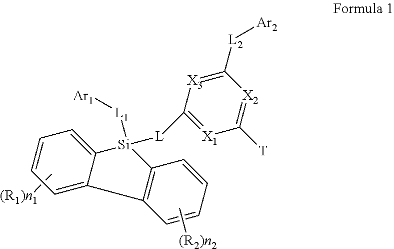
wherein X₁, X₂, and X₃ are each independently selected from N and C(H), and at least two of X₁ to X₃ are N;
Ar₁ and Ar₂ are the same or different, and are each independently selected from a substituted or unsubstituted aryl having 6 to 40 carbon atoms, and a substituted or unsubstituted heteroaryl having 3 to 40 carbon atoms;
L is selected from a single bond, a substituted or unsubstituted arylene having 6 to 30 carbon atoms, and a substituted or unsubstituted heteroarylene having 3 to 30 carbon atoms;
L₁ and L₂ are the same or different, and are each independently a single bond, a substituted or unsubstituted arylene having 6 to 30 carbon atoms, and a substituted or unsubstituted heteroarylene having 3 to 30 carbon atoms;
group T is a substituted or unsubstituted carbazolyl;
substituent(s) in L, L₁, L₂, Ar₁, and Ar₂ are the same or different, and are each independently selected from a deuterium, a cyano, a halogen group, an alkyl having 1 to 10 carbon atoms, a haloalkyl having 1 to 10 carbon atoms, a deuterated alkyl having 1 to 10 carbon atoms, a trialkylsilyl having 3 to 12 carbon atoms, a triphenylsilyl, an aryl having 6 to 20 carbon atoms, an alkylaryl having 6 to 20 carbon atoms, a deuterated aryl having 6 to 20 carbon atoms, a haloaryl having 6 to 20 carbon atoms, a heteroaryl having 3 to 20 carbon atoms, and a cycloalkyl having 3 to 10 carbon atoms; optionally, any two adjacent substituents form a substituted or unsubstituted ring A;
the ring A is a saturated or unsaturated 5 to 13 membered ring;
substituent(s) in the group T, substituent(s) on the ring A, and each of R₁ and R₂ are the same or different, and are each independently selected from a deuterium, a cyano, a halogen group, an alkyl having 1 to 10 carbon atoms, a haloalkyl having 1 to 10 carbon atoms, a deuterated alkyl having 1 to 10 carbon atoms, a trialkylsilyl having 3 to 12 carbon atoms, a triphenylsilyl, an aryl having 6 to 20 carbon atoms, a deuterated aryl having 6 to 20 carbon atoms, a haloaryl having 6 to 20 carbon atoms, a heteroaryl having 3 to 20 carbon atoms, and a cycloalkyl having 3 to 10 carbon atoms;
n₁ is selected from 0, 1, 2, 3, and 4;
n₂ is selected from 0, 1, 2, 3, and 4.

2. The organic compound according to claim 1, wherein Ar₁ and Ar₂ are the same or different, and are each independently selected from a substituted or unsubstituted group V;
wherein the unsubstituted group V is selected from the following groups: 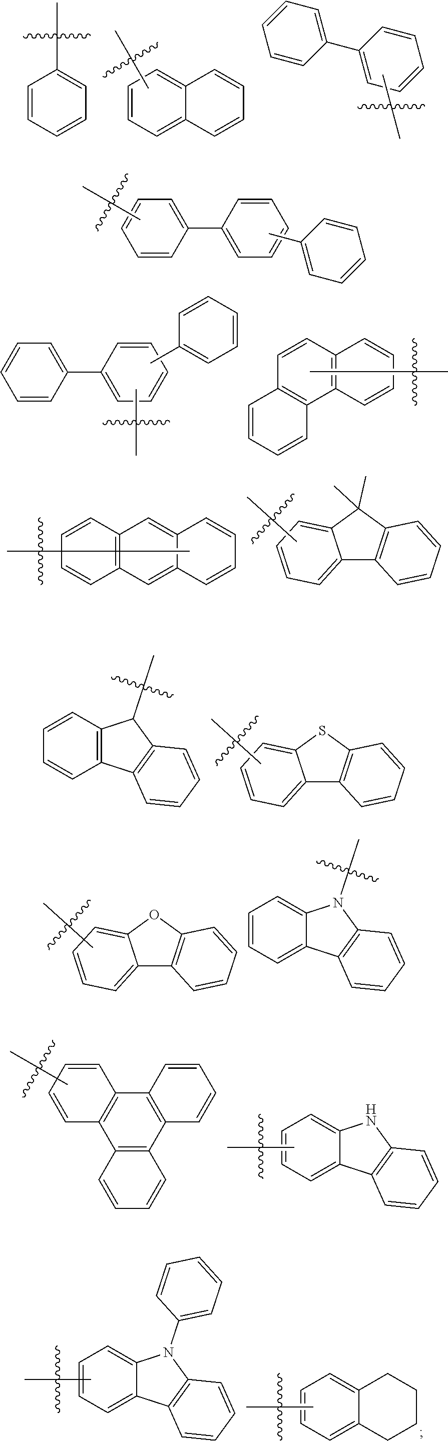
the substituted group V has one or more substituent(s), and the substituent(s) are each independently selected from a deuterium, a fluorine, a cyano, a trimethylsilyl, a trideuterated methyl, a trifluoromethyl, a cyclopentyl, a cyclohexyl, a methyl, an ethyl, an isopropyl, a tert-butyl, a phenyl, a naphthyl, a biphenyl, a fluorenyl, a phenanthryl, a dibenzofuranyl, a dibenzothienyl, and a carbazolyl, and when the number of substituents on the group V is greater than 1, the substituents are the same or different.

3. The organic compound according to claim 1, wherein Ar₁ and Ar₂ are the same or different, and are each independently selected from the group consisting of the following groups:

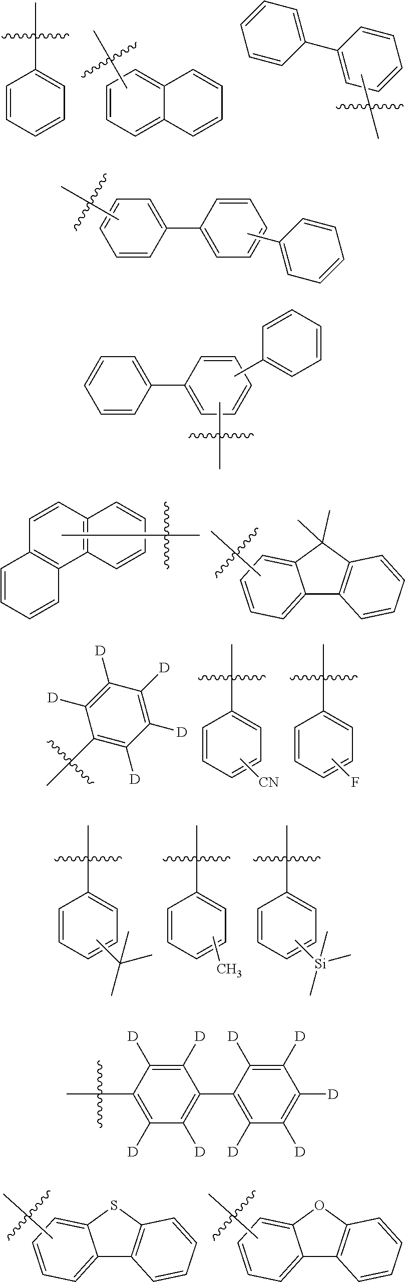

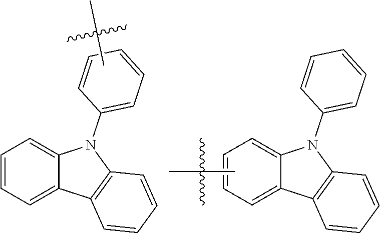

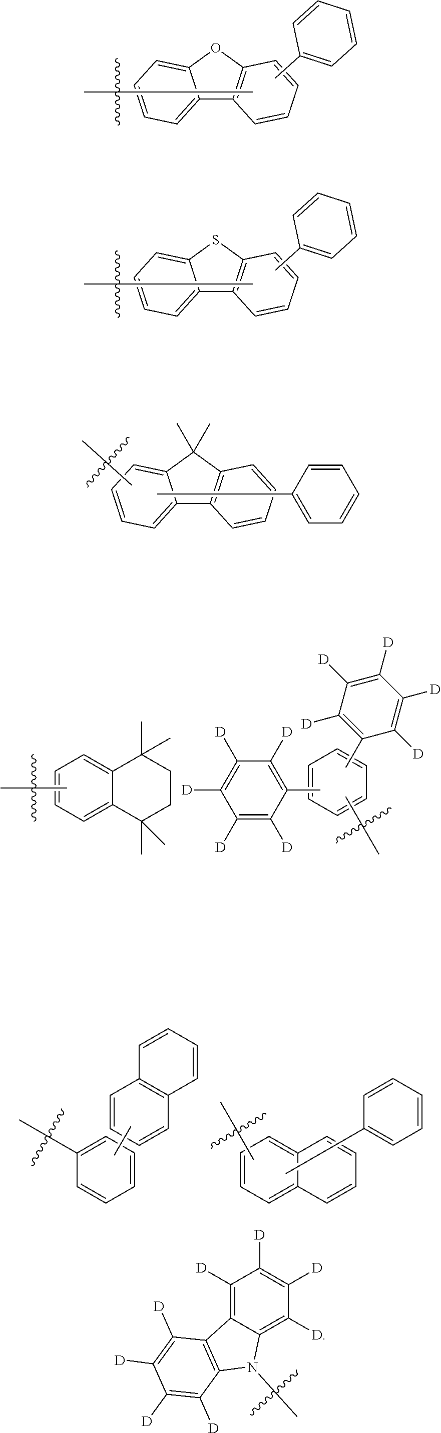

4. The organic compound according to claim 1, wherein L₁ and L₂ are the same or different, and are each independently selected from a single bond, a substituted or unsubstituted phenylene, a substituted or unsubstituted naphthylene, a substituted or unsubstituted biphenylene, a substituted or unsubstituted anthrylene, a substituted or unsubstituted phenanthrylene, a substituted or unsubstituted fluorenylene, a substituted or unsubstituted pyridylene, a substituted or unsubstituted dibenzothienylene, a substituted or unsubstituted dibenzofuranylene, and a substituted or unsubstituted carbazolylene;
optionally, substituent(s) in L₁ and L₂ are the same or different, and are each independently selected from a deuterium, a fluorine, a cyano, a methyl, an ethyl, an isopropyl, a tert-butyl, a trifluoromethyl, a trideuterated methyl, a trimethylsilyl, a phenyl, a methylphenyl, and a pentadeuterated phenyl.

5. The organic compound according to claim 1, wherein L is selected from a substituted or unsubstituted phenylene, a substituted or unsubstituted naphthylene, a substituted or unsubstituted biphenylene, a substituted or unsubstituted anthrylene, a substituted or unsubstituted phenanthrylene, a substituted or unsubstituted fluorenylene, a substituted or unsubstituted pyridylene, a substituted or unsubstituted dibenzothienylene, a substituted or unsubstituted dibenzofuranylene, and a substituted or unsubstituted carbazolylene;
optionally, substituent(s) in L are the same or different, and are each independently selected from a deuterium, a fluorine, a cyano, a methyl, an ethyl, an isopropyl, a tert-butyl, a trifluoromethyl, a trideuterated methyl, a trimethylsilyl, a phenyl, a methylphenyl, and a pentadeuterated phenyl.

6. The organic compound according to claim 1, wherein L is selected from the group consisting of the following groups:

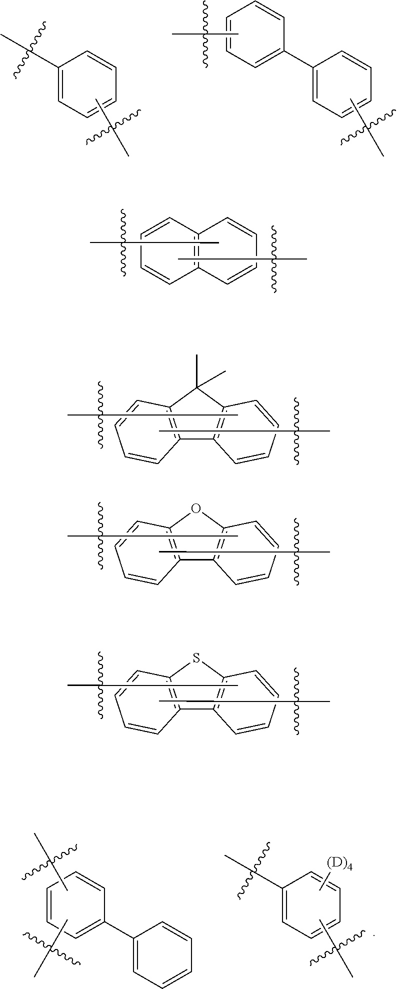

7. The organic compound according to claim 1, wherein L₁ and L₂ are each independently selected from a single bond and the group consisting of the following groups:

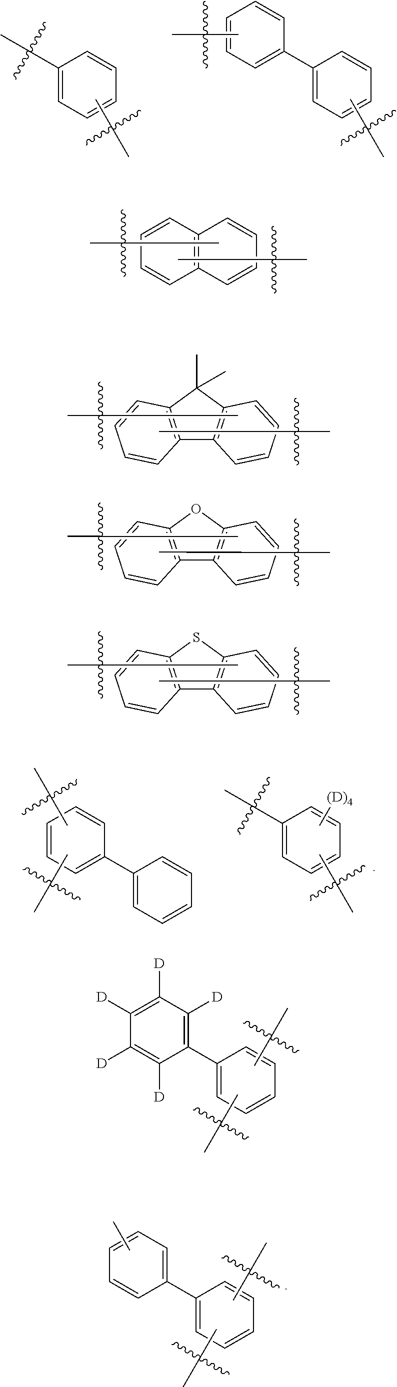

8. The organic compound according to claim 1, wherein —L₁—Ar₁ is selected from the group consisting of the following groups:

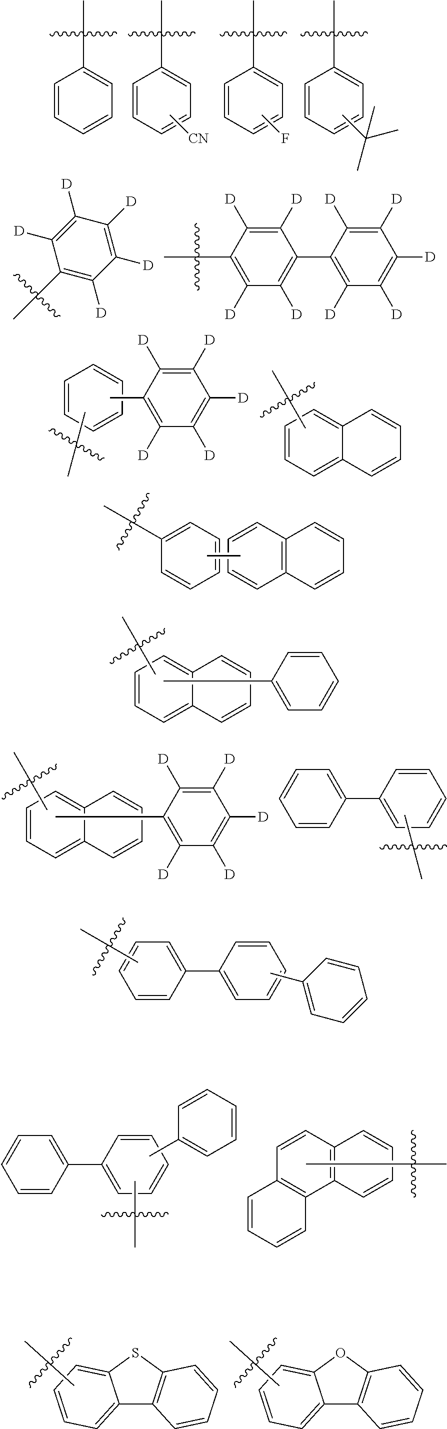

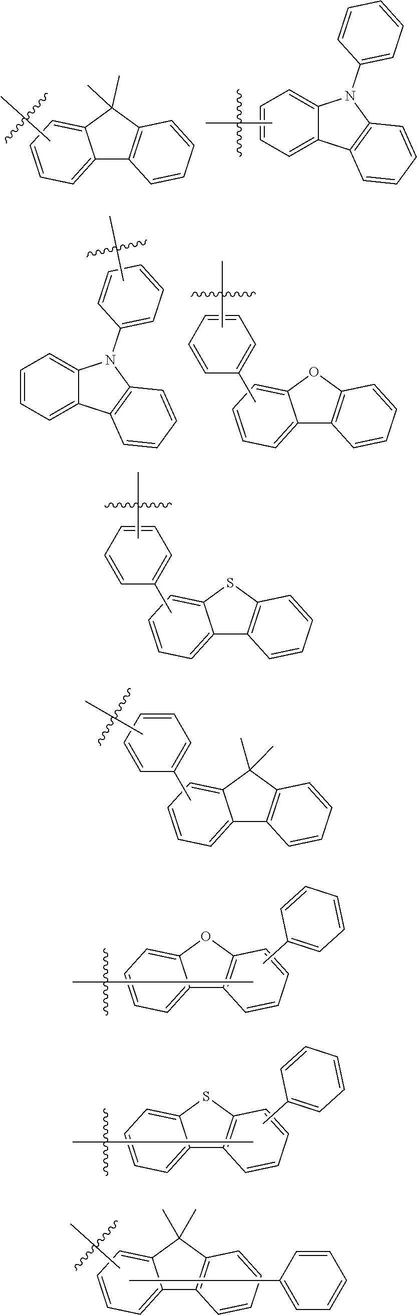

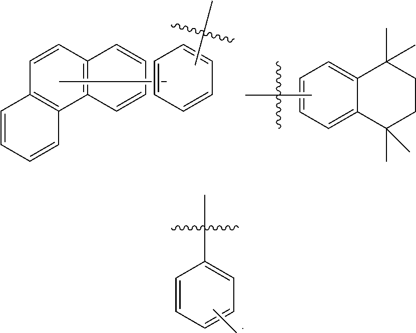

9. The organic compound according to claim 1, wherein 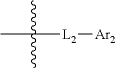
is selected from the group consisting of the following groups:

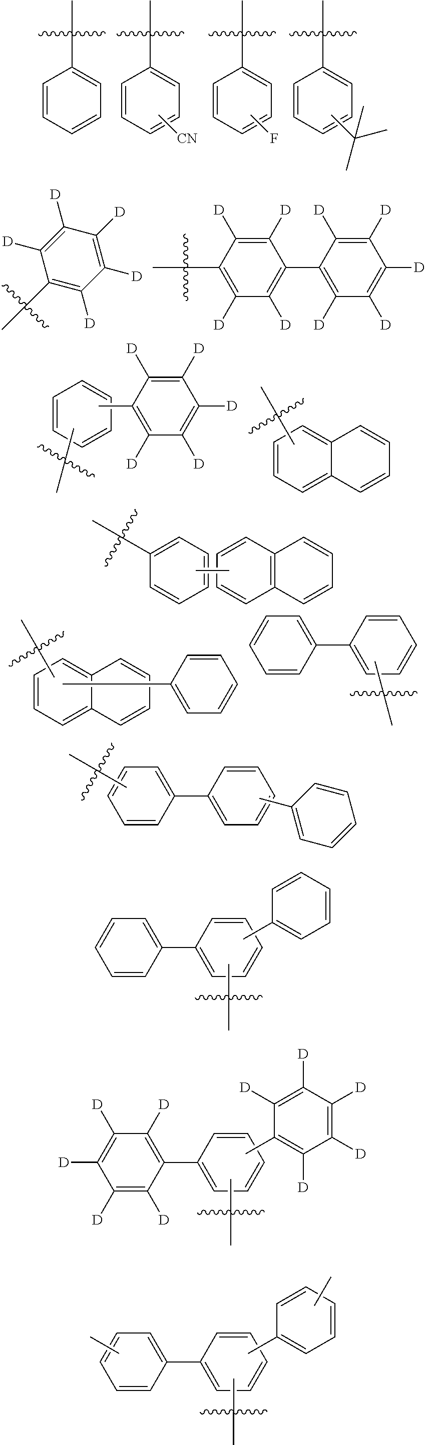

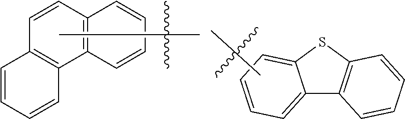

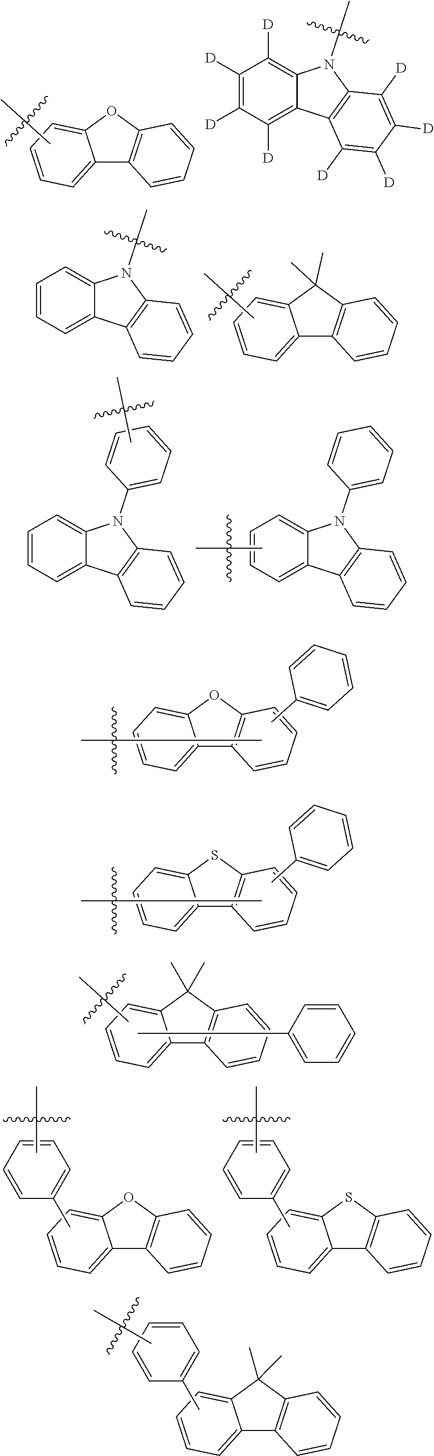

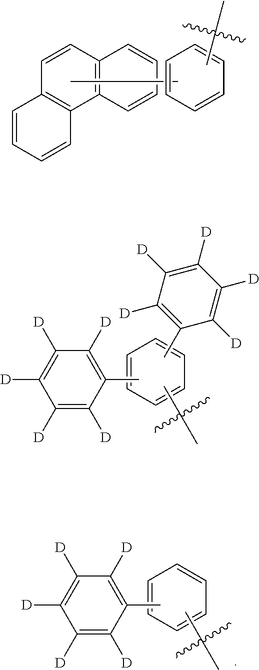

10. The organic compound according to claim 1, wherein the organic compound is selected from the structure represented by the following Formula (1-1): 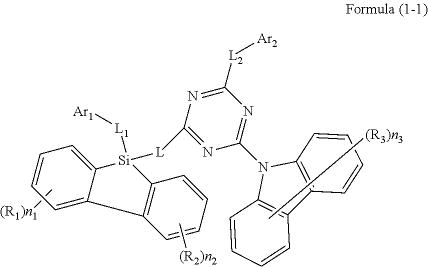
wherein, each R₃ is the same or different, and is independently selected from a deuterium, a cyano, a halogen group, an alkyl having 1 to 5 carbon atoms, a haloalkyl having 1 to 5 carbon atoms, a deuterated alkyl having 1 to 5 carbon atoms, a trialkylsilyl having 3 to 8 carbon atoms, an aryl having 6 to 15 carbon atoms, a deuterated aryl having 6 to 15, a haloaryl having 6 to 15 carbon atoms, a heteroaryl having 5 to 12 carbon atoms, and a cycloalkyl having 3 to 8 carbon atoms; n₃ represents the number of R₃, and n₃ is selected from 0, 1, 2, 3, 4, 5, 6, 7, or 8.

11. The organic compound according to claim 10, wherein each R₃ is the same or different, and is independently selected from a deuterium, a cyano, a fluorine, a methyl, an ethyl, an isopropyl, a tert-butyl, a trifluoromethyl, a trideuterated methyl, a trimethylsilyl, a phenyl, a pentadeuterated phenyl, a biphenyl, and a naphthyl;
optionally, each R₁ and R₂ are the same or different, and are each independently selected from a deuterium, a cyano, a fluorine, a methyl, an ethyl, an isopropyl, a tert-butyl, a trifluoromethyl, a trideuterated methyl, a trimethylsilyl, a phenyl, a pentadeuterated phenyl, a biphenyl, and a naphthyl.

12. The organic compound according to claim 1, wherein the organic compound is selected from the group consisting of the following compounds:

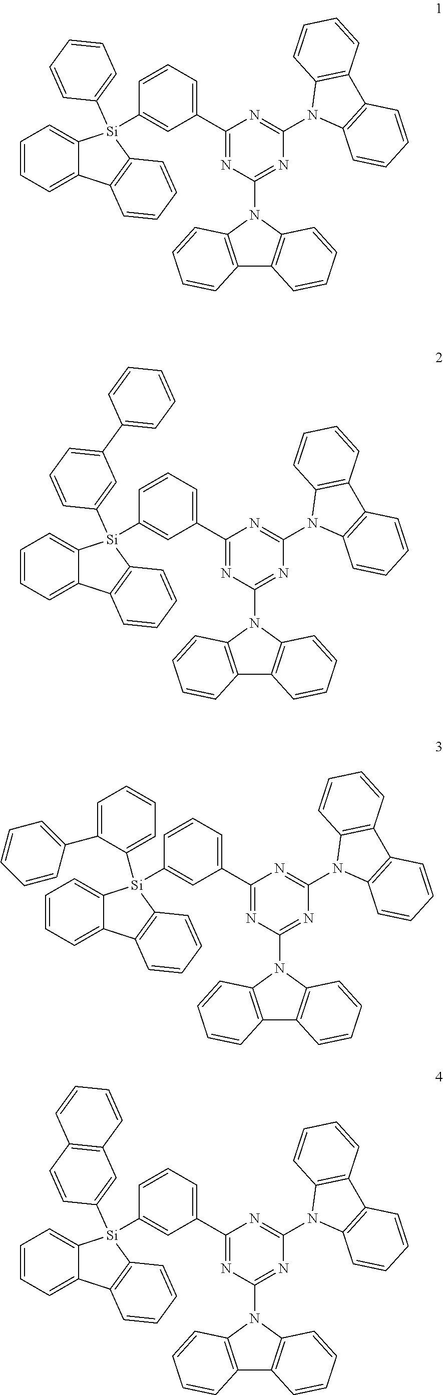 [SMILES: c1ccc([Si]2(c3cccc(-c4nc(-n5c6ccccc6c6ccccc65)nc(-n5c6ccccc6c6ccccc65)n4)c3)c3ccccc3-c3ccccc32)cc1 ; c1ccc(-c2cccc([Si]3(c4cccc(-c5nc(-n6c7ccccc7c7ccccc76)nc(-n6c7ccccc7c7ccccc76)n5)c4)c4ccccc4-c4ccccc43)c2)cc1 ; c1ccc(-c2ccccc2[Si]2(c3cccc(-c4nc(-n5c6ccccc6c6ccccc65)nc(-n5c6ccccc6c6ccccc65)n4)c3)c3ccccc3-c3ccccc32)cc1 ; c1cc(-c2nc(-n3c4ccccc4c4ccccc43)nc(-n3c4ccccc4c4ccccc43)n2)cc([Si]2(c3ccc4ccccc4c3)c3ccccc3-c3ccccc32)c1]

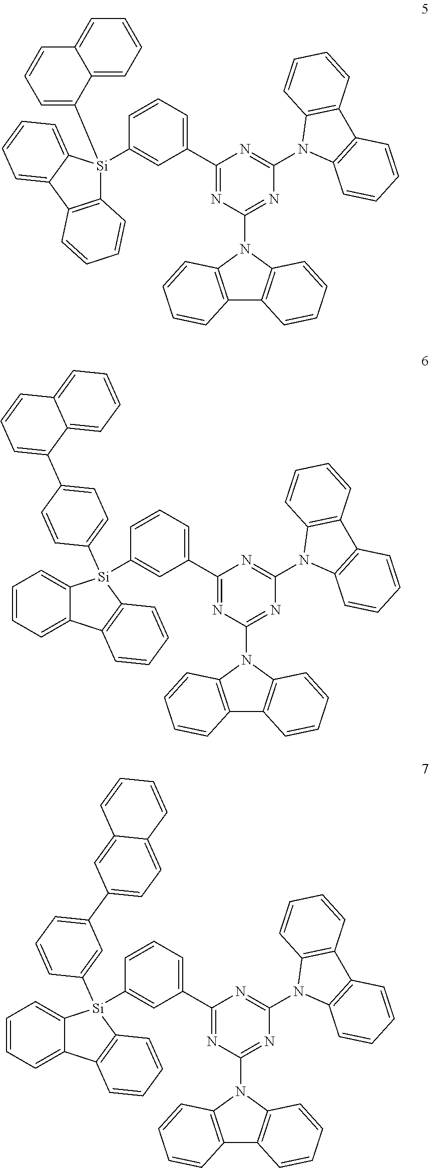 [SMILES: c1cc(-c2nc(-n3c4ccccc4c4ccccc43)nc(-n3c4ccccc4c4ccccc43)n2)cc([Si]2(c3cccc4ccccc34)c3ccccc3-c3ccccc32)c1 ; c1cc(-c2nc(-n3c4ccccc4c4ccccc43)nc(-n3c4ccccc4c4ccccc43)n2)cc([Si]2(c3ccc(-c4cccc5ccccc45)cc3)c3ccccc3-c3ccccc32)c1 ; c1cc(-c2ccc3ccccc3c2)cc([Si]2(c3cccc(-c4nc(-n5c6ccccc6c6ccccc65)nc(-n5c6ccccc6c6ccccc65)n4)c3)c3ccccc3-c3ccccc32)c1]

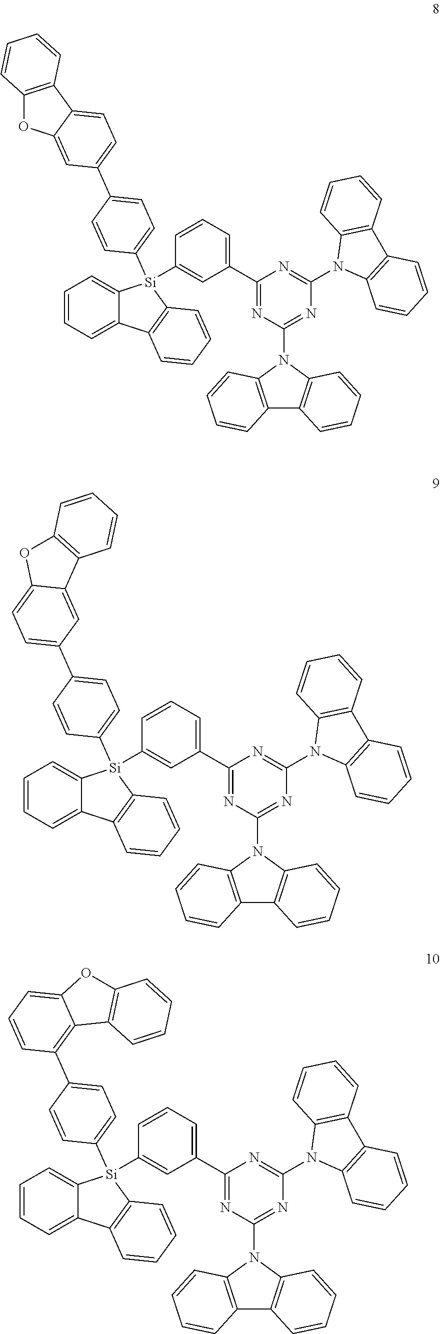 [SMILES: c1cc(-c2nc(-n3c4ccccc4c4ccccc43)nc(-n3c4ccccc4c4ccccc43)n2)cc([Si]2(c3ccc(-c4ccc5c(c4)oc4ccccc45)cc3)c3ccccc3-c3ccccc32)c1 ; c1cc(-c2nc(-n3c4ccccc4c4ccccc43)nc(-n3c4ccccc4c4ccccc43)n2)cc([Si]2(c3ccc(-c4ccc5oc6ccccc6c5c4)cc3)c3ccccc3-c3ccccc32)c1 ; c1cc(-c2nc(-n3c4ccccc4c4ccccc43)nc(-n3c4ccccc4c4ccccc43)n2)cc([Si]2(c3ccc(-c4cccc5oc6ccccc6c45)cc3)c3ccccc3-c3ccccc32)c1]

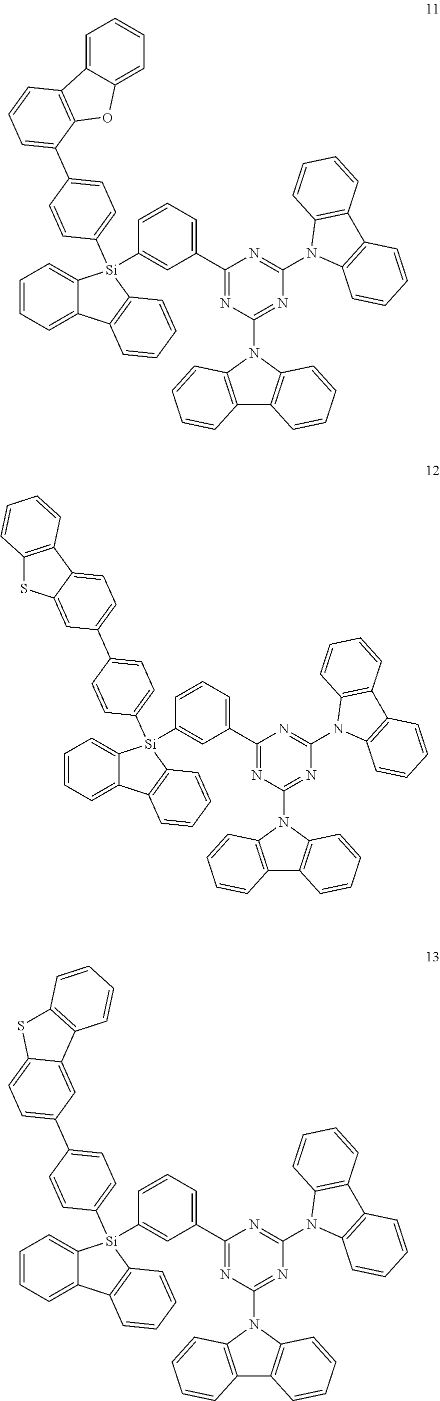 [SMILES: c1cc(-c2nc(-n3c4ccccc4c4ccccc43)nc(-n3c4ccccc4c4ccccc43)n2)cc([Si]2(c3ccc(-c4cccc5c4oc4ccccc45)cc3)c3ccccc3-c3ccccc32)c1 ; c1cc(-c2nc(-n3c4ccccc4c4ccccc43)nc(-n3c4ccccc4c4ccccc43)n2)cc([Si]2(c3ccc(-c4ccc5c(c4)sc4ccccc45)cc3)c3ccccc3-c3ccccc32)c1 ; c1cc(-c2nc(-n3c4ccccc4c4ccccc43)nc(-n3c4ccccc4c4ccccc43)n2)cc([Si]2(c3ccc(-c4ccc5sc6ccccc6c5c4)cc3)c3ccccc3-c3ccccc32)c1]

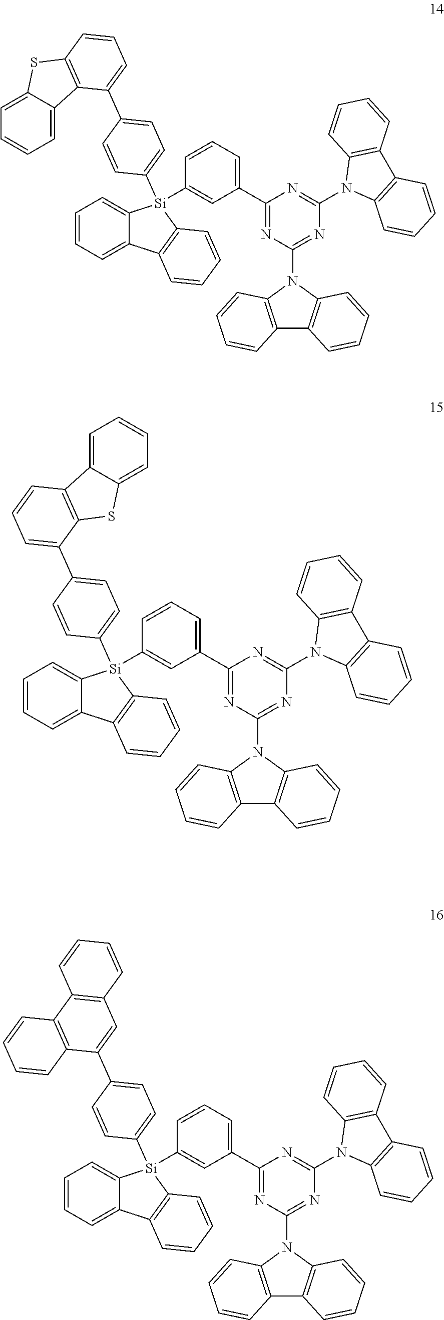 [SMILES: c1cc(-c2nc(-n3c4ccccc4c4ccccc43)nc(-n3c4ccccc4c4ccccc43)n2)cc([Si]2(c3ccc(-c4cccc5sc6ccccc6c45)cc3)c3ccccc3-c3ccccc32)c1 ; c1cc(-c2nc(-n3c4ccccc4c4ccccc43)nc(-n3c4ccccc4c4ccccc43)n2)cc([Si]2(c3ccc(-c4cccc5c4sc4ccccc45)cc3)c3ccccc3-c3ccccc32)c1 ; c1cc(-c2nc(-n3c4ccccc4c4ccccc43)nc(-n3c4ccccc4c4ccccc43)n2)cc([Si]2(c3ccc(-c4cc5ccccc5c5ccccc45)cc3)c3ccccc3-c3ccccc32)c1]

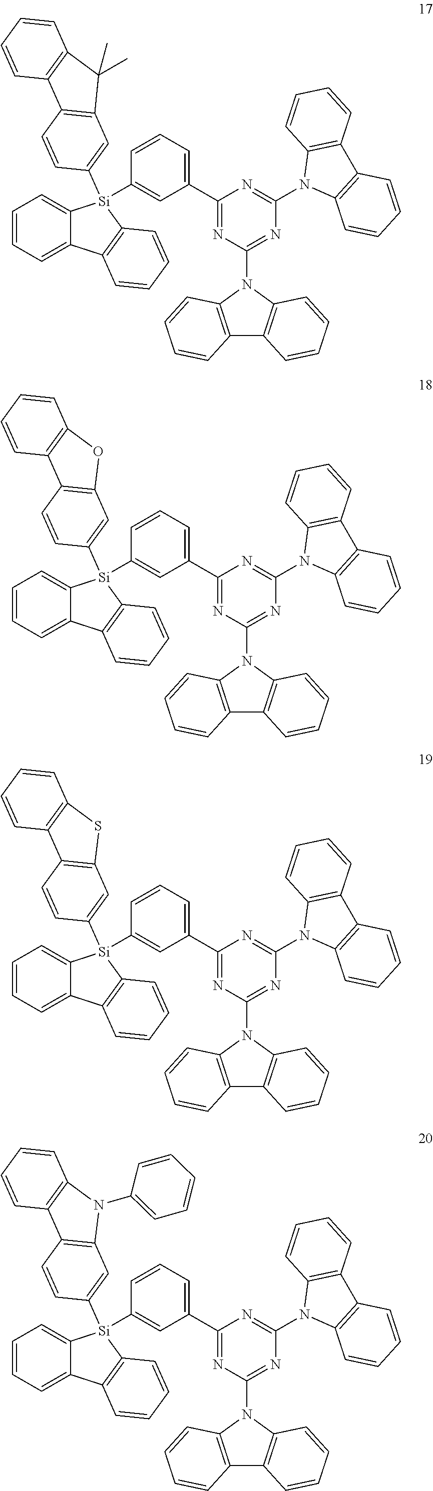 [SMILES: CC1(C)c2ccccc2-c2ccc([Si]3(c4cccc(-c5nc(-n6c7ccccc7c7ccccc76)nc(-n6c7ccccc7c7ccccc76)n5)c4)c4ccccc4-c4ccccc43)cc21 ; c1cc(-c2nc(-n3c4ccccc4c4ccccc43)nc(-n3c4ccccc4c4ccccc43)n2)cc([Si]2(c3ccc4c(c3)oc3ccccc34)c3ccccc3-c3ccccc32)c1 ; c1cc(-c2nc(-n3c4ccccc4c4ccccc43)nc(-n3c4ccccc4c4ccccc43)n2)cc([Si]2(c3ccc4c(c3)sc3ccccc34)c3ccccc3-c3ccccc32)c1 ; c1ccc(-n2c3ccccc3c3ccc([Si]4(c5cccc(-c6nc(-n7c8ccccc8c8ccccc87)nc(-n7c8ccccc8c8ccccc87)n6)c5)c5ccccc5-c5ccccc54)cc32)cc1]

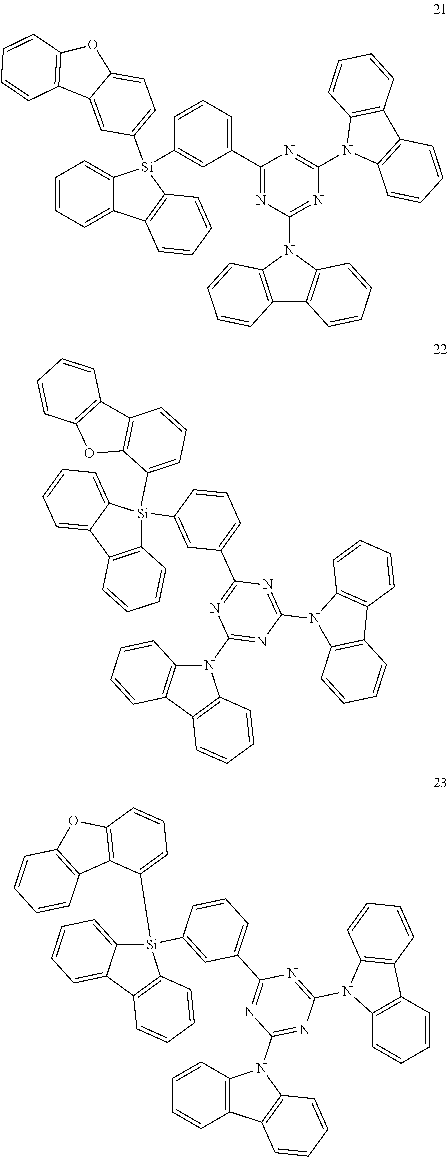 [SMILES: c1cc(-c2nc(-n3c4ccccc4c4ccccc43)nc(-n3c4ccccc4c4ccccc43)n2)cc([Si]2(c3ccc4oc5ccccc5c4c3)c3ccccc3-c3ccccc32)c1 ; c1cc(-c2nc(-n3c4ccccc4c4ccccc43)nc(-n3c4ccccc4c4ccccc43)n2)cc([Si]2(c3cccc4c3oc3ccccc34)c3ccccc3-c3ccccc32)c1 ; c1cc(-c2nc(-n3c4ccccc4c4ccccc43)nc(-n3c4ccccc4c4ccccc43)n2)cc([Si]2(c3cccc4oc5ccccc5c34)c3ccccc3-c3ccccc32)c1]

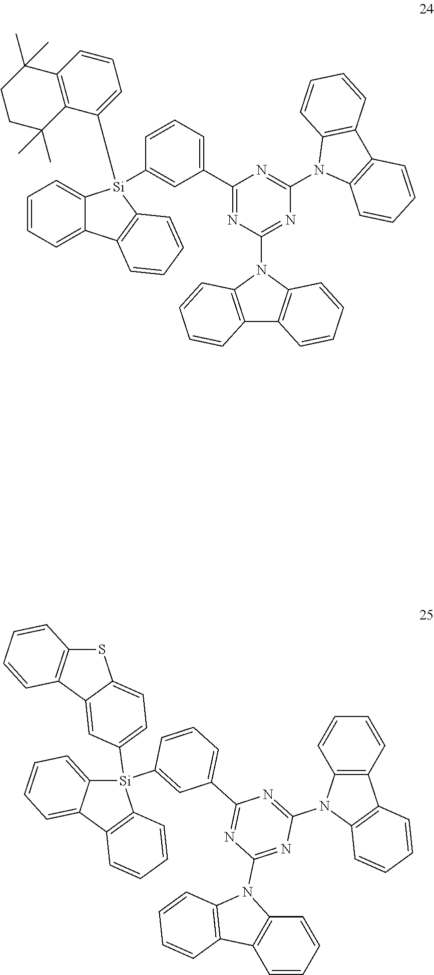 [SMILES: CC1(C)CCC(C)(C)c2c1cccc2[Si]1(c2cccc(-c3nc(-n4c5ccccc5c5ccccc54)nc(-n4c5ccccc5c5ccccc54)n3)c2)c2ccccc2-c2ccccc21 ; c1cc(-c2nc(-n3c4ccccc4c4ccccc43)nc(-n3c4ccccc4c4ccccc43)n2)cc([Si]2(c3ccc4sc5ccccc5c4c3)c3ccccc3-c3ccccc32)c1]

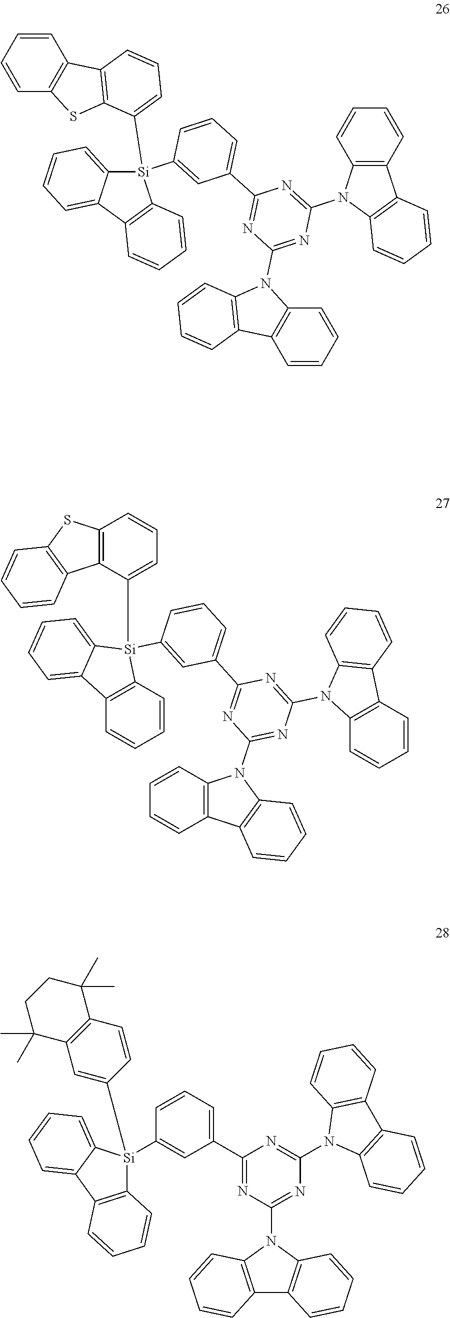 [SMILES: c1cc(-c2nc(-n3c4ccccc4c4ccccc43)nc(-n3c4ccccc4c4ccccc43)n2)cc([Si]2(c3cccc4c3sc3ccccc34)c3ccccc3-c3ccccc32)c1 ; c1cc(-c2nc(-n3c4ccccc4c4ccccc43)nc(-n3c4ccccc4c4ccccc43)n2)cc([Si]2(c3cccc4sc5ccccc5c34)c3ccccc3-c3ccccc32)c1 ; CC1(C)CCC(C)(C)c2cc([Si]3(c4cccc(-c5nc(-n6c7ccccc7c7ccccc76)nc(-n6c7ccccc7c7ccccc76)n5)c4)c4ccccc4-c4ccccc43)ccc21]

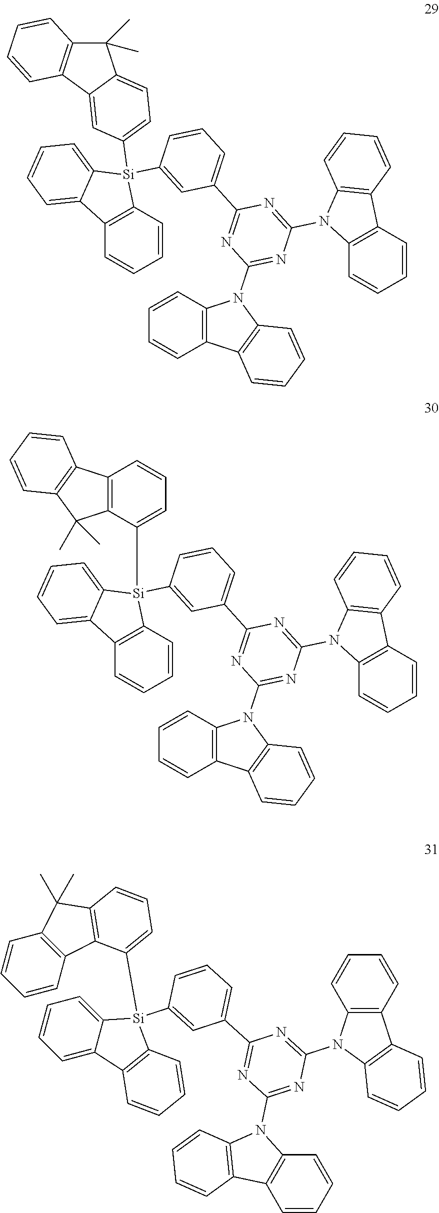 [SMILES: CC1(C)c2ccccc2-c2cc([Si]3(c4cccc(-c5nc(-n6c7ccccc7c7ccccc76)nc(-n6c7ccccc7c7ccccc76)n5)c4)c4ccccc4-c4ccccc43)ccc21 ; CC1(C)c2ccccc2-c2cccc([Si]3(c4cccc(-c5nc(-n6c7ccccc7c7ccccc76)nc(-n6c7ccccc7c7ccccc76)n5)c4)c4ccccc4-c4ccccc43)c21 ; CC1(C)c2ccccc2-c2c1cccc2[Si]1(c2cccc(-c3nc(-n4c5ccccc5c5ccccc54)nc(-n4c5ccccc5c5ccccc54)n3)c2)c2ccccc2-c2ccccc21]

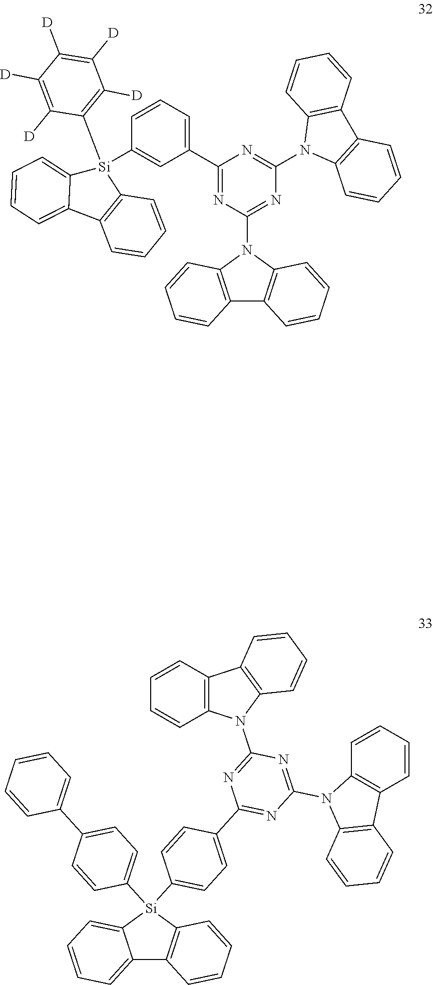 [SMILES: [2H]c1c([2H])c([2H])c([Si]2(c3cccc(-c4nc(-n5c6ccccc6c6ccccc65)nc(-n5c6ccccc6c6ccccc65)n4)c3)c3ccccc3-c3ccccc32)c([2H])c1[2H] ; c1ccc(-c2ccc([Si]3(c4ccc(-c5nc(-n6c7ccccc7c7ccccc76)nc(-n6c7ccccc7c7ccccc76)n5)cc4)c4ccccc4-c4ccccc43)cc2)cc1]

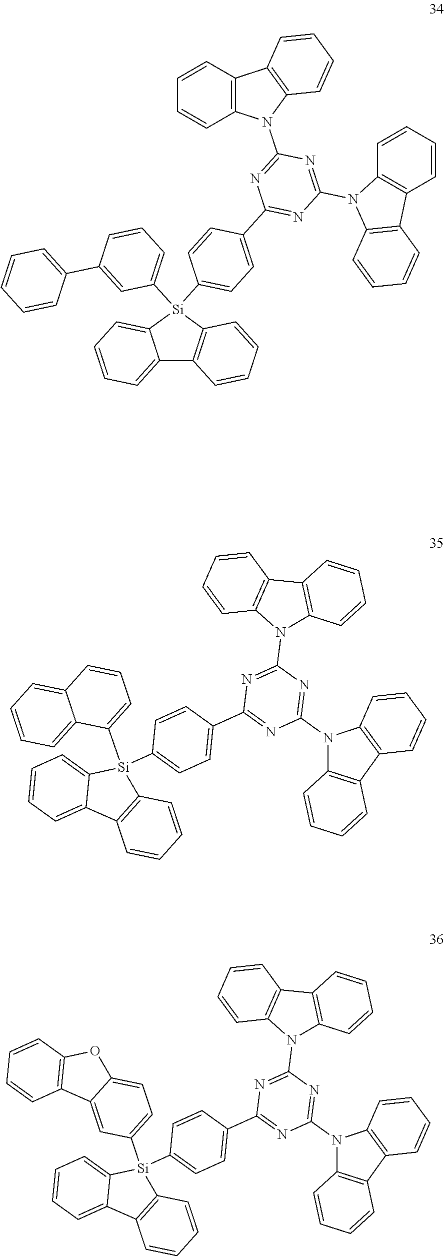 [SMILES: c1ccc(-c2cccc([Si]3(c4ccc(-c5nc(-n6c7ccccc7c7ccccc76)nc(-n6c7ccccc7c7ccccc76)n5)cc4)c4ccccc4-c4ccccc43)c2)cc1 ; c1ccc2c(c1)-c1ccccc1[Si]2(c1ccc(-c2nc(-n3c4ccccc4c4ccccc43)nc(-n3c4ccccc4c4ccccc43)n2)cc1)c1cccc2ccccc12 ; c1ccc2c(c1)-c1ccccc1[Si]2(c1ccc(-c2nc(-n3c4ccccc4c4ccccc43)nc(-n3c4ccccc4c4ccccc43)n2)cc1)c1ccc2oc3ccccc3c2c1]

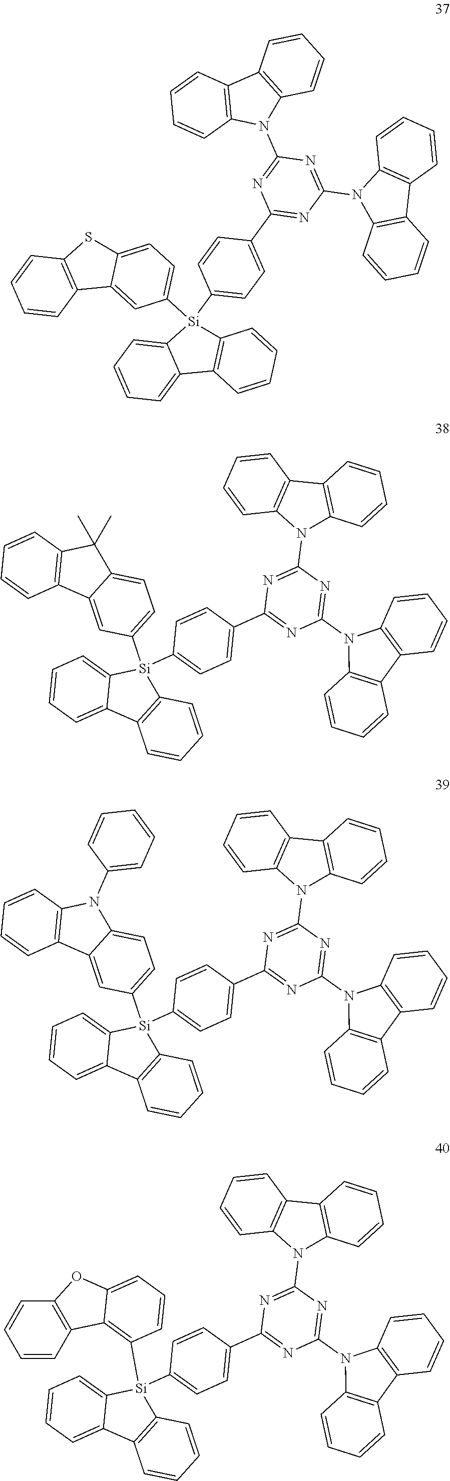 [SMILES: c1ccc2c(c1)-c1ccccc1[Si]2(c1ccc(-c2nc(-n3c4ccccc4c4ccccc43)nc(-n3c4ccccc4c4ccccc43)n2)cc1)c1ccc2sc3ccccc3c2c1 ; CC1(C)c2ccccc2-c2cc([Si]3(c4ccc(-c5nc(-n6c7ccccc7c7ccccc76)nc(-n6c7ccccc7c7ccccc76)n5)cc4)c4ccccc4-c4ccccc43)ccc21 ; c1ccc(-n2c3ccccc3c3cc([Si]4(c5ccc(-c6nc(-n7c8ccccc8c8ccccc87)nc(-n7c8ccccc8c8ccccc87)n6)cc5)c5ccccc5-c5ccccc54)ccc32)cc1 ; c1ccc2c(c1)-c1ccccc1[Si]2(c1ccc(-c2nc(-n3c4ccccc4c4ccccc43)nc(-n3c4ccccc4c4ccccc43)n2)cc1)c1cccc2oc3ccccc3c12]

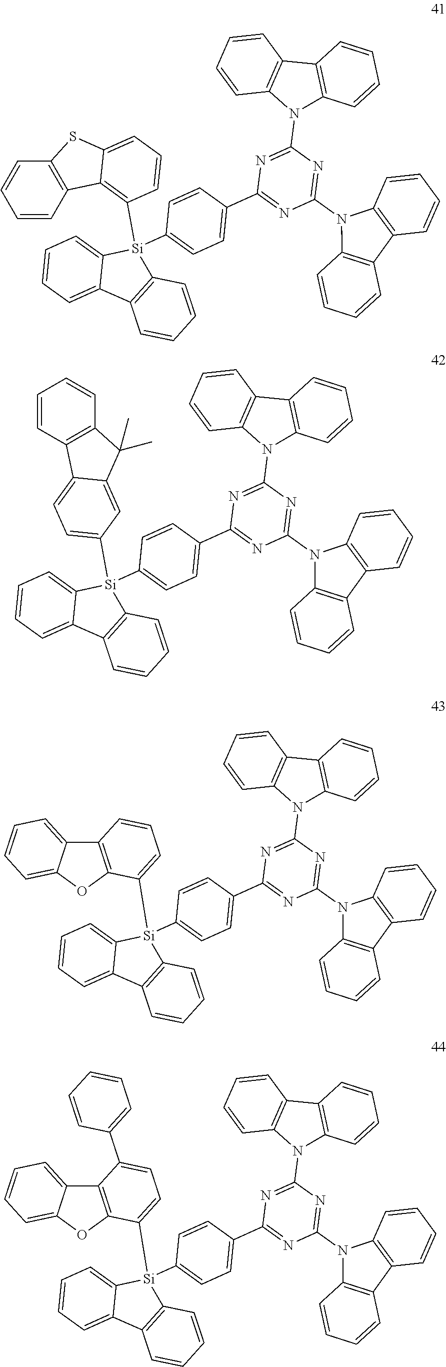

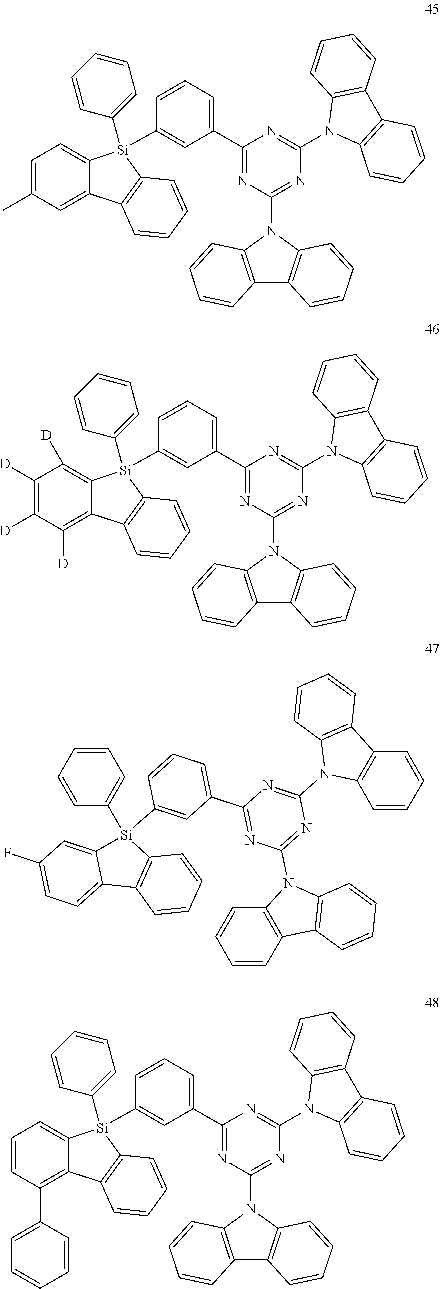 [SMILES: Cc1ccc2c(c1)-c1ccccc1[Si]2(c1ccccc1)c1cccc(-c2nc(-n3c4ccccc4c4ccccc43)nc(-n3c4ccccc4c4ccccc43)n2)c1 ; [2H]c1c([2H])c([2H])c2c(c1[2H])-c1ccccc1[Si]2(c1ccccc1)c1cccc(-c2nc(-n3c4ccccc4c4ccccc43)nc(-n3c4ccccc4c4ccccc43)n2)c1 ; Fc1ccc2c(c1)[Si](c1ccccc1)(c1cccc(-c3nc(-n4c5ccccc5c5ccccc54)nc(-n4c5ccccc5c5ccccc54)n3)c1)c1ccccc1-2 ; c1ccc(-c2cccc3c2-c2ccccc2[Si]3(c2ccccc2)c2cccc(-c3nc(-n4c5ccccc5c5ccccc54)nc(-n4c5ccccc5c5ccccc54)n3)c2)cc1]

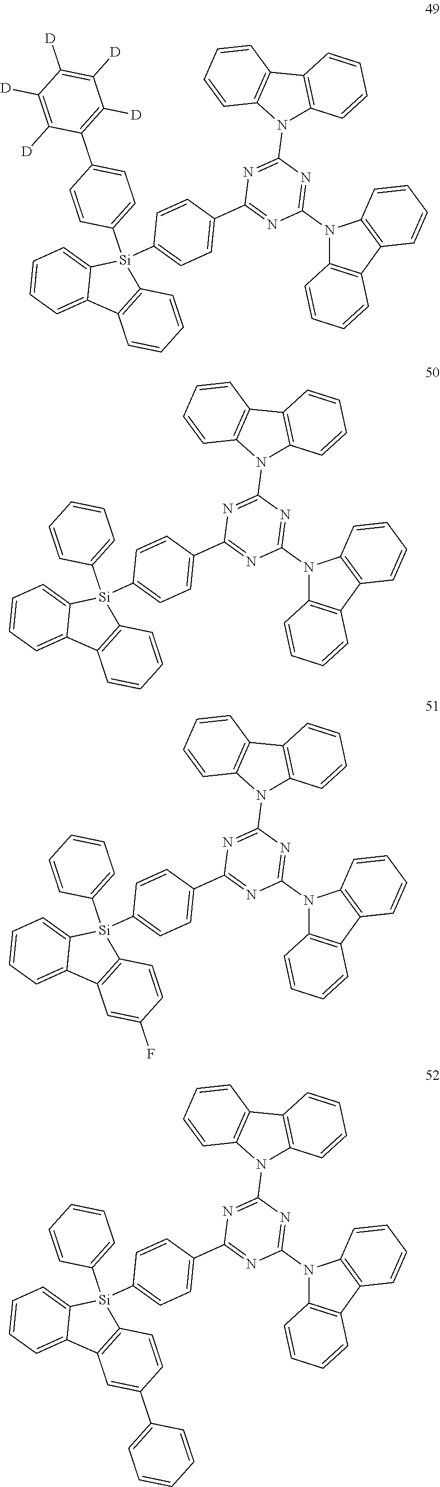 [SMILES: [2H]c1c([2H])c([2H])c(-c2ccc([Si]3(c4ccc(-c5nc(-n6c7ccccc7c7ccccc76)nc(-n6c7ccccc7c7ccccc76)n5)cc4)c4ccccc4-c4ccccc43)cc2)c([2H])c1[2H] ; c1ccc([Si]2(c3ccc(-c4nc(-n5c6ccccc6c6ccccc65)nc(-n5c6ccccc6c6ccccc65)n4)cc3)c3ccccc3-c3ccccc32)cc1 ; Fc1ccc2c(c1)-c1ccccc1[Si]2(c1ccccc1)c1ccc(-c2nc(-n3c4ccccc4c4ccccc43)nc(-n3c4ccccc4c4ccccc43)n2)cc1 ; c1ccc(-c2ccc3c(c2)-c2ccccc2[Si]3(c2ccccc2)c2ccc(-c3nc(-n4c5ccccc5c5ccccc54)nc(-n4c5ccccc5c5ccccc54)n3)cc2)cc1]

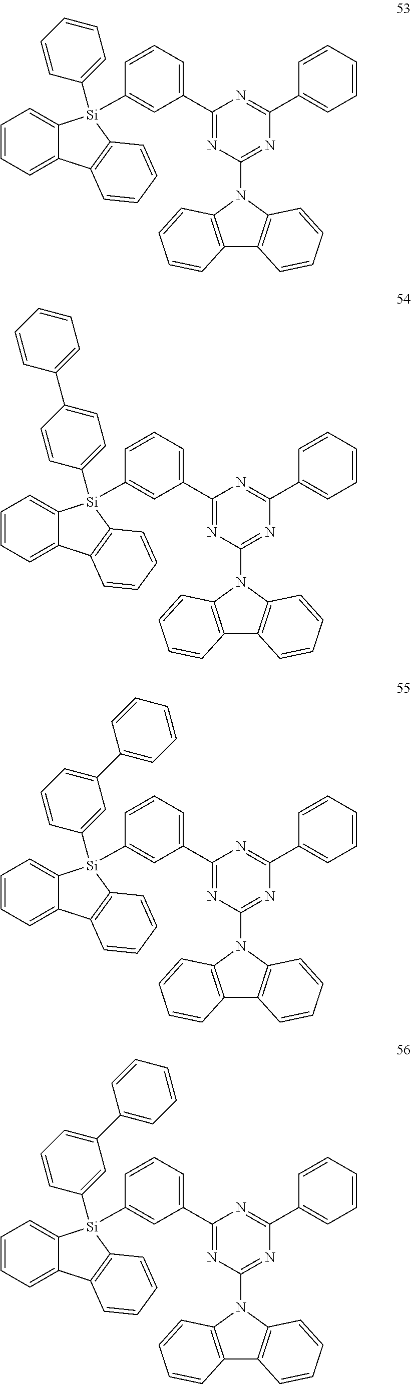 [SMILES: c1ccc(-c2nc(-c3cccc([Si]4(c5ccccc5)c5ccccc5-c5ccccc54)c3)nc(-n3c4ccccc4c4ccccc43)n2)cc1 ; c1ccc(-c2ccc([Si]3(c4cccc(-c5nc(-c6ccccc6)nc(-n6c7ccccc7c7ccccc76)n5)c4)c4ccccc4-c4ccccc43)cc2)cc1 ; c1ccc(-c2cccc([Si]3(c4cccc(-c5nc(-c6ccccc6)nc(-n6c7ccccc7c7ccccc76)n5)c4)c4ccccc4-c4ccccc43)c2)cc1 ; c1ccc(-c2cccc([Si]3(c4cccc(-c5nc(-c6ccccc6)nc(-n6c7ccccc7c7ccccc76)n5)c4)c4ccccc4-c4ccccc43)c2)cc1]

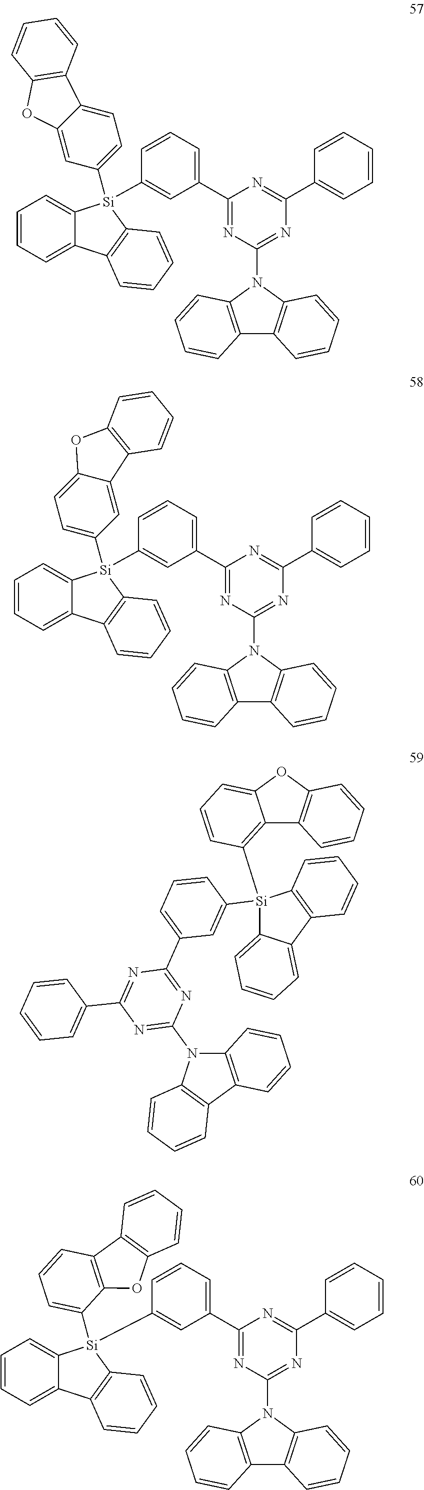

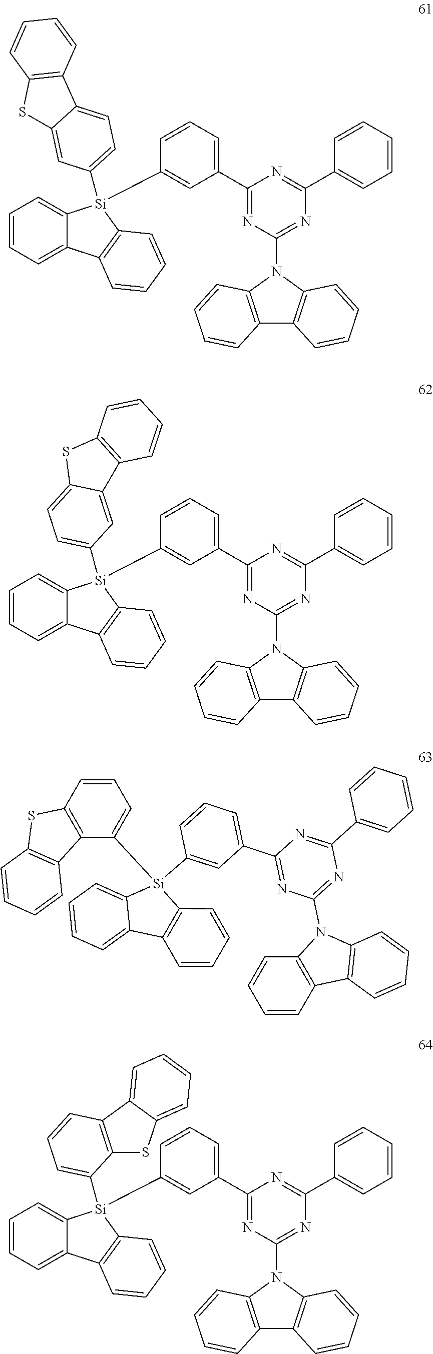 [SMILES: c1ccc(-c2nc(-c3cccc([Si]4(c5ccc6c(c5)sc5ccccc56)c5ccccc5-c5ccccc54)c3)nc(-n3c4ccccc4c4ccccc43)n2)cc1 ; c1ccc(-c2nc(-c3cccc([Si]4(c5ccc6sc7ccccc7c6c5)c5ccccc5-c5ccccc54)c3)nc(-n3c4ccccc4c4ccccc43)n2)cc1 ; c1ccc(-c2nc(-c3cccc([Si]4(c5cccc6sc7ccccc7c56)c5ccccc5-c5ccccc54)c3)nc(-n3c4ccccc4c4ccccc43)n2)cc1 ; c1ccc(-c2nc(-c3cccc([Si]4(c5cccc6c5sc5ccccc56)c5ccccc5-c5ccccc54)c3)nc(-n3c4ccccc4c4ccccc43)n2)cc1]

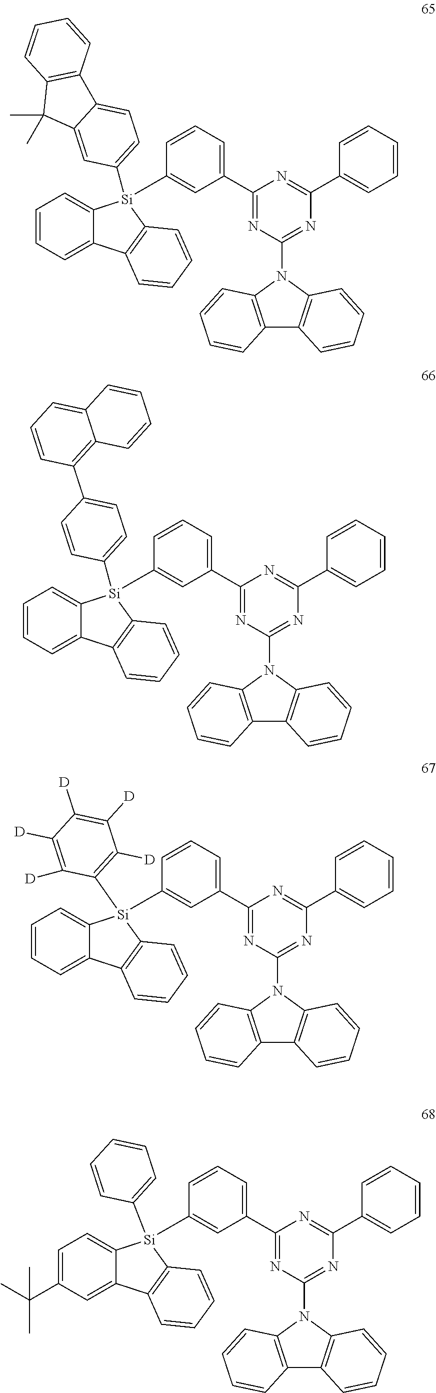 [SMILES: CC1(C)c2ccccc2-c2ccc([Si]3(c4cccc(-c5nc(-c6ccccc6)nc(-n6c7ccccc7c7ccccc76)n5)c4)c4ccccc4-c4ccccc43)cc21 ; c1ccc(-c2nc(-c3cccc([Si]4(c5ccc(-c6cccc7ccccc67)cc5)c5ccccc5-c5ccccc54)c3)nc(-n3c4ccccc4c4ccccc43)n2)cc1 ; [2H]c1c([2H])c([2H])c([Si]2(c3cccc(-c4nc(-c5ccccc5)nc(-n5c6ccccc6c6ccccc65)n4)c3)c3ccccc3-c3ccccc32)c([2H])c1[2H] ; CC(C)(C)c1ccc2c(c1)-c1ccccc1[Si]2(c1ccccc1)c1cccc(-c2nc(-c3ccccc3)nc(-n3c4ccccc4c4ccccc43)n2)c1]

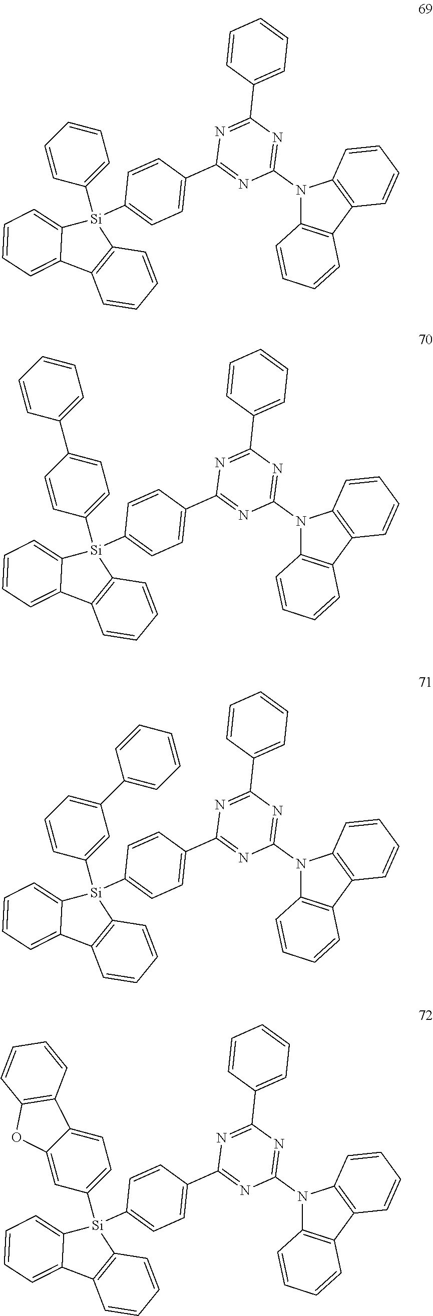 [SMILES: c1ccc(-c2nc(-c3ccc([Si]4(c5ccccc5)c5ccccc5-c5ccccc54)cc3)nc(-n3c4ccccc4c4ccccc43)n2)cc1 ; c1ccc(-c2ccc([Si]3(c4ccc(-c5nc(-c6ccccc6)nc(-n6c7ccccc7c7ccccc76)n5)cc4)c4ccccc4-c4ccccc43)cc2)cc1 ; c1ccc(-c2cccc([Si]3(c4ccc(-c5nc(-c6ccccc6)nc(-n6c7ccccc7c7ccccc76)n5)cc4)c4ccccc4-c4ccccc43)c2)cc1 ; c1ccc(-c2nc(-c3ccc([Si]4(c5ccc6c(c5)oc5ccccc56)c5ccccc5-c5ccccc54)cc3)nc(-n3c4ccccc4c4ccccc43)n2)cc1]

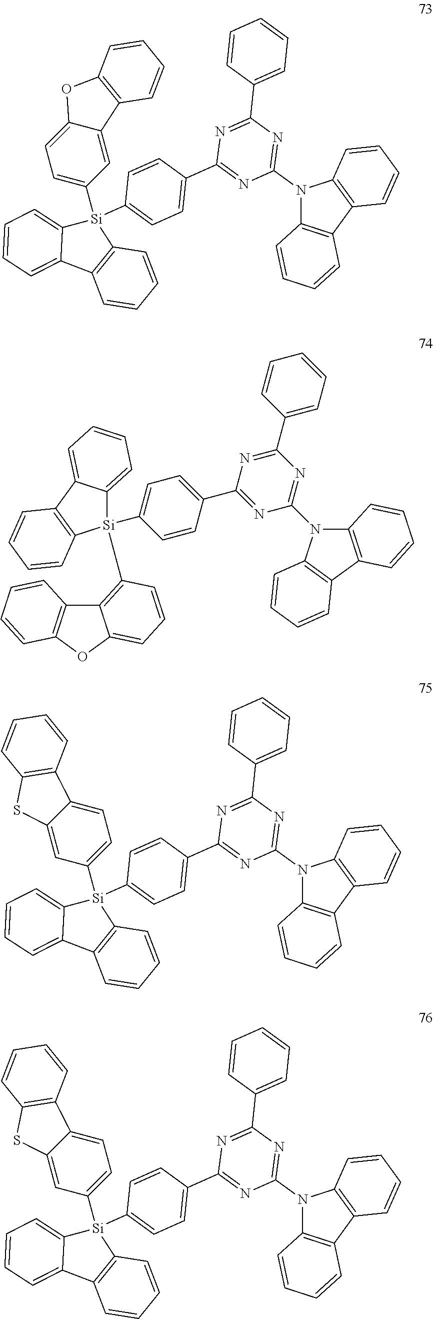 [SMILES: c1ccc(-c2nc(-c3ccc([Si]4(c5ccc6oc7ccccc7c6c5)c5ccccc5-c5ccccc54)cc3)nc(-n3c4ccccc4c4ccccc43)n2)cc1 ; c1ccc(-c2nc(-c3ccc([Si]4(c5cccc6oc7ccccc7c56)c5ccccc5-c5ccccc54)cc3)nc(-n3c4ccccc4c4ccccc43)n2)cc1 ; c1ccc(-c2nc(-c3ccc([Si]4(c5ccc6c(c5)sc5ccccc56)c5ccccc5-c5ccccc54)cc3)nc(-n3c4ccccc4c4ccccc43)n2)cc1 ; c1ccc(-c2nc(-c3ccc([Si]4(c5ccc6c(c5)sc5ccccc56)c5ccccc5-c5ccccc54)cc3)nc(-n3c4ccccc4c4ccccc43)n2)cc1]

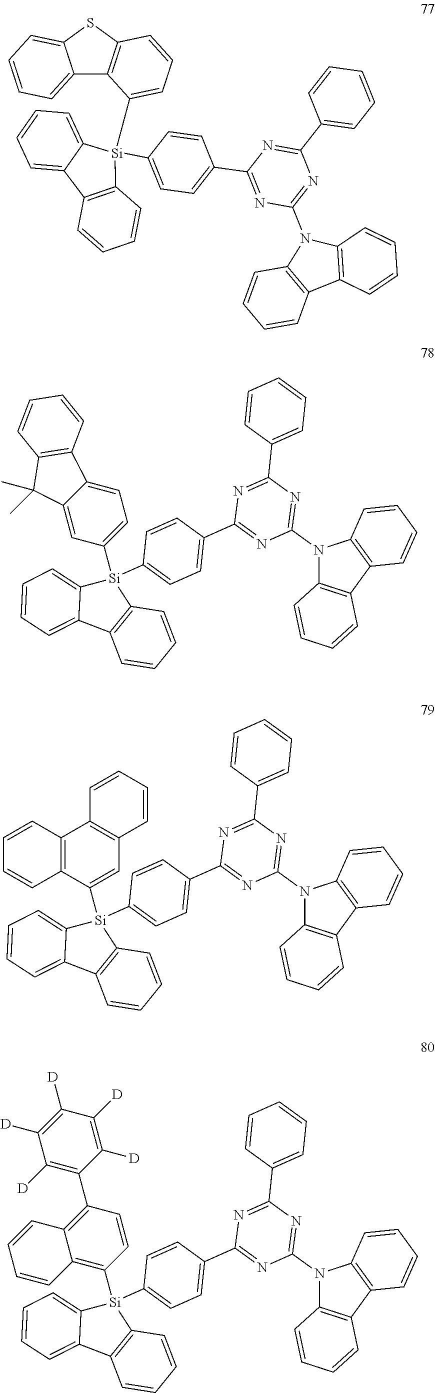 [SMILES: c1ccc(-c2nc(-c3ccc([Si]4(c5cccc6sc7ccccc7c56)c5ccccc5-c5ccccc54)cc3)nc(-n3c4ccccc4c4ccccc43)n2)cc1 ; CC1(C)c2ccccc2-c2ccc([Si]3(c4ccc(-c5nc(-c6ccccc6)nc(-n6c7ccccc7c7ccccc76)n5)cc4)c4ccccc4-c4ccccc43)cc21 ; c1ccc(-c2nc(-c3ccc([Si]4(c5cc6ccccc6c6ccccc56)c5ccccc5-c5ccccc54)cc3)nc(-n3c4ccccc4c4ccccc43)n2)cc1 ; [2H]c1c([2H])c([2H])c(-c2ccc([Si]3(c4ccc(-c5nc(-c6ccccc6)nc(-n6c7ccccc7c7ccccc76)n5)cc4)c4ccccc4-c4ccccc43)c3ccccc23)c([2H])c1[2H]]

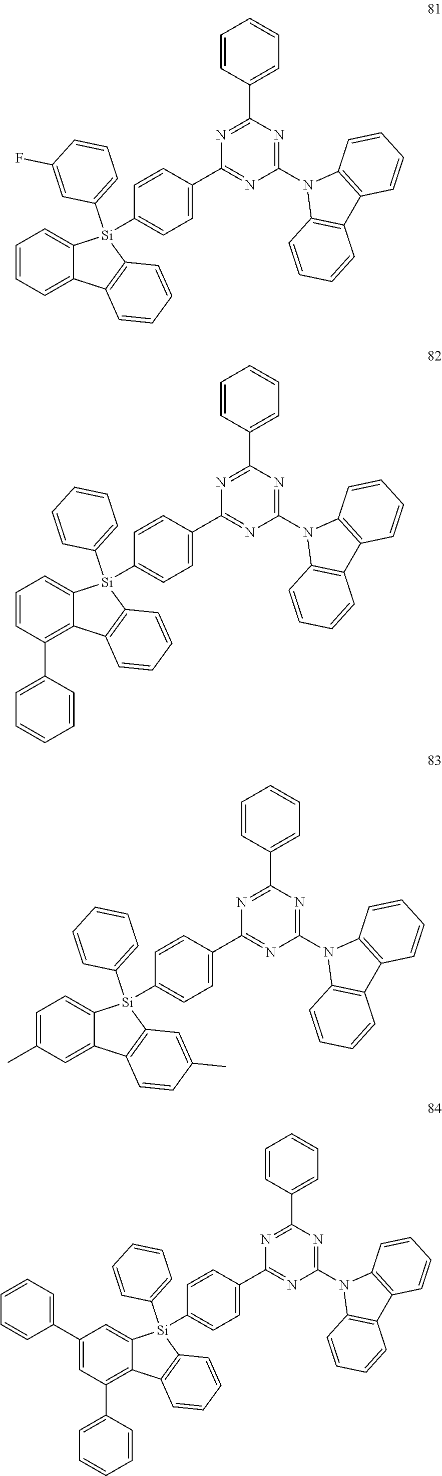 [SMILES: Fc1cccc([Si]2(c3ccc(-c4nc(-c5ccccc5)nc(-n5c6ccccc6c6ccccc65)n4)cc3)c3ccccc3-c3ccccc32)c1 ; c1ccc(-c2nc(-c3ccc([Si]4(c5ccccc5)c5ccccc5-c5c(-c6ccccc6)cccc54)cc3)nc(-n3c4ccccc4c4ccccc43)n2)cc1 ; Cc1ccc2c(c1)-c1ccc(C)cc1[Si]2(c1ccccc1)c1ccc(-c2nc(-c3ccccc3)nc(-n3c4ccccc4c4ccccc43)n2)cc1 ; c1ccc(-c2cc(-c3ccccc3)c3c(c2)[Si](c2ccccc2)(c2ccc(-c4nc(-c5ccccc5)nc(-n5c6ccccc6c6ccccc65)n4)cc2)c2ccccc2-3)cc1]

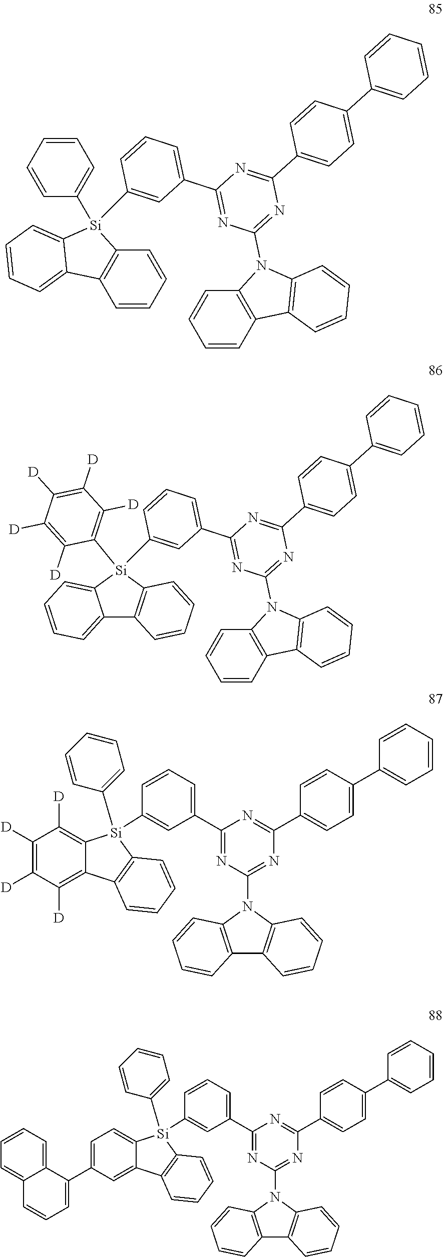 [SMILES: c1ccc(-c2ccc(-c3nc(-c4cccc([Si]5(c6ccccc6)c6ccccc6-c6ccccc65)c4)nc(-n4c5ccccc5c5ccccc54)n3)cc2)cc1 ; [2H]c1c([2H])c([2H])c([Si]2(c3cccc(-c4nc(-c5ccc(-c6ccccc6)cc5)nc(-n5c6ccccc6c6ccccc65)n4)c3)c3ccccc3-c3ccccc32)c([2H])c1[2H] ; [2H]c1c([2H])c([2H])c2c(c1[2H])-c1ccccc1[Si]2(c1ccccc1)c1cccc(-c2nc(-c3ccc(-c4ccccc4)cc3)nc(-n3c4ccccc4c4ccccc43)n2)c1 ; c1ccc(-c2ccc(-c3nc(-c4cccc([Si]5(c6ccccc6)c6ccccc6-c6cc(-c7cccc8ccccc78)ccc65)c4)nc(-n4c5ccccc5c5ccccc54)n3)cc2)cc1]

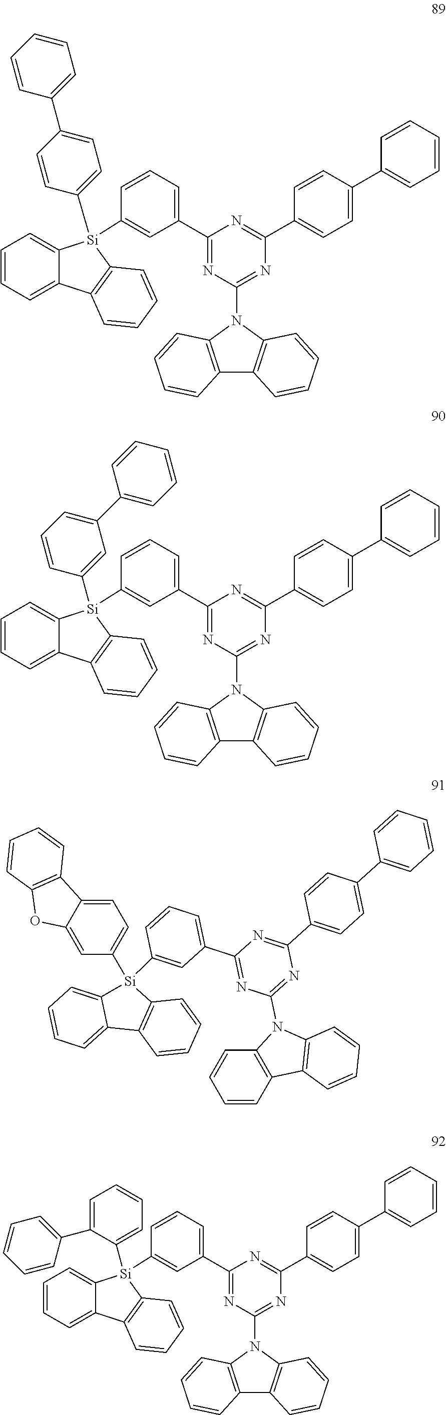 [SMILES: c1ccc(-c2ccc(-c3nc(-c4cccc([Si]5(c6ccc(-c7ccccc7)cc6)c6ccccc6-c6ccccc65)c4)nc(-n4c5ccccc5c5ccccc54)n3)cc2)cc1 ; c1ccc(-c2ccc(-c3nc(-c4cccc([Si]5(c6cccc(-c7ccccc7)c6)c6ccccc6-c6ccccc65)c4)nc(-n4c5ccccc5c5ccccc54)n3)cc2)cc1 ; c1ccc(-c2ccc(-c3nc(-c4cccc([Si]5(c6ccc7c(c6)oc6ccccc67)c6ccccc6-c6ccccc65)c4)nc(-n4c5ccccc5c5ccccc54)n3)cc2)cc1 ; c1ccc(-c2ccc(-c3nc(-c4cccc([Si]5(c6ccccc6-c6ccccc6)c6ccccc6-c6ccccc65)c4)nc(-n4c5ccccc5c5ccccc54)n3)cc2)cc1]

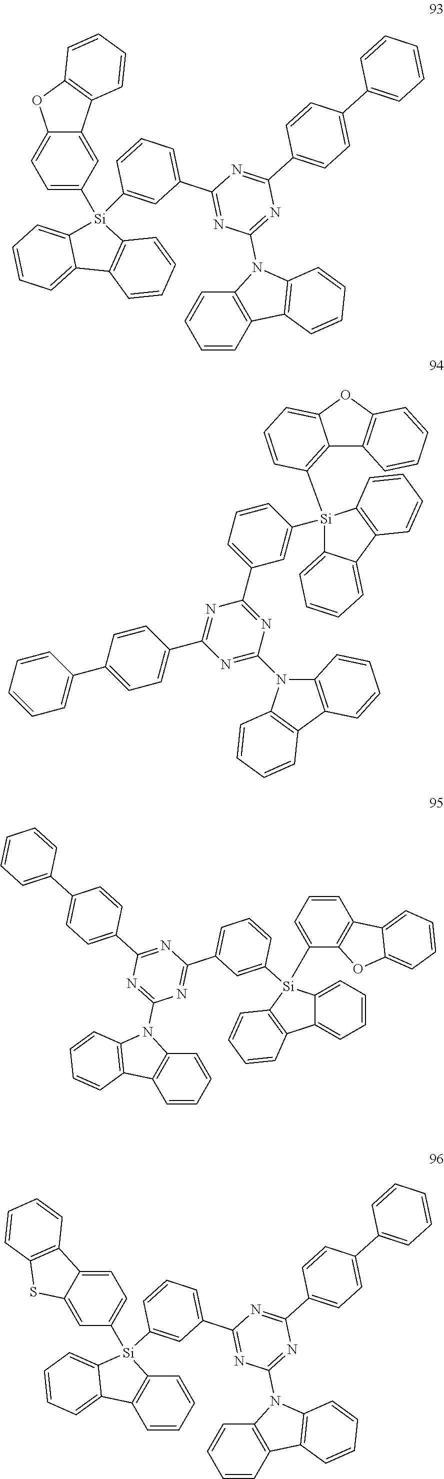 [SMILES: c1ccc(-c2ccc(-c3nc(-c4cccc([Si]5(c6ccc7oc8ccccc8c7c6)c6ccccc6-c6ccccc65)c4)nc(-n4c5ccccc5c5ccccc54)n3)cc2)cc1 ; c1ccc(-c2ccc(-c3nc(-c4cccc([Si]5(c6cccc7oc8ccccc8c67)c6ccccc6-c6ccccc65)c4)nc(-n4c5ccccc5c5ccccc54)n3)cc2)cc1 ; c1ccc(-c2ccc(-c3nc(-c4cccc([Si]5(c6cccc7c6oc6ccccc67)c6ccccc6-c6ccccc65)c4)nc(-n4c5ccccc5c5ccccc54)n3)cc2)cc1 ; c1ccc(-c2ccc(-c3nc(-c4cccc([Si]5(c6ccc7c(c6)sc6ccccc67)c6ccccc6-c6ccccc65)c4)nc(-n4c5ccccc5c5ccccc54)n3)cc2)cc1]

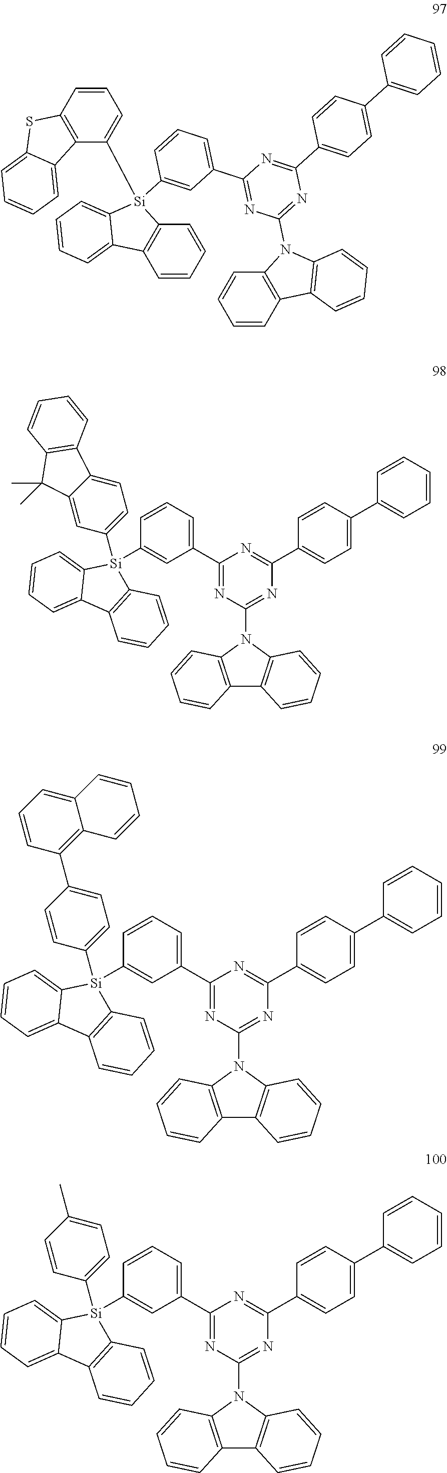 [SMILES: c1ccc(-c2ccc(-c3nc(-c4cccc([Si]5(c6cccc7sc8ccccc8c67)c6ccccc6-c6ccccc65)c4)nc(-n4c5ccccc5c5ccccc54)n3)cc2)cc1 ; CC1(C)c2ccccc2-c2ccc([Si]3(c4cccc(-c5nc(-c6ccc(-c7ccccc7)cc6)nc(-n6c7ccccc7c7ccccc76)n5)c4)c4ccccc4-c4ccccc43)cc21 ; c1ccc(-c2ccc(-c3nc(-c4cccc([Si]5(c6ccc(-c7cccc8ccccc78)cc6)c6ccccc6-c6ccccc65)c4)nc(-n4c5ccccc5c5ccccc54)n3)cc2)cc1 ; Cc1ccc([Si]2(c3cccc(-c4nc(-c5ccc(-c6ccccc6)cc5)nc(-n5c6ccccc6c6ccccc65)n4)c3)c3ccccc3-c3ccccc32)cc1]

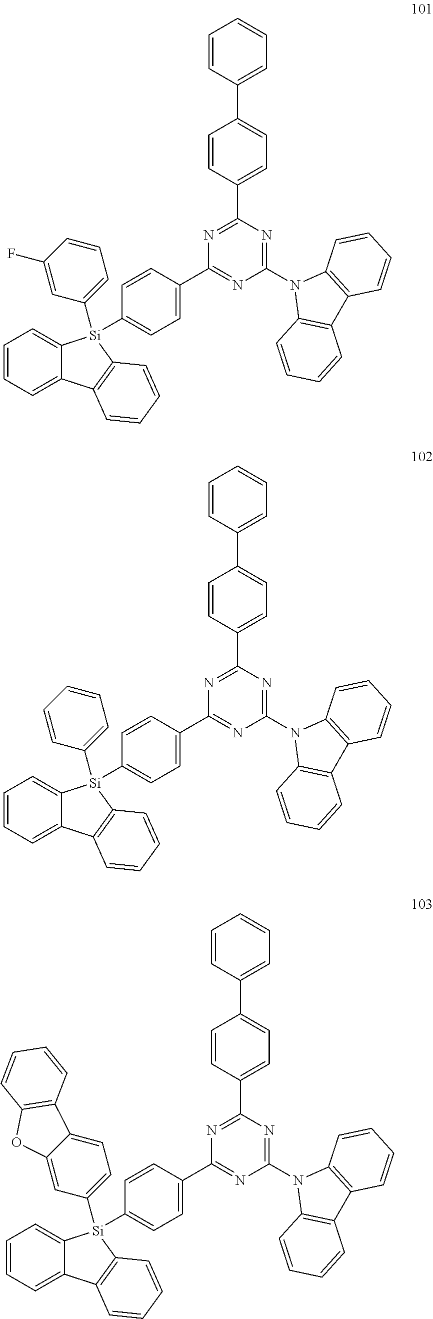 [SMILES: Fc1cccc([Si]2(c3ccc(-c4nc(-c5ccc(-c6ccccc6)cc5)nc(-n5c6ccccc6c6ccccc65)n4)cc3)c3ccccc3-c3ccccc32)c1 ; c1ccc(-c2ccc(-c3nc(-c4ccc([Si]5(c6ccccc6)c6ccccc6-c6ccccc65)cc4)nc(-n4c5ccccc5c5ccccc54)n3)cc2)cc1 ; c1ccc(-c2ccc(-c3nc(-c4ccc([Si]5(c6ccc7c(c6)oc6ccccc67)c6ccccc6-c6ccccc65)cc4)nc(-n4c5ccccc5c5ccccc54)n3)cc2)cc1]

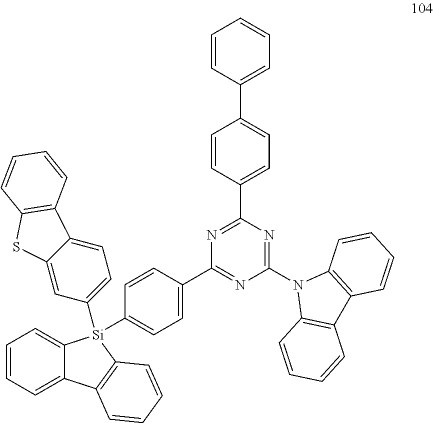 [SMILES: c1ccc(-c2ccc(-c3nc(-c4ccc([Si]5(c6ccc7c(c6)sc6ccccc67)c6ccccc6-c6ccccc65)cc4)nc(-n4c5ccccc5c5ccccc54)n3)cc2)cc1]

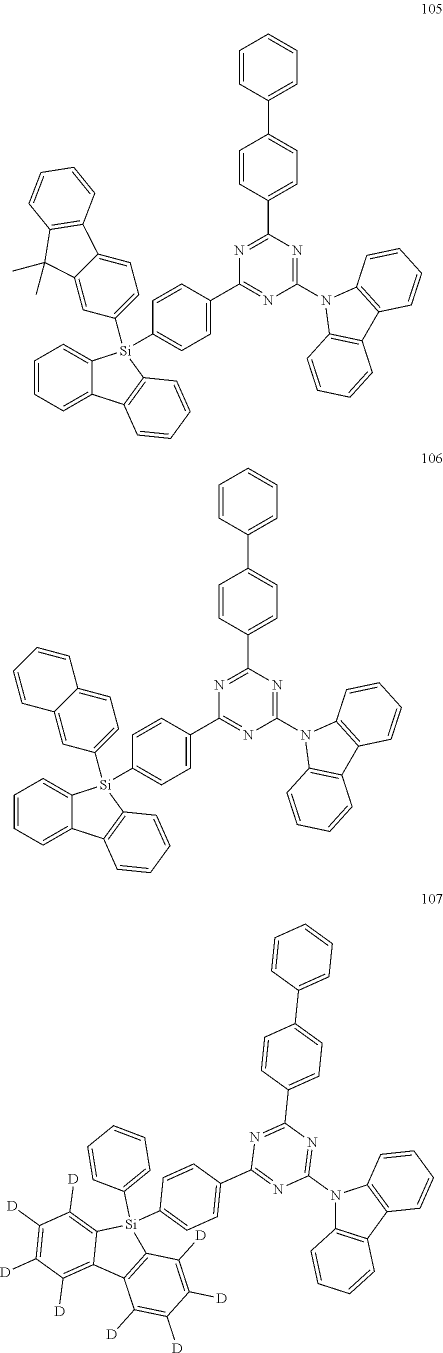 [SMILES: CC1(C)c2ccccc2-c2ccc([Si]3(c4ccc(-c5nc(-c6ccc(-c7ccccc7)cc6)nc(-n6c7ccccc7c7ccccc76)n5)cc4)c4ccccc4-c4ccccc43)cc21 ; c1ccc(-c2ccc(-c3nc(-c4ccc([Si]5(c6ccc7ccccc7c6)c6ccccc6-c6ccccc65)cc4)nc(-n4c5ccccc5c5ccccc54)n3)cc2)cc1 ; [2H]c1c([2H])c([2H])c2c(c1[2H])-c1c([2H])c([2H])c([2H])c([2H])c1[Si]2(c1ccccc1)c1ccc(-c2nc(-c3ccc(-c4ccccc4)cc3)nc(-n3c4ccccc4c4ccccc43)n2)cc1]

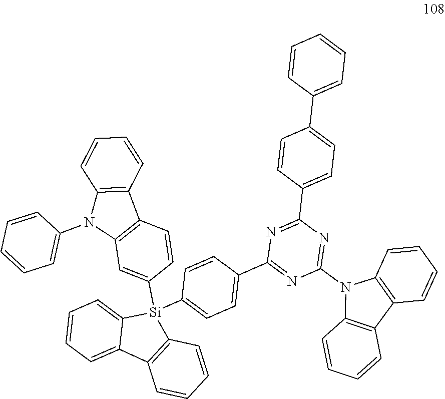 [SMILES: c1ccc(-c2ccc(-c3nc(-c4ccc([Si]5(c6ccc7c8ccccc8n(-c8ccccc8)c7c6)c6ccccc6-c6ccccc65)cc4)nc(-n4c5ccccc5c5ccccc54)n3)cc2)cc1]

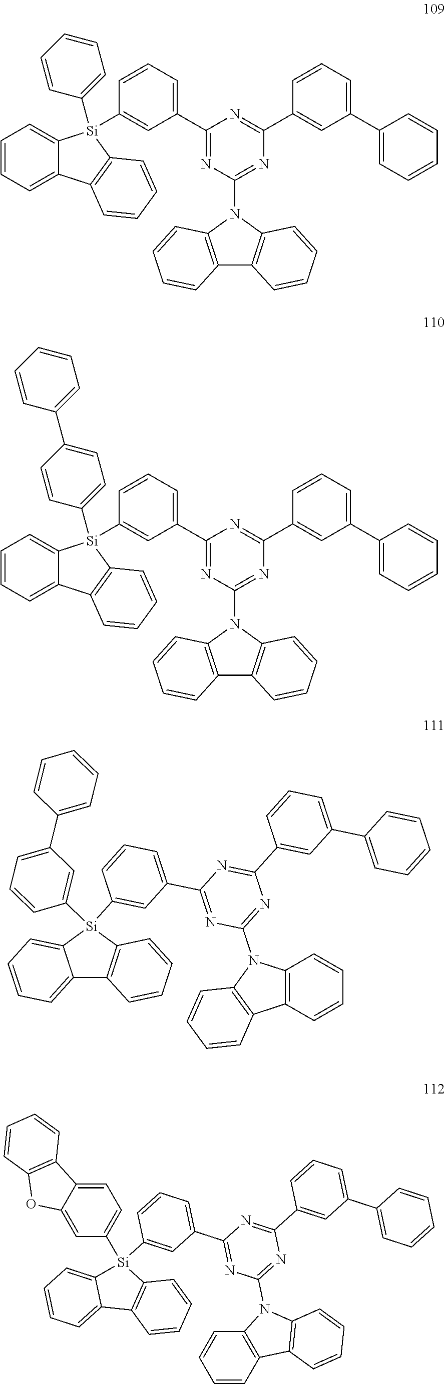 [SMILES: c1ccc(-c2cccc(-c3nc(-c4cccc([Si]5(c6ccccc6)c6ccccc6-c6ccccc65)c4)nc(-n4c5ccccc5c5ccccc54)n3)c2)cc1 ; c1ccc(-c2ccc([Si]3(c4cccc(-c5nc(-c6cccc(-c7ccccc7)c6)nc(-n6c7ccccc7c7ccccc76)n5)c4)c4ccccc4-c4ccccc43)cc2)cc1 ; c1ccc(-c2cccc(-c3nc(-c4cccc([Si]5(c6cccc(-c7ccccc7)c6)c6ccccc6-c6ccccc65)c4)nc(-n4c5ccccc5c5ccccc54)n3)c2)cc1 ; c1ccc(-c2cccc(-c3nc(-c4cccc([Si]5(c6ccc7c(c6)oc6ccccc67)c6ccccc6-c6ccccc65)c4)nc(-n4c5ccccc5c5ccccc54)n3)c2)cc1]

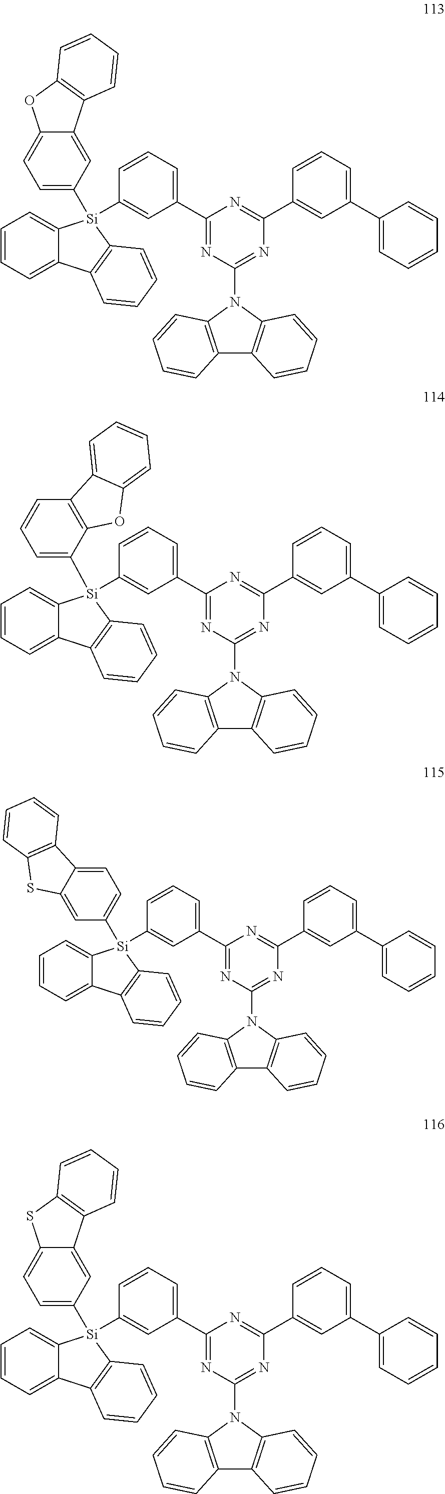 [SMILES: c1ccc(-c2cccc(-c3nc(-c4cccc([Si]5(c6ccc7oc8ccccc8c7c6)c6ccccc6-c6ccccc65)c4)nc(-n4c5ccccc5c5ccccc54)n3)c2)cc1 ; c1ccc(-c2cccc(-c3nc(-c4cccc([Si]5(c6cccc7c6oc6ccccc67)c6ccccc6-c6ccccc65)c4)nc(-n4c5ccccc5c5ccccc54)n3)c2)cc1 ; c1ccc(-c2cccc(-c3nc(-c4cccc([Si]5(c6ccc7c(c6)sc6ccccc67)c6ccccc6-c6ccccc65)c4)nc(-n4c5ccccc5c5ccccc54)n3)c2)cc1 ; c1ccc(-c2cccc(-c3nc(-c4cccc([Si]5(c6ccc7sc8ccccc8c7c6)c6ccccc6-c6ccccc65)c4)nc(-n4c5ccccc5c5ccccc54)n3)c2)cc1]

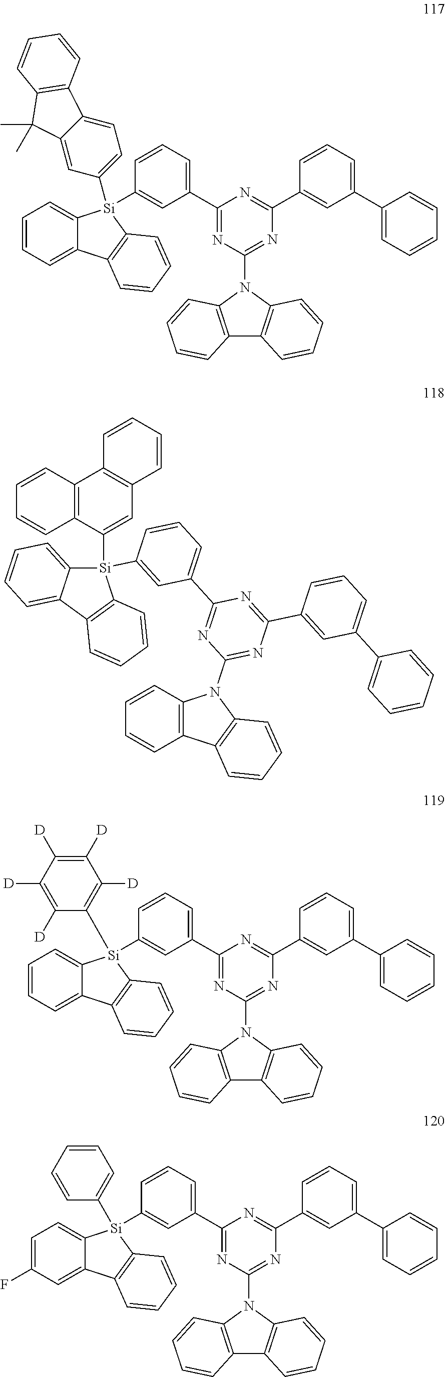 [SMILES: CC1(C)c2ccccc2-c2ccc([Si]3(c4cccc(-c5nc(-c6cccc(-c7ccccc7)c6)nc(-n6c7ccccc7c7ccccc76)n5)c4)c4ccccc4-c4ccccc43)cc21 ; c1ccc(-c2cccc(-c3nc(-c4cccc([Si]5(c6cc7ccccc7c7ccccc67)c6ccccc6-c6ccccc65)c4)nc(-n4c5ccccc5c5ccccc54)n3)c2)cc1 ; [2H]c1c([2H])c([2H])c([Si]2(c3cccc(-c4nc(-c5cccc(-c6ccccc6)c5)nc(-n5c6ccccc6c6ccccc65)n4)c3)c3ccccc3-c3ccccc32)c([2H])c1[2H] ; Fc1ccc2c(c1)-c1ccccc1[Si]2(c1ccccc1)c1cccc(-c2nc(-c3cccc(-c4ccccc4)c3)nc(-n3c4ccccc4c4ccccc43)n2)c1]

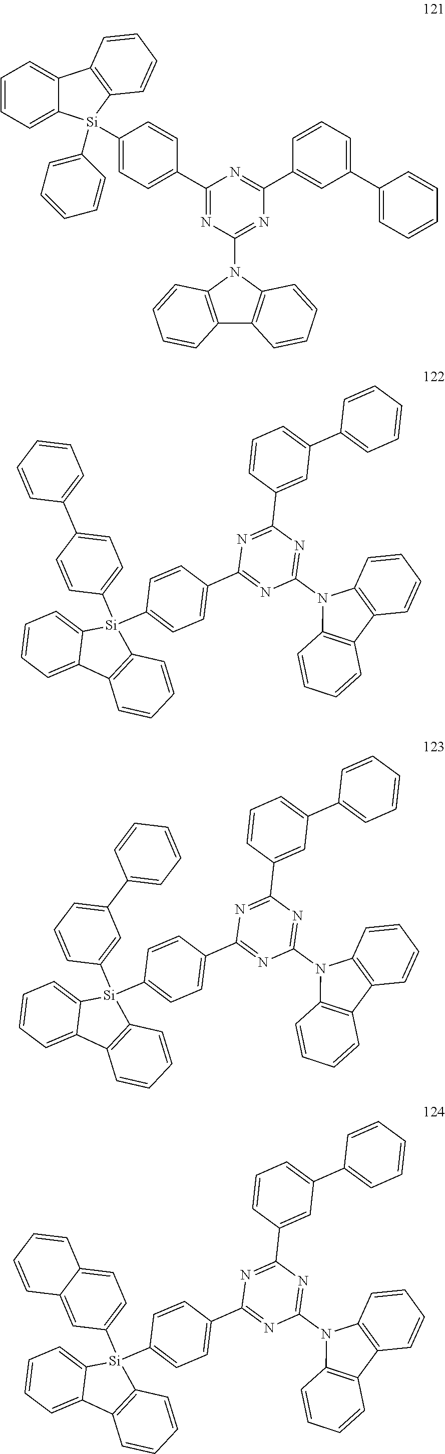 [SMILES: c1ccc(-c2cccc(-c3nc(-c4ccc([Si]5(c6ccccc6)c6ccccc6-c6ccccc65)cc4)nc(-n4c5ccccc5c5ccccc54)n3)c2)cc1 ; c1ccc(-c2ccc([Si]3(c4ccc(-c5nc(-c6cccc(-c7ccccc7)c6)nc(-n6c7ccccc7c7ccccc76)n5)cc4)c4ccccc4-c4ccccc43)cc2)cc1 ; c1ccc(-c2cccc(-c3nc(-c4ccc([Si]5(c6cccc(-c7ccccc7)c6)c6ccccc6-c6ccccc65)cc4)nc(-n4c5ccccc5c5ccccc54)n3)c2)cc1 ; c1ccc(-c2cccc(-c3nc(-c4ccc([Si]5(c6ccc7ccccc7c6)c6ccccc6-c6ccccc65)cc4)nc(-n4c5ccccc5c5ccccc54)n3)c2)cc1]

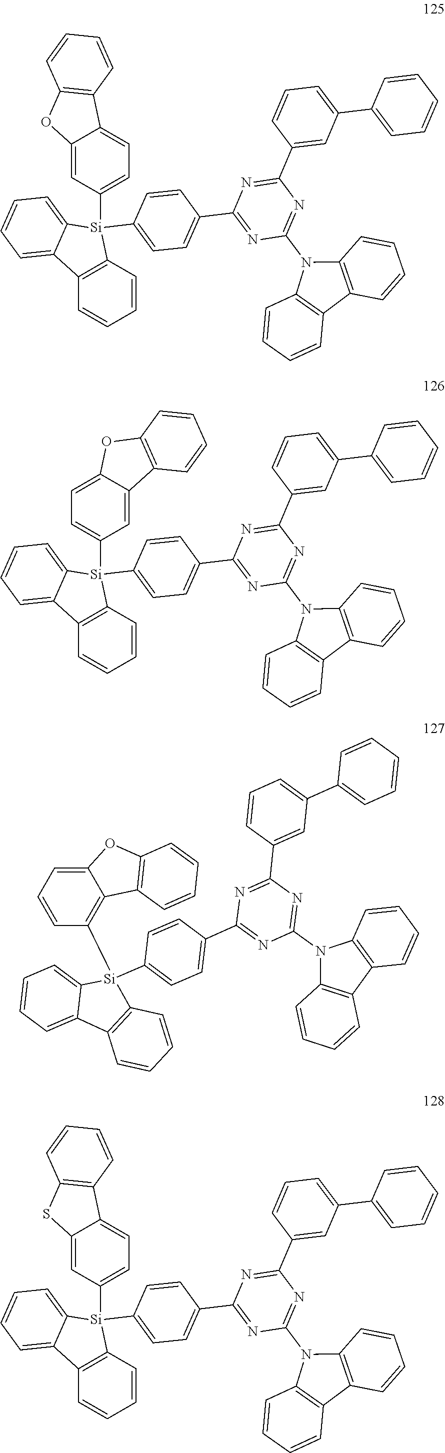 [SMILES: c1ccc(-c2cccc(-c3nc(-c4ccc([Si]5(c6ccc7c(c6)oc6ccccc67)c6ccccc6-c6ccccc65)cc4)nc(-n4c5ccccc5c5ccccc54)n3)c2)cc1 ; c1ccc(-c2cccc(-c3nc(-c4ccc([Si]5(c6ccc7oc8ccccc8c7c6)c6ccccc6-c6ccccc65)cc4)nc(-n4c5ccccc5c5ccccc54)n3)c2)cc1 ; c1ccc(-c2cccc(-c3nc(-c4ccc([Si]5(c6cccc7oc8ccccc8c67)c6ccccc6-c6ccccc65)cc4)nc(-n4c5ccccc5c5ccccc54)n3)c2)cc1 ; c1ccc(-c2cccc(-c3nc(-c4ccc([Si]5(c6ccc7c(c6)sc6ccccc67)c6ccccc6-c6ccccc65)cc4)nc(-n4c5ccccc5c5ccccc54)n3)c2)cc1]

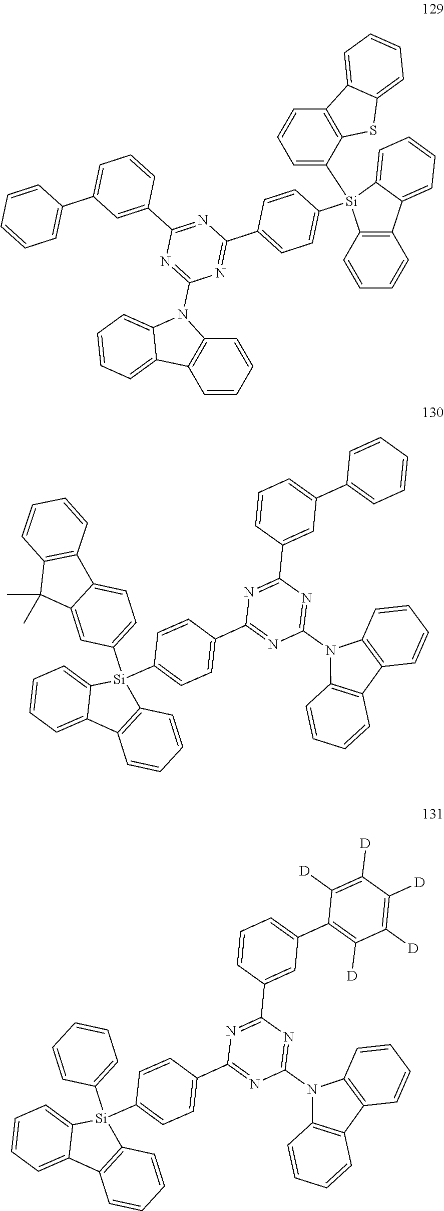 [SMILES: c1ccc(-c2cccc(-c3nc(-c4ccc([Si]5(c6cccc7c6sc6ccccc67)c6ccccc6-c6ccccc65)cc4)nc(-n4c5ccccc5c5ccccc54)n3)c2)cc1 ; CC1(C)c2ccccc2-c2ccc([Si]3(c4ccc(-c5nc(-c6cccc(-c7ccccc7)c6)nc(-n6c7ccccc7c7ccccc76)n5)cc4)c4ccccc4-c4ccccc43)cc21 ; [2H]c1c([2H])c([2H])c(-c2cccc(-c3nc(-c4ccc([Si]5(c6ccccc6)c6ccccc6-c6ccccc65)cc4)nc(-n4c5ccccc5c5ccccc54)n3)c2)c([2H])c1[2H]]

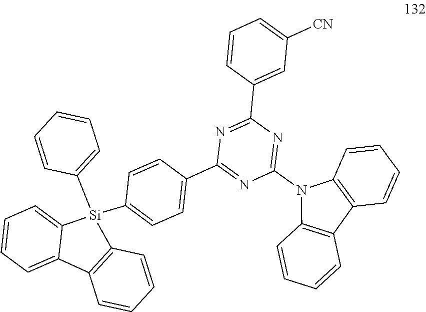 [SMILES: N#Cc1cccc(-c2nc(-c3ccc([Si]4(c5ccccc5)c5ccccc5-c5ccccc54)cc3)nc(-n3c4ccccc4c4ccccc43)n2)c1]

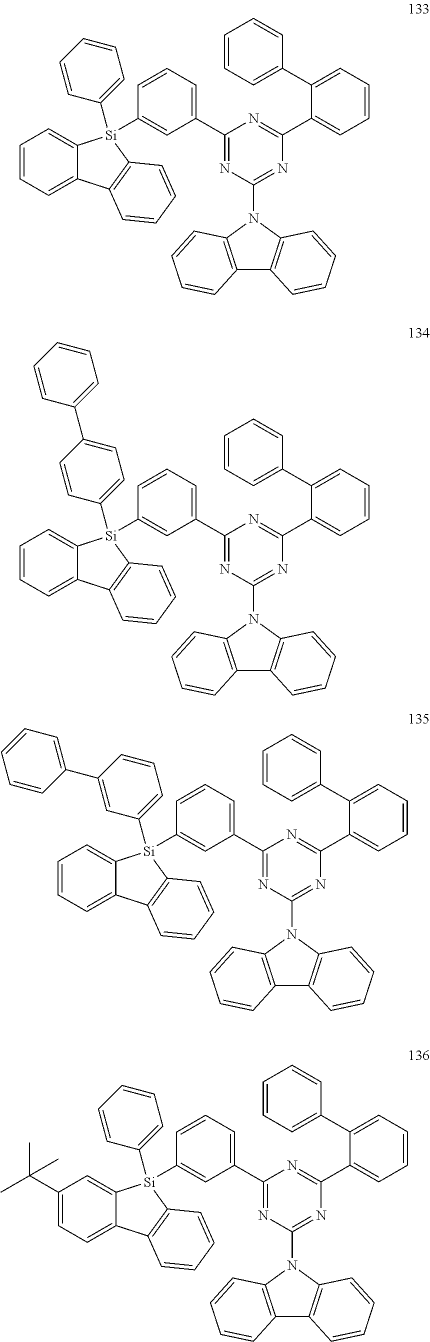

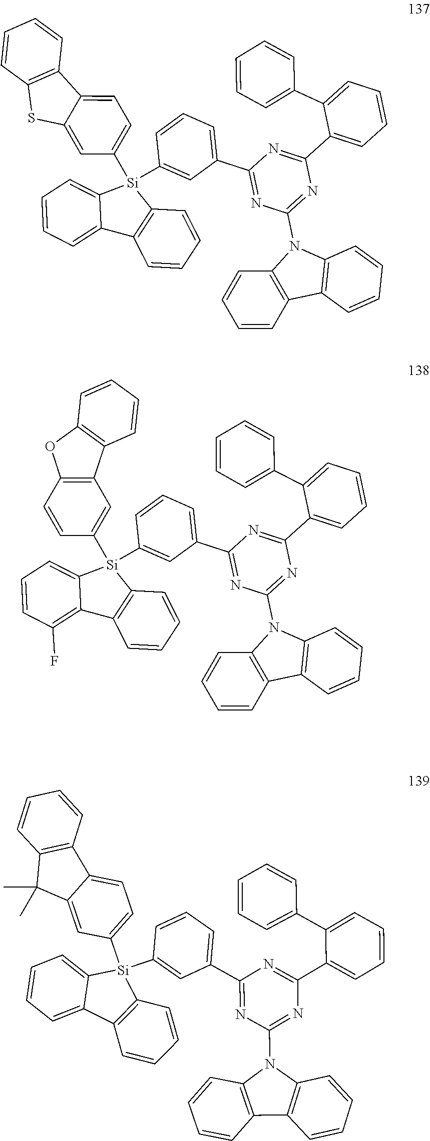 [SMILES: c1ccc(-c2ccccc2-c2nc(-c3cccc([Si]4(c5ccc6c(c5)sc5ccccc56)c5ccccc5-c5ccccc54)c3)nc(-n3c4ccccc4c4ccccc43)n2)cc1 ; Fc1cccc2c1-c1ccccc1[Si]2(c1cccc(-c2nc(-c3ccccc3-c3ccccc3)nc(-n3c4ccccc4c4ccccc43)n2)c1)c1ccc2oc3ccccc3c2c1 ; CC1(C)c2ccccc2-c2ccc([Si]3(c4cccc(-c5nc(-c6ccccc6-c6ccccc6)nc(-n6c7ccccc7c7ccccc76)n5)c4)c4ccccc4-c4ccccc43)cc21]

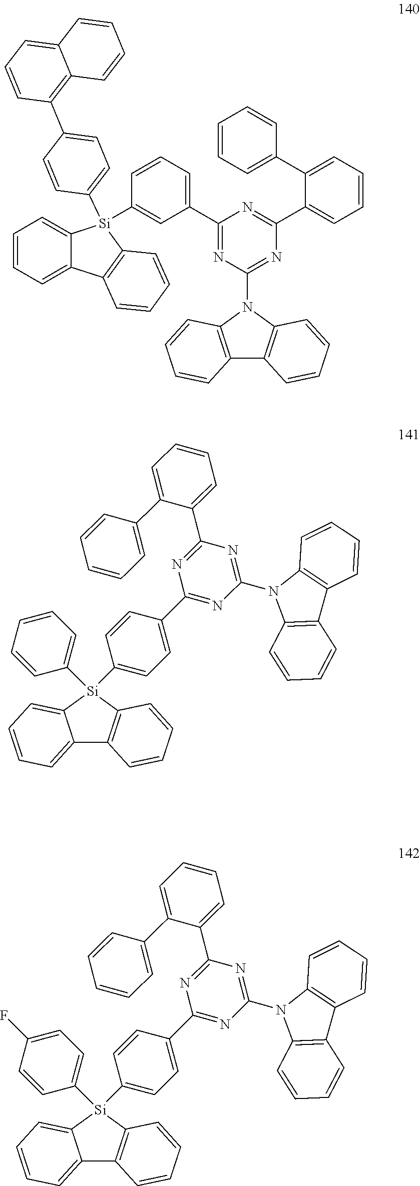 [SMILES: c1ccc(-c2ccccc2-c2nc(-c3cccc([Si]4(c5ccc(-c6cccc7ccccc67)cc5)c5ccccc5-c5ccccc54)c3)nc(-n3c4ccccc4c4ccccc43)n2)cc1 ; c1ccc(-c2ccccc2-c2nc(-c3ccc([Si]4(c5ccccc5)c5ccccc5-c5ccccc54)cc3)nc(-n3c4ccccc4c4ccccc43)n2)cc1 ; Fc1ccc([Si]2(c3ccc(-c4nc(-c5ccccc5-c5ccccc5)nc(-n5c6ccccc6c6ccccc65)n4)cc3)c3ccccc3-c3ccccc32)cc1]

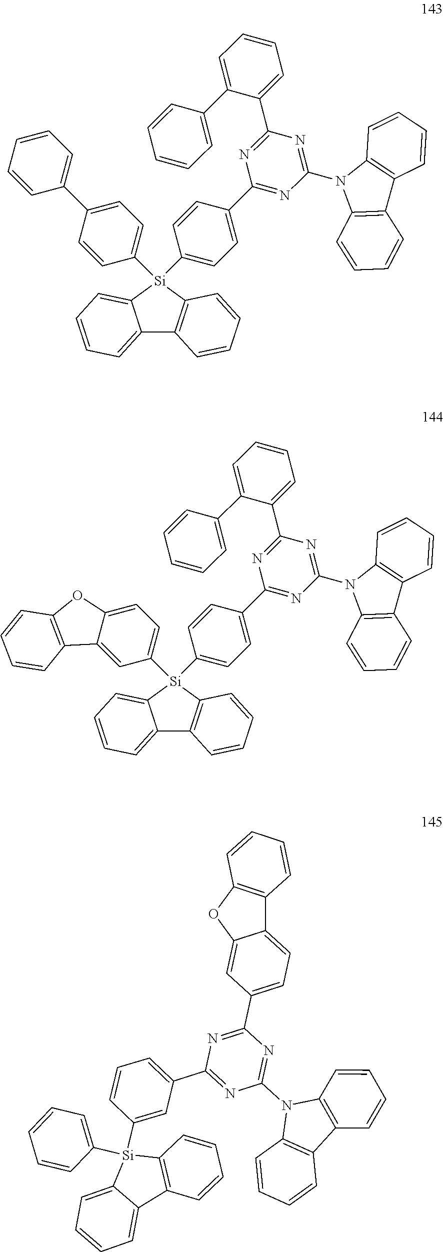 [SMILES: c1ccc(-c2ccc([Si]3(c4ccc(-c5nc(-c6ccccc6-c6ccccc6)nc(-n6c7ccccc7c7ccccc76)n5)cc4)c4ccccc4-c4ccccc43)cc2)cc1 ; c1ccc(-c2ccccc2-c2nc(-c3ccc([Si]4(c5ccc6oc7ccccc7c6c5)c5ccccc5-c5ccccc54)cc3)nc(-n3c4ccccc4c4ccccc43)n2)cc1 ; c1ccc([Si]2(c3cccc(-c4nc(-c5ccc6c(c5)oc5ccccc56)nc(-n5c6ccccc6c6ccccc65)n4)c3)c3ccccc3-c3ccccc32)cc1]

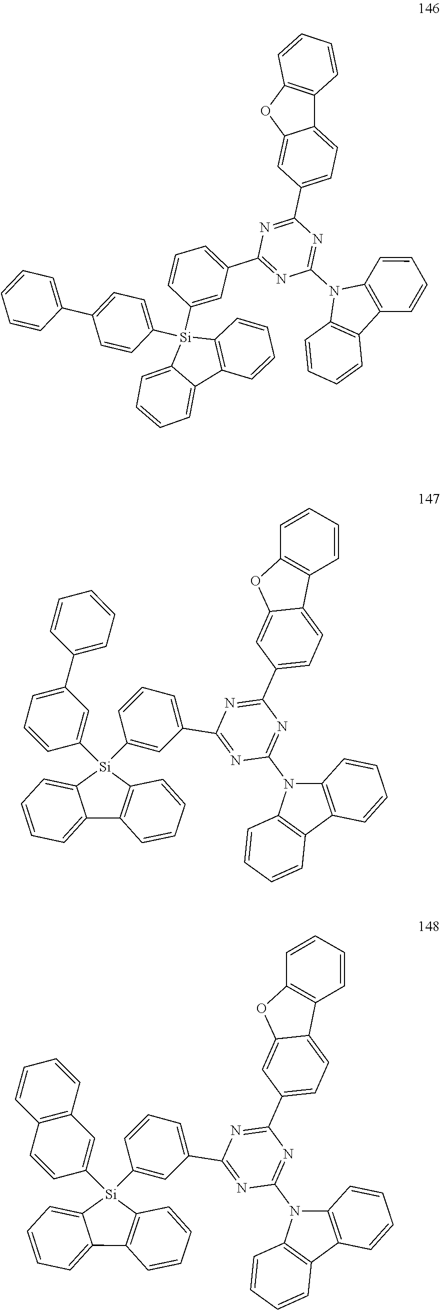 [SMILES: c1ccc(-c2ccc([Si]3(c4cccc(-c5nc(-c6ccc7c(c6)oc6ccccc67)nc(-n6c7ccccc7c7ccccc76)n5)c4)c4ccccc4-c4ccccc43)cc2)cc1 ; c1ccc(-c2cccc([Si]3(c4cccc(-c5nc(-c6ccc7c(c6)oc6ccccc67)nc(-n6c7ccccc7c7ccccc76)n5)c4)c4ccccc4-c4ccccc43)c2)cc1 ; c1cc(-c2nc(-c3ccc4c(c3)oc3ccccc34)nc(-n3c4ccccc4c4ccccc43)n2)cc([Si]2(c3ccc4ccccc4c3)c3ccccc3-c3ccccc32)c1]

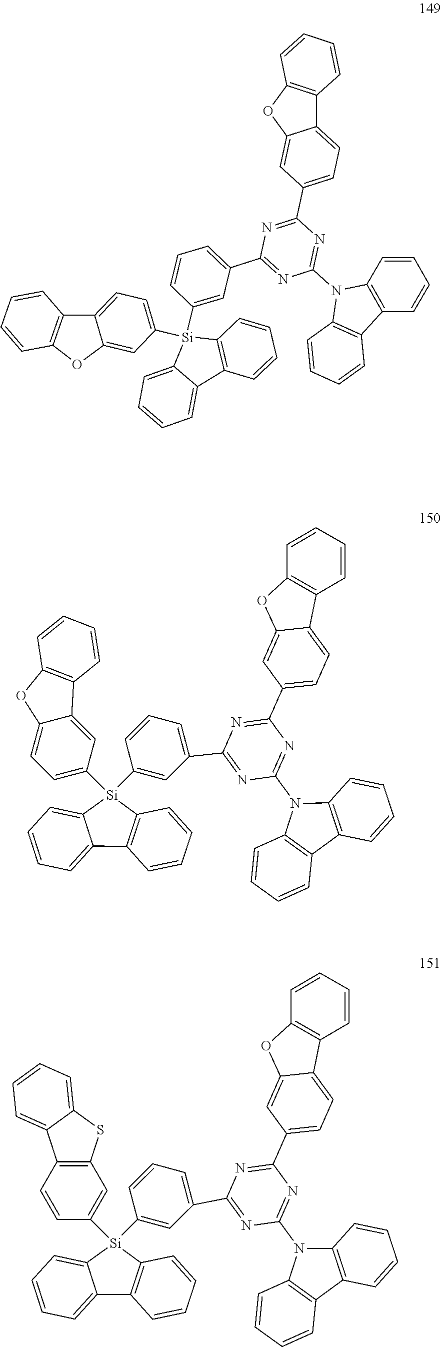 [SMILES: c1cc(-c2nc(-c3ccc4c(c3)oc3ccccc34)nc(-n3c4ccccc4c4ccccc43)n2)cc([Si]2(c3ccc4c(c3)oc3ccccc34)c3ccccc3-c3ccccc32)c1 ; c1cc(-c2nc(-c3ccc4c(c3)oc3ccccc34)nc(-n3c4ccccc4c4ccccc43)n2)cc([Si]2(c3ccc4oc5ccccc5c4c3)c3ccccc3-c3ccccc32)c1 ; c1cc(-c2nc(-c3ccc4c(c3)oc3ccccc34)nc(-n3c4ccccc4c4ccccc43)n2)cc([Si]2(c3ccc4c(c3)sc3ccccc34)c3ccccc3-c3ccccc32)c1]

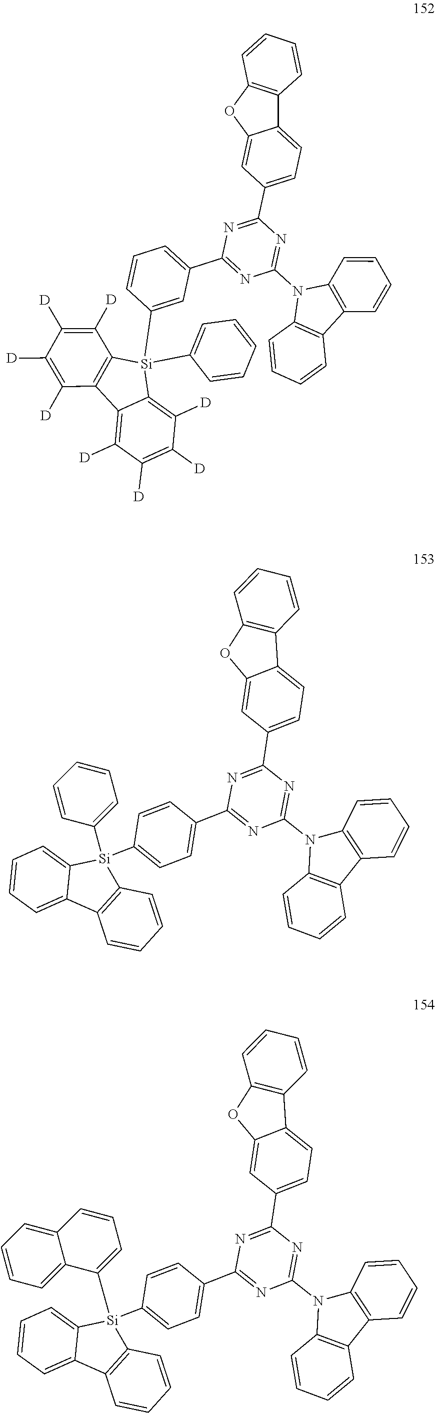 [SMILES: [2H]c1c([2H])c([2H])c2c(c1[2H])-c1c([2H])c([2H])c([2H])c([2H])c1[Si]2(c1ccccc1)c1cccc(-c2nc(-c3ccc4c(c3)oc3ccccc34)nc(-n3c4ccccc4c4ccccc43)n2)c1 ; c1ccc([Si]2(c3ccc(-c4nc(-c5ccc6c(c5)oc5ccccc56)nc(-n5c6ccccc6c6ccccc65)n4)cc3)c3ccccc3-c3ccccc32)cc1 ; c1ccc2c(c1)-c1ccccc1[Si]2(c1ccc(-c2nc(-c3ccc4c(c3)oc3ccccc34)nc(-n3c4ccccc4c4ccccc43)n2)cc1)c1cccc2ccccc12]

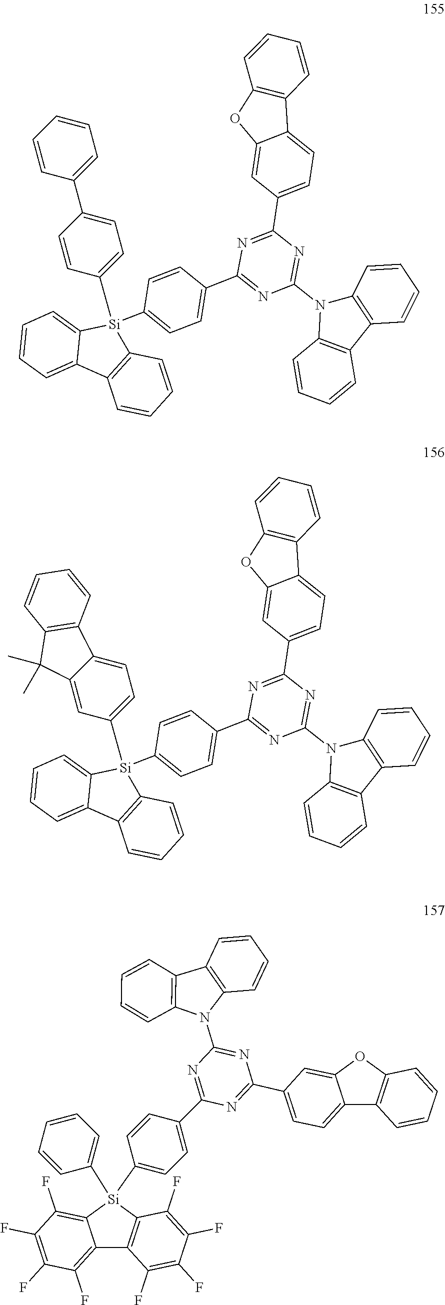 [SMILES: c1ccc(-c2ccc([Si]3(c4ccc(-c5nc(-c6ccc7c(c6)oc6ccccc67)nc(-n6c7ccccc7c7ccccc76)n5)cc4)c4ccccc4-c4ccccc43)cc2)cc1 ; CC1(C)c2ccccc2-c2ccc([Si]3(c4ccc(-c5nc(-c6ccc7c(c6)oc6ccccc67)nc(-n6c7ccccc7c7ccccc76)n5)cc4)c4ccccc4-c4ccccc43)cc21 ; Fc1c(F)c(F)c2c(c1F)-c1c(F)c(F)c(F)c(F)c1[Si]2(c1ccccc1)c1ccc(-c2nc(-c3ccc4c(c3)oc3ccccc34)nc(-n3c4ccccc4c4ccccc43)n2)cc1]

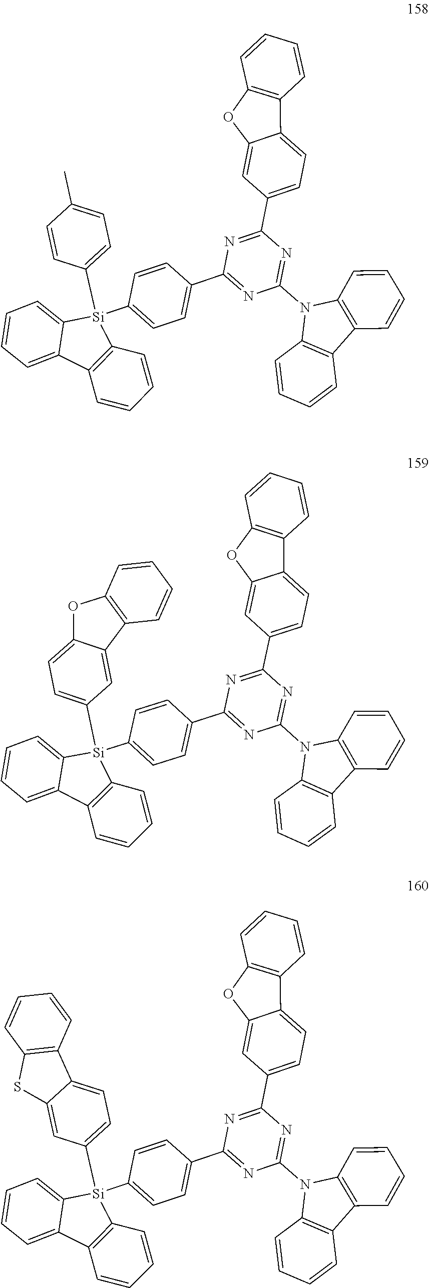 [SMILES: Cc1ccc([Si]2(c3ccc(-c4nc(-c5ccc6c(c5)oc5ccccc56)nc(-n5c6ccccc6c6ccccc65)n4)cc3)c3ccccc3-c3ccccc32)cc1 ; c1ccc2c(c1)-c1ccccc1[Si]2(c1ccc(-c2nc(-c3ccc4c(c3)oc3ccccc34)nc(-n3c4ccccc4c4ccccc43)n2)cc1)c1ccc2oc3ccccc3c2c1 ; c1ccc2c(c1)-c1ccccc1[Si]2(c1ccc(-c2nc(-c3ccc4c(c3)oc3ccccc34)nc(-n3c4ccccc4c4ccccc43)n2)cc1)c1ccc2c(c1)sc1ccccc12]

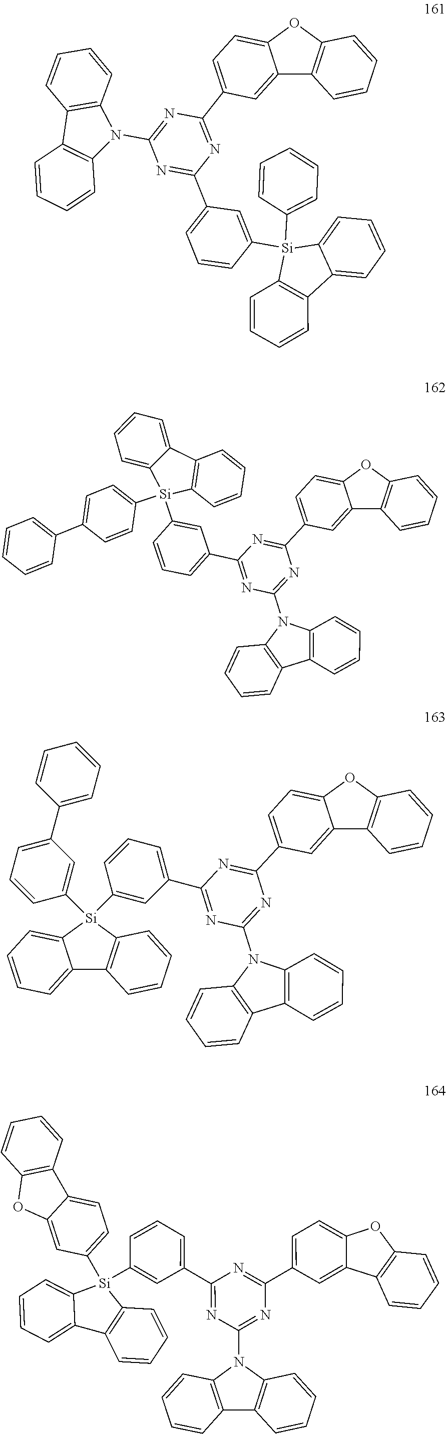 [SMILES: c1ccc([Si]2(c3cccc(-c4nc(-c5ccc6oc7ccccc7c6c5)nc(-n5c6ccccc6c6ccccc65)n4)c3)c3ccccc3-c3ccccc32)cc1 ; c1ccc(-c2ccc([Si]3(c4cccc(-c5nc(-c6ccc7oc8ccccc8c7c6)nc(-n6c7ccccc7c7ccccc76)n5)c4)c4ccccc4-c4ccccc43)cc2)cc1 ; c1ccc(-c2cccc([Si]3(c4cccc(-c5nc(-c6ccc7oc8ccccc8c7c6)nc(-n6c7ccccc7c7ccccc76)n5)c4)c4ccccc4-c4ccccc43)c2)cc1 ; c1cc(-c2nc(-c3ccc4oc5ccccc5c4c3)nc(-n3c4ccccc4c4ccccc43)n2)cc([Si]2(c3ccc4c(c3)oc3ccccc34)c3ccccc3-c3ccccc32)c1]

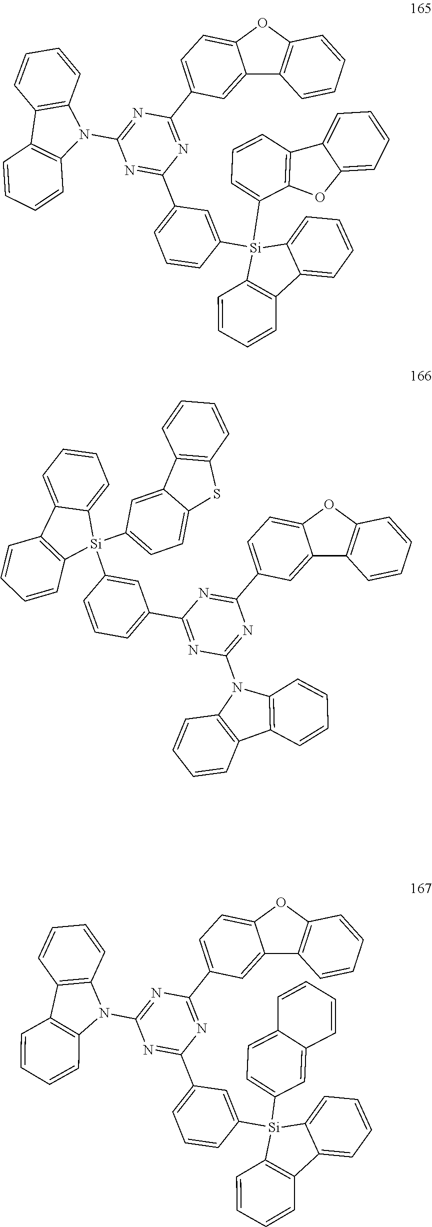 [SMILES: c1cc(-c2nc(-c3ccc4oc5ccccc5c4c3)nc(-n3c4ccccc4c4ccccc43)n2)cc([Si]2(c3cccc4c3oc3ccccc34)c3ccccc3-c3ccccc32)c1 ; c1cc(-c2nc(-c3ccc4oc5ccccc5c4c3)nc(-n3c4ccccc4c4ccccc43)n2)cc([Si]2(c3ccc4sc5ccccc5c4c3)c3ccccc3-c3ccccc32)c1 ; c1cc(-c2nc(-c3ccc4oc5ccccc5c4c3)nc(-n3c4ccccc4c4ccccc43)n2)cc([Si]2(c3ccc4ccccc4c3)c3ccccc3-c3ccccc32)c1]

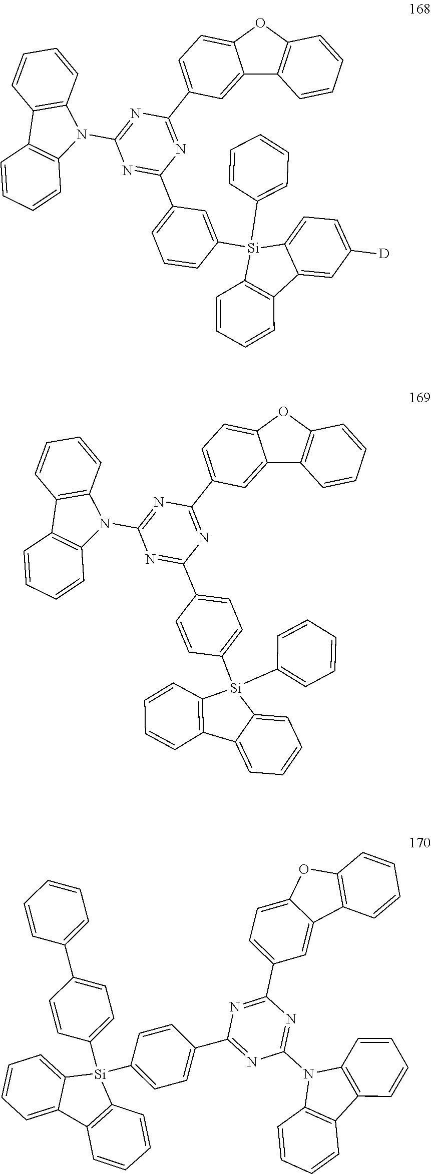 [SMILES: [2H]c1ccc2c(c1)-c1ccccc1[Si]2(c1ccccc1)c1cccc(-c2nc(-c3ccc4oc5ccccc5c4c3)nc(-n3c4ccccc4c4ccccc43)n2)c1 ; c1ccc([Si]2(c3ccc(-c4nc(-c5ccc6oc7ccccc7c6c5)nc(-n5c6ccccc6c6ccccc65)n4)cc3)c3ccccc3-c3ccccc32)cc1 ; c1ccc(-c2ccc([Si]3(c4ccc(-c5nc(-c6ccc7oc8ccccc8c7c6)nc(-n6c7ccccc7c7ccccc76)n5)cc4)c4ccccc4-c4ccccc43)cc2)cc1]

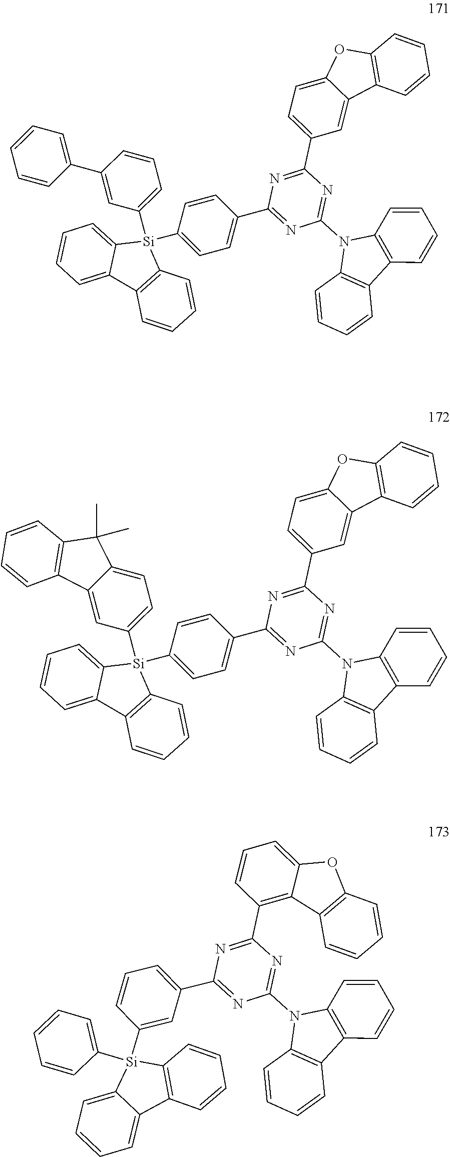 [SMILES: c1ccc(-c2cccc([Si]3(c4ccc(-c5nc(-c6ccc7oc8ccccc8c7c6)nc(-n6c7ccccc7c7ccccc76)n5)cc4)c4ccccc4-c4ccccc43)c2)cc1 ; CC1(C)c2ccccc2-c2cc([Si]3(c4ccc(-c5nc(-c6ccc7oc8ccccc8c7c6)nc(-n6c7ccccc7c7ccccc76)n5)cc4)c4ccccc4-c4ccccc43)ccc21 ; c1ccc([Si]2(c3cccc(-c4nc(-c5cccc6oc7ccccc7c56)nc(-n5c6ccccc6c6ccccc65)n4)c3)c3ccccc3-c3ccccc32)cc1]

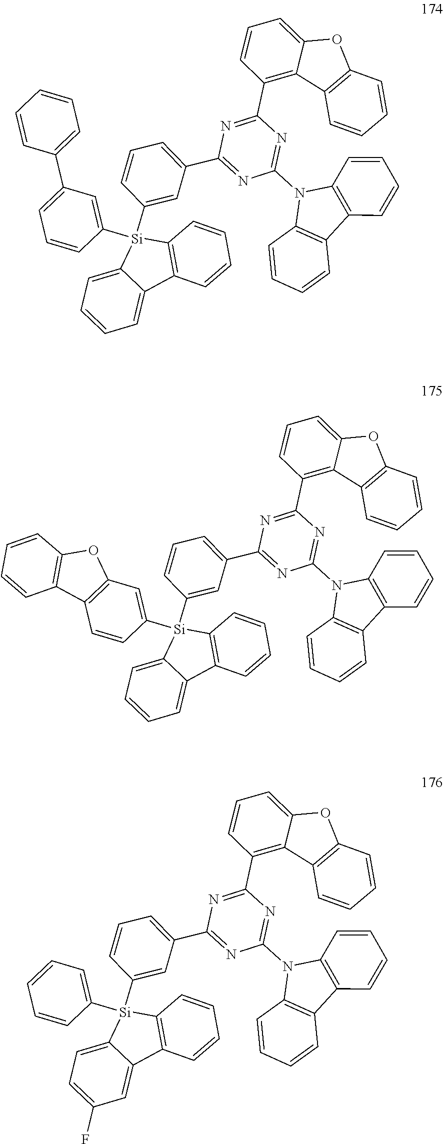 [SMILES: c1ccc(-c2cccc([Si]3(c4cccc(-c5nc(-c6cccc7oc8ccccc8c67)nc(-n6c7ccccc7c7ccccc76)n5)c4)c4ccccc4-c4ccccc43)c2)cc1 ; c1cc(-c2nc(-c3cccc4oc5ccccc5c34)nc(-n3c4ccccc4c4ccccc43)n2)cc([Si]2(c3ccc4c(c3)oc3ccccc34)c3ccccc3-c3ccccc32)c1 ; Fc1ccc2c(c1)-c1ccccc1[Si]2(c1ccccc1)c1cccc(-c2nc(-c3cccc4oc5ccccc5c34)nc(-n3c4ccccc4c4ccccc43)n2)c1]

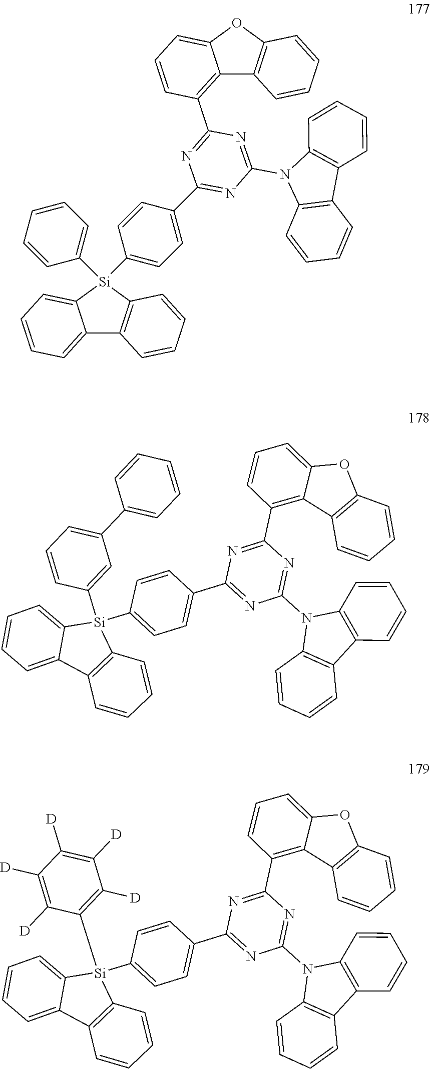 [SMILES: c1ccc([Si]2(c3ccc(-c4nc(-c5cccc6oc7ccccc7c56)nc(-n5c6ccccc6c6ccccc65)n4)cc3)c3ccccc3-c3ccccc32)cc1 ; c1ccc(-c2cccc([Si]3(c4ccc(-c5nc(-c6cccc7oc8ccccc8c67)nc(-n6c7ccccc7c7ccccc76)n5)cc4)c4ccccc4-c4ccccc43)c2)cc1 ; [2H]c1c([2H])c([2H])c([Si]2(c3ccc(-c4nc(-c5cccc6oc7ccccc7c56)nc(-n5c6ccccc6c6ccccc65)n4)cc3)c3ccccc3-c3ccccc32)c([2H])c1[2H]]

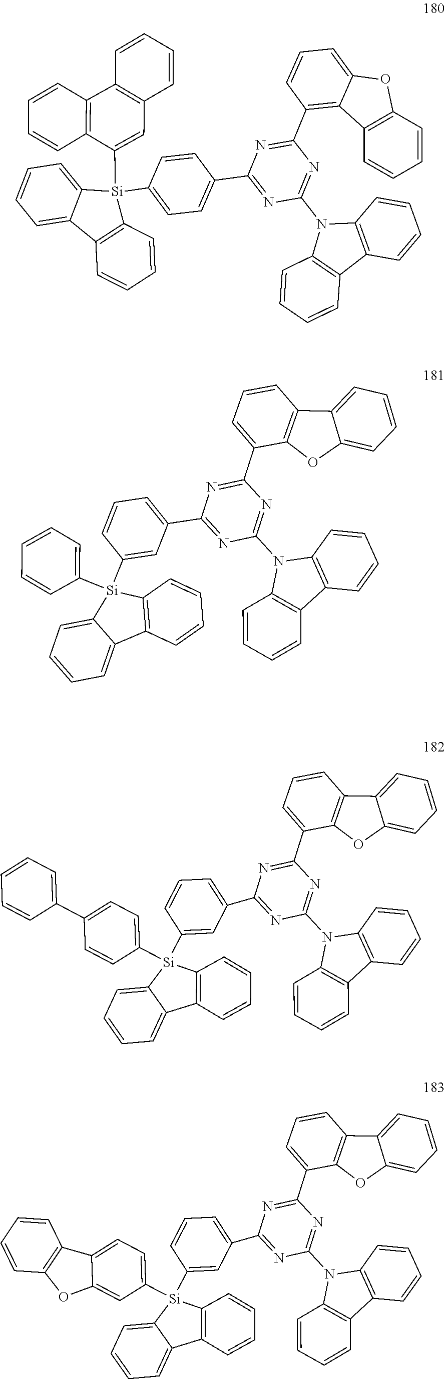 [SMILES: c1ccc2c(c1)-c1ccccc1[Si]2(c1ccc(-c2nc(-c3cccc4oc5ccccc5c34)nc(-n3c4ccccc4c4ccccc43)n2)cc1)c1cc2ccccc2c2ccccc12 ; c1ccc([Si]2(c3cccc(-c4nc(-c5cccc6c5oc5ccccc56)nc(-n5c6ccccc6c6ccccc65)n4)c3)c3ccccc3-c3ccccc32)cc1 ; c1ccc(-c2ccc([Si]3(c4cccc(-c5nc(-c6cccc7c6oc6ccccc67)nc(-n6c7ccccc7c7ccccc76)n5)c4)c4ccccc4-c4ccccc43)cc2)cc1 ; c1cc(-c2nc(-c3cccc4c3oc3ccccc34)nc(-n3c4ccccc4c4ccccc43)n2)cc([Si]2(c3ccc4c(c3)oc3ccccc34)c3ccccc3-c3ccccc32)c1]

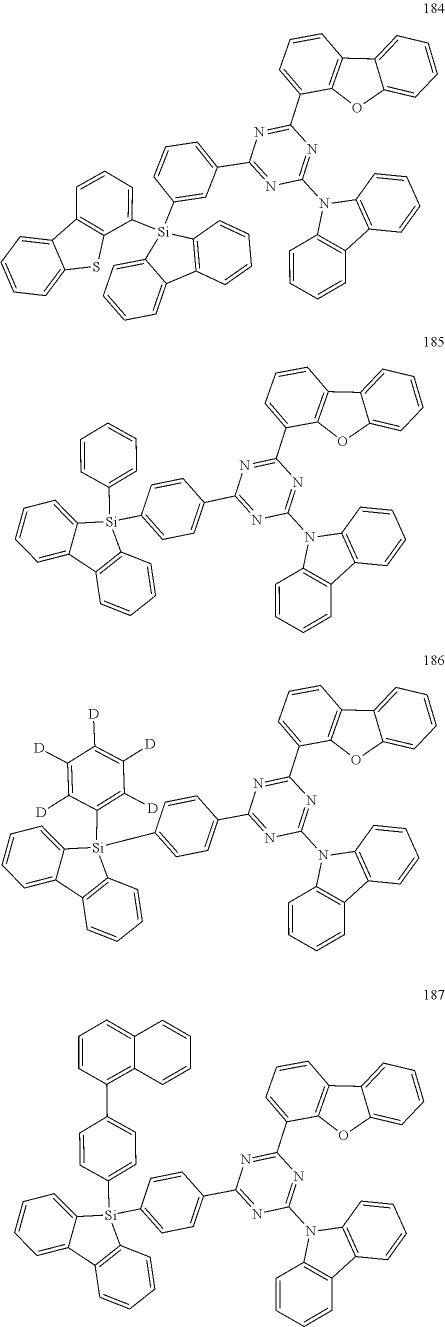 [SMILES: c1cc(-c2nc(-c3cccc4c3oc3ccccc34)nc(-n3c4ccccc4c4ccccc43)n2)cc([Si]2(c3cccc4c3sc3ccccc34)c3ccccc3-c3ccccc32)c1 ; c1ccc([Si]2(c3ccc(-c4nc(-c5cccc6c5oc5ccccc56)nc(-n5c6ccccc6c6ccccc65)n4)cc3)c3ccccc3-c3ccccc32)cc1 ; [2H]c1c([2H])c([2H])c([Si]2(c3ccc(-c4nc(-c5cccc6c5oc5ccccc56)nc(-n5c6ccccc6c6ccccc65)n4)cc3)c3ccccc3-c3ccccc32)c([2H])c1[2H] ; c1ccc2c(c1)-c1ccccc1[Si]2(c1ccc(-c2nc(-c3cccc4c3oc3ccccc34)nc(-n3c4ccccc4c4ccccc43)n2)cc1)c1ccc(-c2cccc3ccccc23)cc1]

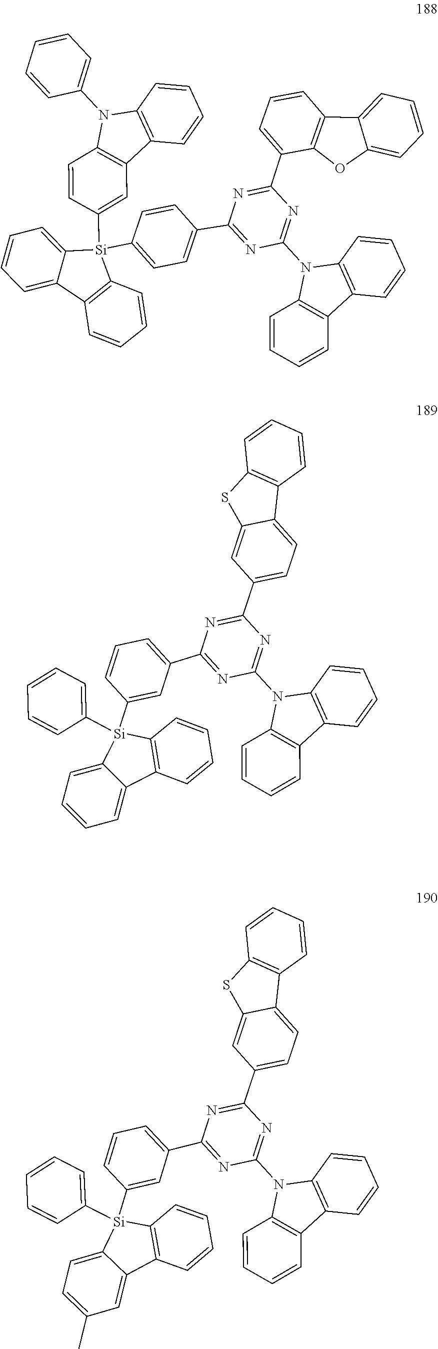 [SMILES: c1ccc(-n2c3ccccc3c3cc([Si]4(c5ccc(-c6nc(-c7cccc8c7oc7ccccc78)nc(-n7c8ccccc8c8ccccc87)n6)cc5)c5ccccc5-c5ccccc54)ccc32)cc1 ; c1ccc([Si]2(c3cccc(-c4nc(-c5ccc6c(c5)sc5ccccc56)nc(-n5c6ccccc6c6ccccc65)n4)c3)c3ccccc3-c3ccccc32)cc1 ; Cc1ccc2c(c1)-c1ccccc1[Si]2(c1ccccc1)c1cccc(-c2nc(-c3ccc4c(c3)sc3ccccc34)nc(-n3c4ccccc4c4ccccc43)n2)c1]

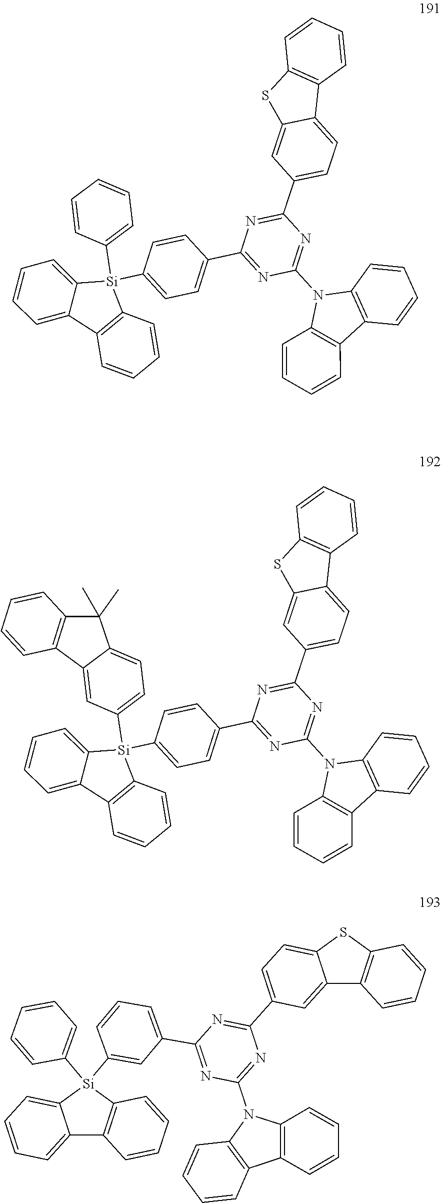 [SMILES: c1ccc([Si]2(c3ccc(-c4nc(-c5ccc6c(c5)sc5ccccc56)nc(-n5c6ccccc6c6ccccc65)n4)cc3)c3ccccc3-c3ccccc32)cc1 ; CC1(C)c2ccccc2-c2cc([Si]3(c4ccc(-c5nc(-c6ccc7c(c6)sc6ccccc67)nc(-n6c7ccccc7c7ccccc76)n5)cc4)c4ccccc4-c4ccccc43)ccc21 ; c1ccc([Si]2(c3cccc(-c4nc(-c5ccc6sc7ccccc7c6c5)nc(-n5c6ccccc6c6ccccc65)n4)c3)c3ccccc3-c3ccccc32)cc1]

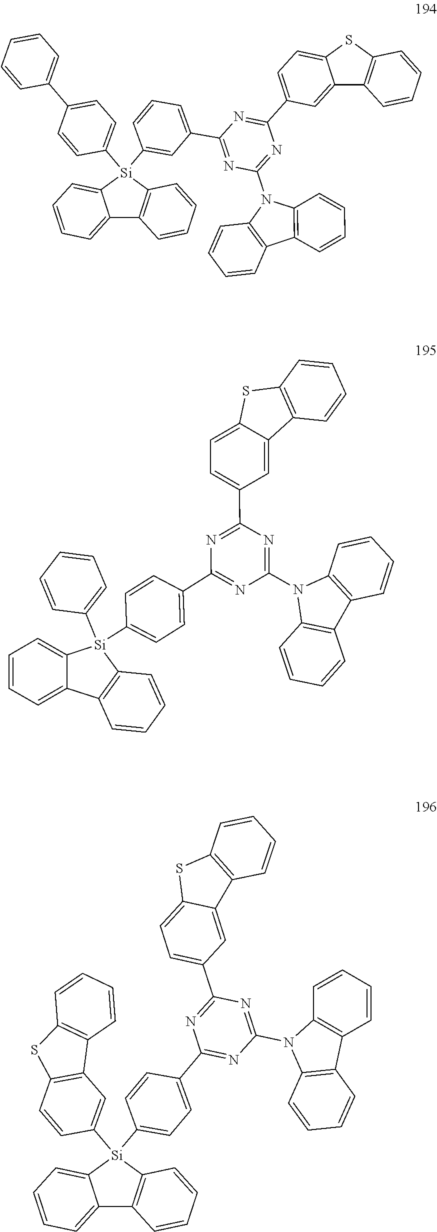 [SMILES: c1ccc(-c2ccc([Si]3(c4cccc(-c5nc(-c6ccc7sc8ccccc8c7c6)nc(-n6c7ccccc7c7ccccc76)n5)c4)c4ccccc4-c4ccccc43)cc2)cc1 ; c1ccc([Si]2(c3ccc(-c4nc(-c5ccc6sc7ccccc7c6c5)nc(-n5c6ccccc6c6ccccc65)n4)cc3)c3ccccc3-c3ccccc32)cc1 ; c1ccc2c(c1)-c1ccccc1[Si]2(c1ccc(-c2nc(-c3ccc4sc5ccccc5c4c3)nc(-n3c4ccccc4c4ccccc43)n2)cc1)c1ccc2sc3ccccc3c2c1]

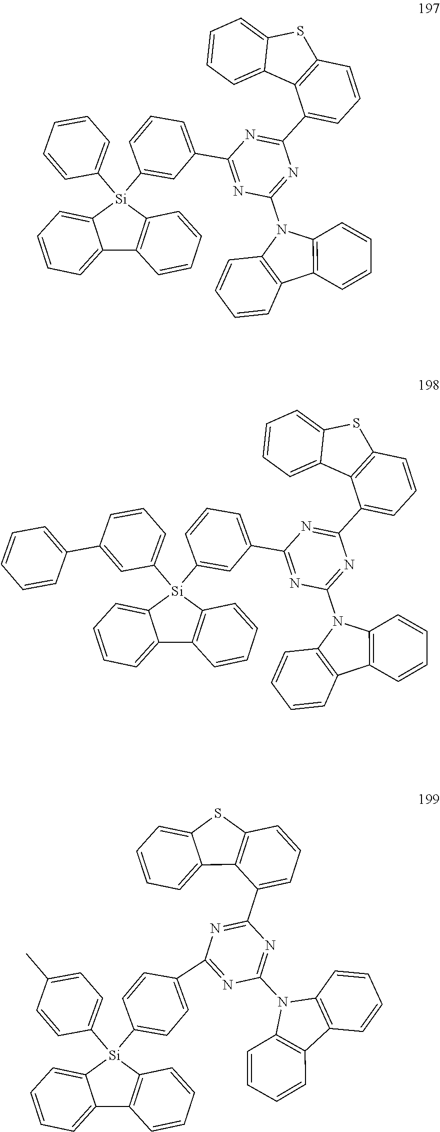 [SMILES: c1ccc([Si]2(c3cccc(-c4nc(-c5cccc6sc7ccccc7c56)nc(-n5c6ccccc6c6ccccc65)n4)c3)c3ccccc3-c3ccccc32)cc1 ; c1ccc(-c2cccc([Si]3(c4cccc(-c5nc(-c6cccc7sc8ccccc8c67)nc(-n6c7ccccc7c7ccccc76)n5)c4)c4ccccc4-c4ccccc43)c2)cc1 ; Cc1ccc([Si]2(c3ccc(-c4nc(-c5cccc6sc7ccccc7c56)nc(-n5c6ccccc6c6ccccc65)n4)cc3)c3ccccc3-c3ccccc32)cc1]

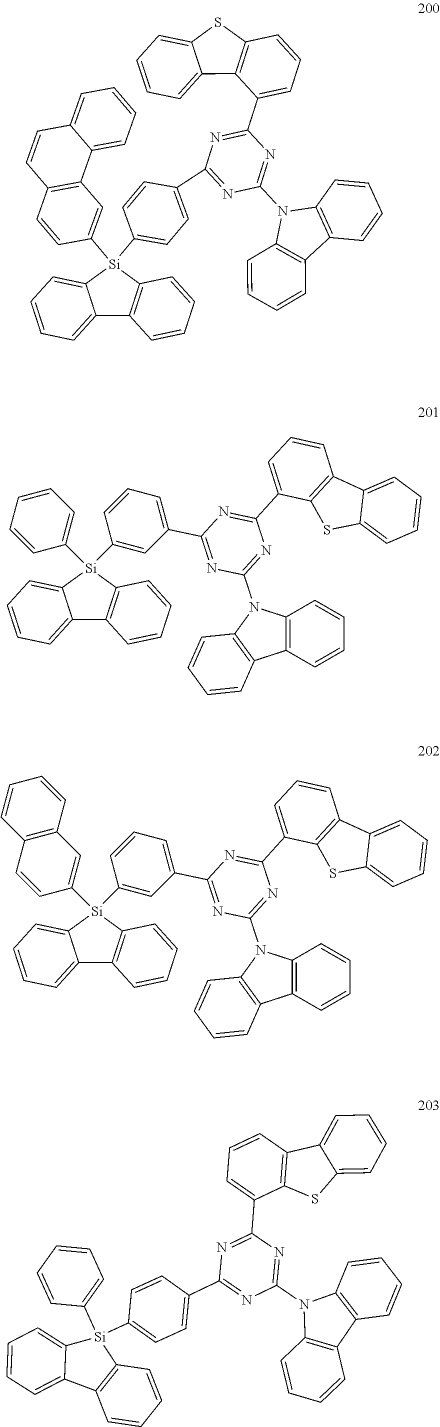 [SMILES: c1ccc2c(c1)-c1ccccc1[Si]2(c1ccc(-c2nc(-c3cccc4sc5ccccc5c34)nc(-n3c4ccccc4c4ccccc43)n2)cc1)c1ccc2ccc3ccccc3c2c1 ; c1ccc([Si]2(c3cccc(-c4nc(-c5cccc6c5sc5ccccc56)nc(-n5c6ccccc6c6ccccc65)n4)c3)c3ccccc3-c3ccccc32)cc1 ; c1cc(-c2nc(-c3cccc4c3sc3ccccc34)nc(-n3c4ccccc4c4ccccc43)n2)cc([Si]2(c3ccc4ccccc4c3)c3ccccc3-c3ccccc32)c1 ; c1ccc([Si]2(c3ccc(-c4nc(-c5cccc6c5sc5ccccc56)nc(-n5c6ccccc6c6ccccc65)n4)cc3)c3ccccc3-c3ccccc32)cc1]

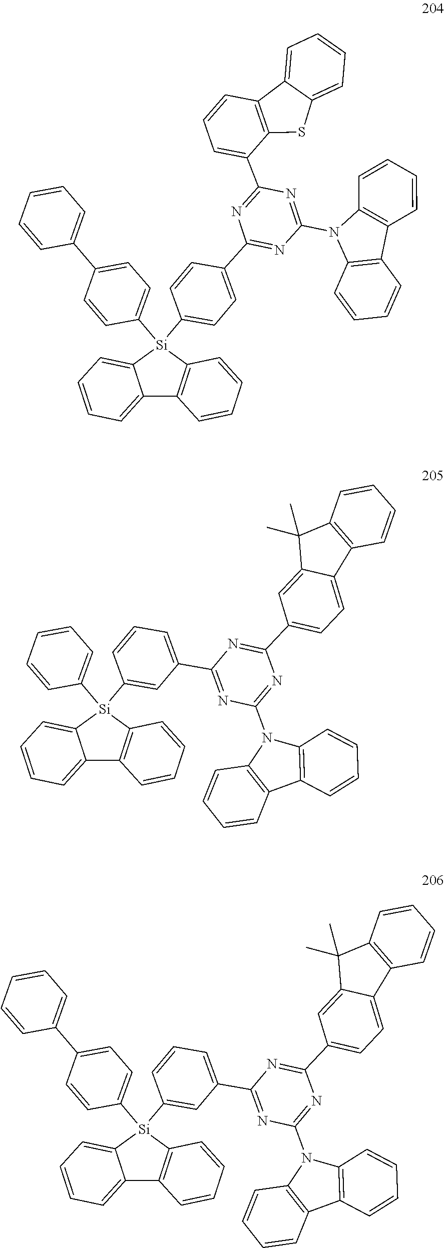 [SMILES: c1ccc(-c2ccc([Si]3(c4ccc(-c5nc(-c6cccc7c6sc6ccccc67)nc(-n6c7ccccc7c7ccccc76)n5)cc4)c4ccccc4-c4ccccc43)cc2)cc1 ; CC1(C)c2ccccc2-c2ccc(-c3nc(-c4cccc([Si]5(c6ccccc6)c6ccccc6-c6ccccc65)c4)nc(-n4c5ccccc5c5ccccc54)n3)cc21 ; CC1(C)c2ccccc2-c2ccc(-c3nc(-c4cccc([Si]5(c6ccc(-c7ccccc7)cc6)c6ccccc6-c6ccccc65)c4)nc(-n4c5ccccc5c5ccccc54)n3)cc21]

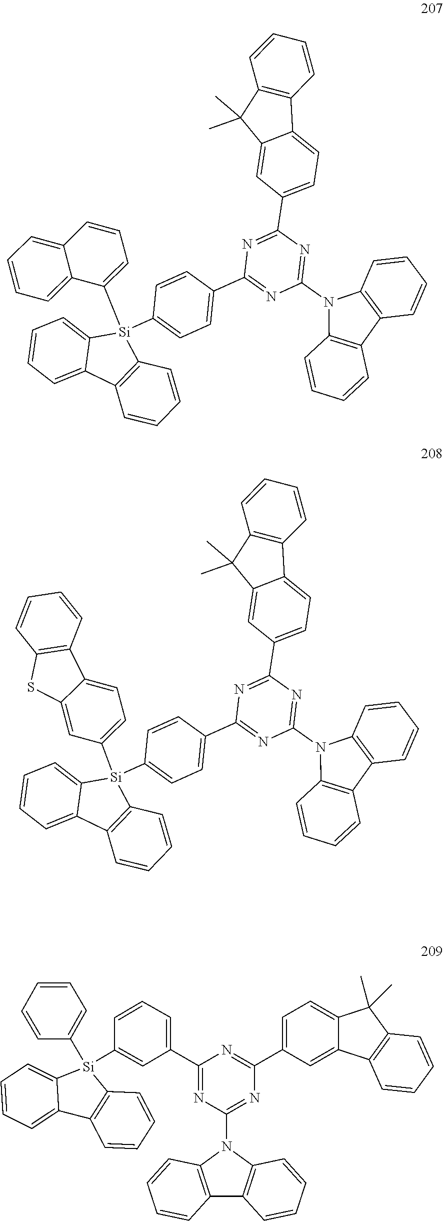 [SMILES: CC1(C)c2ccccc2-c2ccc(-c3nc(-c4ccc([Si]5(c6cccc7ccccc67)c6ccccc6-c6ccccc65)cc4)nc(-n4c5ccccc5c5ccccc54)n3)cc21 ; CC1(C)c2ccccc2-c2ccc(-c3nc(-c4ccc([Si]5(c6ccc7c(c6)sc6ccccc67)c6ccccc6-c6ccccc65)cc4)nc(-n4c5ccccc5c5ccccc54)n3)cc21 ; CC1(C)c2ccccc2-c2cc(-c3nc(-c4cccc([Si]5(c6ccccc6)c6ccccc6-c6ccccc65)c4)nc(-n4c5ccccc5c5ccccc54)n3)ccc21]

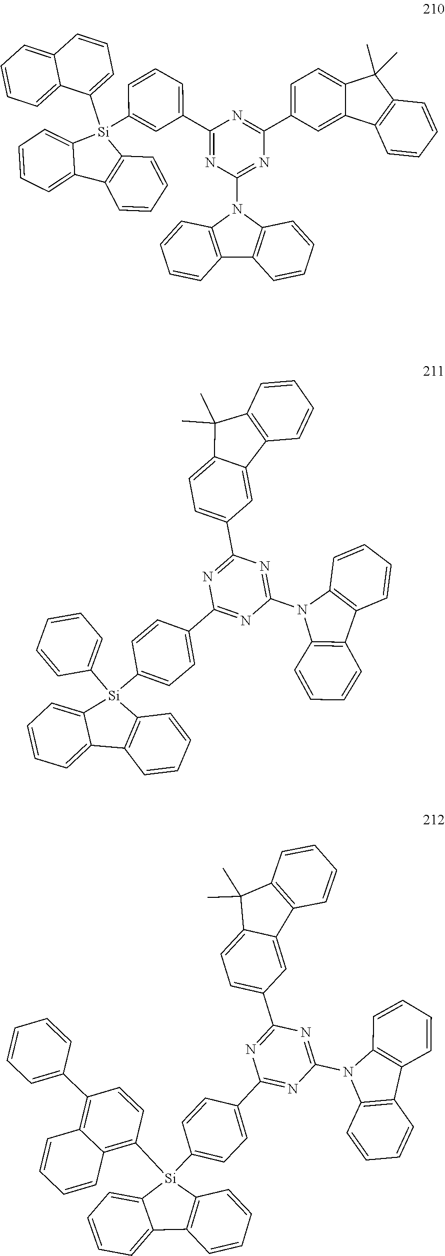 [SMILES: CC1(C)c2ccccc2-c2cc(-c3nc(-c4cccc([Si]5(c6cccc7ccccc67)c6ccccc6-c6ccccc65)c4)nc(-n4c5ccccc5c5ccccc54)n3)ccc21 ; CC1(C)c2ccccc2-c2cc(-c3nc(-c4ccc([Si]5(c6ccccc6)c6ccccc6-c6ccccc65)cc4)nc(-n4c5ccccc5c5ccccc54)n3)ccc21 ; CC1(C)c2ccccc2-c2cc(-c3nc(-c4ccc([Si]5(c6ccc(-c7ccccc7)c7ccccc67)c6ccccc6-c6ccccc65)cc4)nc(-n4c5ccccc5c5ccccc54)n3)ccc21]

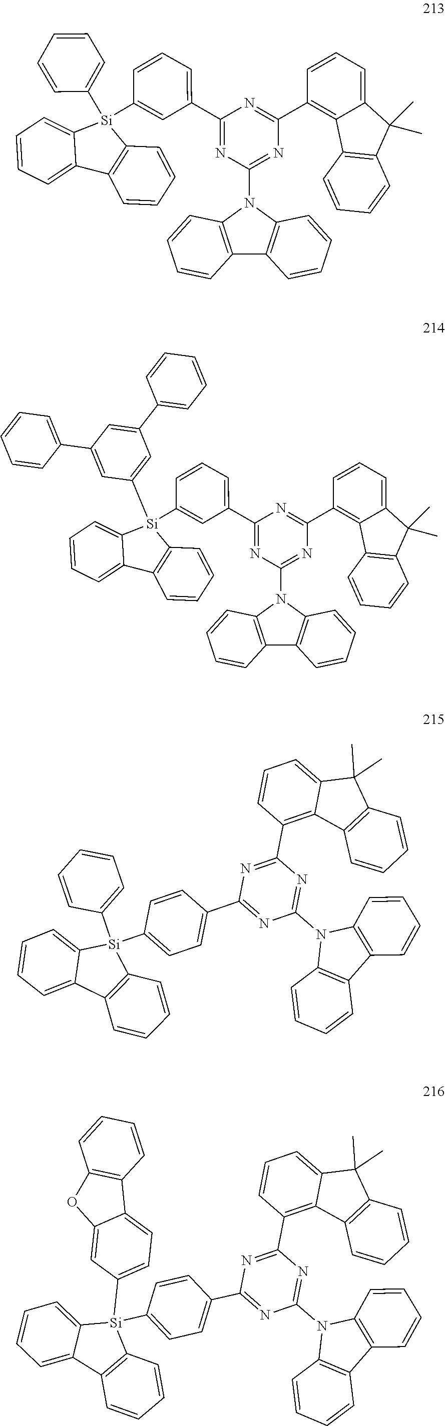 [SMILES: CC1(C)c2ccccc2-c2c(-c3nc(-c4cccc([Si]5(c6ccccc6)c6ccccc6-c6ccccc65)c4)nc(-n4c5ccccc5c5ccccc54)n3)cccc21 ; CC1(C)c2ccccc2-c2c(-c3nc(-c4cccc([Si]5(c6cc(-c7ccccc7)cc(-c7ccccc7)c6)c6ccccc6-c6ccccc65)c4)nc(-n4c5ccccc5c5ccccc54)n3)cccc21 ; CC1(C)c2ccccc2-c2c(-c3nc(-c4ccc([Si]5(c6ccccc6)c6ccccc6-c6ccccc65)cc4)nc(-n4c5ccccc5c5ccccc54)n3)cccc21 ; CC1(C)c2ccccc2-c2c(-c3nc(-c4ccc([Si]5(c6ccc7c(c6)oc6ccccc67)c6ccccc6-c6ccccc65)cc4)nc(-n4c5ccccc5c5ccccc54)n3)cccc21]

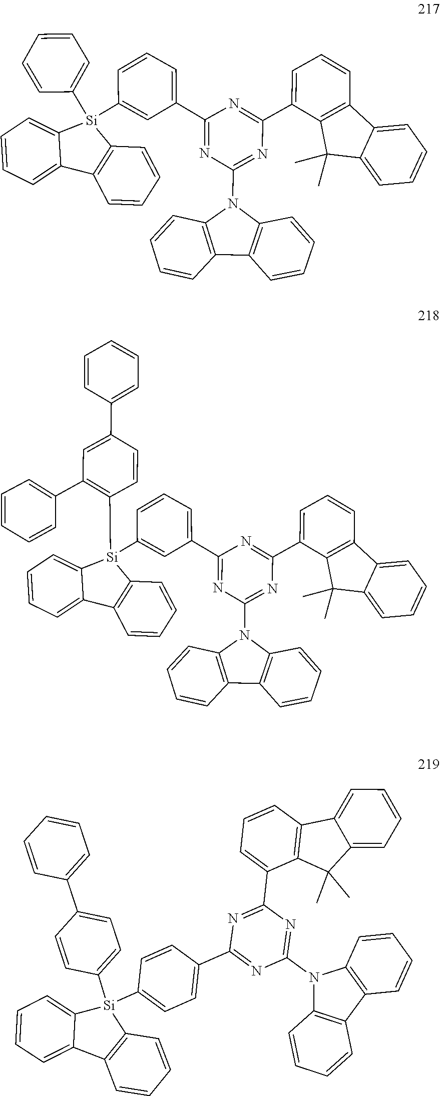 [SMILES: CC1(C)c2ccccc2-c2cccc(-c3nc(-c4cccc([Si]5(c6ccccc6)c6ccccc6-c6ccccc65)c4)nc(-n4c5ccccc5c5ccccc54)n3)c21 ; CC1(C)c2ccccc2-c2cccc(-c3nc(-c4cccc([Si]5(c6ccc(-c7ccccc7)cc6-c6ccccc6)c6ccccc6-c6ccccc65)c4)nc(-n4c5ccccc5c5ccccc54)n3)c21 ; CC1(C)c2ccccc2-c2cccc(-c3nc(-c4ccc([Si]5(c6ccc(-c7ccccc7)cc6)c6ccccc6-c6ccccc65)cc4)nc(-n4c5ccccc5c5ccccc54)n3)c21]

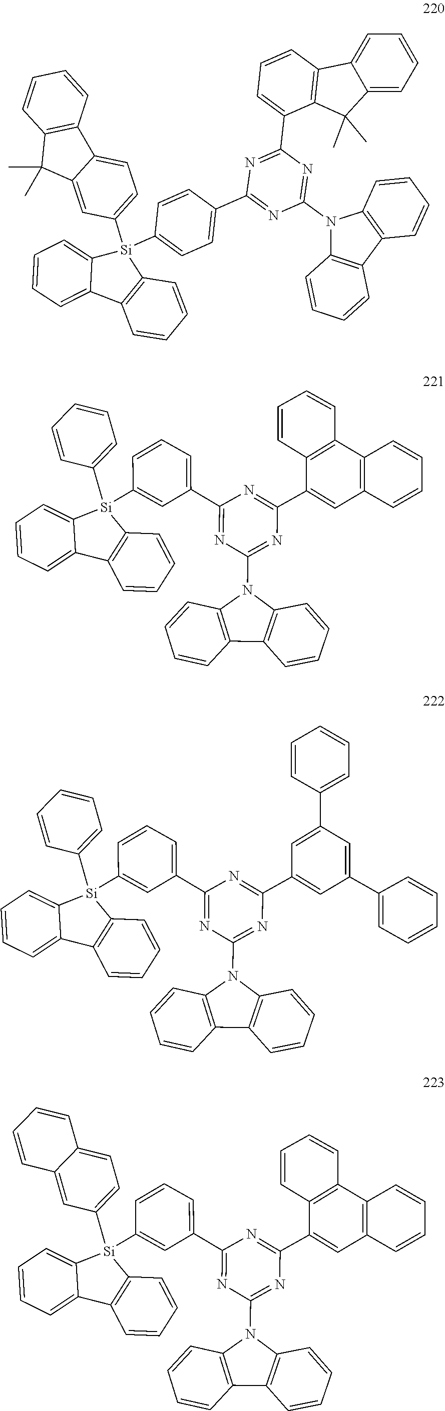 [SMILES: CC1(C)c2ccccc2-c2ccc([Si]3(c4ccc(-c5nc(-c6cccc7c6C(C)(C)c6ccccc6-7)nc(-n6c7ccccc7c7ccccc76)n5)cc4)c4ccccc4-c4ccccc43)cc21 ; c1ccc([Si]2(c3cccc(-c4nc(-c5cc6ccccc6c6ccccc56)nc(-n5c6ccccc6c6ccccc65)n4)c3)c3ccccc3-c3ccccc32)cc1 ; c1ccc(-c2cc(-c3ccccc3)cc(-c3nc(-c4cccc([Si]5(c6ccccc6)c6ccccc6-c6ccccc65)c4)nc(-n4c5ccccc5c5ccccc54)n3)c2)cc1 ; c1cc(-c2nc(-c3cc4ccccc4c4ccccc34)nc(-n3c4ccccc4c4ccccc43)n2)cc([Si]2(c3ccc4ccccc4c3)c3ccccc3-c3ccccc32)c1]

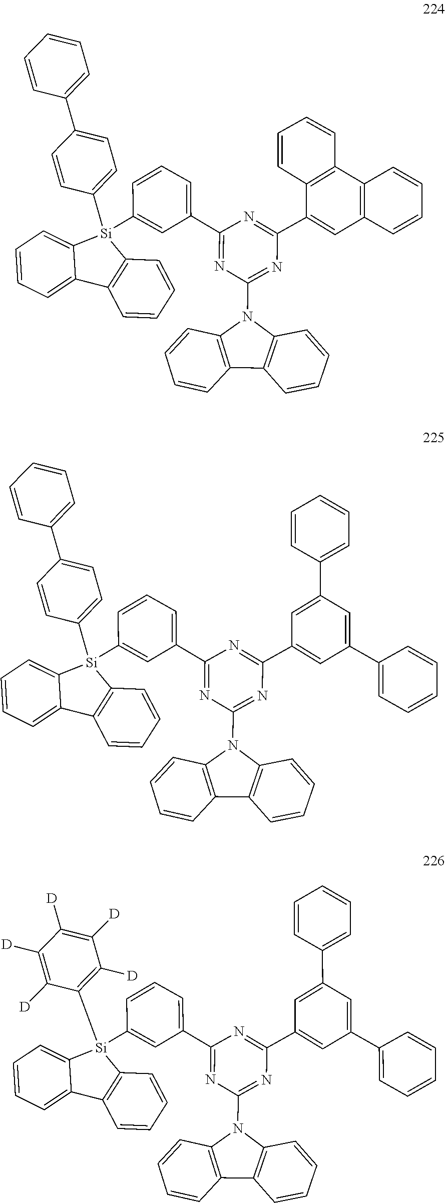 [SMILES: c1ccc(-c2ccc([Si]3(c4cccc(-c5nc(-c6cc7ccccc7c7ccccc67)nc(-n6c7ccccc7c7ccccc76)n5)c4)c4ccccc4-c4ccccc43)cc2)cc1 ; c1ccc(-c2ccc([Si]3(c4cccc(-c5nc(-c6cc(-c7ccccc7)cc(-c7ccccc7)c6)nc(-n6c7ccccc7c7ccccc76)n5)c4)c4ccccc4-c4ccccc43)cc2)cc1 ; [2H]c1c([2H])c([2H])c([Si]2(c3cccc(-c4nc(-c5cc(-c6ccccc6)cc(-c6ccccc6)c5)nc(-n5c6ccccc6c6ccccc65)n4)c3)c3ccccc3-c3ccccc32)c([2H])c1[2H]]

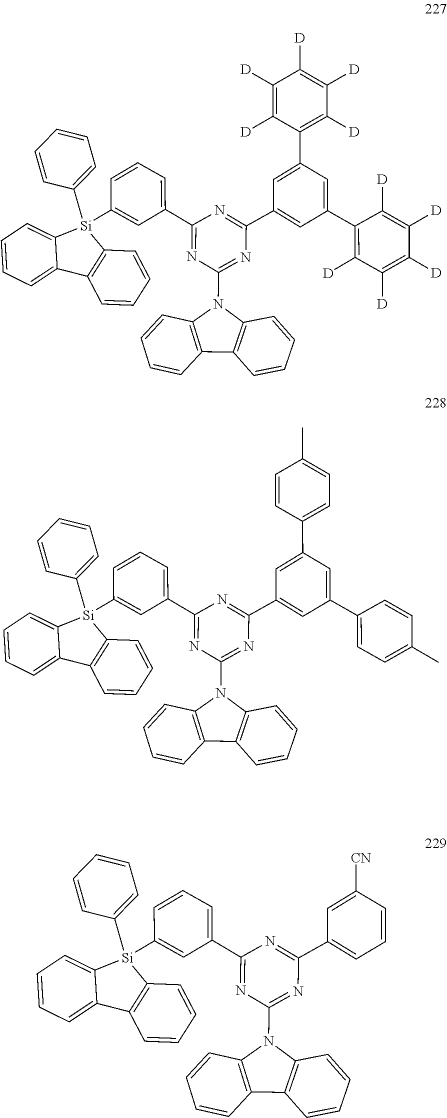 [SMILES: [2H]c1c([2H])c([2H])c(-c2cc(-c3nc(-c4cccc([Si]5(c6ccccc6)c6ccccc6-c6ccccc65)c4)nc(-n4c5ccccc5c5ccccc54)n3)cc(-c3c([2H])c([2H])c([2H])c([2H])c3[2H])c2)c([2H])c1[2H] ; Cc1ccc(-c2cc(-c3ccc(C)cc3)cc(-c3nc(-c4cccc([Si]5(c6ccccc6)c6ccccc6-c6ccccc65)c4)nc(-n4c5ccccc5c5ccccc54)n3)c2)cc1 ; N#Cc1cccc(-c2nc(-c3cccc([Si]4(c5ccccc5)c5ccccc5-c5ccccc54)c3)nc(-n3c4ccccc4c4ccccc43)n2)c1]

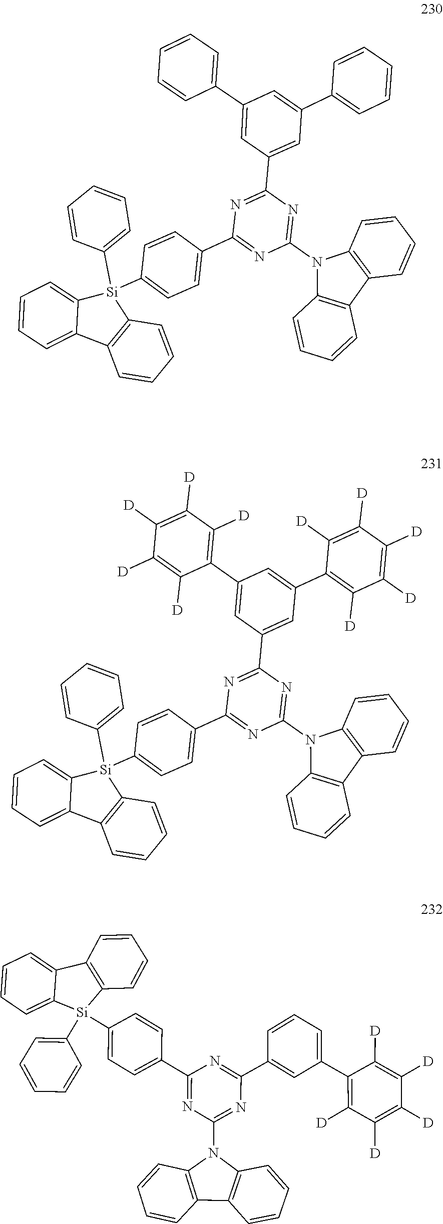 [SMILES: c1ccc(-c2cc(-c3ccccc3)cc(-c3nc(-c4ccc([Si]5(c6ccccc6)c6ccccc6-c6ccccc65)cc4)nc(-n4c5ccccc5c5ccccc54)n3)c2)cc1 ; [2H]c1c([2H])c([2H])c(-c2cc(-c3nc(-c4ccc([Si]5(c6ccccc6)c6ccccc6-c6ccccc65)cc4)nc(-n4c5ccccc5c5ccccc54)n3)cc(-c3c([2H])c([2H])c([2H])c([2H])c3[2H])c2)c([2H])c1[2H] ; [2H]c1c([2H])c([2H])c(-c2cccc(-c3nc(-c4ccc([Si]5(c6ccccc6)c6ccccc6-c6ccccc65)cc4)nc(-n4c5ccccc5c5ccccc54)n3)c2)c([2H])c1[2H]]

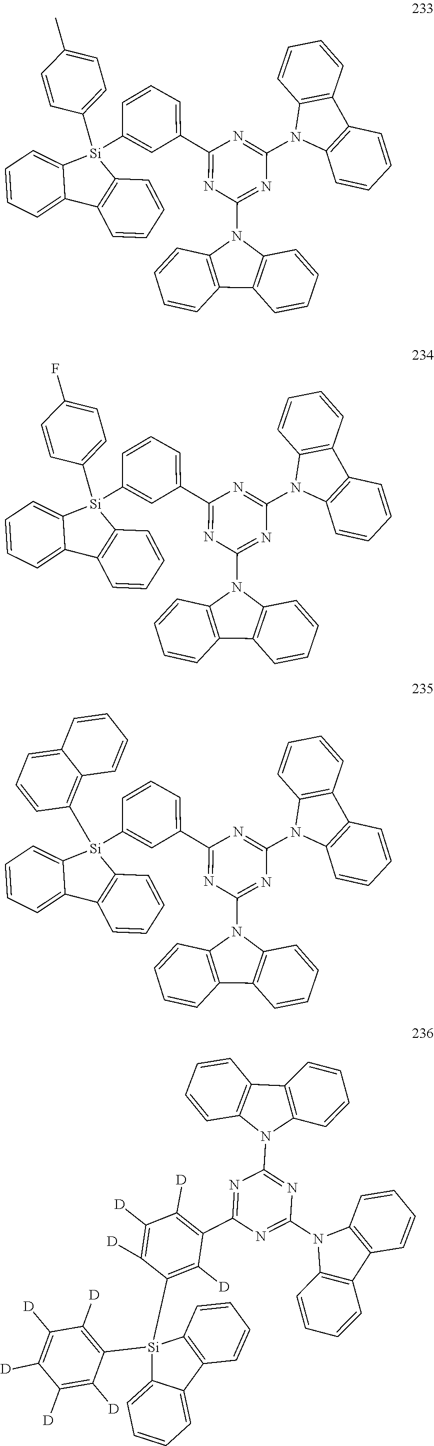 [SMILES: Cc1ccc([Si]2(c3cccc(-c4nc(-n5c6ccccc6c6ccccc65)nc(-n5c6ccccc6c6ccccc65)n4)c3)c3ccccc3-c3ccccc32)cc1 ; Fc1ccc([Si]2(c3cccc(-c4nc(-n5c6ccccc6c6ccccc65)nc(-n5c6ccccc6c6ccccc65)n4)c3)c3ccccc3-c3ccccc32)cc1 ; c1cc(-c2nc(-n3c4ccccc4c4ccccc43)nc(-n3c4ccccc4c4ccccc43)n2)cc([Si]2(c3cccc4ccccc34)c3ccccc3-c3ccccc32)c1 ; [2H]c1c([2H])c([2H])c([Si]2(c3c([2H])c([2H])c([2H])c(-c4nc(-n5c6ccccc6c6ccccc65)nc(-n5c6ccccc6c6ccccc65)n4)c3[2H])c3ccccc3-c3ccccc32)c([2H])c1[2H]]

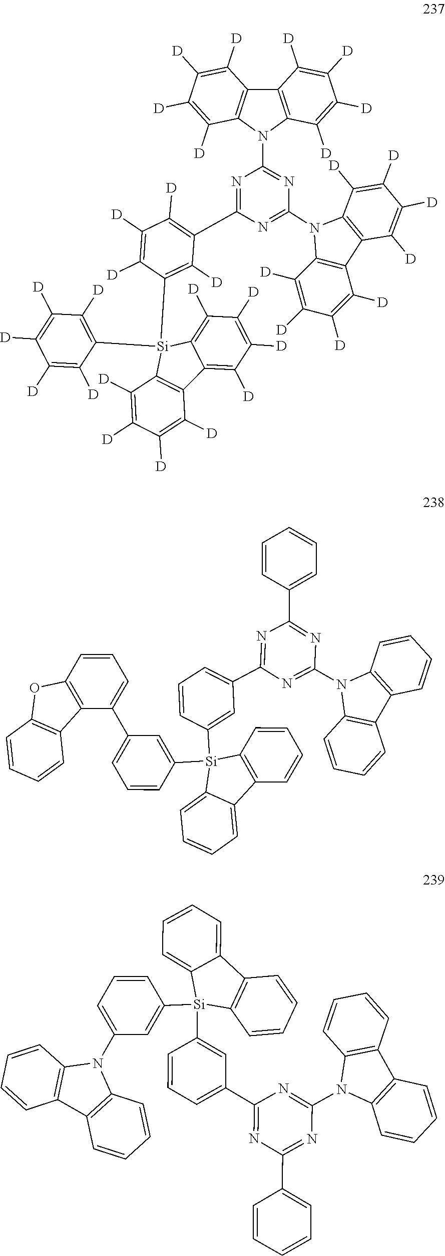

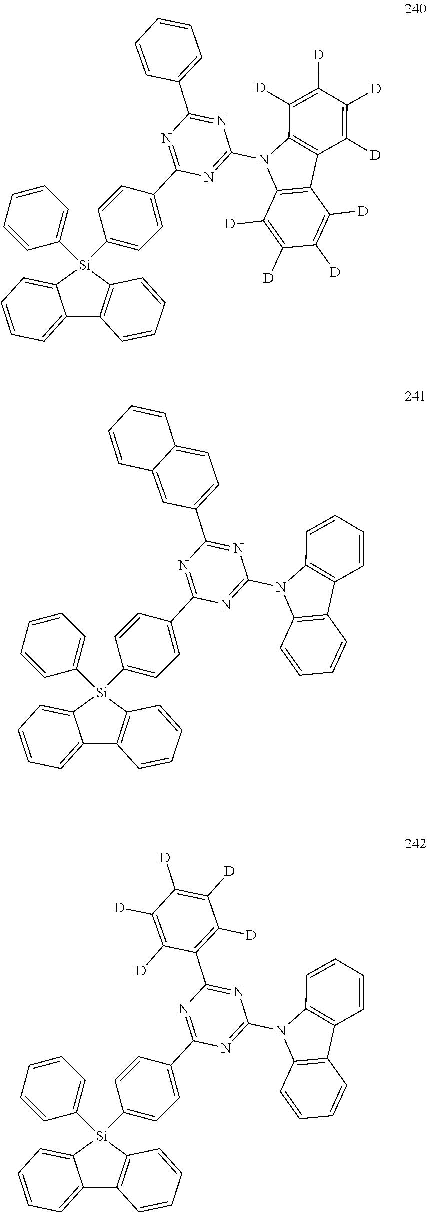 [SMILES: [2H]c1c([2H])c([2H])c2c(c1[2H])c1c([2H])c([2H])c([2H])c([2H])c1n2-c1nc(-c2ccccc2)nc(-c2ccc([Si]3(c4ccccc4)c4ccccc4-c4ccccc43)cc2)n1 ; c1ccc([Si]2(c3ccc(-c4nc(-c5ccc6ccccc6c5)nc(-n5c6ccccc6c6ccccc65)n4)cc3)c3ccccc3-c3ccccc32)cc1 ; [2H]c1c([2H])c([2H])c(-c2nc(-c3ccc([Si]4(c5ccccc5)c5ccccc5-c5ccccc54)cc3)nc(-n3c4ccccc4c4ccccc43)n2)c([2H])c1[2H]]

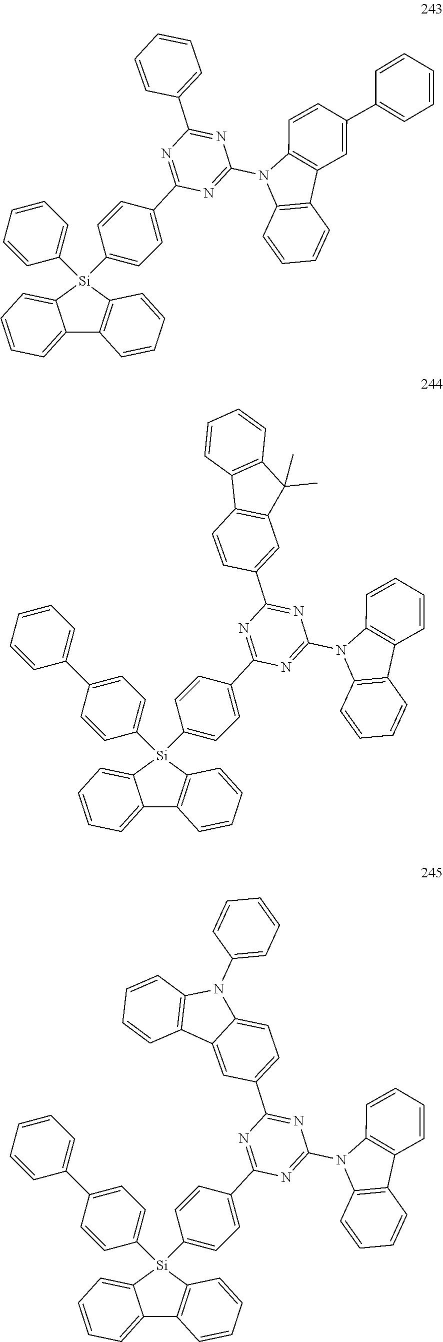 [SMILES: c1ccc(-c2ccc3c(c2)c2ccccc2n3-c2nc(-c3ccccc3)nc(-c3ccc([Si]4(c5ccccc5)c5ccccc5-c5ccccc54)cc3)n2)cc1 ; CC1(C)c2ccccc2-c2ccc(-c3nc(-c4ccc([Si]5(c6ccc(-c7ccccc7)cc6)c6ccccc6-c6ccccc65)cc4)nc(-n4c5ccccc5c5ccccc54)n3)cc21 ; c1ccc(-c2ccc([Si]3(c4ccc(-c5nc(-c6ccc7c(c6)c6ccccc6n7-c6ccccc6)nc(-n6c7ccccc7c7ccccc76)n5)cc4)c4ccccc4-c4ccccc43)cc2)cc1]

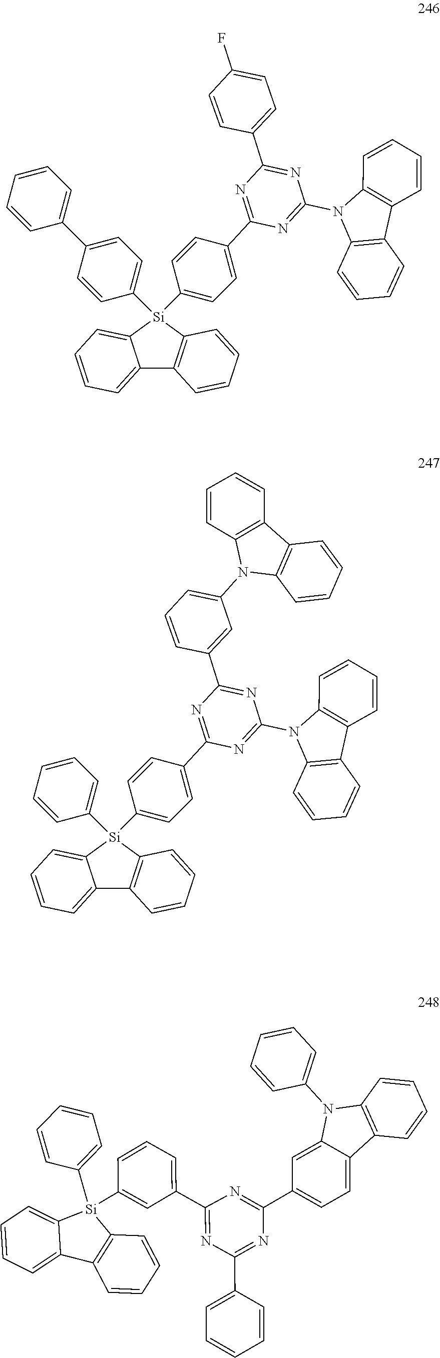 [SMILES: Fc1ccc(-c2nc(-c3ccc([Si]4(c5ccc(-c6ccccc6)cc5)c5ccccc5-c5ccccc54)cc3)nc(-n3c4ccccc4c4ccccc43)n2)cc1 ; c1ccc([Si]2(c3ccc(-c4nc(-c5cccc(-n6c7ccccc7c7ccccc76)c5)nc(-n5c6ccccc6c6ccccc65)n4)cc3)c3ccccc3-c3ccccc32)cc1 ; c1ccc(-c2nc(-c3cccc([Si]4(c5ccccc5)c5ccccc5-c5ccccc54)c3)nc(-c3ccc4c5ccccc5n(-c5ccccc5)c4c3)n2)cc1]

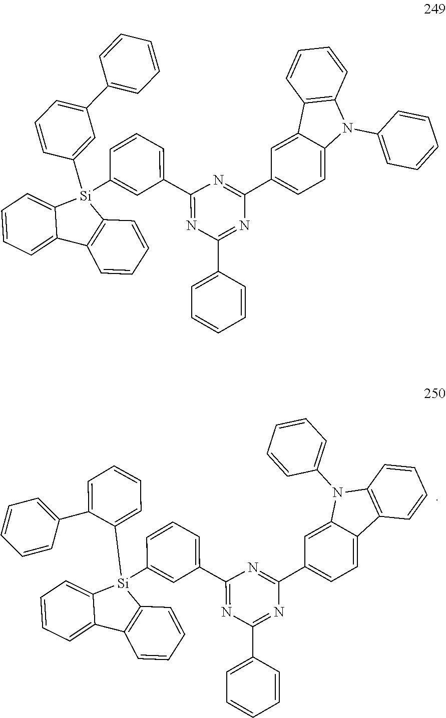 [SMILES: c1ccc(-c2cccc([Si]3(c4cccc(-c5nc(-c6ccccc6)nc(-c6ccc7c(c6)c6ccccc6n7-c6ccccc6)n5)c4)c4ccccc4-c4ccccc43)c2)cc1 ; c1ccc(-c2nc(-c3cccc([Si]4(c5ccccc5-c5ccccc5)c5ccccc5-c5ccccc54)c3)nc(-c3ccc4c5ccccc5n(-c5ccccc5)c4c3)n2)cc1]

13. An organic electroluminescent device, comprising an anode and a cathode disposed opposite to each other, and a functional layer disposed between the anode and the cathode; wherein the functional layer comprises the organic compound according to claim 1.

14. The organic electroluminescent device according to claim 13, wherein the functional layer comprises an organic light-emitting layer, and the organic light-emitting layer comprises the organic compound.

15. An electronic apparatus, comprising the organic electroluminescent device of claim 13.
